SEQUENCE LISTING (153 sequences)

SEQ ID NO: 1 - protein, 429 aa, Podocoryna carnea
GPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLRGKPGAKGIAGSDGEAGESGAPGQSGPTGPRGQRGPSGEDGNPGLQGLPGSDGEPGEEGQPGRSGQPGQQGPRGSPGEVGPRGSKGPSGDRGDRGERGVPGQTGSAGNVGEDGEQGGKGVDGASGPSGALGARGPPGSRGDTGAVGPPGPTGRSGLPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 2 - DNA, 1289 bp, Podocoryna carnea
  source 1..1289: mol_type = unassigned DNA
ggaccacaaggtgttgtaggagctgatggcaaagatggaacaccgggagagaaaggtgagcaaggacgaaccggagctgcaggaaaacagggaagccctggagcagatggagcaagaggccctcttggatcaattggacaacaaggtgctcgtggagaacctggtgatccaggatctcccggcttaagaggagatactggattggctggagtcaaaggagtagcaggaccatctggtcgacctggacaacccggtgcaaatggattacctggtgtgaatggcagaggcggtttgagaggcaaacctggtgctaaaggaattgctggcagtgatggagaagcgggagaatctggcgcacctggacagtccggacctaccggtccacgtggtcaacgaggaccaagtggtgaggatggtaatcctggattacagggattgcctggttctgatggagagcccggagaggaaggacaacctggaagatctggtcaaccaggacagcaaggaccacgtggttcccctggagaggtaggaccaagaggatctaaaggtccatcaggagatcgtggtgacaggggagagagaggtgttcctggacaaacaggttcggctggaaatgtaggagaagatggagagcaaggaggcaaaggtgtcgatggagcgagtggaccaagtggagctcttggtgctcgtggtcccccaggaagtagaggtgacaccggggcagtgggacctcccggacctactgggcgatctggtttacctggaaacgcaggacaaaagggaccaagtggtgaaccaggtagtccaggaaaagcaggatcagctggtgaacagggtcctcctggtaaagacggatcaaatggtgaacctggatctcctggcaaagagggtgaacgtggtcttgctggtccaccaggtccagatggcagacgtggtgaaacgggatctccaggtatcgctggtgctcttggtaaaccaggtttggaaggacctaaaggttatccaggattaagaggaagagatggaaccaatggcaaacgaggagaacaaggagaaactggtcctgatggagtcagaggtattcctggaaatgatggacaatctggcaaaccaggtattgatggtattgacggaacaaatggtcaaccaggtgaggctggataccaaggtggtagaggtacacgtggtcagttaggtgaaactggtgatgtcggacagaatggagatcgaggagctcctggtcctgatggatctaaaggttctgctggtagaccaggacttcgtgg

SEQ ID NO: 3 - DNA, 1425 bp, synthetic construct
  misc_feature 1..1425: note = source = /note="Description of Artificial Sequence: Synthetic polynucleotide"
  source 1..1425: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgggtccgcagggtgttgttggtgcagatggtaaagacggtaccccgggtgaaaaaggagaacagggacgtacaggtgcagcaggtaaacagggcagcccgggtgccgatggtgcccgtggcccgctgggtagcattggtcagcagggtgcaagaggcgaaccgggcgatccgggtagtccgggcctgcgtggtgatacgggtctggccggtgttaaaggcgttgcaggtccttcaggtcgtccaggtcaaccgggtgcaaatggtctgccgggtgttaatggtcgtggcggtctgcgtggcaaaccgggagcaaaaggtattgcaggtagcgatggagaagccggtgaaagcggtgccccgggtcagagtggtccgaccggtccgcgcggtcagcgtggtccgtctggtgaagatggcaatccgggtctgcagggtctgcctggtagtgatggcgaaccaggtgaagaaggtcagccgggtcgttcaggccagccgggccagcagggcccgcgtggtagcccgggcgaagttggcccgcggggtagtaaaggtcctagtggcgatcgcggtgatcgtggtgaacgcggtgttcctggtcagaccggtagcgcaggtaatgttggcgaagatggtgaacagggtggcaaaggtgttgatggtgcaagcggtccgagcggtgcactgggtgcacgtggtcctccgggcagccgtggtgacaccggtgcagttggtccgcctggcccgaccggccgtagtggcttaccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 4 - DNA, 1425 bp, synthetic construct
  misc_feature 1..1425: note = source = /note="Description of Artificial Sequence: Synthetic polynucleotide"
  source 1..1425: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgggtccgcagggtgttgttggtgcagatggtaaagacggtaccccgggtgaaaaaggtgaacagggtcgtaccggtgcagcaggtaaacagggcagcccgggtgccgatggtgcccgtggcccgctgggtagcattggtcagcagggtgcacgtggcgaaccgggcgatccgggtagcccgggcctgcgtggtgatacgggtctggccggtgttaaaggcgttgcaggtccttctggtcgtccaggtcaaccgggtgcaaatggtctgccgggtgttaatggtcgtggcggtctgcgtggcaaaccgggtgcaaaaggtattgcaggtagcgatggcgaagccggtgaaagcggtgccccgggtcagagcggtccgaccggtccgcgcggtcagcgtggtccgtctggtgaagatggcaatccgggtctgcagggtctgcctggtagcgatggcgaaccaggtgaagaaggtcagccgggtcgttctggccagccgggccagcagggcccgcgtggtagcccgggcgaagttggcccgcgcggttctaaaggtcctagcggcgatcgcggtgatcgtggtgaacgcggtgttcctggtcagaccggtagcgcaggtaatgttggcgaagatggtgaacagggtggcaaaggtgttgatggtgcaagcggtccgagcggtgcactgggtgcacgtggtcctccgggcagccgtggtgacaccggtgcagttggtccgcctggcccgaccggccgtagcggcctgccgggtaatgcaggtcagaaaggtccgtctggtgaacctggcagccctggtaaagcaggtagcgccggtgagcagggtccgccgggcaaagatggtagcaatggtgagccgggtagccctggcaaagaaggtgaacgtggtctggcaggtccgccgggtcctgatggtcgccgcggtgaaacgggttctccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaaccggtgatgttggtcagaatggtgatcgcggcgcaccgggtccggatggtagcaaaggtagcgccggtcgtccgggtctgcgttaa

SEQ ID NO: 5 - protein, 474 aa, synthetic construct
  REGION 1..474: note = source = /note="Description of Artificial Sequence: Synthetic polypeptide"
MKKIWLALAGLVLAFSASAAQYEDHHHHHHHHHSGSSLVPRGSHMGPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLRGKPGAKGIAGSDGEAGESGAPGQSGPTGPRGQRGPSGEDGNPGLQGLPGSDGEPGEEGQPGRSGQPGQQGPRGSPGEVGPRGSKGPSGDRGDRGERGVPGQTGSAGNVGEDGEQGGKGVDGASGPSGALGARGPPGSRGDTGAVGPPGPTGRSGLPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 6 - DNA, 1362 bp, synthetic construct
  misc_feature 1..1362: note = source = /note="Description of Artificial Sequence: Synthetic polynucleotide"
  source 1..1362: mol_type = other DNA
atgaaaaagatttggctggcgctggctggtttagttttagcgtttagcgcatcggcggcgcagtatgaagatggtccgcagggtgttgttggtgcagatggtaaagacggtaccccgggtgaaaaaggagaacagggacgtacaggtgcagcaggtaaacagggcagcccgggtgccgatggtgcccgtggcccgctgggtagcattggtcagcagggtgcaagaggcgaaccgggcgatccgggtagtccgggcctgcgtggtgatacgggtctggccggtgttaaaggcgttgcaggtccttcaggtcgtccaggtcaaccgggtgcaaatggtctgccgggtgttaatggtcgtggcggtctgcgtggcaaaccgggagcaaaaggtattgcaggtagcgatggagaagccggtgaaagcggtgccccgggtcagagtggtccgaccggtccgcgcggtcagcgtggtccgtctggtgaagatggcaatccgggtctgcagggtctgcctggtagtgatggcgaaccaggtgaagaaggtcagccgggtcgttcaggccagccgggccagcagggcccgcgtggtagcccgggcgaagttggcccgcggggtagtaaaggtcctagtggcgatcgcggtgatcgtggtgaacgcggtgttcctggtcagaccggtagcgcaggtaatgttggcgaagatggtgaacagggtggcaaaggtgttgatggtgcaagcggtccgagcggtgcactgggtgcacgtggtcctccgggcagccgtggtgacaccggtgcagttggtccgcctggcccgaccggccgtagtggcttaccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 7 - DNA, 1362 bp, synthetic construct
  misc_feature 1..1362: note = source = /note="Description of Artificial Sequence: Synthetic polynucleotide"
  source 1..1362: mol_type = other DNA
atgaaaaagatttggctggcgctggctggtttagttttagcgtttagcgcatcggcggcgcagtatgaagatggtccgcagggtgttgttggtgcagatggtaaagacggtaccccgggtgaaaaaggtgaacagggtcgtaccggtgcagcaggtaaacagggcagcccgggtgccgatggtgcccgtggcccgctgggtagcattggtcagcagggtgcacgtggcgaaccgggcgatccgggtagcccgggcctgcgtggtgatacgggtctggccggtgttaaaggcgttgcaggtccttctggtcgtccaggtcaaccgggtgcaaatggtctgccgggtgttaatggtcgtggcggtctgcgtggcaaaccgggtgcaaaaggtattgcaggtagcgatggcgaagccggtgaaagcggtgccccgggtcagagcggtccgaccggtccgcgcggtcagcgtggtccgtctggtgaagatggcaatccgggtctgcagggtctgcctggtagcgatggcgaaccaggtgaagaaggtcagccgggtcgttctggccagccgggccagcagggcccgcgtggtagcccgggcgaagttggcccgcgcggttctaaaggtcctagcggcgatcgcggtgatcgtggtgaacgcggtgttcctggtcagaccggtagcgcaggtaatgttggcgaagatggtgaacagggtggcaaaggtgttgatggtgcaagcggtccgagcggtgcactgggtgcacgtggtcctccgggcagccgtggtgacaccggtgcagttggtccgcctggcccgaccggccgtagcggcctgccgggtaatgcaggtcagaaaggtccgtctggtgaacctggcagccctggtaaagcaggtagcgccggtgagcagggtccgccgggcaaagatggtagcaatggtgagccgggtagccctggcaaagaaggtgaacgtggtctggcaggtccgccgggtcctgatggtcgccgcggtgaaacgggttctccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaaccggtgatgttggtcagaatggtgatcgcggcgcaccgggtccggatggtagcaaaggtagcgccggtcgtccgggtctgcgttaa

SEQ ID NO: 8 - protein, 453 aa, synthetic construct
  REGION 1..453: note = source = /note="Description of Artificial Sequence: Synthetic polypeptide"
MKKIWLALAGLVLAFSASAAQYEDGPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLRGKPGAKGIAGSDGEAGESGAPGQSGPTGPRGQRGPSGEDGNPGLQGLPGSDGEPGEEGQPGRSGQPGQQGPRGSPGEVGPRGSKGPSGDRGDRGERGVPGQTGSAGNVGEDGEQGGKGVDGASGPSGALGARGPPGSRGDTGAVGPPGPTGRSGLPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 9 - DNA, 825 bp, synthetic construct
  misc_feature 1..825: note = source = /note="Description of Artificial Sequence: Synthetic polynucleotide"
  source 1..825: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgggtccgcagggtgttgttggtgcagatggtaaagacggtaccccgggtgaaaaaggagaacagggacgtacaggtgcagcaggtaaacagggcagcccgggtgccgatggtgcccgtggcccgctgggtagcattggtcagcagggtgcaagaggcgaaccgggcgatccgggtagtccgggcctgcgtggtgatacgggtctggccggtgttaaaggcgttgcaggtccttcaggtcgtccaggtcaaccgggtgcaaatggtctgccgggtgttaatggtcgtggcggtctggaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 10 - protein, 274 aa, synthetic construct
  REGION 1..274: note = source = /note="Description of Artificial Sequence: Synthetic polypeptide"
MKKIWLALAGLVLAFSASAAQYEDHHHHHHHHHSGSSLVPRGSHMGPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 11 - DNA, 642 bp, synthetic construct
  misc_feature 1..642: note = source = /note="Description of Artificial Sequence: Synthetic polynucleotide"
  source 1..642: mol_type = other DNA
atgaaaaagatttggctggcgctggctggtttagttttagcgtttagcgcatcggcggcgcagtatgaagatggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 12 - protein, 213 aa, synthetic construct
  REGION 1..213: note = source = /note="Description of Artificial Sequence: Synthetic polypeptide"
MKKIWLALAGLVLAFSASAAQYEDGPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 13 - DNA, 696 bp, synthetic construct
  misc_feature 1..696: note = source = /note="Description of Artificial Sequence: Synthetic polynucleotide"
  source 1..696: mol_type = other DNA
atgaaaaagatttggctggcgctggctggtttagttttagcgtttagcgcatcggcggcgcagtatgaagatggtgaaaaaggtgaaaagggcgagaaaggtgagaaaggcgaaaagggtgaaaaaggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 14 - protein, 231 aa, synthetic construct
  REGION 1..231: note = source = /note="Description of Artificial Sequence: Synthetic polypeptide"
MKKIWLALAGLVLAFSASAAQYEDGEKGEKGEKGEKGEKGEKGPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 15 - DNA, 696 bp, synthetic construct
  misc_feature 1..696: note = source = /note="Description of Artificial Sequence: Synthetic polynucleotide"
  source 1..696: mol_type = other DNA
atgaaaaagatttggctggcgctggctggtttagttttagcgtttagcgcatcggcggcgcagtatgaagatggtgataaaggtgataagggcgacaaaggtgacaaaggcgataagggtgataaaggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 16 - protein, 231 aa, synthetic construct
  REGION 1..231: note = source = /note="Description of Artificial Sequence: Synthetic polypeptide"
MKKIWLALAGLVLAFSASAAQYEDGDKGDKGDKGDKGDKGDKGPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 17 - DNA, 2232 bp, synthetic construct
  misc_feature 1..2232: note = source = /note="Description of Artificial Sequence: Synthetic polynucleotide"
  source 1..2232: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgtctggctcgagcagtaaaggtgaagaactgttcaccggtgttgttccgatcctggttgaactggatggtgatgttaacggccacaaattctctgttcgtggtgaaggtgaaggtgatgcaaccaacggtaaactgaccctgaaattcatctgcactaccggtaaactgccggttccatggccgactctggtgactaccctgacctatggtgttcagtgtttttctcgttacccggatcacatgaagcagcatgatttcttcaaatctgcaatgccggaaggttatgtacaggagcgcaccatttctttcaaagacgatggcacctacaaaacccgtgcagaggttaaatttgaaggtgatactctggtgaaccgtattgaactgaaaggcattgatttcaaagaggacggcaacatcctgggccacaaactggaatataacttcaactcccataacgtttacatcaccgcagacaaacagaagaacggtatcaaagctaacttcaaaattcgccataacgttgaagacggtagcgtacagctggcggaccactaccagcagaacactccgatcggtgatggtccggttctgctgccggataaccactacctgtccacccagtctaaactgtccaaagacccgaacgaaaagcgcgaccacatggtgctgctggagttcgttactgcagcaggtatcacgcacggcatggatgaactctacaaatctggcgcgccgggcggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgtcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 18 - protein, 743 aa, synthetic construct
  REGION 1..743: note = source = /note="Description of Artificial Sequence: Synthetic polypeptide"
MKKIWLALAGLVLAFSASAAQYEDHHHHHHHHHSGSSLVPRGSHMSGSSSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGAPGGPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLRHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 19 - DNA, 2232 bp, synthetic construct; identical to SEQ ID NO: 17
  misc_feature 1..2232: note = source = /note="Description of Artificial Sequence: Synthetic polynucleotide"
  source 1..2232: mol_type = other DNA

SEQ ID NO: 20 - protein, 743 aa, synthetic construct; identical to SEQ ID NO: 18
  REGION 1..743: note = source = /note="Description of Artificial Sequence: Synthetic polypeptide"

SEQ ID NO: 21 - protein, 786 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 22 - DNA, 2361 bp, Homo sapiens
  source 1..2361: mol_type = unassigned DNA
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctggtgtcggaggtgttcccggagtcggaggtgtcccgggagttggcatttcccccgaagctcaggcagcagctgccgccaaggctgccaagtacggtgctgcaggagcaggagtgctgggtgggctagtgccaggtccccaggcggcagtcccaggtgtgccgggcacgggaggagtgccaggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcaggtgcagatgagggagttaggcggagcctgtcccctgagctcagggaaggagatccctcctcctctcagcacctccccagcaccccctcatcacccagggtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 23 - DNA, 2418 bp, synthetic construct
  misc_feature 1..2418: note = source = /note="Description of Artificial Sequence: Synthetic polynucleotide"
  source 1..2418: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 24 - protein, 805 aa, synthetic construct
  REGION 1..805: note = source = /note="Description of Artificial Sequence: Synthetic polypeptide"
MKKIWLALAGLVLAFSASAAQYEDHHHHHHHHHSGSSLVPRGSHMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 25 - DNA, 2358 bp, synthetic construct
  misc_feature 1..2358: note = source = /note="Description of Artificial Sequence: Synthetic polynucleotide"
  source 1..2358: mol_type = other DNA
atgaaaaagatttggctggcgctggctggtttagttttagcgtttagcgcatcggcggcgcagtatgaagatatgggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 26 - protein, 785 aa, synthetic construct
  REGION 1..785: note = source = /note="Description of Artificial Sequence: Synthetic polypeptide"
MKKIWLALAGLVLAFSASAAQYEDMGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 27 - DNA, 3945 bp, synthetic construct
  misc_feature 1..3945: note = source = /note="Description of Artificial Sequence: Synthetic polynucleotide"
  source 1..3945: mol_type = other DNA
atgaaaaagatttggctggcgctggctggtttagttttagcgtttagcgcatcggcggcgcagtatgaagatcaccatcaccaccaccaccatcaccactctggctcgagcctggtgccgcgcggcagccatatgtctggctcgagcagtaaaggtgaagaactgttcaccggtgttgttccgatcctggttgaactggatggtgatgttaacggccacaaattctctgttcgtggtgaaggtgaaggtgatgcaaccaacggtaaactgaccctgaaattcatctgcactaccggtaaactgccggttccatggccgactctggtgactaccctgacctatggtgttcagtgtttttctcgttacccggatcacatgaagcagcatgatttcttcaaatctgcaatgccggaaggttatgtacaggagcgcaccatttctttcaaagacgatggcacctacaaaacccgtgcagaggttaaatttgaaggtgatactctggtgaaccgtattgaactgaaaggcattgatttcaaagaggacggcaacatcctgggccacaaactggaatataacttcaactcccataacgtttacatcaccgcagacaaacagaagaacggtatcaaagctaacttcaaaattcgccataacgttgaagacggtagcgtacagctggcggaccactaccagcagaacactccgatcggtgatggtccggttctgctgccggataaccactacctgtccacccagtctaaactgtccaaagacccgaacgaaaagcgcgaccacatggtgctgctggagttcgttactgcagcaggtatcacgcacggcatggatgaactctacaaatctggcgcgccgggcggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaacacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 28 - protein, 1314 aa, synthetic construct
  REGION 1..1314: note = source = /note="Description of Artificial Sequence: Synthetic polypeptide"
MKKIWLALAGLVLAFSASAAQYEDHHHHHHHHHSGSSLVPRGSHMSGSSSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGAPGGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRKHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 29 - protein, 760 aa, synthetic construct
  REGION 1..760: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 30 - DNA, 2115 bp, synthetic construct
  misc_feature 1..2115: note = source = /note="Description of Artificial Sequence: Synthetic polynucleotide"
  source 1..2115: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgccctaa

SEQ ID NO: 31 - protein, 704 aa, synthetic construct
  REGION 1..704: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPP

SEQ ID NO: 32 - DNA, 2115 bp, synthetic construct; identical to SEQ ID NO: 30
  misc_feature 1..2115: note = source = /note="Description of Artificial Sequence: Synthetic polynucleotide"
  source 1..2115: mol_type = other DNA

SEQ ID NO: 33 - protein, 676 aa, synthetic construct
  REGION 1..676: note = source = /note="Description of Artificial Sequence: Synthetic polypeptide"
GLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 34 - DNA, 2031 bp, synthetic construct
  misc_feature 1..2031: note = source = /note="Description of Artificial Sequence: Synthetic polynucleotide"
  source 1..2031: mol_type = other DNA
ggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 35 - protein, 659 aa, synthetic construct
  REGION 1..659: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGP

SEQ ID NO: 36 - DNA, 1980 bp, synthetic construct
  misc_feature 1..1980: note = source = /note="Description of Artificial Sequence: Synthetic polynucleotide"
  source 1..1980: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccgtaa

SEQ ID NO: 37 - protein, 622 aa, synthetic construct
  REGION 1..622: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGA

SEQ ID NO: 38 - DNA, 1869 bp, synthetic construct
  misc_feature 1..1869: note = source = /note="Description of Artificial Sequence: Synthetic polynucleotide"
  source 1..1869: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcataa

SEQ ID NO: 39 - protein, 527 aa, synthetic construct
  REGION 1..527: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGV

SEQ ID NO: 40 - DNA, 1584 bp, synthetic construct
  misc_feature 1..1584: note = source = /note="Description of Artificial Sequence: Synthetic polynucleotide"
  source 1..1584: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtctaa

SEQ ID NO: 41 - protein, 515 aa, synthetic construct
  REGION 1..515: note = source = /note="Description of Artificial Sequence: Synthetic polypeptide"
GVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 42 - DNA, 1548 bp, synthetic construct
  misc_feature 1..1548: note = source = /note="Description of Artificial Sequence: Synthetic polynucleotide"
  source 1..1548: mol_type = other DNA
ggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 43 - protein, 466 aa, synthetic construct
  REGION 1..466: note = source = /note="Description of Artificial Sequence: Synthetic polypeptide"
GLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 44 - DNA, 1401 bp, synthetic construct
  misc_feature 1..1401: note = source = /note="Description of Artificial Sequence: Synthetic polynucleotide"
  source 1..1401: mol_type = other DNA
ggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 45 - protein, 460 aa, synthetic construct
  REGION 1..460: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTP

SEQ ID NO: 46 - DNA, 1383 bp, synthetic construct
  misc_feature 1..1383: note = source = /note="Description of Artificial Sequence: Synthetic polynucleotide"
  source 1..1383: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccgtaa

SEQ ID NO: 47 - protein, 416 aa, synthetic construct
  REGION 1..416: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQ

SEQ ID NO: 48 - DNA, 1251 bp, synthetic construct
  misc_feature 1..1251: note = source = /note="Description of Artificial Sequence: Synthetic polynucleotide"
  source 1..1251: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacagtaa

SEQ ID NO: 49 - protein, 401 aa, synthetic construct
  REGION 1..401: note = source = /note="Description of Artificial Sequence: Synthetic polypeptide"
RPGVGVGGIPTYGVGAGGFPGFGVGVGGIPGVAGVPGVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 50 - DNA, 1206 bp, synthetic construct
  misc_feature 1..1206: note = source = /note="Description of Artificial Sequence: Synthetic polynucleotide"
  source 1..1206: mol_type = other DNA
cgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 51 - protein, 371 aa, synthetic construct
  REGION 1..371: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTY

SEQ ID NO: 52 - DNA, 1116 bp, synthetic construct
  misc_feature 1..1116: note = source = /note="Description of Artificial Sequence: Synthetic polynucleotide"
  source 1..1116: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctattaa

SEQ ID NO: 53 - protein, 345 aa, synthetic construct
  REGION 1..345: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPE

SEQ ID NO: 54 - DNA, 1038 bp, synthetic construct
  misc_feature 1..1038: note = source = /note="Description of Artificial Sequence: Synthetic polynucleotide"
  source 1..1038: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaataa

SEQ ID NO: 55 - protein, 336 aa, synthetic construct
  REGION 1..336: note = source = /note="Description of Artificial Sequence: Synthetic polypeptide"
KYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 56 - DNA, 1011 bp, synthetic construct
  misc_feature 1..1011: note = source = /note="Description of Artificial Sequence: Synthetic polynucleotide"
  source 1..1011: mol_type = other DNA
aaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 57 - protein, 288 aa, synthetic construct
  REGION 1..288: note = source = /note="Description of Artificial Sequence: Synthetic polypeptide"
QFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 58 - DNA, 867 bp, synthetic construct
  misc_feature 1..867: note = source = /note="Description of Artificial Sequence: Synthetic polynucleotide"
  source 1..867: mol_type = other DNA
cagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 59 - protein, 275 aa, synthetic construct
  REGION 1..275: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTP

SEQ ID NO: 60 - DNA, 828 bp, synthetic construct
  misc_feature 1..828: note = source = /note="Description of Artificial Sequence: Synthetic polynucleotide"
  source 1..828: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccgtaa

SEQ ID NO: 61 - protein, 227 aa, synthetic construct
  REGION 1..227: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQ

SEQ ID NO: 62 - DNA, 684 bp, synthetic construct
  misc_feature 1..684: note = source = /note="Description of Artificial Sequence: Synthetic polynucleotide"
  source 1..684: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcagtaa

SEQ ID NO: 63 - protein, 219 aa, synthetic construct
  REGION 1..219: note = source = /note="Description of Artificial Sequence: Synthetic polypeptide"
QLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 64 - DNA, 660 bp, synthetic construct
  misc_feature 1..660: note = source = /note="Description of Artificial Sequence: Synthetic polynucleotide"
  source 1..660: mol_type = other DNA
caactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 65 - protein, 126 aa, synthetic construct
  REGION 1..126: note = source = /note="Description of Artificial Sequence: Synthetic polypeptide"
VPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 66 - DNA, 381 bp, synthetic construct
  misc_feature 1..381: note = source = /note="Description of Artificial Sequence: Synthetic polynucleotide"
  source 1..381: mol_type = other DNA
gtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 67 - protein, 87 aa, synthetic construct
  REGION 1..87: note = source = /note="Description of Artificial Sequence: Synthetic polypeptide"
QFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 68 - DNA, 264 bp, synthetic construct
  misc_feature 1..264: note = source = /note="Description of Artificial Sequence: Synthetic polynucleotide"
  source 1..264: mol_type = other DNA
cagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 69 - protein, 72 aa, synthetic construct
  REGION 1..72: note = source = /note="Description of Artificial Sequence: Synthetic polypeptide"
GGVPGAIPGGVPGGVFYPGAGLGALGGGALGPGGKPLKPVPGGLAGAGLGAGLGAFPAVTFPGALVPGGVAD

SEQ ID NO: 70 - DNA, 219 bp, synthetic construct
  misc_feature 1..219: note = source = /note="Description of Artificial Sequence: Synthetic polynucleotide"
  source 1..219: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgattaa

SEQ ID NO: 71 - protein, 44 aa, synthetic construct
  REGION 1..44: note = source = /note="Description of Artificial Sequence: Synthetic polypeptide"
GLGGVLGGAGQFPLGGVAARPGFGLSPIFPGGACLGKACGRKRK

SEQ ID NO: 72 - DNA, 135 bp, synthetic construct
  misc_feature 1..135: note = source = /note="Description of Artificial Sequence: Synthetic polynucleotide"
  source 1..135: mol_type = other DNA
ggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 73 - DNA, 636 bp, synthetic construct
  misc_feature 1..636: note = source = /note="Description of Artificial Sequence: Synthetic polynucleotide"
  source 1..636: mol_type = other DNA
atgaaaaagatttggctggcgctggctggtttagttttagcgtttagcgcatcggcggcgcagtatgaagatgcaggttttccgggtctgcctggtccggcaggcgaaccgggtcgtcatggtaaagatggtctgatgggtagtccgggttttaaaggtgaagcaggttcaccgggtgcacctggtcaggatggcacccgtggtgaaccgggtattccgggatttccgggtaatcgtggcctgatgggtcagaaaggtgaaattggtccgcctggtcagcagggtaaaaaaggcgcaccgggtatgccaggactgatgggttcaaatggcagtccgggtcagccaggcacaccgggttcaaaaggtagcaaaggcgaacctggtattcagggtatgcctggtgcaagcggtctgaaaggcgagccaggtgccaccggttctccgggtgaaccaggttatatgggtctgccaggtatccaaggcaaaaaaggtgataaaggtaatcagggcgaaaaaggcattcagggccagaaaggcgaaaatggccgtcagggtattccaggccagcagggcatccagggtcatcatggtgcaaaaggtgaacgtggtgaaaagggcgaaccaggtgttcgttaa

SEQ ID NO: 74 - protein, 211 aa, synthetic construct
  REGION 1..211: note = source = /note="Description of Artificial Sequence: Synthetic polypeptide"
MKKIWLALAGLVLAFSASAAQYEDAGFPGLPGPAGEPGRHGKDGLMGSPGFKGEAGSPGAPGQDGTRGEPGIPGFPGNRGLMGQKGEIGPPGQQGKKGAPGMPGLMGSNGSPGQPGTPGSKGSKGEPGIQGMPGASGLKGEPGATGSPGEPGYMGLPGIQGKKGDKGNQGEKGIQGQKGENGRQGIPGQQGIQGHHGAKGERGEKGEPGVR

SEQ ID NO: 75 - DNA, 565 bp, synthetic construct
  misc_feature 1..565: note = source = /note="Description of Artificial Sequence: Synthetic polynucleotide"
  source 1..565: mol_type = other DNA
tgcaggttttccgggtctgcctggtccggcaggcgaaccgggtcgtcatggtaaagatggtctgatgggtagtccgggttttaaaggtgaagcaggttcaccgggtgcacctggtcaggatggcacccgtggtgaaccgggtattccgggatttccgggtaatcgtggcctgatgggtcagaaaggtgaaattggtccgcctggtcagcagggtaaaaaaggcgcaccgggtatgccaggactgatgggttcaaatggcagtccgggtcagccaggcacaccgggttcaaaaggtagcaaaggcgaacctggtattcagggtatgcctggtgcaagcggtctgaaaggcgagccaggtgccaccggttctccgggtgaaccaggttatatgggtctgccaggtatccaaggcaaaaaaggtgataaaggtaatcagggcgaaaaaggcattcagggccagaaaggcgaaaatggccgtcagggtattccaggccagcagggcatccagggtcatcatggtgcaaaaggtgaacgtggtgaaaagggcgaaccaggtgttcgttaa

SEQ ID NO: 76 - protein, 187 aa, synthetic construct
  REGION 1..187: note = source = /note="Description of Artificial Sequence: Synthetic polypeptide"
AGFPGLPGPAGEPGRHGKDGLMGSPGFKGEAGSPGAPGQDGTRGEPGIPGFPGNRGLMGQKGEIGPPGQQGKKGAPGMPGLMGSNGSPGQPGTPGSKGSKGEPGIQGMPGASGLKGEPGATGSPGEPGYMGLPGIQGKKGDKGNQGEKGIQGQKGENGRQGIPGQQGIQGHHGAKGERGEKGEPGVR

SEQ ID NO: 77 - DNA, 639 bp, synthetic construct
  misc_feature 1..639: note = source = /note="Description of Artificial Sequence: Synthetic polynucleotide"
  source 1..639: mol_type = other DNA
atgaaaaagatttggctggcgctggctggtttagttttagcgtttagcgcatcggcggcgcagtatgaagatatgggtccgcctggtagccgtggtgcaagtggtccggcaggcgttcgtggtccgaatggtgatgcaggtcgtccgggtgaaccgggtctgatgggtcctcgtggtctgcctggttcaccgggtaatattggtcctgcaggtaaagaaggtccggttggtctgccaggtattgatggccgtccgggtccgattggtccagccggtgcacgtggtgaacctggcaatattggttttccgggtcctaaaggtccgaccggtgatccgggtaaaaatggtgataaaggtcatgcaggtctggcaggcgcacgcggtgcacctggtccggatggtaataatggtgcacagggtccaccgggtccgcagggtgttcaaggtggtaaaggcgaacagggtcctgccggtcctccgggttttcagggactgcctggtccgagcggtcctgcgggtgaagttggtaaacctggtgaacgcggtctgcatggtgaatttggcctgcctgggcctgcaggtccgcgtggcgaacgtggtccgccaggtgaaagcggtgcagcaggtccgacaggttaa

SEQ ID NO: 78 - protein, 212 aa, synthetic construct
  REGION 1..212: note = source = /note="Description of Artificial Sequence: Synthetic polypeptide"
MKKIWLALAGLVLAFSASAAQYEDMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTG

SEQ ID NO: 79 - DNA, 567 bp, synthetic construct
  misc_feature 1..567: note = source = /note="Description of Artificial Sequence: Synthetic polynucleotide"
  source 1..567: mol_type = other DNA
atgggtccgcctggtagccgtggtgcaagtggtccggcaggcgttcgtggtccgaatggtgatgcaggtcgtccgggtgaaccgggtctgatgggtcctcgtggtctgcctggttcaccgggtaatattggtcctgcaggtaaagaaggtccggttggtctgccaggtattgatggccgtccgggtccgattggtccagccggtgcacgtggtgaacctggcaatattggttttccgggtcctaaaggtccgaccggtgatccgggtaaaaatggtgataaaggtcatgcaggtctggcaggcgcacgcggtgcacctggtccggatggtaataatggtgcacagggtccaccgggtccgcagggtgttcaaggtggtaaaggcgaacagggtcctgccggtcctccgggttttcagggactgcctggtccgagcggtcctgcgggtgaagttggtaaacctggtgaacgcggtctgcatggtgaatttggcctgcctgggcctgcaggtccgcgtggcgaacgtggtccgccaggtgaaagcggtgcagcaggtccgacaggttaa

SEQ ID NO: 80 - protein, 188 aa, synthetic construct
  REGION 1..188: note = source = /note="Description of Artificial Sequence: Synthetic polypeptide"
MGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTG

SEQ ID NO: 81 - DNA, 612 bp, synthetic construct
  misc_feature 1..612: note = source = /note="Description of Artificial Sequence: Synthetic polynucleotide"
  source 1..612: mol_type = other DNA
atgaaaaagatttggctggcgctggctggtttagttttagcgtttagcgcatcggcggcgcagtatgaagatggttttcagggtcctgccggtgaaccgggtgaacctggtcagacaggtccggcaggcgcacgtggtcctgcaggtcctcctggtaaagccggtgaagatggtcatccgggtaaaccgggtcgtcctggtgaacgtggtgttgttggtccgcagggtgcccgtggttttccgggtactccgggtctgccaggttttaaaggtattcgtggtcataatggtctggatggtctgaaaggtcagcctggtgcaccgggtgttaaaggtgaaccaggtgctccgggtgaaaatggcacaccgggtcagaccggtgcgcgtggtctgcctggcgaacgcggtcgtgttggtgcacctggtccagccggtgcacgcggtagtgatggtagcgttggtccggttggtccagcgggtccgattggtagcgcaggtccaccgggttttccaggcgcaccgggtccgaaaggtgaaattggtgcagttggtaatgcaggccctgccggtccagcaggaccgcgtggtgaagttggcctgcctggtctgtaa

SEQ ID NO: 82 - protein, 203 aa, synthetic construct
  REGION 1..203: note = source = /note="Description of Artificial Sequence: Synthetic polypeptide"
MKKIWLALAGLVLAFSASAAQYEDGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGL

SEQ ID NO: 83 - DNA, 540 bp, synthetic construct
  misc_feature 1..540: note = source = /note="Description of Artificial Sequence: Synthetic polynucleotide"
  source 1..540: mol_type = other DNA
ggttttcagggtcctgccggtgaaccgggtgaacctggtcagacaggtccggcaggcgcacgtggtcctgcaggtcctcctggtaaagccggtgaagatggtcatccgggtaaaccgggtcgtcctggtgaacgtggtgttgttggtccgcagggtgcccgtggttttccgggtactccgggtctgccaggttttaaaggtattcgtggtcataatggtctggatggtctgaaaggtcagcctggtgcaccgggtgttaaaggtgaaccaggtgctccgggtgaaaatggcacaccgggtcagaccggtgcgcgtggtctgcctggcgaacgcggtcgtgttggtgcacctggtccagccggtgcacgcggtagtgatggtagcgttggtccggttggtccagcgggtccgattggtagcgcaggtccaccgggttttccaggcgcaccgggtccgaaaggtgaaattggtgcagttggtaatgcaggccctgccggtccagcaggaccgcgtggtgaagttggcctgcctggtctgtaa

SEQ ID NO: 84 - protein, 179 aa, synthetic construct
  REGION 1..179: note = source = /note="Description of Artificial Sequence: Synthetic polypeptide"
GFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGL

SEQ ID NO: 85 - DNA, 689 bp, synthetic construct
  misc_feature 1..689: note = source = /note="Description of Artificial Sequence: Synthetic polynucleotide"
  source 1..689: mol_type = other DNA
gtccgcagggtgttgttggtgcagatggtaaagacggtaccccgggtgaaaaaggagaacagggacgtacaggtgcagcaggtaaacagggcagcccgggtgccgatggtgcccgtggcccgctgggtagcattggtcagcagggtgcaagaggcgaaccgggcgatccgggtagtccgggcctgcgtggtgatacgggtctggccggtgttaaaggcgttgcaggtccttcaggtcgtccaggtcaaccgggtgcaaatggtctgccgggtgttaatggtcgtggcggtctggaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 86 - protein, 229 aa, synthetic construct
  REGION 1..229: note = source = /note="Description of Artificial Sequence: Synthetic polypeptide"
GPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 87 - DNA, 2283 bp, synthetic construct
  misc_feature 1..2283: note = source = /note="Description of Artificial Sequence: Synthetic polynucleotide"
  source 1..2283: mol_type = other DNA
ggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtccctggtgttggcggtgtgccaggtgttggtatttcaccggaagcacaggcagcagccgcagctaaggcagcgaaatatggtgccgccggcgcaggagttttaggtgggctggttccgggcccgcaggcagctgtgccgggggttccaggcaccggtggtgtccctggagtcggtacgccggctgcagcggcagccaaagcggctgcgaaagcagcacagtttggcttagtaccgggtgtgggagttgcccccggcgttggcgttgctccaggggtgggtgttgctcctggcgtcggtctggctcctggagtgggcgtagcacccggtgtgggggtggccccgggtgttggggttgcaccgggtatcggtccgggcggtgtcgcagcagcagctaaaagcgcggcgaaagttgcggccaaagcccaactgcgcgccgccgcgggcctcggtgcaggtattccggggctgggtgtcggagttggagtcccgggtttgggcgtgggcgcgggagttccgggactgggagtgggtgccggagttcctggctttggtgcaggcgcagatgaaggtgttcgtcgtagcctgagtccggaactgcgtgaaggtgatccgagtagcagccagcatctgccgagcaccccgagcagcccgcgtgttccgggtgcattagctgcagcaaaagccgccaagtatggtgcagccgtgccgggcgtcttaggtggtctgggcgccctgggtggtgtaggcattccgggaggtgttgtgggtgcaggaccggccgccgcagctgcggccgccaaagcagctgcaaaagcggcccagtttggtttagtgggcgccgcaggtttaggcggtttaggtgtgggtggactgggtgtacctggcgtaggcggtctgggtggaattccgcccgcagcggccgcgaaagcggcaaaatatggcgcggcaggcctgggcggcgtgctgggtggggcaggtcagtttccgctgggcggggttgccgcacgtccgggatttggtctgagcccgattttccctggcggcgcatgtctgggtaaagcatgtggtcgtaaacgtaaataa

SEQ ID NO: 88 - protein, 760 aa, synthetic construct; identical to SEQ ID NO: 29
  REGION 1..760: note = source = /note="Description of Artificial Sequence: Synthetic polypeptide"

SEQ ID NO: 89 - protein, 429 aa, Podocoryna carnea; identical to SEQ ID NO: 1

SEQ ID NO: 90 - DNA, 570 bp, synthetic construct
  misc_feature 1..570: note = source = /note="Description of Artificial Sequence: Synthetic polynucleotide"
  source 1..570: mol_type = other DNA
ggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 91 - protein, 189 aa, synthetic construct
  REGION 1..189: note = source = /note="Description of Artificial Sequence: Synthetic polypeptide"
GPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 92 - protein, 228 aa, synthetic construct
  REGION 1..228: note = source = /note="Description of Artificial Sequence: Synthetic polypeptide"
MKKIWLALAGLVLAFSASAGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLGDYKDDDDK

SEQ ID NO: 93 - DNA, 687 bp, synthetic construct
  misc_feature 1..687: note = source = /note="Description of Artificial Sequence: Synthetic polynucleotide"
  source 1..687: mol_type = other DNA
atgaaaaagatttggctggcgctggctggtttagttttagcgtttagcgcatcggcgggtgatcagggtccggttggtcgtaccggtgaagttggtgcagtcgggccgccgggttttgcgggtgaaaaaggcccgtcaggtgaagcaggcaccgctggccctcctggcacgcctggcccacagggtttactgggcgcacctggaattctgggactgccgggcagccgtggagaacgcggtttaccaggtgttgccggtgccgttggtgaacctggtccactgggcattgcagggccgcctggcgcacggggaccgcctggtgctgttggtagtccgggtgtgaatggtgctccgggtgaagccggtcgtgacggtaatccgggaaatgacggcccgccaggccgcgatggtcagccgggtcataaaggtgagcgtggttacccaggtaatattggtccagtcggtgccgccggtgcgccgggtcctcatggccctgtcggtccagccggtaaacatggtaatcgcggtgagacaggtccgtcaggaccagtgggccctgctggcgcagtcggtccgcgcgggccgagtggccctcagggtattcgtggcgataaaggggaaccgggcgaaaaagggccgcggggtctgccaggcctgggtgactacaaagacgacgacgacaaataa

SEQ ID NO: 94 - protein, 228 aa, synthetic construct
  REGION 1..228: note = source = /note="Description of Artificial Sequence: Synthetic polypeptide"
MKKIWLALAGLVLAFSASAKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDGDYKDDDDK

SEQ ID NO: 95 - DNA, 687 bp, synthetic construct
  misc_feature 1..687: note = source = /note="Description of Artificial Sequence: Synthetic polynucleotide"
  source 1..687: mol_type = other DNA
atgaaaaagatttggctggcgctggctggtttagttttagcgtttagcgcatcggcgaaaggtcacaatggactgcaaggcctgccaggtattgcaggtcatcatggtgatcaaggtgccccgggaagcgttggtccggcggggccgagaggccctgcgggaccttcaggtccggcaggcaaagatggtcggacaggccatccgggcaccgttggccctgcaggaattcgtggaccgcagggtcatcagggacctgctggtccgccaggtcccccgggccctccgggaccaccgggtgttagtggtggtggttatgattttggctatgatggtgatttttatcgtgcagatcagccgcgtagcgcaccgagcctgcgtcctaaagattatgaagttgatgcaaccctgaaaagcctgaataatcagattgaaacactgctgacaccggaaggtagccgtaaaaatccggcccgtacctgtcgtgatctgcgtctgagccacccggaatggagcagcggttattattggattgatccgaatcaaggttgtaccatggatgcaattaaagtttattgtgattttagcacaggtgaaacatgtatccgtgcacagccggaaaatattccggccaaaaattggtatcgtagtagcaaagatggtgactacaaagacgacgacgacaaataa

SEQ ID NO: 96 - protein, 262 aa, synthetic construct
  REGION 1..262: note = source = /note="Description of Artificial Sequence: Synthetic polypeptide"
MKKIWLALAGLVLAFSASAYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFKGDYKDDDDK

SEQ ID NO: 97 - DNA, 788 bp, synthetic construct
  misc_feature 1..788: note = source = /note="Description of Artificial Sequence: Synthetic polynucleotide"
  source 1..788: mol_type = other DNA
tgaaaaagatttggctggcgctggctggtttagttttagcgtttagcgcatcggcgtatgaagttgatgcaaccctgaaaagcctgaataatcagattgaaacactgctgacaccggaaggtagccgtaaaaatccggcccgtacctgtcgtgatctgcgtctgagccacccggaatggagcagcggttattattggattgatccgaatcaaggttgtaccatggatgcaattaaagtttattgtgattttagcacaggtgaaacatgtatccgtgcacagccggaaaatattccggccaaaaattggtatcgtagtagcaaagataaaaaacatgtgtggctgggtgaaaccattaatgcaggtagccagtttgaatacaatgttgaaggtgttaccagcaaagaaatggcaacacagctggcatttatgcgtctgctggcaaattatgcaagccagaatattacatatcattgtaaaaatagcattgcatatatggatgaagaaaccggtaatctgaaaaaagcagttattctgcagggtagcaatgatgttgaactggttgccgaaggtaatagccgttttacatataccgttctggttgatggttgtagcaaaaaaaccaatgaatggggtaaaaccatcattgaatataaaaccaacaaaccgagccgtctgccgtttctggatatcgctccgctggatattggtggtgccgatcaggaattttttgtcgatatcggtcctgtgtgttttaaaggtgactacaaagacgacgacgacaaataa

SEQ ID NO: 98 - protein, 228 aa, synthetic construct
  REGION 1..228: note = source = /note="Description of Artificial Sequence: Synthetic polypeptide"
MKKIWLALAGLVLAFSASAGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLGDYKDDDDK

SEQ ID NO: 99 - DNA, 687 bp, synthetic construct
  misc_feature 1..687: note = source = /note="Description of Artificial Sequence: Synthetic polynucleotide"
  source 1..687: mol_type = other DNA
atgaaaaagatttggctggcgctggctggtttagttttagcgtttagcgcatcggcgggtggcgtaccaggcgcaattcctgggggtgtcccaggcggtgttttttatccgggcgccggtcttggcgcactgggtggcggtgcactgggcccgggcggcaaaccgctgaaaccggtaccaggtggtttagcaggcgccggcttaggcgcaggtctgggagcatttccggcagttacctttccaggggcactggttcctggaggtgtggccgatgcagccgcggcatataaagccgctaaagccggtgcgggtttaggaggcgtcccaggtgtcggtggcctgggtgttagcgccggtgcagttgttccgcagccgggagcaggggttaaacctggtaaagtgccgggagtaggtctgccaggcgtttatcctggtggtgttttgccgggtgcccgttttccgggcgttggtgttcttccaggcgtgccgaccggagccggtgttaaaccgaaagcccccggtgttggaggtgcatttgcaggcatcccgggagttggcccgtttggtggtccgcaacctggggttccgttaggttatccgattaaagcaccgaaactgcccggcggttatggtctgccgtacacaaccggtaaactgggtgactacaaagacgacgacgacaaataa

SEQ ID NO: 100 - protein, 228 aa, synthetic construct
  REGION 1..228: note = source = /note="Description of Artificial Sequence: Synthetic polypeptide"
MKKIWLALAGLVLAFSASAPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVGDYKDDDDK

SEQ ID NO: 101 - DNA, 687 bp, synthetic construct
  misc_feature 1..687: note = source = /note="Description of Artificial Sequence: Synthetic polynucleotide"
  source 1..687: mol_type = other DNA
atgaaaaagatttggctggcgctggctggtttagttttagcgtttagcgcatcggcgccgtatggttatggcccgggtggagttgcgggtgcagcaggtaaagcgggttatcctaccggaaccggtgtaggtccgcaggccgctgctgccgccgccgcaaaagcagcggctaaatttggcgccggagcagcgggtgttctgcctggagttggtggtgcgggcgtgccaggggtacctggtgcaattccgggtattggtggtattgccggtgtcggcaccccggccgcggcagctgcggcagcggcggctgccaaagctgctaaatacggtgccgcggcgggtctggtgccaggaggtccgggttttggtccgggagtggttggcgtgcctggcgcaggcgttcctggtgtgggcgttccaggtgcagggattcctgttgtgcctggtgccggtattcccggcgcggccgttccgggggtggttagcccggaagccgcagcgaaggctgcggcaaaggcagcaaagtatggcgcacgcccaggagtcggcgtgggtggtatcccgacctatggggtgggcgcagggggttttcctggtttcggcgtaggtgtaggaggtataccgggcgtggccggtgtaccaggggttggtggcgtcggtgactacaaagacgacgacgacaaataa

SEQ ID NO: 102 - protein, 273 aa, synthetic construct
  REGION 1..273: note = source = /note="Description of Artificial Sequence: Synthetic polypeptide"
MKKIWLALAGLVLAFSASAPVGRRGPKGSRGDPGDGGAAGPKGPEGVDGLIGEPGQPGPIGAEGSSGLEGFLGDKGSKGARGGPGNRGRPGQDGVPGQDGRAGEKGEGGETGDRGQQGLRGKVGDPGLVGDLGAQGPQGSQGLVGPPGIPGEPGSGGEPGDQGPRGPEGPQGSPGVRGGRGERGTPGAVGPKGPPGKNGADGPRGLPGASGPPGSPGNQGPEGSRGADGNNGFPGDDGENGLVGIPGEPGPKGARGTRGELGKTGDYKDDDDK

SEQ ID NO: 103 - DNA, 822 bp, synthetic construct
  misc_feature 1..822: note = source = /note="Description of Artificial Sequence: Synthetic polynucleotide"
  source 1..822: mol_type = other DNA
atgaaaaagatttggctggcgctggctggtttagttttagcgtttagcgcatcggcgccggttggtcgtcgtggtccgaaaggtagccgtggtgatcctggtgatggtggtgcagcaggtcctaaaggtccggaaggtgttgatggtctgattggtgaaccgggtcagcctggtccgattggcgcagaaggtagcagcggtctggaaggttttctgggtgataaaggtagcaaaggtgcacgtggtggtccgggtaatcgcggtcgtcctggtcaggatggtgttccgggtcaagatggtcgtgccggtgaaaaaggtgaaggtggtgaaaccggtgatcgcggtcagcagggtctgcgtggtaaagttggtgatccaggtctggtgggtgatctgggtgcacagggtccgcagggtagccaaggtctggttggtccgcctggtattccgggtgaacctggtagcggtggcgaaccgggtgatcagggtcctcgcggtccagaaggtcctcagggttcaccgggtgttcgcggtggtcgtggtgaacgtggtacaccgggtgcagttggaccgaaaggtccgccaggtaaaaatggtgcagatggtccgcgtggtctgcctggtgcaagcggtcctccgggtagtcctggtaaccagggtcctgaaggttctcgtggtgccgatggtaataatggttttccaggtgatgatggtgaaaatggcctggttggtatccctggcgaaccaggtccaaaaggcgcacgcggtacacgcggtgaactgggtaaaaccggtgactacaaagacgacgacgacaaataa

SEQ ID NO: 104 - protein, 450 aa, synthetic construct
  REGION 1..450: note = source = /note="Description of Artificial Sequence: Synthetic polypeptide"
MKKIWLALAGLVLAFSASAGRGGPAGLQGAAGNPGDPGDRGQAGEIGLPGTEGQRGQGGSRGDDGIGGQSGTDGDPGNDGVAGIRGARGEPGATGPEGAAGQKGDRGRFGEQGRPGNDGPPGRRGRVGNLGETGAEGDEGTRGYTGDRGPEGAIGISGVTGNPGPQGIKGPPGDTGHPGRQGPSGPQGPPGIPGTDGLTIHNLIKPPSQFFDATSSSDPLTDAVVESILKSFQYAELEIDLTKKPDGTMKYPAISCDDLHKDYPQLPSGNYTLDPNGGCKNDAFETYCEFNNSVKMCLTPKIPTLLPMGTYKYYVNSEGYYSPNDFGLNLRFFEYYGSVTQLKFLQTKATRVTQTIRVLCKNYDPLHKQPVFIGMNDETVMDEPRMEENQCQYFNGLSAHVELELSSNDPSYLPIYEMRLYLGRKTNEELGIELGDLCFEYGDYKDDDDK

SEQ ID NO: 105 - DNA, 1353 bp, synthetic construct
  misc_feature 1..1353: note = source = /note="Description of Artificial Sequence: Synthetic polynucleotide"
  source 1..1353: mol_type = other DNA
atgaaaaagatttggctggcgctggctggtttagttttagcgtttagcgcatcggcgggtcgtggcggtccggcaggtctgcagggtgctgcaggtaatcctggcgaccctggcgatcgtggtcaggcaggcgaaattggtctgccaggcaccgaaggtcagcgtggtcaaggtggttcacgtggtgatgacggtattggtggtcagagcggcaccgatggcgatccgggtaacgatggtgttgcaggtattcgtggtgcacgcggagaacctggtgccaccggacctgaaggtgcagccggtcagaaaggtgatcgtggccgttttggcgaacagggtcgtccgggaaatgatggtccaccgggtcgccgtggccgtgtgggcaatctgggtgaaacaggtgccgaaggtgatgaaggcacccgtggttatacaggtgaccgtggaccggaaggcgcaattggtattagcggtgtgaccggtaatccgggtccacagggcattaaaggccctccgggtgatacgggtcatccgggtcgtcagggaccgagcggtccgcaaggaccaccgggtattccaggtacagatggcctgaccattcataatctgattaaaccgcctagccagttttttgatgcaaccagcagcagcgatccgctgaccgatgcagttgttgaaagcattctgaaatcttttcagtatgccgagctggaaattgacctgaccaaaaaaccggatggcaccatgaaatatccggcaattagctgtgatgatctgcacaaagattatccgcagctgccgagcggtaattataccctggatccgaatggtggttgtaaaaatgatgcctttgaaacctattgcgagttcaacaatagcgtgaaaatgtgtctgaccccgaaaattccgacactgctgccgatgggcacctataaatactatgttaatagcgagggttactacagcccgaatgattttggtctgaatctgcgcttttttgagtattatggtagcgttacccagctgaaatttctgcagaccaaagcaacccgtgttacccagaccattcgtgttctgtgtaaaaactatgatccgctgcataaacagccggtttttattggtatgaatgacgaaaccgttatggatgaaccgcgtatggaagaaaatcagtgccagtattttaacggtctgagcgcacatgttgaactggaactgagcagcaatgatccgagctatctgccgatttatgaaatgcgtctgtatctgggtcgtaaaaccaatgaagaactgggcattgaactgggcgatctgtgttttgaatatggtgactacaaagacgacgacgacaaataa

SEQ ID NO: 106 - protein, 286 aa, synthetic construct
  REGION 1..286: note = source = /note="Description of Artificial Sequence: Synthetic polypeptide"
MKKIWLALAGLVLAFSASAEKTSSKVALMTVLVVITGALIIEGTSITRGSTHVNRGLRKRQTSEDNCEAVKVGLPGRDGREGPPGPPGPAGRDGRDAVCSNQTTGLGAKGDRGPPGTPGFPGEVGRPGPPGADGIPGPQGERGAVGPGGKPGPRGEVGTPGADGADGATGATGVQGPDGAKGEKGASGTAGLKGEKGDTCIPDSNSTLGMPGTPGAGGSKGQKGESGIVGPKGERGEIGTPGHPGFRGADGEPGHKGVPGRAGAQGDRGDPGDDGLTGDYKDDDDK

SEQ ID NO: 107 - DNA, 861 bp, synthetic construct
  misc_feature 1..861: note = source = /note="Description of Artificial Sequence: Synthetic polynucleotide"
  source 1..861: mol_type = other DNA
atgaaaaagatttggctggcgctggctggtttagttttagcgtttagcgcatcggcggaaaaaaccagcagcaaagttgcactgatgaccgttctggttgttattaccggtgcactgattattgaaggcaccagcattacccgtggtagcacccatgttaatcgtggtctgcgtaaacgtcagaccagcgaagataattgtgaagcagttaaagttggtctgccaggtcgtgatggtcgtgaaggtcctccgggtccgcctggtccggctggcagagatggccgtgatgcagtttgtagcaatcagaccaccggtctgggtgcaaaaggtgatcgtggtccgccaggtacaccgggttttccgggtgaagttggccgtccgggtccaccgggtgcagatggtattccgggtcctcagggtgaacgtggtgcagttggtcctggtggtaaacctggtccgcgtggtgaagtgggcacccctggtgccgatggcgcagatggtgcaaccggtgcgaccggtgttcagggtcctgatggtgccaaaggcgaaaaaggtgcaagcggcaccgcaggtctgaaaggtgagaaaggcgatacctgtattccggatagcaatagcaccctgggtatgcctggtacaccaggtgccggtggtagcaaaggccagaaaggtgaaagtggtattgttggtccgaaaggcgaacgcggtgaaattggcacaccgggtcatcctggttttcgtggtgcggatggtgaaccaggtcataaaggtgttccgggtcgtgccggtgcgcagggtgatcgcggtgatccgggtgatgatggtctgaccggtgactacaaagacgacgacgacaaataa

SEQ ID NO: 108 - protein, 512 aa, synthetic construct
  REGION 1..512: note = source = /note="Description of Artificial Sequence: Synthetic polypeptide"
MKKIWLALAGLVLAFSASAGFPGAPGADGAPGQKGELGAVGPQGTPGLSGPSGPTGPPGPKGVRGAPGSSGAKGDAGNPGDDGPVGPQGVPGVDGSPGQKGETGRVGPRGHDGINGTPGEDGATGFPGPDGAKGEKGTSGTAGLKGEKGDTCIPDSNSTLGMPGTPGAGWSKGQKGESGIVGPKGEKGEIGTPGPPGFRGADGEPGQRGEPGRAGAQGERGAPGNNGRDGFPGDPGADGAPGQKGELGAIGHPGFSGPSGPSGPTGPPGPKGVRGAQGRPGDRGSPGDVGPIGAPGPPGADGVPGLTGVQGRDGPKGESASSGAVYVRWGRTTCPSGADVVYSGRAAGAKYDHSGGTSDHHCLPNNPQYLSEDDTNALGAQLYGVEYEIRDRSSPYNSLDQSDMPCVVCNANGRSQLLMVPARYTCPTGWSREYYGYMMSEGKAKNREGRKTTICMDFSAEAVPGSGANTNPSPGIMMRANCNGLACPPYQSNTPLTCAVCTKGDYKDDDDK

SEQ ID NO: 109 - DNA, 1539 bp, synthetic construct
  misc_feature 1..1539: note = source = /note="Description of Artificial Sequence: Synthetic polynucleotide"
  source 1..1539: mol_type = other DNA
atgaaaaagatttggctggcgctggctggtttagttttagcgtttagcgcatcggcgggttttcctggcgctccgggtgccgacggtgctccgggtcaaaaaggtgaactgggtgccgtgggtccgcagggcactccgggtctgagtggtcctagtggtccgaccggtccaccaggtccaaaaggcgtgcgtggtgcaccgggtagcagcggagccaaaggtgatgcaggtaaccctggtgatgacggtccggttggtccacagggcgttccaggtgttgatggtagccctggccaaaagggtgaaaccggtcgtgtgggtcctcgtggtcatgatggtattaatggcaccccaggtgaagatggtgcgacaggctttccaggtccggatggcgcaaagggtgagaagggcaccagcggtacagctggcctgaagggcgaaaagggcgatacatgcatcccggattcaaattcaacactgggcatgccaggtacgcctggcgcaggttggagtaaaggacaaaaaggcgaatcaggcattgtgggacctaaaggcgagaagggtgagattggtactccgggaccgccaggctttcgcggtgcagacggcgaaccgggtcagcgtggcgaacctggtcgtgcaggcgcacaaggtgaacgcggagcccctggtaataatggacgtgatggctttcctggtgatccaggtgcagatggcgcacctggccagaaaggcgaactgggagcaattggtcatccgggatttagcggtccgtcaggtccgagcggaccgacaggtcctcctggaccgaaaggtgtacgtggcgcacagggtcgtcctggcgatcgtggcagtccaggtgatgtgggtccgattggtgcacctggtcctccaggtgcggacggcgtgcctggtttaacaggtgtgcagggtcgcgacggtcctaaaggtgaatcagcaagcagcggtgcagtttatgttcgttggggtcgtaccacctgtcctagcggagcagatgttgtttatagcggtcgcgcagccggtgcaaaatatgatcattcaggtggcacctcagatcatcattgtctgccgaataatccgcagtatctgagcgaagatgataccaatgcactgggtgcacagctgtatggtgtggaatatgaaattcgtgatcgtagcagcccgtataatagcctggatcagagcgatatgccgtgtgttgtttgtaatgcaaatggtcgtagccagctgctgatggttccggcacgttatacatgcccgaccggttggagccgtgaatattatggttatatgatgagcgaaggcaaagccaaaaatcgcgaaggtcgtaaaaccaccatttgtatggattttagcgcagaagcagttcctggtagcggtgcaaataccaatccgagtccgggtattatgatgcgtgcaaattgtaatggtctggcatgtccgccttatcagagcaatacaccgctgacctgtgccgtttgtaccaaaggtgactacaaagacgacgacgacaaataa

SEQ ID NO: 110 - protein, 219 aa, synthetic construct
  REGION 1..219: note = source = /note="Description of Artificial Sequence: Synthetic polypeptide"
MKKIWLALAGLVLAFSASAGPAGAKGPSGDIGRPGESGSPGARGHSGQPGRTGIAGNQGLPGTAGEEGRTGPPGPAGLRGQAGMMGFPGPKGAAGLPGKPGDRGNVGLAGPRGAPGKDGEVGAQGPPGVAGPTGPRGETGLAGSVGFQGMPGPSGAAGEPGKPGNQGLRGDAGSPGMIGPRGERGLPGERGASGAQGLLGPRGTSGAPGLGDYKDDDDK

SEQ ID NO: 111 - DNA, 660 bp, synthetic construct
  misc_feature 1..660: note = source = /note="Description of Artificial Sequence: Synthetic polynucleotide"
  source 1..660: mol_type = other DNA
atgaaaaagatttggctggcgctggctggtttagttttagcgtttagcgcatcggcgggtccggcaggcgcaaaaggtccgagcggtgatattggtcgtccgggtgaaagcggtagtccgggtgcacgtggtcatagcggtcagcctggtcgtaccggtattgcaggtaatcagggtctgcctggtacagccggtgaagaaggtcgcaccggtccgccaggtcctgcaggtctgcgtggtcaggcaggtatgatgggttttccgggtccgaaaggtgcagcgggtctgccaggcaaaccgggtgatcgtggtaatgttggtctggctggtccgcgtggtgcaccgggtaaagatggtgaagttggtgcacagggtcctccgggtgttgcaggtccgaccggtcctcgtggtgaaaccggtctggcaggtagcgttggttttcagggtatgccaggtccgtcaggtgcagcaggcgaacctggtaaaccgggtaaccagggcctgcgtggtgatgccggttcaccgggtatgattggtccacgcggtgaacgtggcctgcctggcgaacgtggtgcaagcggtgcacaaggtctgctgggtccacgtggcacctcaggcgcaccaggtctgggtgactacaaagacgacgacgacaaataa

SEQ ID NO: 112 - protein, 237 aa, synthetic construct
  REGION 1..237: note = source = /note="Description of Artificial Sequence: Synthetic polypeptide"
MKKIWLALAGLVLAFSASAQGIPGSAGKEGGKGDPGPLGSPGKPGPDGLRGFAGARGLPGAAGPPGLKGAEGPMGAPGLTGSTGERGPNGPAGAIGLPGRPGGPGPPGPVGEKGDPGDKGLPGPAGDDGVQGAMGLPGPIGSQGPPGDYGDKGELGKPGQKGSKGDKGESGPPGPIGIQGPIGHPGPIGSDGSPGLRGYLGMRGQKGDDGIRGLPGSAGPVGLQGLPGGDYKDDDDK

SEQ ID NO: 113 - DNA, 714 bp, synthetic construct
  misc_feature 1..714: note = source = /note="Description of Artificial Sequence: Synthetic polynucleotide"
  source 1..714: mol_type = other DNA
atgaaaaagatttggctggcgctggctggtttagttttagcgtttagcgcatcggcgcagggtattccgggtagcgcaggtaaagaaggtggtaaaggcgatccgggtccgctgggttcaccgggtaaaccgggtcctgatggtctgcgtggttttgccggtgcacgtggtctgcctggtgcagcaggtccgcctggtctgaaaggtgccgaaggtccgatgggtgctccgggtctgaccggtagcaccggtgaacgcggtccgaatggtccggcaggcgcaattggtctgccaggtcgtcctggtggtccgggtcctcctggtccggttggtgaaaaaggtgatcctggtgataaaggcctgcctggtcctgccggtgatgatggtgttcagggtgccatgggcttaccgggtccgattggtagccagggtcctccgggtgattatggcgataaaggtgaactgggtaaacctggccagaaaggtagcaaaggtgacaaaggcgaaagcggtccgccaggtccgatcggcattcagggtcctattggtcatccaggtccaattggttcagatggctcaccgggactgcgtggctatctgggtatgcgtggacagaaaggtgatgacggtattcgtggcctgccaggtagtgcaggtccggtgggtctgcagggactgcctggtggtgactacaaagacgacgacgacaaataa

SEQ ID NO: 114 - protein, 254 aa, synthetic construct
  REGION 1..254: note = source = /note="Description of Artificial Sequence: Synthetic polypeptide"
MKKIWLALAGLVLAFSASAKGETGEAGDPGTPGEPGIAGPKGDVGDKGDAGPPGAAGPAGVKGPPGEDGAKGDVGPAGFPGDPGPTGEPGVPGMDGGVGEKGSLGDPGLTGPRGASGEPGPPGSPGKRGPPGPAGPEGREGLKGSKGSPGQEGPVGRTGPIGPQGSPGNVGPKGLRGIPGPTGEQGLLGPPGQAGPPGPMGPPGMPGLRGAQGLKGDKGHVGLIGLIGPPGEMGEKGDQGLPGIQGDYKDDDDK

SEQ ID NO: 115 - DNA, 765 bp, synthetic construct
  misc_feature 1..765: note = source = /note="Description of Artificial Sequence: Synthetic polynucleotide"
  source 1..765: mol_type = other DNA
atgaaaaagatttggctggcgctggctggtttagttttagcgtttagcgcatcggcgaaaggtgaaaccggtgaagcgggtgatccgggtacaccgggtgaacctggtattgcaggtccgaaaggtgatgttggtgataaaggtgacgcaggtccgcctggtgcagcaggtccggcaggcgttaaaggtcctccgggtgaagatggtgcaaaaggcgacgttggtcctgcaggttttcctggcgatccgggtccgactggtgaaccgggtgtgccaggtatggatggtggtgtgggtgaaaaaggtagcctgggtgatcctggtctgaccggtccgcgtggcgcaagtggtgaaccaggtccaccgggtagtccgggtaaacgtggtcctcctggaccggctggtccggaaggtcgtgaaggtctgaaaggtagcaaaggttcaccgggtcaagaaggtccggttggtcgtaccggtccgattggtccgcagggctcaccgggtaatgttggtcctaaaggtctgcgtggtattccgggtcctacaggcgaacagggtctgctgggtccgccaggccaagcaggtcctccaggtcctatgggtccacctggtatgcctggcctgcgtggtgcccagggcctgaaaggcgataaaggccatgttggtctgattggcctgattggtccaccaggtgaaatgggagaaaaaggcgatcagggcctgcctggtattcagggtgactacaaagacgacgacgacaaataa

SEQ ID NO: 116 - protein, 30 aa, synthetic construct
  SITE 1..30: note = /note="This sequence may encompass 2-30 residues"
  REGION 1..30: note = source = /note="Description of Artificial Sequence: Synthetic His tag"
HHHHHHHHHHHHHHHHHHHHHHHHHHHHHH

SEQ ID NO: 117 - protein, 20 aa, synthetic construct
  SITE 1..20: note = /note="This sequence may encompass 2-20 residues"
  REGION 1..20: note = source = /note="Description of Artificial Sequence: Synthetic His tag"
HHHHHHHHHHHHHHHHHHHH

SEQ ID NO: 118 - protein, 15 aa, synthetic construct
  SITE 1..15: note = /note="This sequence may encompass 5-15 residues"
  REGION 1..15: note = source = /note="Description of Artificial Sequence: Synthetic His tag"
HHHHHHHHHHHHHHH

SEQ ID NO: 119 - protein, 18 aa, synthetic construct
  SITE 1..18: note = /note="This sequence may encompass 5-18 residues"
  REGION 1..18: note = source = /note="Description of Artificial Sequence: Synthetic His tag"
HHHHHHHHHHHHHHHHHH

SEQ ID NO: 120 - protein, 16 aa, synthetic construct
  SITE 1..16: note = /note="This sequence may encompass 5-16 residues"
  REGION 1..16: note = source = /note="Description of Artificial Sequence: Synthetic His tag"
HHHHHHHHHHHHHHHH

SEQ ID NO: 121 - protein, 14 aa, synthetic construct
  SITE 1..14: note = /note="This sequence may encompass 5-14 residues"
  REGION 1..14: note = source = /note="Description of Artificial Sequence: Synthetic His tag"
HHHHHHHHHHHHHH

SEQ ID NO: 122 - protein, 13 aa, synthetic construct
  SITE 1..13: note = /note="This sequence may encompass 5-13 residues"
  REGION 1..13: note = source = /note="Description of Artificial Sequence: Synthetic His tag"
HHHHHHHHHHHHH

SEQ ID NO: 123 - protein, 12 aa, synthetic construct
  SITE 1..12: note = /note="This sequence may encompass 5-12 residues"
  REGION 1..12: note = source = /note="Description of Artificial Sequence: Synthetic His tag"
HHHHHHHHHHHH

SEQ ID NO: 124 - protein, 11 aa, synthetic construct
  SITE 1..11: note = /note="This sequence may encompass 5-11 residues"
  REGION 1..11: note = source = /note="Description of Artificial Sequence: Synthetic His tag"
HHHHHHHHHHH

SEQ ID NO: 125 - protein, 10 aa, synthetic construct
  SITE 1..10: note = /note="This sequence may encompass 5-10 residues"
  REGION 1..10: note = source = /note="Description of Artificial Sequence: Synthetic His tag"
HHHHHHHHHH

SEQ ID NO: 126 - protein, 12 aa, synthetic construct
  SITE 1..12: note = /note="This sequence may encompass 6-12 residues"
  REGION 1..12: note = source = /note="Description of Artificial Sequence: Synthetic His tag"
HHHHHHHHHHHH

SEQ ID NO: 127 - protein, 11 aa, synthetic construct
  SITE 1..11: note = /note="This sequence may encompass 6-11 residues"
  REGION 1..11: note = source = /note="Description of Artificial Sequence: Synthetic His tag"
HHHHHHHHHHH

SEQ ID NO: 128 - protein, 10 aa, synthetic construct
  SITE 1..10: note = /note="This sequence may encompass 7-10 residues"
  REGION 1..10: note = source = /note="Description of Artificial Sequence: Synthetic His tag"
HHHHHHHHHH

SEQ ID NO: 129 - protein, 9 aa, synthetic construct
  REGION 1..9: note = source = /note="Description of Artificial Sequence: Synthetic 9xHis tag"
HHHHHHHHH

SEQ ID NO: 130 - protein, 150 aa, synthetic construct
  SITE 1..150: note = /note="This sequence may encompass 2-50 'Gly Glu Lys' repeating units"
  REGION 1..150: note = source = /note="Description of Artificial Sequence: Synthetic polypeptide"
GEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEK

SEQ ID NO: 131 - protein, 150 aa, synthetic construct
  SITE 1..150: note = /note="This sequence may encompass 2-50 'Gly Asp Lys' repeating units"
  REGION 1..150: note = source = /note="Description of Artificial Sequence: Synthetic polypeptide"
GDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDK

SEQ ID NO: 132 - protein, 120 aa, synthetic construct
  SITE 1..120: note = /note="This sequence may encompass 2-40 'Gly Glu Lys' repeating units"
  REGION 1..120: note = source = /note="Description of Artificial Sequence: Synthetic polypeptide"
GEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEK

SEQ ID NO: 133 - protein, 120 aa, synthetic construct
  SITE 1..120: note = /note="This sequence may encompass 2-40 'Gly Asp Lys' repeating units"
  REGION 1..120: note = source = /note="Description of Artificial Sequence: Synthetic polypeptide"
GDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDKGDK

SEQ ID NO: 134 - protein, 90 aa, synthetic construct
  SITE 1..90: note = /note="This sequence may encompass 2-30 'Gly Glu Lys' repeating units"
  REGION 1..90: note = source = /note="Description of Artificial Sequence: Synthetic polypeptide"
GEKGEKGEKGEKGEKGEKGEKGEKGEKGEKGEKGEKGEKGEKGEKGEKGEKGEKGEKGEKGEKGEKGEKGEKGEKGEKGEKGEKGEKGEK

SEQ ID NO: 135 - protein, 90 aa, synthetic construct
  SITE 1..90: note = /note="This sequence may encompass 2-30 'Gly Asp Lys' repeating units"
  REGION 1..90: note = source = /note="Description of Artificial Sequence: Synthetic polypeptide"
GDKGDKGDKGDKGDKGDKGDKGDKGDKGDKGDKGDKGDKGDKGDKGDKGDKGDKGDKGDKGDKGDKGDKGDKGDKGDKGDKGDKGDKGDK

SEQ ID NO: 136 - protein, 60 aa, synthetic construct
  SITE 1..60: note = /note="This sequence may encompass 2-20 'Gly Glu Lys' repeating units"
  REGION 1..60: note = source = /note="Description of Artificial Sequence: Synthetic polypeptide"
GEKGEKGEKGEKGEKGEKGEKGEKGEKGEKGEKGEKGEKGEKGEKGEKGEKGEKGEKGEK

SEQ ID NO: 137 - protein, 60 aa, synthetic construct
  SITE 1..60: note = /note="This sequence may encompass 2-20 'Gly Asp Lys' repeating units"
  REGION 1..60: note = source = /note="Description of Artificial Sequence: Synthetic polypeptide"
GDKGDKGDKGDKGDKGDKGDKGDKGDKGDKGDKGDKGDKGDKGDKGDKGDKGDKGDKGDK

SEQ ID NO: 138 - protein, 45 aa, synthetic construct
  SITE 1..45: note = /note="This sequence may encompass 2-15 'Gly Glu Lys' repeating units"
  REGION 1..45: note = source = /note="Description of Artificial Sequence: Synthetic polypeptide"
GEKGEKGEKGEKGEKGEKGEKGEKGEKGEKGEKGEKGEKGEKGEK

SEQ ID NO: 139 - protein, 45 aa, synthetic construct
  SITE 1..45: note = /note="This sequence may encompass 2-15 'Gly Asp Lys' repeating units"
  REGION 1..45: note = source = /note="Description of Artificial Sequence: Synthetic polypeptide"
GDKGDKGDKGDKGDKGDKGDKGDKGDKGDKGDKGDKGDKGDKGDK

SEQ ID NO: 140 - protein, 30 aa, synthetic construct
  SITE 1..30: note = /note="This sequence may encompass 2-10 'Gly Glu Lys' repeating units"
  REGION 1..30: note = source = /note="Description of Artificial Sequence: Synthetic polypeptide"
GEKGEKGEKGEKGEKGEKGEKGEKGEKGEK

SEQ ID NO: 141 - protein, 30 aa, synthetic construct
  SITE 1..30: note = /note="This sequence may encompass 2-10 'Gly Asp Lys' repeating units"
  REGION 1..30: note = source = /note="Description of Artificial Sequence: Synthetic polypeptide"
GDKGDKGDKGDKGDKGDKGDKGDKGDKGDK

SEQ ID NO: 142 - protein, 27 aa, synthetic construct
  SITE 1..27: note = /note="This sequence may encompass 2-9 'Gly Glu Lys' repeating units"
  REGION 1..27: note = source = /note="Description of Artificial Sequence: Synthetic peptide"
GEKGEKGEKGEKGEKGEKGEKGEKGEK

SEQ ID NO: 143 - protein, 27 aa, synthetic construct
  SITE 1..27: note = /note="This sequence may encompass 2-9 'Gly Asp Lys' repeating units"
  REGION 1..27: note = source = /note="Description of Artificial Sequence: Synthetic peptide"
GDKGDKGDKGDKGDKGDKGDKGDKGDK

SEQ ID NO: 144 - protein, 24 aa, synthetic construct
  SITE 1..24: note = /note="This sequence may encompass 2-8 'Gly Glu Lys' repeating units"
  REGION 1..24: note = source = /note="Description of Artificial Sequence: Synthetic peptide"
GEKGEKGEKGEKGEKGEKGEKGEK

SEQ ID NO: 145 - protein, 24 aa, synthetic construct
  SITE 1..24: note = /note="This sequence may encompass 2-8 'Gly Asp Lys' repeating units"
  REGION 1..24: note = source = /note="Description of Artificial Sequence: Synthetic peptide"
GDKGDKGDKGDKGDKGDKGDKGDK

SEQ ID NO: 146 - protein, 21 aa, synthetic construct
  SITE 1..21: note = /note="This sequence may encompass 2-7 'Gly Glu Lys' repeating units"
  REGION 1..21: note = source = /note="Description of Artificial Sequence: Synthetic peptide"
GEKGEKGEKGEKGEKGEKGEK

SEQ ID NO: 147 - protein, 21 aa, synthetic construct
  SITE 1..21: note = /note="This sequence may encompass 2-7 'Gly Asp Lys' repeating units"
  REGION 1..21: note = source = /note="Description of Artificial Sequence: Synthetic peptide"
GDKGDKGDKGDKGDKGDKGDK

SEQ ID NO: 148 - protein, 18 aa, synthetic construct
  SITE 1..18: note = /note="This sequence may encompass 2-6 'Gly Glu Lys' repeating units"
  REGION 1..18: note = source = /note="Description of Artificial Sequence: Synthetic peptide"
GEKGEKGEKGEKGEKGEK

SEQ ID NO: 149 - protein, 18 aa, synthetic construct
  SITE 1..18: note = /note="This sequence may encompass 2-6 'Gly Asp Lys' repeating units"
  REGION 1..18: note = source = /note="Description of Artificial Sequence: Synthetic peptide"
GDKGDKGDKGDKGDKGDK

SEQ ID NO: 150 - protein, 15 aa, synthetic construct
  SITE 1..15: note = /note="This sequence may encompass 2-5 'Gly Glu Lys' repeating units"
  REGION 1..15: note = source = /note="Description of Artificial Sequence: Synthetic peptide"
GEKGEKGEKGEKGEK

SEQ ID NO: 151 - protein, 15 aa, synthetic construct
  SITE 1..15: note = /note="This sequence may encompass 2-5 'Gly Asp Lys' repeating units"
  REGION 1..15: note = source = /note="Description of Artificial Sequence: Synthetic peptide"
GDKGDKGDKGDKGDK

SEQ ID NO: 152 - protein, 12 aa, synthetic construct
  SITE 1..12: note = /note="This sequence may encompass 2-4 'Gly Glu Lys' repeating units"
  REGION 1..12: note = source = /note="Description of Artificial Sequence: Synthetic peptide"
GEKGEKGEKGEK

SEQ ID NO: 153 - protein, 12 aa, synthetic construct
  SITE 1..12: note = /note="This sequence may encompass 2-4 'Gly Asp Lys' repeating units"
  REGION 1..12: note = source = /note="Description of Artificial Sequence: Synthetic peptide"
GDKGDKGDKGDK

(19) United States
(12) Patent Application Publication
(10) Pub. No.: US 2025/0019416 A1
(43) Pub. Date: Jan. 16, 2025

(54) RECOMBINANT COLLAGEN AND ELASTIN MOLECULES AND USES THEREOF

(71) Applicant: Geltor, Inc., Emeryville, CA (US)

(72) Inventors: Nikolay Ouzounov, Lafayette, CA (US); Alexander Lorestani, Oakland, CA (US); Monica Bhatia, San Ramon, CA (US)

(21) Appl. No.: 18/750,919

(22) Filed: Jun. 21, 2024

Related U.S. Application Data
(63) Continuation of application No. 17/532,978, filed on Nov. 22, 2021, now abandoned.
Application No. 17/532,978 is a continuation of application No. 16/839,035, filed on Apr. 2, 2020, now Pat. No. 11,214,609.
Application No. 16/839,035 is a continuation of application No. 16/144,914, filed on Sep. 27, 2018, now Pat. No. 11,180,541.
(60) Provisional application No. 62/657,591, filed on Apr. 13, 2018.
(60) Provisional application No. 62/564,964, filed on Sep. 28, 2017.

(51) Int. Cl. C07K 14/78; A61K 8/65; A61K 38/39; A61Q 17/04; A61Q 19/08

(52) U.S. Cl. CPC C07K 14/78; A61K 8/65; A61K 38/39; A61Q 17/04; A61Q 19/08

(57) ABSTRACT

This disclosure provides non-naturally occurring collagen and elastin molecules. The non-naturally occurring collagens and elastins include truncated collagens, truncated elastins, as well as fusion proteins thereof. The non-naturally occurring collagen and elastin are useful in foods, cosmetics and many other products and uses.

### CROSS-REFERENCE

This application is a continuation of U.S. patent application Ser. No. 17/532,978, filed Nov. 22, 2021, which is a continuation of U.S. patent application Ser. No. 16/839,035, filed Apr. 2, 2020, now U.S. Pat. No. 11,214,609, issued Jan. 4, 2022, which is a continuation of U.S. patent application Ser. No. 16/144,914, filed Sep. 27, 2018, now U.S. Pat. No. 11,180,541, issued Nov. 23, 2021, which claims priority from U.S. Provisional Patent Application No. 62/564,964, filed Sep. 28, 2017, and U.S. Provisional Patent Application No. 62/657,591, filed Apr. 13, 2018, each of which is incorporated by reference herein in its entirety.

### SEQUENCE LISTING

The instant application contains a Sequence Listing which has been submitted electronically in XML format and is hereby incorporated by reference in its entirety. Said XML copy, created on Jun. 21, 2024, is named 57607_702_306_SL.xml and is 306,954 bytes in size.

### FIELD

The present disclosure relates to non-naturally occurring full-length and truncated collagen molecules and full-length and truncated elastin molecules and uses thereof.

### BACKGROUND

Collagens and similar proteins are the most abundant proteins in the biosphere. Collagens and elastins are structural proteins found in the skin, connective tissue and bone of animals and other tissues. In humans, the amount of collagen present in the body is approximately one third of the total proteins and accounts for about three fourths of the dry weight of skin. Elastin is a highly elastic protein found in connective tissue and other types of tissue.

The structure of collagen is a triple helix in which three polypeptide strands together form a helical coil. The individual polypeptide strands are composed of repeating triplet amino acid sequences designated as GLY-X-Y. X and Y can be any amino acid and the third amino acid is glycine. The amino acids proline and hydroxyproline are found in high concentrations in collagen. The most common triplet is proline-hydroxyproline-glycine (Gly-Pro-Hyp) accounting for approximately 10.5% of the triplets in collagen.

Gelatin is a product obtained by partial hydrolysis of collagen. Typically, gelatin is produced by acid hydrolysis, alkaline hydrolysis, and enzymatic hydrolysis or by exposing collagen to heat in an aqueous solution (e.g., boiling the bones and skins of animal, boiling fish scales, etc.).

Gelatin is used in many products including cosmetics, foods, pharmaceuticals, medical devices, photographic films, adhesives, binders and many others. The physical and chemical properties of gelatin are tuned to the particular application. These physical/chemical properties include gel strength, melting point temperature, viscosity, color, turbidity, pH, isoelectric point and others.

Elastin is an elastic protein that is crucial for the proper functioning of arteries, lung, tendons, ligament, skin and other tissue. Elastin provides the tissues with the ability to stretch and return to its original shape. The protein tropoelastin is the building block of elastin. In contrast to collagen that include a family of genes, there is one tropoelastin gene in humans. When expressed, the single elastin gene is spliced to produce different forms of the tropoelastin protein. Many tropoelastin molecules associate together to form elastin.

L-form bacteria, or L-forms, are bacterial strains derived from parent species (N-forms) that are able to grow as cell wall-deficient (spheroplast type) or as cell wall-less (protoplast type) cells. See, Madoff S (Ed): The Bacterial L-Forms. New York: Marcel Dekker Inc., 1986; Mattmann LH (Ed): Cell Wall Deficient Forms. Boca Raton: CRC Press; 1993; and Gumpert J, Taubeneck U: Characteristic properties and biological significance of stable protoplast type L-forms. In Protoplasts, Lecture Proceedings of the 6th International Protoplast Symposium: Basel. Experientia 1983, 46(suppl):227-241.

Protoplast type L-forms have been cultivated in the cell wall-less state and represent genetically stable mutants showing extreme pleiotropic changes, including the inability to form cell walls, capsules, flagella, pili, spores and mesosomes, altered colony and cell morphology, qualitative and quantitative changes in the lipid and protein components of the cytoplasmic membrane, the absence of extracellular proteolytic activities, resistance against bacteriophages and the incapability to propagate outside laboratory conditions. See, Gumpert and Taubeneck (supra); and Hoischen et al., Lipid and fatty acid composition of cytoplasmic membranes from Streptomyces hygroscopic and its stable protoplast type L-form. J Bacteriol 1997, 179:3430-3436.

### SUMMARY

In one aspect, a non-naturally occurring collagen produced by a host cell is provided. The non-naturally occurring collagen is jellyfish (Hydrozoan) collagen, human collagen, Chondrosia reniformis (kidney sponge) collagen, or Rhincodon typus (whale shark) collagen. In an embodiment, the non-naturally occurring collagen is a full-length or a truncated collagen. In one embodiment, the collagen is truncated by an internal truncation of between 50 amino acids and 500 amino acids. In another embodiment, the truncation is at the C-terminal end or the N-terminal end of the collagen polypeptide. The non-naturally occurring collagens are SEQ ID NO: 10, SEQ ID NO: 12, SEQ ID NO: 14, SEQ ID NO: 16, SEQ ID NO: 18, SEQ ID NO: 20, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 91, SEQ ID NO: 92, SEQ ID NO: 94, SEQ ID NO: 96, SEQ ID NO: 102, SEQ ID NO: 104, SEQ ID NO: 106, SEQ ID NO: 108, SEQ ID NO:110, or SEQ ID NO: 112.

In another aspect, the non-naturally occurring collagen further comprises amino acid sequences including a secretion tag, a histidine tag, a green fluorescent protein tag, a protease cleavage site, a Beta-lactamase and/or GEK amino acid trimer repeats and/or GDK amino acid trimer repeats. When the non-naturally occurring collagen comprises one or more amino acid trimer repeats of the sequence glycine-glutamic acid-lysine (GEK) and/or glycine-aspartic acid-lysine (GDK), the number of GEK and/or GDK trimer repeats can range from 2 to 50 trimer repeats (SEQ ID NOS: 130-131, respectively). In one aspect, the secretion tag is DsbA, PelB, OmpA, TolB, MalE, lpp, TorA, or HylA, or a hybrid secretion tag that comprises a portion of one secretion tag fused to a portion of a second secretion tag. An exemplary secretion tag is DsbA.

In one aspect, provided are compositions that comprise between 0.005% and 30% w/w non-naturally occurring collagen. The compositions can further comprise at least one additional ingredient comprising a topical carrier or a preservative.

Compositions comprising non-naturally occurring collagen are in one aspect topical compositions for applying to skin. The topical compositions are used for decreasing skin damage or promoting the repair of damaged skin.

One aspect provides methods for decreasing skin damage or promoting the repair of damaged skin. The method comprises applying the composition comprising elastin to the skin of a subject. The method increases the viability of the fibroblast cells or keratinocytes of the skin of the subject. In another aspect the application of the composition increases the synthesis of procollagen by the fibroblast cells of the subject's skin. In another aspect the topical application of the composition protects skin or keratinocytes against UV damage. In yet another embodiment, thymine-thymine (TT) dimer formation is decreased by the collagens or elastins disclosed herein.

Another aspect provided herein are methods of increasing the viability of skin cells. The method comprises applying collagen or elastin molecules to the skin or skin cell. The collagen or elastin as provided increases the viability of keratinocytes and/or fibroblasts is increased upon exposure to UV radiation, urban dust or other damaging stimuli.

In another aspect provided herein are methods for decreasing the production of inflammatory cytokines in a skin cell. In one embodiment the skin cell is a keratinocyte. The method comprises applying a collagen or elastin molecule to a skin cell. The production of inflammatory cytokines including TNFα, IL-1α, IL-10, IL-3, IL-6, IL-7, IL-8, IL-10, IL-18, and IL-1RA.

In another aspect, provided are methods of protecting skin cells against the effect of exposure to urban dust. The method comprises the step of applying the collagen or elastin disclosed herein to the skin cell. The exposure of skin cell to collagen or elastin increases the viability of the skin cell. In an embodiment, the skin cell is a keratinocyte or a fibroblast.

In one aspect, a non-naturally occurring elastin produced by a host cell is provided. The non-naturally occurring elastin is jellyfish elastin, human elastin, Chondrosia reniformis (kidney sponge) elastin, or Rhincodon typus elastin. In an embodiment, the non-naturally occurring elastin is a full-length or truncated elastin. In one embodiment, the elastin is truncated by an internal truncation of between 50 amino acids and 500 amino acids. In another embodiment, the truncation is at the C-terminal end or the N-terminal end of the elastin polypeptide. The non-naturally occurring elastins are SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 98, or SEQ ID NO: 110.

In another aspect, the non-naturally occurring elastin further comprises amino acid sequences including a secretion tag, a histidine tag, a green fluorescent protein tag, a protease cleavage site, a Beta-lactamase and/or GEK amino acid trimer repeats and/or GDK amino acid trimer repeats. When the non-naturally occurring collagen comprises one or more amino acid trimer repeats of the sequence glycine-glutamic acid-lysine (GEK) and/or glycine-aspartic acid-lysine (GDK), the number of GEK and/or GDK trimer repeats can range from 2 to 50 trimer repeats (SEQ ID NOS: 130-131, respectively). In one aspect, the secretion tag is DsbA, PelB, OmpA, TolB, MalE, lpp, TorA, or Hy1A, or a hybrid secretion tag that comprises a portion of one secretion tag fused to a portion of a second secretion tag. An exemplary secretion tag is DsbA.

In another embodiment, compositions that comprise between 0.005% and 30% w/w non-naturally occurring elastin are provided. The compositions can further comprise at least one additional ingredient comprising a topical carrier or a preservative.

Compositions comprising non-naturally occurring elastin are in one aspect topical compositions for applying to skin. The topical compositions are used for decreasing skin damage or promoting the repair of damaged skin.

One embodiment provides methods for decreasing skin damage or promoting the repair of damaged skin. The method comprises applying the composition comprising elastin to the skin of a subject. The method increases the viability of the fibroblast cells of the skin of the subject. In another aspect the application of the composition increases the synthesis of procollagen by the fibroblast cells of the subject's skin. In another aspect the topical application of the compositions protects skin or keratinocytes against UV damage. In yet another embodiment, thymine-thymine (TT) dimer formation is decreased by the collagens or elastins disclosed herein.

Another embodiment provides polynucleotides that encode a non-naturally occurring collagen or a non-naturally occurring elastin. The polynucleotides encode collagen or elastin from jellyfish, human, Chondrosia reniformis (kidney sponge), or Rhincodon typus. The encoded collagen or elastin may be full length or truncated. In one embodiment, the collagen or elastin is truncated by an internal truncation of between 50 amino acids and 500 amino acids.

In one embodiment polynucleotides that encode fusion proteins comprising a secretion tag, a histidine tag, a green fluorescent protein tag, a protease cleavage site, a Beta-lactamase along and/or GEK amino acid trimer repeat and/or GDK amino acid trimer repeats together with collagen or elastin are provided. The non-naturally occurring collagen or elastin may comprise one or more amino acid trimer repeats of the sequence glycine-glutamic acid-lysine (GEK) and/or glycine-aspartic acid-lysine (GDK), the number of GEK and/or GDK trimer repeats can range from 2 to 50 trimer repeats (SEQ ID NOS: 130-131, respectively). In one aspect, the secretion tag is DsbA, PelB, OmpA, TolB, MalE, lpp, TorA, or Hy1A, or a hybrid secretion tag that comprises a portion of one secretion tag fused to a portion of a second secretion tag. An exemplary embodiment secretion tag is DsbA.

The polynucleotides and vectors can be used to transform host cells and express the polynucleotides. Polynucleotides encoding a non-naturally occurring collagen, wherein the polynucleotide is SEQ ID NO: 9, SEQ ID NO: 11, SEQ ID NO: 13, SEQ ID NO: 15, SEQ ID NO: 17, SEQ ID NO: 19 SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 90, SEQ ID NO: 93, SEQ ID NO: 95, SEQ ID NO: 97, SEQ ID NO: 103, SEQ ID NO: 105, SEQ ID NO: 107, SEQ ID NO: 109, SEQ ID NO:111, SEQ ID NO: 113, or SEQ ID NO:105 are provided. Polynucleotides encoding a non-naturally occurring elastin, wherein the polynucleotide is SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, and SEQ ID NO: 72, SEQ ID NO: 99, or SEQ ID NO:101 provided.

Host cells that express the polynucleotides of the invention are disclosed. Host cells can be any host cell including bacterial cells, yeast cells, fungal cells, insect cells, mammalian cells, plant cells and any other cells used to express exogenous polynucleotides.

Bacterial host cells in which the cells have been modified to inhibit cell division and the periplasmic space is increased are provided. An exemplary host cell is E. coli.

One embodiment provides a method of producing a non-naturally occurring collagen or a non-naturally occurring elastin. The method comprises the steps of inoculating a culture medium with a recombinant host cell comprising polynucleotides that encode the collagen or elastin, cultivating the host cell, and isolating the non-naturally occurring collagen or the non-naturally occurring elastin from the host cell.

### BRIEF DESCRIPTION OF THE DRAWINGS

FIG. 1 depicts the physiological state difference between switched and unswitched cells. A) Unswitched Escherichia coli cells. B) Same Escherichia coli population as figure A but has undergone the physiological switch. C) Phase contrast of switched Escherichia coli cell containing cytoplasmic RFP and periplasmic GFP. D) Fluorescent imaging of cell in figure C illustrates targeted protein localization.

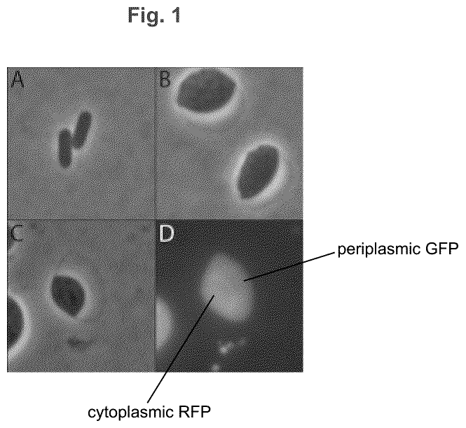

FIG. 2 depicts enhanced protein production in switched cells. A-B) Target protein for T7 inducible protein production is periplasmic expressed GFP, produced in Escherichia coli BL21. The same population of cells was used and induced at OD 1.1. A) Protein ladder (lane 1), IPTG induced protein production (lane 2), IPTG induced protein production with physiological switch (lane 3). B) Two vials of the cell GFP induced cultures with IPTG only on left and IPTG+Switch on right. C) Expression of a 22 kDa collagen using switched cells showing protein ladder (lane 1), supernatant after protein production (lane 2), cell pellet (lane 3).

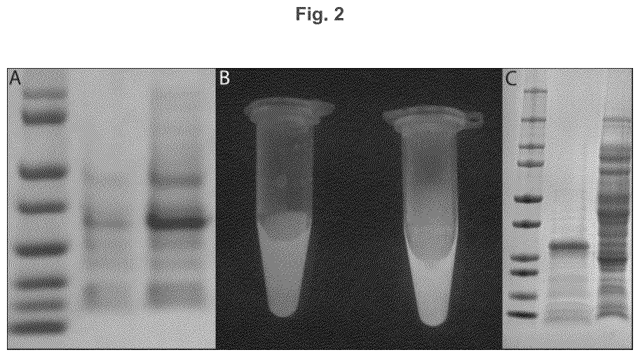

FIG. 3 depicts a time-lapse of Escherichia coli cell switching over time.

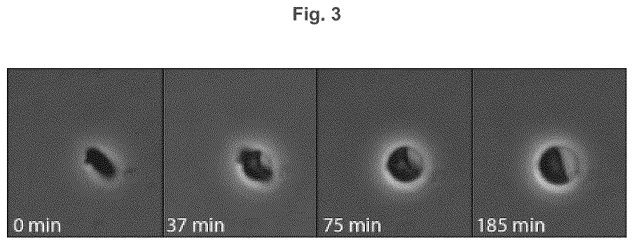

FIG. 4 illustrates other organisms undergoing the physiological switch. A) Agrobacterium tumefaciens normal physiology. B) Agrobacterium tumefaciens switched physiology. C) Pseudomonas aeruginosa PAO1 normal physiology. D) Pseudomonas aeruginosa PAO1 switched physiology. E) Brevundimonas diminuta normal physiology. F) Brevundimonas diminuta switched physiology. G) Agrobacterium tumefaciens normal physiology. H) Agrobacterium tumefaciens switched physiology.

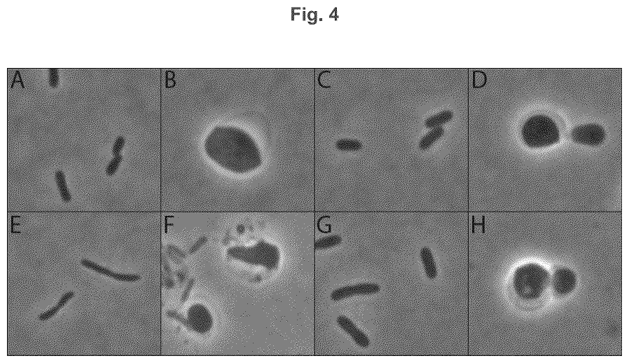

FIG. 5 illustrates the reduction in TT dimer formation by treatment of human keratinocytes with truncated collagen.

### DESCRIPTION

In the following description, certain specific details are set forth in order to provide a thorough understanding of various embodiments of the disclosure. However, one skilled in the art will understand that the disclosure may be practiced without these details.

As used herein the term “about” refers to ±10%.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “collagen” or “collagen-like” as used herein refers to a monomeric polypeptide that can associate with one or more collagen or collagen-like polypeptides to form a quaternary structure. Collagen can be treated with acid, base or heat to prepare gelatin. The quaternary structure of natural collagen is a triple helix typically composed of three polypeptides. Of the three polypeptides that form natural collagen, two are usually identical and are designated as the alpha chain. The third polypeptide is designated as the beta chain. Thus a typical natural collagen can be designated as AAB, wherein the collagen is composed of two alpha (“A”) strands and one beta (“B”) strand. The term “procollagen” as used herein refers to polypeptides produced by cells that can be processed to naturally occurring collagen.

The terms “elastin” as used herein refers to a polypeptide that is elastic and functions to stretch or contract and return to its original shape. Elastin is found naturally in connective tissue.

The term “expression vector” or “vector” as used herein refers to a nucleic acid assembly which is capable of directing the expression of the exogenous gene. The expression vector may include a promoter which is operably linked to the exogenous gene, restriction endonuclease sites, nucleic acids that encode one or more selection markers, and other nucleic acids useful in the practice of recombinant technologies.

The term “fibroblast” as used herein refers to a cell that synthesizes procollagen and other structural proteins. Fibroblasts are widely distributed in the body and found in skin, connective tissue and other tissues.

The term “fluorescent protein” is a protein that is commonly used in genetic engineering technologies used as a reporter of expression of an exogenous polynucleotide. The protein when exposed to ultraviolet or blue light fluoresces and emits a bright visible light. Proteins that emit green light is green fluorescent protein (GFP) and proteins that emit red light is red fluorescent protein (RFP).

The term “gelatin” as used herein refers to collagen that has been further processed by exposure to acid, base or heat. While not wishing to be bound by theory or mechanism, treatment of collagen with acid, base or heat is thought to denature the collagen polypeptides. Aqueous denatured collagen solutions form reversible gels used in foods, cosmetics, pharmaceuticals, industrial products, medical products, laboratory culture growth media, and many other applications.

The term “gene” as used herein refers to a polynucleotide that encodes a specific protein, and which may refer to the coding region alone or may include regulatory sequences preceding (5′ non-coding sequences) and following (3′ non-coding sequences) the coding sequence.

The term “histidine tag” is a 2-30 contiguous series of histidine residues on a recombinant polypeptide.

The term “host cell” is a cell that is engineered to express an introduced exogenous polynucleotide.

The term“keratinocyte” is a cell that produces keratins found in the epidermal layer of the skin.

The term “lactamase” as used herein refer to enzymes that hydrolyze antibiotics that contain a lactam (cyclic amide) moiety. “Beta-lactamase” or “β-lactamase” are enzymes that hydrolyze antibiotics that contain a β-lactam moiety.

The term “non-naturally occurring” as used herein refers to collagen or elastin that is not normally found in nature. The non-naturally occurring collagen or elastin are recombinantly prepared. The non-naturally occurring collagen or elastin is a recombinant collagen or recombinant elastin. The non-naturally occurring collagen is in one embodiment a truncated collagen. Other non-naturally occurring collagen polypeptides include chimeric collagens. A chimeric collagen is a polypeptide wherein one portion of a collagen polypeptide is contiguous with a portion of a second collagen polypeptide. For example, a collagen molecule comprising a portion of a jellyfish collagen contiguous with a portion of a human collagen is a chimeric collagen. In another embodiment, the non-naturally occurring collagen comprises a fusion polypeptide that includes additional amino acids such as a secretion tag, histidine tag, green fluorescent protein, protease cleavage site, GEK repeats, GDK repeats, and/or beta-lactamase. The non-naturally occurring elastin in one embodiment a truncated elastin. Other non-naturally occurring elastin polypeptides include chimeric elastins. A chimeric elastin is a polypeptide wherein one portion of an elastin polypeptide is contiguous with a portion of a second elastin polypeptide. For example, a collagen molecule comprising a portion of a jellyfish elastin contiguous with a portion of a human elastin is a chimeric elastin. In another embodiment, the non-naturally occurring elastin comprises a fusion polypeptide that includes additional amino acids such as a secretion tag, histidine tag, green fluorescent protein, protease cleavage site and/or beta-lactamase. The chimeric gelatin or the chimeric elastin can comprise additional amino acids such as a secretion tag, histidine tag, green fluorescent protein, protease cleavage site, GEK repeats, GDK repeats, and/or beta-lactamase.

The term “protease cleavage site” is an amino acid sequence that is cleaved by a specific protease.

The term “secretion tag” or “signal peptide” refers to an amino acid sequence that recruits the host cell's cellular machinery to transport an expressed protein to a particular location or cellular organelle of the host cell.

The term “truncated collagen” refers to a monomeric polypeptide that is smaller than a full-length collagen wherein one or more portions of the full-length collagen is not present. Collagen polypeptides are truncated at the C-terminal end, the N-terminal end, or truncated by removal of internal portion(s) of the full-length collagen polypeptide.

The term “truncated elastin” refers to a monomeric polypeptide that is smaller than a full-length elastin wherein one or more portions of the full-length elastin is not present. Elastin polypeptides are truncated at the C-terminal end, the N-terminal end, or truncated by removal of internal portion(s) of the full-length elastin polypeptide.

In co-owned application PCT/US17/24857, incorporated by reference, an expression system that uses modified bacterial cells (switched cells) in which cell division is inhibited and growth of the periplasmic space is greatly enhanced was disclosed. In this expression system, the expressed proteins are targeted to the periplasmic space. Recombinant protein production in these switched cells is dramatically increased compared with that in non-switched cells. Structurally, the cells comprise both inner and outer membranes but lack a functional peptidoglycan cell wall, while the cell shape is spherical and increases in volume over time. Notably, while the periplasmic space normally comprises only 10-20% of the total cell volume, the periplasmic compartment of the switched state described herein can comprise more than 20%, 30%, 40% or 50% and up to 60%, 70%, 80% or 90% of the total cell volume.

The modified bacterial cells of PCT/US17/24857 are derived from Gram-negative bacteria, e.g. selected from: gammaproteobacteria and alphaproteobacteria. In some embodiments, the bacterium is selected from: Escherichia coli, Vibrio natriegens, Pseudomonas fluorescens, Caulobacter crescentus, Agrobacterium tumefaciens, and Brevundimonas diminuta. In specific embodiments, the bacterium is Escherichia coli, e.g. strain BL21(DE3).

In another aspect, the host bacterial cells have an enlarged periplasmic space in a culture medium comprising a magnesium salt, wherein the concentration of magnesium ions in the medium is at least about 3, 4, 5 or 6 mM. In further embodiments, the concentration of magnesium ions in the medium is at least about 7, 8, 9 or 10 mM. In some embodiments, the concentration of magnesium ions in the medium is between about 5 mM and 25 mM, between about 6 mM and/or about 20, 15 or 10 mM. In some embodiments, the magnesium salt is selected from: magnesium sulfate and magnesium chloride.

In other embodiments, the culture medium further comprises an osmotic stabilizer, including, e.g. sugars (e.g., arabinose, glucose, sucrose, glycerol, sorbitol, mannitol, fructose, galactose, saccharose, maltotrioseerythritol, ribitol, pentaerythritol, arabitol, galactitol, xylitol, iditol, maltotriose, and the like), betaines (e.g., trimethylglycine), proline, sodium chloride, wherein the concentration of the osmotic stabilizer in the medium is at least about 4%, 5%, 6%, or 7% (w/v). In further embodiments, the concentration of osmotic stabilizer is at least about 8%, 9%, or 10% (w/v). In some embodiments, the concentration of the osmotic stabilizer in the medium is between about 5% to about 20% (w/v).

In some embodiments, the cell culture may further comprise ammonium chloride, ammonium sulfate, calcium chloride, amino acids, iron(II) sulfate, magnesium sulfate, peptone, potassium phosphate, sodium chloride, sodium phosphate, and yeast extract.

The host bacterial cell may be cultured continuously or discontinuously; in a batch process, a fed-batch process or a repeated fed-batch process.

In some embodiments, the antibiotic is selected from: β-lactam antibiotics (e.g. penicillins, cephalosporins, carbapenems, and monobactams), phosphonic acid antibiotics, polypeptide antibiotics, and glycopeptide antibiotics. In particular embodiments, the antibiotic is selected from alafosfalin, amoxicillin, ampicillin, aztreonam, bacitracin, carbenicillin, cefamandole, cefotaxime, cefsulodin, cephalothin, fosmidomycin, methicillin, nafcillin, oxacillin, penicillin g, penicillin v, fosfomycin, primaxin, and vancomycin.

Without being bound by theory, the cell morphology that promotes recombinant protein production and inhibits cell division appears to be driven by the removal of the cell wall under the media conditions stated above. In some embodiments, the methods for removal/inhibition of cell wall synthesis can be through the use of antibiotics that inhibit peptidoglycan synthesis (such as ampicillin, carbenicillin, penicillins or fosfomycin), or other methods known in the art.

When having an appropriate periplasmic targeting signal sequence, recombinantly produced polypeptides can be secreted into the periplasmic space of bacterial cells. Joly, J. C. and Laird, M. W., in The Periplasm ed. Ehrmann, M., ASM Press, Washington D.C., (2007) 345-360. In the chemically oxidizing environment of the periplasm the formation of disulfide bonds and thereby the functionally correct folding of polypeptides is favored.

In general, the signal sequence may be a component of the expression vector, or it may be a part of the exogenous gene that is inserted into the vector. The signal sequence selected should be one that is recognized and processed (i.e., cleaved by a signal peptidase) by the host cell. For bacterial host cells that do not recognize and process the native signal sequence of the exogenous gene, the signal sequence is substituted by any commonly known bacterial signal sequence. In some embodiments, recombinantly produced polypeptides can be targeted to the periplasmic space using the DsbA signal sequence. Dinh and Bernhardt, J Bacteriol, September 2011, 4984-4987.

In one aspect, a non-naturally occurring collagen or elastin is produced by a host cell is provided. The non-naturally occurring collagen or elastin is jellyfish collagen or elastin, human collagen or elastin, or Chondrosia reniformis (kidney sponge) collagen or elastin, or Rhincodon typus collagen or elastin. The non-naturally occurring collagen or elastin is a truncated collagen. The truncation is an internal truncation, a truncation at the N-terminal portion of the collagen or elastin, or a truncation at the C-terminal portion of the collagen or elastin. The collagen or elastin is truncated by a truncation of between 50 amino acids and 1000 amino acids, between, 50 amino acids and 950 amino acids, between 50 amino acids and 900 amino acids, between 50 amino acids and 850 amino acids, between 50 amino acids and 800 amino acids, between 50 amino acids and 850 amino acids, between 50 amino acids and 800 amino acids, between 50 amino acids and 750 amino acids, between 50 amino acids and 700 amino acids, between 50 amino acids and 650 amino acids, between 50 amino acids and 600 amino acids, between 50 amino acids and 650 amino acids, between 50 amino acids and 500 amino acids, between 50 amino acids and 450 amino acids, between 50 amino acids and 400 amino acids, between 50 amino acids and 350 amino acids, between 50 amino acids and 300 amino acids, between 50 amino acids and 250 amino acids, between 50 amino acids and 200 amino acids, between 50 amino acids and 150 amino acids, or between 50 amino acids and 100 amino acids. In another embodiment, the collagen or elastin is truncated by 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900, 950, or 1000 amino acids. The non-naturally occurring collagen or elastin are encoded by a portion of a polynucleotide sequence or the entire polynucleotide sequence disclosed herein.

The non-naturally occurring collagen or elastin further comprises amino acid sequences including a secretion tag. The secretion tag directs the collagen or elastin to the periplasmic space of the host cell. In particular embodiments, the signal peptide is derived from DsbA, PelB, OmpA, TolB, MalE, lpp, TorA, or Hy1A, or a hybrid secretion tag that comprises a portion of one secretion tag fused to a portion of a second secretion tag. In one aspect the secretion tag is attached to the non-naturally occurring collagen or elastin. In another aspect the secretion tag is cleaved from the non-naturally occurring collagen or elastin.

The non-naturally occurring collagen or the non-naturally occurring elastin o further comprises a histidine tag. The histidine tag or polyhistidine tag is a sequence of 2 to 20 histidine residues (SEQ ID NO: 117) that are attached to the collagen or elastin. The histidine tag comprises 2 to 20 histidine residues (SEQ ID NO: 117), 5 to 15 histidine residues (SEQ ID NO: 118), 5 to 18 histidine residues (SEQ ID NO: 119), 5 to 16 histidine residues (SEQ ID NO: 120), 5 to 15 histidine residues (SEQ ID NO: 118), 5 to 14 histidine residues (SEQ ID NO: 121), 5 to 13 histidine residues (SEQ ID NO: 122), 5 to 12 histidine residues (SEQ ID NO: 123), 5 to 11 (SEQ ID NO: 124), 5 to 10 histidine residues (SEQ ID NO: 125), 6 to 12 histidine residues (SEQ ID NO: 126), 6 to 11 histidine residues (SEQ ID NO: 127), or 7 to 10 histidine residues (SEQ ID NO: 128). The histidine tags are useful in purification of proteins by chromatographic methods utilizing nickel based chromatographic media. Exemplary fluorescent proteins include green fluorescent protein (GFP) or red fluorescent protein (RFP). Fluorescent proteins are well known in the art. In one embodiment the non-naturally occurring collagen or the on-naturally occurring elastin comprises a GFP and/or RFP. In one embodiment a superfolder GFP is fused to the on-naturally occurring collagen or elastin. The superfolder GFP is a GFP that folds properly even when fused to a poorly folded polypeptide. In one aspect the histidine tag is attached to the non-naturally occurring collagen or elastin. In another aspect the histidine tag is cleaved from the non-naturally occurring collagen or elastin.

The non-naturally occurring collagen or non-naturally occurring elastin further comprises a protease cleavage site. The protease cleavage site is useful to cleave the recombinantly produced collagen or elastin to remove portions of the polypeptide. The portions of the polypeptide that may be removed include the secretion tag, the histidine tag, the fluorescent protein tag and/or the Beta-lactamase. The proteases comprise endoproteases, exoproteases, serine proteases, cysteine proteases, threonine proteases, aspartic proteases, glutamic proteases, and metalloproteases. Exemplary protease cleavage sites include amino acids that are cleaved by Thrombin, TEV protease, Factor Xa, Enteropeptidase, and Rhinovirus 3C Protease. In one aspect the cleavage tag is attached to the non-naturally occurring collagen or elastin. In another aspect the cleavage tag is removed by an appropriate protease from the non-naturally occurring collagen or elastin.

The non-naturally occurring collagen or non-naturally occurring elastin further comprises an enzyme that is a Beta-lactamase. The beta-lactamase is useful as a selection marker. In one aspect the beta-lactamase is attached to the non-naturally occurring collagen or elastin. In another aspect the beta-lactamase is cleaved from the non-naturally occurring collagen or elastin.

The non-naturally occurring collagen or non-naturally occurring elastin further comprises GEK amino acid trimer repeats and/or GDK amino acid trimer repeats. The GEK and the GDK trimer repeats facilitate the gelling of the collagen and/or the gelatin. In one embodiment, the non-naturally occurring collagen or the non-naturally occurring elastin comprises 2-50 GEK and/or 2-50 GDK trimer repeats (SEQ ID NOS: 130-131, respectively), 2-40 GEK and/or 2-40 GDK trimer repeats (SEQ ID NOS: 132-133, respectively), 2-30 GEK and/or 2-30 GDK trimer repeats (SEQ ID NOS: 134-135, respectively), 2-20 GEK and/or 2-20 GDK trimer repeats (SEQ ID NOS: 136-137, respectively), 2-15 GEK and/or 2-15 GDK trimer repeats (SEQ ID NOS: 138-139, respectively). 2-10 GEK and/or 2-10 GDK trimer repeats (SEQ ID NOS: 140-141, respectively), 2-9 GEK and/or 2-9 GDK trimer repeats (SEQ ID NOS: 142-143, respectively), 2-8 GEK and/or 2-8 GDK trimer repeats (SEQ ID NOS: 144-145, respectively), 2-7 GEK and/or 2-7 GDK trimer repeats (SEQ ID NOS: 146-147, respectively), 2-6 GEK and/or 2-6 GDK trimer repeats (SEQ ID NOS: 148-149, respectively), 2-5 GEK and/or 2-5 GDK trimer repeats (SEQ ID NOS: 150-151, respectively), or 2-4 GEK and/or 2-4 GDK trimer repeats (SEQ ID NOS: 152-153, respectively). In one aspect the GEK trimer repeat or the GDK trimer repeat is attached to the non-naturally occurring collagen or elastin. In another aspect the GEK trimer repeat or the GDK trimer repeat is cleaved from the non-naturally occurring collagen or elastin.

Provided herein are compositions that comprise between 0.005% and 30% w/w non-naturally occurring collagen and/or non-naturally occurring elastin. The composition comprises between 0.005% and 20% w/w non-naturally occurring collagen and/or non-naturally occurring elastin, between 0.005% and 10% w/w non-naturally occurring collagen and/or non-naturally occurring elastin, between 0.005% and 5% w/w non-naturally occurring collagen and/or non-naturally occurring elastin, between 0.005% and 2% w/w non-naturally occurring collagen and/or non-naturally occurring elastin, between 0.005% and 1% w/w non-naturally occurring collagen and/or non-naturally occurring elastin, between 0.005% and 0.5% w/w non-naturally occurring collagen and/or non-naturally occurring elastin, and between 0.005% and 0.2% w/w non-naturally occurring collagen and/or non-naturally occurring elastin.

The compositions that comprise the between non-naturally occurring collagen and/or non-naturally occurring elastin are personal care products. In some embodiments the compositions are formulated for topical administration. The compositions can contain other cosmetic ingredients suitable for human use. The personal care products are useful for preventing or treating ultraviolet radiation damage to human skin or hair. The personal care products are applied to skin or hair. The compositions include, for example, masks, skin cleaners such as soap, cleansing creams, cleansing lotions, cleansing milks, cleansing pads, facial washes, hair shampoo, hair conditioner and body shampoos.

The compositions that comprise the non-naturally occurring collagen and/or non-naturally occurring elastin can further comprise at least one additional ingredient comprising a topical carrier or a preservative. The topical carrier comprises a topical carrier selected from the group consisting of liposome, biodegradable microcapsule, lotion, spray, aerosol, dusting powder, biodegradable polymer, mineral oil, triglyceride oil, silicone oil, glycerin, glycerin monostearate, alcohols, emulsifying agents, liquid petroleum, white petrolatum, propylene glycol, polyoxyethylene, polyoxypropylene, wax, sorbitan monostearate, polysorbate, cetyl ester wax, cetearyl alcohol, 2-octyldodecanol, benzyl alcohol, cyclomethicone, cyclopentasiloxane, and water. The preservative comprises a preservative selected from the group consisting of tocopherol, diiodomethyl-p-tolylsulfone, 2-Bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-Ethylbicyclooxazolidine, methyl paraben, sorbic acid, GERMABEN® II, rosemary extract, and EDTA.

Provided are methods of decreasing skin damage, promoting the repair of damaged skin, protecting skin against UV damage, protecting skin cells against the effects of exposure to urban dust. The method comprises the step of applying the composition comprising the non-naturally occurring collagen and/or non-naturally occurring elastin to the skin of a subject. Without being bound to a particular theory or mechanism, the collagen and/or the elastin in the composition decrease skin damage by protecting against UV damage, and/or promotes the repair of damaged skin by increasing the viability of cells and/or increasing procollagen synthesis when applied to skin, and/or promotes the viability of skin cells. The collagens and elastins in one aspect decrease the formation of thymine-thymine (TT) dimer formation.

One aspect provides polynucleotides that encode a non-naturally occurring collagen or a non-naturally occurring elastin. The polynucleotides encode collagen or elastin from jellyfish, human, Chondrosia reniformis (kidney sponge), or Rhincodon typus. The polynucleotides encode for collagen or elastin that is full length or truncated.

Another aspect provides polynucleotides that encode collagen or elastin fusion proteins. The elastin or collagen fusion proteins comprise a secretion tag, a histidine tag, a fluorescent protein tag, a protease cleavage site, a Beta-lactamase along and/or GEK amino acid trimer repeats and/or GDK amino acid trimer repeats together with collagen or elastin.

The polynucleotides are in one aspect vectors used to transform host cells and express the polynucleotides. The polynucleotides further comprise nucleic acids that encode enzymes that permit the host organism to grow in the presence of a selection agent. The selection agents include certain sugars including galactose containing sugars or antibiotics including ampicillin, hygromycin, G418 and others. Enzymes that are used to confer resistance to the selection agent include β-galactosidase or a β-lactamase.

In one aspect host cells that express the polynucleotides of the invention are provided. Host cells can be any host cell including gram negative bacterial cells, gram positive bacterial cells, yeast cells, insect cells, mammalian cells, plant cells or any other cells used to express exogenous polynucleotides. An exemplary gram-negative host cell is E. coli.

Bacterial host cells in which the cells have been modified to inhibit cell division and the periplasmic space is increased are taught. As discussed herein and taught in example 1, Beta-lactam antibiotics are useful as a switch to convert wild-type bacterial cells to a modified bacterial cell in which cell replication is inhibited and the periplasmic space is increased. Exemplary Beta-lactam antibiotics including penicillins, cephalosporins, carbapenems, and monobactams.

The switched form of bacteria (L-form) are cultivated in culture media that include certain salts and other nutrients. Salts and media compositions that support the physiological switch physiology that have been tested are M63 salt media, M9 salt media, PYE media, and Luria-Bertani (LB) media. Any necessary supplements besides carbon, nitrogen, and inorganic phosphate sources may also be included at appropriate concentrations introduced alone or as a mixture with another supplement or medium such as a complex nitrogen source. In certain embodiments, the medium further comprises one or more ingredients selected from: ammonium chloride, ammonium sulfate, calcium chloride, casamino acids, iron(II) sulfate, magnesium sulfate, peptone, potassium phosphate, sodium chloride, sodium phosphate, and yeast extract.

Beta-lactamases are enzymes that confer resistance to lactam antibiotics in prokaryotic cells. Typically when Beta-lactamases are expressed in bacterial host cells, the expressed Beta-lactamase protein also includes targeting sequences (secretion tag) that direct the Beta-lactamase protein to the periplasmic space. Beta-lactamases are not functional unless they are transported to the periplasmic space. Beta-lactamase targeted to the periplasmic without the use of an independent secretion tag that targets the enzyme to the periplasmic space are provided. By creating a fusion protein in which a periplasmic secretion tag added to the N-terminus of a protein such as GFP, collagen, or GFP/collagen chimeras, the functionality of the Beta-lactamase lacking a native secretion tag can be used to select for full translation and secretion of the N-terminal fusion proteins. Using this approach, we have used a DsbA-GFP-Collagen-Beta-lactamase fusion to select for truncation products in the target collagens that favor translation and secretion.

Another embodiment provides methods of producing a non-naturally occurring collagen or a non-naturally occurring elastin. The method comprises the steps of inoculating a culture medium with a recombinant host cell comprising polynucleotides that encode the collagen or elastin, cultivating the host cell, and isolating the non-naturally occurring collagen or the non-naturally occurring elastin from the host cell.

A process for fermentative preparation of a protein is provided. The process comprises the steps of.
a) culturing a recombinant Gram-negative bacterial cell in a medium comprising a magnesium salt, wherein the concentration of magnesium ions in the medium is at least about 6 mM, and wherein the bacterial cell comprises an exogenous gene encoding the protein;
b) adding an antibiotic to the medium, wherein the antibiotic inhibits peptidoglycan biogenesis in the bacterial cell; and
c) harvesting the protein from the medium.

The bacteria may be cultured continuously—as described, for example, in WO 05/021772—or discontinuously in a batch process (batch cultivation) or in a fed-batch or repeated fed-batch process for the purpose of producing the target protein. In some embodiments, protein production is conducted on a large-scale. Various large-scale fermentation procedures are available for production of recombinant proteins. Large-scale fermentations have at least 1,000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 20 liters in volumetric capacity.

For accumulation of the target protein, the host cell is cultured under conditions sufficient for accumulation of the target protein. Such conditions include, e.g., temperature, nutrient, and cell-density conditions that permit protein expression and accumulation by the cell. Moreover, such conditions are those under which the cell can perform basic cellular functions of transcription, translation, and passage of proteins from one cellular compartment to another for the secreted proteins, as are known to those skilled in the art.

The bacterial cells are cultured at suitable temperatures. For E. coli growth, for example, the typical temperature ranges from about 20° C. to about 39° C. In one embodiment, the temperature is from about 20° C. to about 37° C. In another embodiment, the temperature is at about 30° C. In one embodiment, the host cells, in the non-switched state or switched state are cultivated at one temperature and switched to a different temperature to induce protein production. The host cells are cultivated first at one temperature to propagate the cells, then to induce protein production the cell are cultivated at a lower temperature. The first temperature is 23°, 24°, 25°, 26°, 27°, 28°, 29°, 30°, 31°, 32°, 33°, 34°, 35°, 36°, or 37° C. The second temperature is 20°, 21°, 22°, 23°, 24°, 25°, 26°, 27°, 28°, 29°, 30°, 31°, 32°, 33°, 34°, 35° or 36° C. The cultivation at the second temperature is conducted between 1 hour and 100 hours, between 5 hours and 90 hours, between 5 hours and 80 hours, between 5 hours and 80 hours, between 5 hours and 70 hours, between 10 hours and 70 hours, between 15 hours and 70 hours, between 15 hours and 65 hours, between 15 hours and 60 hours, between 20 hours and 60 hours, between 20 hours and 55 hours, between 20 hours and 50 hours, between 24 hours and 50 hours, between 24 hours and 48 hours, between 30 hours and 50 hours, between 30 hours and 45 hours, or between 30 hours and 40 hours.

The pH of the culture medium may be any pH from about 5-9, depending mainly on the host organism. For E. coli, the pH is from about 6.8 to about 7.4, or about 7.0.

For induction of gene expression, typically the cells are cultured until a certain optical density is achieved, e.g., an OD600 of about 1.1, at which point induction is initiated (e.g., by addition of an inducer, by depletion of a repressor, suppressor, or medium component, etc.) to induce expression of the exogenous gene encoding the target protein. In some embodiments, expression of the exogenous gene is inducible by an inducer selected from, e.g. isopropyl-β-d-1-thiogalactopyranoside, lactose, arabinose, maltose, tetracycline, anhydrotetracycline, vavlycin, xylose, copper, zinc, and the like. The induction of gene expression can also be accomplished by decreasing the dissolved oxygen levels during fermentation. The dissolved oxygen levels of the fermentation during cell propagation is between 10% and 30%. To induce gene expression the dissolved oxygen level is reduced to below 10%, 9%, 8%, 7%, 6%, 5%, 4%, 3%, 2%, 1%, or 0%. In host cells, in either the physiological state or the switched state, protein production can be induced by lowering the temperature of the fermentation as disclosed herein.

After product accumulation, the cells are vortexed and centrifuged in order to induce lysis and release of recombinant proteins. The majority of the proteins is found in the supernatant but any remaining membrane bound proteins can be released using detergents (such as TRITON™ X-100).

In a subsequent step, the target protein, as a soluble or insoluble product released from the cellular matrix, is recovered in a manner that minimizes co-recovery of cellular debris with the product. The recovery may be done by any means, but in one embodiment, can comprise histidine tag purification through a nickel column. See, e.g., Purification of Proteins Using Polyhistidine Affinity Tags, Methods Enzymology. 2000; 326: 245-254.

### EXAMPLES

### Example 1: Expression System

### Materials and Methods:

### Strains:

### Tested Physiological Switch and Protein Production:

E. coli BL21(DE3)—From NEB, product #c2527
E. coli K12 NCM3722—From The Coli Genetic Stock Center, CGSC #12355

### Tested Physiological Switch:

### Gammaproteobacteria:

Vibrio natriegens—From ATCC, product #14048
Pseudomonas fluorescens—From ATCC, product #31948
Pseudomonas aeruginosa PAO1—From ATCC, product #BAA-47

### Alphaproteobacteria:

Caulobacter crescentus—From ATCC, product #19089
Agrobacterium tumefaciens Rhizobium radiobacter—From ATCC, product #33970
Brevundimonas diminuta—From ATCC, product #13184

### Media Compositions:

### 1 Liter 5×m63 Salts:

10 g (NH4)2SO4—From P212121, product #7783-20-2
68 g KH2PO4—From P212121, product #7778-77-0
2.5 mg FeSO4·7H2O—From Sigma Aldrich, product #F7002
Bring volume up to 1 liter with milliQ water
Adjust to pH 7 with KOH (From P212121, product #1310-58-3)
Autoclave mixture

### 1 Liter of 1M MgSO4:

246.5 g MgSO4 7H2O—From P212121, (Sigma Aldrich, product #10034-99-8)
Bring volume up to 1 liter with milliQ water.
Autoclave mixture.

### 1 Liter of Switch Media 1:

133.4 mL 5×m63 salts
10 mL 1M MgSO4
38.6 g Glucose—From P212121, product #50-99-7
66.6 g Sucrose—From P212121, product #57-50-1
8.33 g LB mix—From P212121, product #lb-miller
Bring volume up to 1 liter with milliQ water.
Filter sterilize mixture through a 0.22 μM pore vacuum filter (Sigma Aldrich, product #CLS430517).

### 1 Liter of Switch Media 2:

133.4 mL 5×m63 salts
10 mL 1M MgSO4
38.6 g Glucose—From P212121, product #50-99-7
66.6 g Sucrose—From P212121, product #57-50-1
10 g Yeast Extract—From FisherSci.com, product #J60287A1
Bring volume up to 1 liter with milliQ water.
Filter sterilize mixture through a 0.22 μM pore vacuum filter (Sigma Aldrich, product #CLS430517).

### For Bioreactor Growth:

5 liter of bioreactor media MGZ12:
1) Autoclave 1 L of Glucose at concentration of 500 g/L in DI water. (VWR, product #97061-170).
2) Autoclave 1 L of Sucrose at concentration of 500 g/L in DI water. (Geneseesci.com, product #62-112).
3) Autoclave in 3946 mL of DI water;
20 g (NH4)2HPO4. (VWR, product #97061-932).
66.5 g KH2PO4. (VWR, product #97062-348).
22.5 g H3C6H507. (VWR, product #BDH9228-2.5 KG).
2.95 g MgSO4.7H2O. (VWR, product #97062-134).
10 mL Trace Metals (Teknova), 1000×. (Teknova, product #T1001).
After autoclaving add 400 mL of (1) to (3), 65 mL of 10M NaOH (VWR, product #97064-480) to (3), and 666 mL of (2) to (3).
A feed of 500 g/L of glucose can be used during fermentation run as needed.
At induction add:
50 mL of 1M MgSO4.7H2O to a 5 L bioreactor
1 to 10 mM concentration of IPTG. (carbosynth.com, product #EI05931).
Add Fosfomycin (50 μg/mL or higher) and Carbenicillin (100 μg/mL or higher).

### Physiological Switch:

The physiological switch is optimally flipped at an OD 600 of 1 to 1.1 for E. coli for growth in shake flasks at volumes up to 1 L. For the other species tested, cultures were grown in switch media and subcultured once cultures reached maximal OD 600. In all cases the physiological switch is flipped through the addition of 100-200 ug/mL Carbenicillin (From P212121, product #4800-94-6) and 50-100 ug/mL Fosfomycin (From P212121, product #26016-99-9). The majority of the population is in the switched state within a few hours. To confirm that cells underwent a physiological switch, cells were imaged on a Nikon Ti-E with perfect focus system, Nikon CFI60 Plan Apo 100×NA 1.45 objective, Prior automated filter wheels and stage, LED-CFP/YFP/mCherry and LED-DA/FI/TX filter sets (Semrock), a Lumencor Sola II SE LED illumination system, and a Hamamatsu Flash 4.0 V2 CMOS camera.

### Image Analysis of Physiological Switch:

Images were analyzed using ImageJ to measure dimensions. In the switched state, the spherical outline of the outer membrane is treated as a sphere to calculate total volume (V=(4/3)πr3). The cytoplasmic volume is calculated as an ellipsoid that exists within the sphere (V=(4/3)π*(longest radius)*(short radius)2). To calculate the periplasmic volume, the cytoplasmic volume is subtracted from the total volume of the cell.

### Protein Expression and Quantification:

E. coli BL21(DE3) (NEB product #c2527) containing pET28a (emd Millipore product #69864) and its derivatives carrying GFP or collagen derivatives were grown in a shaking incubator at 37° C. overnight in switch media containing 50 mg/mL kanamycin (p212121 product #2251180). Next day, subcultures are started with a 1:10 dilution of the overnight culture into fresh switch media containing 50 mg/mL kanamycin. The culture is then physiologically switched and protein production is induced simultaneously at an OD 600 of 1 to 1.1 (Read on a Molecular Devices SPECTRAMAX® M2 microplate reader). The physiologically switch and protein production are flipped through the addition of 100 ug/mL Carbenicillin, 50 ug/mL Fosfomycin, and 100 ug/mL IPTG (p212121 product #367-93-1). Protein expression is continued in the switched state from between 8 hours to overnight at room temperature (approximately 22° C.) on an orbital shaker. In order to quantify total protein levels, QUICK START™ Bradford Protein Assay was used on mixed portion of culture and standard curves are quantitated on a Molecular Devices Spectramax M2 microplate reader. In order to quantitate the relative intensity of target protein production relative to the rest of the protein population the mixed portion of the cultures were run on MINI-PROTEAN® TGX™ Gels and stained with BIO-SAFE™ Coomassie Stain.

### Induction of Protein Production:

Standard procedures have been followed to induce protein production in the physiological state. We have been using the strain BL21(DE3) containing the plasmid pET28a driving the IPTG/lactose inducible production of recombinant proteins and targeting them to the periplasmic space using the DsbA signal sequence. Using the GFP protein, targeted to the periplasmic space as described above, we have demonstrated the ability to gain and increase of 5-fold in protein production when compared to un-switched cell populations induced at the same optical density, for the same amount of time (figures). The induction was optimal at an OD600 of 1.1 and induction was continued for 10 hours at which point the protein produced was measured at about 200 mg/mL.

### Example 2: Production of Full-Length Collagen

Full length jellyfish collagen was produced using the expression system discussed in Example 1 herein. The wild-type amino acid sequence of Podocoryna carnea (jellyfish or Hydrozoan) collagen is provided in SEQ ID NO: 1.

(SEQ ID NO: 1)
GPQGVVGADGKDGTPGEKGEQGRTGAAGKQGSPGADGARGPLGSIGQQGA
RGEPGDPGSPGLRGDTGLAGVKGVAGPSGRPGQPGANGLPGVNGRGGLRG
KPGAKGIAGSDGEAGESGAPGQSGPTGPRGQRGPSGEDGNPGLQGLPGSD
GEPGEEGQPGRSGQPGQQGPRGSPGEVGPRGSKGPSGDRGDRGERGVPGQ
TGSAGNVGEDGEQGGKGVDGASGPSGALGARGPPGSRGDTGAVGPPGPTG
RSGLPGNAGQKGPSGEPGSPGKAGSAGEQGPPGKDGSNGEPGSPGKEGER
GLAGPPGPDGRRGETGSPGIAGALGKPGLEGPKGYPGLRGRDGTNGKRGE
QGETGPDGVRGIPGNDGQSGKPGIDGIDGTNGQPGEAGYQGGRGTRGQLG
ETGDVGQNGDRGAPGPDGSKGSAGRPGLR
>/www.ncbi.nlm.nih.gov/protein/4379341?report=
genbank&log$ = protalign&blast_rank =
1&RID = T1N9ZEUW014>

The non-codon optimized polynucleotide sequence encoding the full length jellyfish collagen is disclosed in SEQ ID NO: 2.

(SEQ ID NO: 2)
ggaccacaaggtgttgtaggagctgatggcaaagatggaacaccgggaga
gaaaggtgagcaaggacgaaccggagctgcaggaaaacagggaagccctg
gagcagatggagcaagaggccctcttggatcaattggacaacaaggtgct
cgtggagaacctggtgatccaggatctcccggcttaagaggagatactgg
attggctggagtcaaaggagtagcaggaccatctggtcgacctggacaac
ccggtgcaaatggattacctggtgtgaatggcagaggcggtttgagaggc
aaacctggtgctaaaggaattgctggcagtgatggagaagcgggagaatc
tggcgcacctggacagtccggacctaccggtccacgtggtcaacgaggac
caagtggtgaggatggtaatcctggattacagggattgcctggttctgat
ggagagcccggagaggaaggacaacctggaagatctggtcaaccaggaca
gcaaggaccacgtggttcccctggagaggtaggaccaagaggatctaaag
gtccatcaggagatcgtggtgacaggggagagagaggtgttcctggacaa
acaggttcggctggaaatgtaggagaagatggagagcaaggaggcaaagg
tgtcgatggagcgagtggaccaagtggagctcttggtgctcgtggtcccc
caggaagtagaggtgacaccggggcagtgggacctcccggacctactggg
cgatctggtttacctggaaacgcaggacaaaagggaccaagtggtgaacc
aggtagtccaggaaaagcaggatcagctggtgaacagggtcctcctggta
aagacggatcaaatggtgaacctggatctcctggcaaagagggtgaacgt
ggtcttgctggtccaccaggtccagatggcagacgtggtgaaacgggatc
tccaggtatcgctggtgctcttggtaaaccaggtttggaaggacctaaag
gttatccaggattaagaggaagagatggaaccaatggcaaacgaggagaa
caaggagaaactggtcctgatggagtcagaggtattcctggaaatgatgg
acaatctggcaaaccaggtattgatggtattgacggaacaaatggtcaac
caggtgaggctggataccaaggtggtagaggtacacgtggtcagttaggt
gaaactggtgatgtcggacagaatggagatcgaggagctcctggtcctga
tggatctaaaggttctgctggtagaccaggacttcgtgg
<https://www.ncbi.nlm.nih.gov/nucleotide/
3355656?report = genbank&log$ =
nuclalign&blastrank = 1&RID = TSYP7CMV014>

Two different codon optimized polynucleotide sequences encoding the wild-type, full-length jellyfish collagen were synthesized. The two polynucleotide sequences were slightly different due to slightly different codon optimization methods. In addition to the non-truncated, full-length jellyfish collagen, the polynucleotides also encoded a secretion tag, a 9 amino acid his tag (SEQ ID NO: 129), a short linker, and a thrombin cleavage site. The DsbA secretion tag is encoded by nucleotides 1-71. The histidine tag comprising 9 histidine residues (SEQ ID NO: 129) is encoded by nucleotides 73-99 and encodes amino acids 25-33. The linker is encoded by nucleotides 100-111. The thrombin cleavage tag is encoded by nucleotides 112-135 and encodes amino acids 38-45. The truncated collagen is encoded by nucleotides 136-1422. The two polynucleotides are disclosed below in SEQ ID NO: 3 and 4.

(SEQ ID NO: 3)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGGGTCCGCAGGGTGTT
GTTGGTGCAGATGGTAAAGACGGTACCCCGGGTGAAAAAGGAGAACAGGG
ACGTACAGGTGCAGCAGGTAAACAGGGCAGCCCGGGTGCCGATGGTGCCC
GTGGCCCGCTGGGTAGCATTGGTCAGCAGGGTGCAAGAGGCGAACCGGGC
GATCCGGGTAGTCCGGGCCTGCGTGGTGATACGGGTCTGGCCGGTGTTAA
AGGCGTTGCAGGTCCTTCAGGTCGTCCAGGTCAACCGGGTGCAAATGGTC
TGCCGGGTGTTAATGGTCGTGGCGGTCTGCGTGGCAAACCGGGAGCAAAA
GGTATTGCAGGTAGCGATGGAGAAGCCGGTGAAAGCGGTGCCCCGGGTCA
GAGTGGTCCGACCGGTCCGCGCGGTCAGCGTGGTCCGTCTGGTGAAGATG
GCAATCCGGGTCTGCAGGGTCTGCCTGGTAGTGATGGCGAACCAGGTGAA
GAAGGTCAGCCGGGTCGTTCAGGCCAGCCGGGCCAGCAGGGCCCGCGTGG
TAGCCCGGGCGAAGTTGGCCCGCGGGGTAGTAAAGGTCCTAGTGGCGATC
GCGGTGATCGTGGTGAACGCGGTGTTCCTGGTCAGACCGGTAGCGCAGGT
AATGTTGGCGAAGATGGTGAACAGGGTGGCAAAGGTGTTGATGGTGCAAG
CGGTCCGAGCGGTGCACTGGGTGCACGTGGTCCTCCGGGCAGCCGTGGTG
ACACCGGTGCAGTTGGTCCGCCTGGCCCGACCGGCCGTAGTGGCTTACCG
GGTAATGCAGGTCAGAAAGGTCCGTCAGGTGAACCTGGCAGCCCTGGTAA
AGCAGGTAGTGCCGGTGAGCAGGGTCCGCCGGGCAAAGATGGTAGTAATG
GTGAGCCGGGTAGCCCTGGCAAAGAAGGTGAACGTGGTCTGGCAGGACCG
CCGGGTCCTGATGGTCGCCGCGGTGAAACGGGTTCACCGGGTATTGCCGG
TGCCCTGGGTAAACCAGGTCTGGAAGGTCCGAAAGGTTATCCTGGTCTGC
GCGGTCGTGATGGTACCAATGGCAAACGTGGCGAACAGGGCGAAACCGGT
CCAGATGGTGTTCGTGGTATTCCGGGTAACGATGGTCAGAGCGGTAAACC
GGGCATTGATGGTATTGATGGCACCAATGGTCAGCCTGGCGAAGCAGGTT
ATCAGGGTGGTCGCGGTACCCGTGGTCAGCTGGGTGAAACAGGTGATGTT
GGTCAGAATGGTGATCGCGGCGCACCGGGTCCGGATGGTAGCAAAGGTAG
CGCCGGTCGTCCGGGTTTACGTtaa
(SEQ ID NO: 4)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGGGTCCGCAGGGTGTT
GTTGGTGCAGATGGTAAAGACGGTACCCCGGGTGAAAAAGGtGAACAGGG
tCGTACcGGTGCAGCAGGTAAACAGGGCAGCCCGGGTGCCGATGGTGCCC
GTGGCCCGCTGGGTAGCATTGGTCAGCAGGGTGCAcgtGGCGAACCGGGC
GATCCGGGTAGcCCGGGCCTGCGTGGTGATACGGGTCTGGCCGGTGTTAA
AGGCGTTGCAGGTCCTTCtGGTCGTCCAGGTCAACCGGGTGCAAATGGTC
TGCCGGGTGTTAATGGTCGTGGCGGTCTGCGTGGCAAACCGGGtGCAAAA
GGTATTGCAGGTAGCGATGGcGAAGCCGGTGAAAGCGGTGCCCCGGGTCA
GAGcGGTCCGACCGGTCCGCGCGGTCAGCGTGGTCCGTCTGGTGAAGATG
GCAATCCGGGTCTGCAGGGTCTGCCTGGTagcGATGGCGAACCAGGTGAA
GAAGGTCAGCCGGGTCGTTCtGGCCAGCCGGGCCAGCAGGGCCCGCGTGG
TAGCCCGGGCGAAGTTGGCCCGCGcGGTtcTAAAGGTCCTAGcGGCGATC
GCGGTGATCGTGGTGAACGCGGTGTTCCTGGTCAGACCGGTAGCGCAGGT
AATGTTGGCGAAGATGGTGAACAGGGTGGCAAAGGTGTTGATGGTGCAAG
CGGTCCGAGCGGTGCACTGGGTGCACGTGGTCCTCCGGGCAGCCGTGGTG
ACACCGGTGCAGTTGGTCCGCCTGGCCCGACCGGCCGTAGcGGCctgCCG
GGTAATGCAGGTCAGAAAGGTCCGTCtGGTGAACCTGGCAGCCCTGGTAA
AGCAGGTAGcGCCGGTGAGCAGGGTCCGCCGGGCAAAGATGGTAGcAATG
GTGAGCCGGGTAGCCCTGGCAAAGAAGGTGAACGTGGTCTGGCAGGtCCG
CCGGGTCCTGATGGTCGCCGCGGTGAAACGGGTTCtCCGGGTATTGCCGG
TGCCCTGGGTAAACCAGGTCTGGAAGGTCCGAAAGGTTATCCTGGTCTGC
GCGGTCGTGATGGTACCAATGGCAAACGTGGCGAACAGGGCGAAACCGGT
CCAGATGGTGTTCGTGGTATTCCGGGTAACGATGGTCAGAGCGGTAAACC
GGGCATTGATGGTATTGATGGCACCAATGGTCAGCCTGGCGAAGCAGGTT
ATCAGGGTGGTCGCGGTACCCGTGGTCAGCTGGGTGAAACcGGTGATGTT
GGTCAGAATGGTGATCGCGGCGCACCGGGTCCGGATGGTAGCAAAGGTAG
CGCCGGTCGTCCGGGTctgCGTtaa

The amino acid sequence encoded by the polynucleotides of SEQ ID NO: 3 and SEQ ID NO:4 is disclosed in SEQ ID NO:5 below. In SEQ ID NO: 5 the DsbA secretion tag is encoded by nucleotides 1-71 and encodes amino acids 1-24; the histidine tag comprising 9 histidine residues (SEQ ID NO: 129) is encoded by nucleotides 73-99 and encodes amino acids 25-33; the linker is encoded by nucleotides 100-111 and encodes amino acids 34-37; the thrombin cleavage tag is encoded by nucleotides 112-135 and encodes amino acids 38-45; the full-length collagen is encoded by nucleotides 136-1422 and encodes amino acids 46-474.

(SEQ ID NO: 5)
MKKIWLALAGLVLAFSASAAQYEDHHHHHHHHHSGSSLVPRGSHMGPQGV
VGADGKDGTPGEKGEQGRTGAAGKQGSPGADGARGPLGSIGQQGARGEPG
DPGSPGLRGDTGLAGVKGVAGPSGRPGQPGANGLPGVNGRGGLRGKPGAK
GIAGSDGEAGESGAPGQSGPTGPRGQRGPSGEDGNPGLQGLPGSDGEPGE
EGQPGRSGQPGQQGPRGSPGEVGPRGSKGPSGDRGDRGERGVPGQTGSAG
NVGEDGEQGGKGVDGASGPSGALGARGPPGSRGDTGAVGPPGPTGRSGLP
GNAGQKGPSGEPGSPGKAGSAGEQGPPGKDGSNGEPGSPGKEGERGLAGP
PGPDGRRGETGSPGIAGALGKPGLEGPKGYPGLRGRDGTNGKRGEQGETG
PDGVRGIPGNDGQSGKPGIDGIDGTNGQPGEAGYQGGRGTRGQLGETGDV
GQNGDRGAPGPDGSKGSAGRPGLR

A jellyfish collagen without the DsbA secretion tag, the histidine tag, linker and thrombin cleavage site is disclosed in SEQ ID NO: 89.

(SEQ ID NO: 89)
GPQGVVGADGKDGTPGEKGEQGRTGAAGKQGSPGADGARGPLGSIGQQGA
RGEPGDPGSPGLRGDTGLAGVKGVAGPSGRPGQPGANGLPGVNGRGGLRG
KPGAKGIAGSDGEAGESGAPGQSGPTGPRGQRGPSGEDGNPGLQGLPGSD
GEPGEEGQPGRSGQPGQQGPRGSPGEVGPRGSKGPSGDRGDRGERGVPGQ
TGSAGNVGEDGEQGGKGVDGASGPSGALGARGPPGSRGDTGAVGPPGPTG
RSGLPGNAGQKGPSGEPGSPGKAGSAGEQGPPGKDGSNGEPGSPGKEGER
GLAGPPGPDGRRGETGSPGIAGALGKPGLEGPKGYPGLRGRDGTNGKRGE
QGETGPDGVRGIPGNDGQSGKPGIDGIDGTNGQPGEAGYQGGRGTRGQLG
ETGDVGQNGDRGAPGPDGSKGSAGRPGLR

The polynucleotides of SEQ ID NO: 3 and SEQ ID NO: 4 were synthesized by Gen9 DNA, now Ginkgo Bioworks internal synthesis. Overlaps between the pET28 vector and SEQ ID NO: 3 and SEQ ID NO: 4 were designed to be between 30 and 40 bp long and added using PCR with the enzyme PRIMESTAR® GXL polymerase (<http://www.clontech.com/US/Products/PCR/GC_Rich/PrimeSTAR_GXL_DNA_Polymerase?sitex=10020:22372:US>). The opened pET28a vector and insert DNA (SEQ ID NO: 3 or SEQ ID NO: 4) were then assembled together into the final plasmid using SGI GIBSON ASSEMBLY® (<https://us.vwr.com/store/product/17613857/gibson-assembly-hifi-1-step-kit-synthetic-genomics-inc>). Sequence of plasmid was then verified through sanger sequencing through Eurofins Genomics (<www.eurofinsgenomics.com>).

The transformed cells were cultivated in minimal media and frozen in 1.5 aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

Minimal media used in this example and throughout this application is prepared as follows. The minimal media (Table 1) was autoclaved in several separate fractions, Salts mix (Ammonium Phosphate dibasic, Potassium phosphate monobasic, Citric acid anhydrous, Magnesium sulfate heptahydrate), the Sucrose at 500 g/L, the Glucose at 55%, the Trace Metals TM5 (table 2), and Sodium Hydroxide 10M. The minimal media was then mixed together at the above concentrations post-autoclaving in the hood.

TABLE 1
Minimal media recipe for shake flask cultures

| chemical | Formula | MW | Conc (g/L) |
|---|---|---|---|
| Ammonium Phosphate dibasic | (NH₄)₂HPO₄ | 133 | 4 |
| Potassium phosphate monobasic | KH₂PO₄ | 137 | 13.3 |
| Citric acid anhydrous | H₃C₆H₅O₇ | 192.14 | 4.5 |
| Magnesium sulfate heptahydrate | MgSO₄•7H₂O | 246 | 0.59 |
| Trace Metals TM5 | | | 2 |
| Glucose | C₆H₁₂O₆ | 500 | 40 |
| Sodium Hydroxide 10M | NaOH | 400 | 5.2 |
| Sucrose 500 g/L | C₁₂H₂₂O₁₁ | 500 | 66.6 |

TABLE 2
Trace Metals TM5 composition

| chemical | Formula | MW | Conc (g/L) |
|---|---|---|---|
| Ferrous Sulfate Heptahydrate | FeSO₄•7H₂0 | 278.02 | 27.8 |
| Calcium Chloride | CaC₁₂•2H₂0 | 147 | 2.94 |
| Manganese Chloride | MnC₁₂ | 125.84 | 1.26 |
| Zinc Sulfate | ZnSO₄•H₂0 | 179.5 | 1.8 |
| Nickel Chloride | NiC₁₂•6H₂0 | 237.69 | 0.48 |
| Sodium Molybate | Na₂MoO₄•2H₂0 | 241.95 | 0.48 |
| Sodium Selenite | Na₂SeO₃ | 172.94 | 0.35 |
| Boric Acid | H₃BO₃ | 61.83 | 0.12 |

The harvested cells were disrupted in a homogenizer at 14,000 psi pressure in 2 passes. Resulting slurry contained the collagen protein along with other proteins.

The collagen was purified by acid treatment of homogenized cell broth. The pH of the homogenized slurry was decreased to 3 using 6M Hydrochloric acid. Acidified cell slurry was incubated overnight at 4° C. with mixing, followed by centrifugation. Supernatant of the acidified slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m² each. Total area of filtration was 0.2 m² using 2 cassettes in parallel. A volume reduction of 5× and a salt reduction of 19× was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified collagen was analyzed on an SDS-PAGE gel and a thick and clear band was observed at the expected size of 42 kilodaltons. The purified collagen was also analyzed by mass spectrometry and it was confirmed that the 42 kilodalton protein was jellyfish collagen.

The fermentations were performed at various temperature ranging from 25° to 28° C. For some fermentations, the temperature of the fermentation was maintained at a constant temperature and immediately upon completion of fermentation (OD600 of 5-10) the collagen was purified. For other fermentations, the temperature of the fermentations was maintained for a desired period of time and when cell densities of OD600 of 5-10 were reached, the temperature was reduced to induce protein production. Typically, the temperature was reduced from 28° C. to 25° C. After the fermentation at 25° C. was continued for 40-60 hours, the collagen was isolated.

### Additional Full Length Jellyfish Collagens

A full length jellyfish collagen without a His tag, linker, and thrombin cleavage site is disclosed below. Two codon-optimized nucleotide sequence encoding this collagen are provided in SEQ ID NO: 6 and SEQ ID NO: 7. The differences in the nucleotide sequences are due to different codon-optimization strategies but encode the same protein. The amino acid sequence is disclosed in SEQ ID NO: 8. The DsbA secretion tag is encoded by nucleotides 1-72 and encodes amino acids 1-24. The collagen sequence is encoded by nucleotides 73-1359 and encodes amino acids 25-453.

(SEQ ID NO: 6)
ATGAAAAAGATTTGGCTGGCGCTGGCTGGTTTAGTTTTAGCGTTTAGCGC
ATCGGCGGCGCAGTATGAAGATGGTCCGCAGGGTGTTGTTGGTGCAGATG
GTAAAGACGGTACCCCGGGTGAAAAAGGAGAACAGGGACGTACAGGTGCA
GCAGGTAAACAGGGCAGCCCGGGTGCCGATGGTGCCCGTGGCCCGCTGGG
TAGCATTGGTCAGCAGGGTGCAAGAGGCGAACCGGGCGATCCGGGTAGTC
CGGGCCTGCGTGGTGATACGGGTCTGGCCGGTGTTAAAGGCGTTGCAGGT
CCTTCAGGTCGTCCAGGTCAACCGGGTGCAAATGGTCTGCCGGGTGTTAA
TGGTCGTGGCGGTCTGCGTGGCAAACCGGGAGCAAAAGGTATTGCAGGTA
GCGATGGAGAAGCCGGTGAAAGCGGTGCCCCGGGTCAGAGTGGTCCGACC
GGTCCGCGCGGTCAGCGTGGTCCGTCTGGTGAAGATGGCAATCCGGGTCT
GCAGGGTCTGCCTGGTAGTGATGGCGAACCAGGTGAAGAAGGTCAGCCGG
GTCGTTCAGGCCAGCCGGGCCAGCAGGGCCCGCGTGGTAGCCCGGGCGAA
GTTGGCCCGCGGGGTAGTAAAGGTCCTAGTGGCGATCGCGGTGATCGTGG
TGAACGCGGTGTTCCTGGTCAGACCGGTAGCGCAGGTAATGTTGGCGAAG
ATGGTGAACAGGGTGGCAAAGGTGTTGATGGTGCAAGCGGTCCGAGCGGT
GCACTGGGTGCACGTGGTCCTCCGGGCAGCCGTGGTGACACCGGTGCAGT
TGGTCCGCCTGGCCCGACCGGCCGTAGTGGCTTACCGGGTAATGCAGGTC
AGAAAGGTCCGTCAGGTGAACCTGGCAGCCCTGGTAAAGCAGGTAGTGCC
GGTGAGCAGGGTCCGCCGGGCAAAGATGGTAGTAATGGTGAGCCGGGTAG
CCCTGGCAAAGAAGGTGAACGTGGTCTGGCAGGACCGCCGGGTCCTGATG
GTCGCCGCGGTGAAACGGGTTCACCGGGTATTGCCGGTGCCCTGGGTAAA
CCAGGTCTGGAAGGTCCGAAAGGTTATCCTGGTCTGCGCGGTCGTGATGG
TACCAATGGCAAACGTGGCGAACAGGGCGAAACCGGTCCAGATGGTGTTC
GTGGTATTCCGGGTAACGATGGTCAGAGCGGTAAACCGGGCATTGATGGT
ATTGATGGCACCAATGGTCAGCCTGGCGAAGCAGGTTATCAGGGTGGTCG
CGGTACCCGTGGTCAGCTGGGTGAAACAGGTGATGTTGGTCAGAATGGTG
ATCGCGGCGCACCGGGTCCGGATGGTAGCAAAGGTAGCGCCGGTCGTCCG
GGTTTACGTtaa
(SEQ ID NO: 7)
ATGAAAAAGATTTGGCTGGCGCTGGCTGGTTTAGTTTTAGCGTTTAGCGC
ATCGGCGGCGCAGTATGAAGATGGTCCGCAGGGTGTTGTTGGTGCAGATG
GTAAAGACGGTACCCCGGGTGAAAAAGGtGAACAGGGtCGTACcGGTGCA
GCAGGTAAACAGGGCAGCCCGGGTGCCGATGGTGCCCGTGGCCCGCTGGG
TAGCATTGGTCAGCAGGGTGCAcgtGGCGAACCGGGCGATCCGGGTAGcC
CGGGCCTGCGTGGTGATACGGGTCTGGCCGGTGTTAAAGGCGTTGCAGGT
CCTTCtGGTCGTCCAGGTCAACCGGGTGCAAATGGTCTGCCGGGTGTTAA
TGGTCGTGGCGGTCTGCGTGGCAAACCGGGtGCAAAAGGTATTGCAGGTA
GCGATGGcGAAGCCGGTGAAAGCGGTGCCCCGGGTCAGAGcGGTCCGACC
GGTCCGCGCGGTCAGCGTGGTCCGTCTGGTGAAGATGGCAATCCGGGTCT
GCAGGGTCTGCCTGGTagcGATGGCGAACCAGGTGAAGAAGGTCAGCCGG
GTCGTTCtGGCCAGCCGGGCCAGCAGGGCCCGCGTGGTAGCCCGGGCGAA
GTTGGCCCGCGcGGTtcTAAAGGTCCTAGcGGCGATCGCGGTGATCGTGG
TGAACGCGGTGTTCCTGGTCAGACCGGTAGCGCAGGTAATGTTGGCGAAG
ATGGTGAACAGGGTGGCAAAGGTGTTGATGGTGCAAGCGGTCCGAGCGGT
GCACTGGGTGCACGTGGTCCTCCGGGCAGCCGTGGTGACACCGGTGCAGT
TGGTCCGCCTGGCCCGACCGGCCGTAGcGGCctgCCGGGTAATGCAGGTC
AGAAAGGTCCGTCtGGTGAACCTGGCAGCCCTGGTAAAGCAGGTAGcGCC
GGTGAGCAGGGTCCGCCGGGCAAAGATGGTAGcAATGGTGAGCCGGGTAG
CCCTGGCAAAGAAGGTGAACGTGGTCTGGCAGGtCCGCCGGGTCCTGATG
GTCGCCGCGGTGAAACGGGTTCtCCGGGTATTGCCGGTGCCCTGGGTAAA
CCAGGTCTGGAAGGTCCGAAAGGTTATCCTGGTCTGCGCGGTCGTGATGG
TACCAATGGCAAACGTGGCGAACAGGGCGAAACCGGTCCAGATGGTGTTC
GTGGTATTCCGGGTAACGATGGTCAGAGCGGTAAACCGGGCATTGATGGT
ATTGATGGCACCAATGGTCAGCCTGGCGAAGCAGGTTATCAGGGTGGTCG
CGGTACCCGTGGTCAGCTGGGTGAAACcGGTGATGTTGGTCAGAATGGTG
ATCGCGGCGCACCGGGTCCGGATGGTAGCAAAGGTAGCGCCGGTCGTCCG
GGTctgCGTtaa
(SEQ ID NO: 8)
MKKIWLALAGLVLAFSASAAQYEDGPQGVVGADGKDGTPGEKGEQGRTGA
AGKQGSPGADGARGPLGSIGQQGARGEPGDPGSPGLRGDTGLAGVKGVAG
PSGRPGQPGANGLPGVNGRGGLRGKPGAKGIAGSDGEAGESGAPGQSGPT
GPRGQRGPSGEDGNPGLQGLPGSDGEPGEEGQPGRSGQPGQQGPRGSPGE
VGPRGSKGPSGDRGDRGERGVPGQTGSAGNVGEDGEQGGKGVDGASGPSG
ALGARGPPGSRGDTGAVGPPGPTGRSGLPGNAGQKGPSGEPGSPGKAGSA
GEQGPPGKDGSNGEPGSPGKEGERGLAGPPGPDGRRGETGSPGIAGALGK
PGLEGPKGYPGLRGRDGTNGKRGEQGETGPDGVRGIPGNDGQSGKPGIDG
IDGTNGQPGEAGYQGGRGTRGQLGETGDVGQNGDRGAPGPDGSKGSAGRP
GLR

### Example 3: Production of Truncated Collagen

A codon optimized DNA sequence, optimized for expression in E. coli, encoding a jellyfish collagen with a truncation of 240 internal amino acids was synthesized and expressed. The DNA sequence is shown below in SEQ ID NO: 9. In SEQ ID NO: 9, The DsbA secretion tag is encoded by nucleotides 1-72 and encodes amino acids 1-24 of SEQ ID NO: 10. The histidine tag comprising 9 histidine (SEQ ID NO: 129) residues is encoded by nucleotides 73-99 and encodes amino acids 25-33 of SEQ ID NO: 10. The linker is encoded by nucleotides 100-111 and encodes amino acids 34-37 of SEQ ID NO: 10. The thrombin cleavage site is encoded by nucleotides 112-135 and encodes amino acids 38-45 of SEQ ID NO: 10. The truncated collagen is encoded by nucleotides 136-822 and encodes amino acids 46-274 of SEQ ID NO: 10.

(SEQ ID NO: 9)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGGGTCCGCAGGGTGTT
GTTGGTGCAGATGGTAAAGACGGTACCCCGGGTGAAAAAGGAGAACAGGG
ACGTACAGGTGCAGCAGGTAAACAGGGCAGCCCGGGTGCCGATGGTGCCC
GTGGCCCGCTGGGTAGCATTGGTCAGCAGGGTGCAAGAGGCGAACCGGGC
GATCCGGGTAGTCCGGGCCTGCGTGGTGATACGGGTCTGGCCGGTGTTAA
AGGCGTTGCAGGTCCTTCAGGTCGTCCAGGTCAACCGGGTGCAAATGGTC
TGCCGGGTGTTAATGGTCGTGGCGGTCTGGAACGTGGTCTGGCAGGACCG
CCGGGTCCTGATGGTCGCCGCGGTGAAACGGGTTCACCGGGTATTGCCGG
TGCCCTGGGTAAACCAGGTCTGGAAGGTCCGAAAGGTTATCCTGGTCTGC
GCGGTCGTGATGGTACCAATGGCAAACGTGGCGAACAGGGCGAAACCGGT
CCAGATGGTGTTCGTGGTATTCCGGGTAACGATGGTCAGAGCGGTAAACC
GGGCATTGATGGTATTGATGGCACCAATGGTCAGCCTGGCGAAGCAGGTT
ATCAGGGTGGTCGCGGTACCCGTGGTCAGCTGGGTGAAACAGGTGATGTT
GGTCAGAATGGTGATCGCGGCGCACCGGGTCCGGATGGTAGCAAAGGTAG
CGCCGGTCGTCCGGGTTTACGTtaa

The truncated collagen is approximately 54% of the full length collagen and is disclosed below in SEQ ID NO: 10.

(SEQ ID NO: 10)
MKKIWLALAGLVLAFSASAAQYEDHHHHHHHHHSGSSLVPRGSHMGPQGV
VGADGKDGTPGEKGEQGRTGAAGKQGSPGADGARGPLGSIGQQGARGEPG
DPGSPGLRGDTGLAGVKGVAGPSGRPGQPGANGLPGVNGRGGLERGLAGP
PGPDGRRGETGSPGIAGALGKPGLEGPKGYPGLRGRDGTNGKRGEQGETG
PDGVRGIPGNDGQSGKPGIDGIDGTNGQPGEAGYQGGRGTRGQLGETGDV
GQNGDRGAPGPDGSKGSAGRPGLR

The polynucleotide encoding the truncated jellyfish collagen without the DsbA secretion tag, the histidine tag, linker and thrombin cleavage site is disclosed in SEQ ID NO: 85

(SEQ ID NO: 85)
GTCCGCAGGGTGTTGTTGGTGCAGATGGTAAAGACGGTACCCCGGGTGAA
AAAGGAGAACAGGGACGTACAGGTGCAGCAGGTAAACAGGGCAGCCCGGG
TGCCGATGGTGCCCGTGGCCCGCTGGGTAGCATTGGTCAGCAGGGTGCAA
GAGGCGAACCGGGCGATCCGGGTAGTCCGGGCCTGCGTGGTGATACGGGT
CTGGCCGGTGTTAAAGGCGTTGCAGGTCCTTCAGGTCGTCCAGGTCAACC
GGGTGCAAATGGTCTGCCGGGTGTTAATGGTCGTGGCGGTCTGGAACGTG
GTCTGGCAGGACCGCCGGGTCCTGATGGTCGCCGCGGTGAAACGGGTTCA
CCGGGTATTGCCGGTGCCCTGGGTAAACCAGGTCTGGAAGGTCCGAAAGG
TTATCCTGGTCTGCGCGGTCGTGATGGTACCAATGGCAAACGTGGCGAAC
AGGGCGAAACCGGTCCAGATGGTGTTCGTGGTATTCCGGGTAACGATGGT
CAGAGCGGTAAACCGGGCATTGATGGTATTGATGGCACCAATGGTCAGCC
TGGCGAAGCAGGTTATCAGGGTGGTCGCGGTACCCGTGGTCAGCTGGGTG
AAACAGGTGATGTTGGTCAGAATGGTGATCGCGGCGCACCGGGTCCGGAT
GGTAGCAAAGGTAGCGCCGGTCGTCCGGGTTTACGTtaa

The truncated jellyfish collagen without the DsbA secretion tag, the histidine tag, linker and thrombin cleavage site is disclosed in SEQ ID NO: 86

(SEQ ID NO: 86)
GPQGVVGADGKDGTPGEKGEQGRTGAAGKQGSPGADGARGPLGSIGQQGA
RGEPGDPGSPGLRGDTGLAGVKGVAGPSGRPGQPGANGLPGVNGRGGLER
GLAGPPGPDGRRGETGSPGIAGALGKPGLEGPKGYPGLRGRDGTNGKRGE
QGETGPDGVRGIPGNDGQSGKPGIDGIDGTNGQPGEAGYQGGRGTRGQLG
ETGDVGQNGDRGAPGPDGSKGSAGRPGLR

The polynucleotides of SEQ ID NO: 9 was codon optimized and synthesized by Gen9 DNA, now Ginkgo Bioworks internal synthesis. Overlaps between the pET28 vector and SEQ ID NO: 9 were designed to be between 30 and 40 bp long and added using PCR with the enzyme PRIMESTAR® GXL polymerase (<http://www.clontech.com/US/Products/PCR/GC_Rich/PrimeSTAR_GXL_DNA_Polymerase?sitex=10020:22372:US>). The opened pET28a vector and insert DNA (SEQ ID NO: 9) was then assembled together into the final plasmid using SGI GIBSON ASSEMBLY® (<https://us.vwr.com/store/product/17613857/gibson-assembly-hifi-1-step-kit-synthetic-genomics-inc>). Sequence of plasmid was then verified through sanger sequencing through Eurofins Genomics (<www.eurofinsgenomics.com>).

The transformed cells were cultivated in minimal media and frozen in 1.5 aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

A bioreactor was prepared with 2.7 L of minimal media+glucose and 300 ml of OD600 of 5-10 culture was added to bring the starting volume to 3 L. Cells were grown at 28° C., pH7 with Dissolved Oxygen maintained at 20% saturation using a cascade containing agitation, air and oxygen. pH was controlled using 28% w/w Ammonium hydroxide solution. Fermentation was run in a fed-batch mode using a DO-stat based feeding algorithm once the initial bolus of 40 g/L was depleted around 13 hours. After 24-26 hours of initial growth, the OD600 reached above 100. At this point, 300 mL of 500 g/L sucrose was added and temperature was reduced to 25 C. High density culture was induced for protein production using 1 mM IPTG. Fermentation was continued for another 20-24 hours and cells were harvested using a bench top centrifuge at 9000 rcf, 15C for 60 minutes. Cell pellet recovered from centrifugation was resuspended in a buffer containing 0.5M NaCl and 0.1 M KH2PO4 at pH8 in a weight by weight ratio of 2× buffer to 1× cells.

The harvested cells were disrupted in a homogenizer at 14,000 psi pressure in 2 passes. Resulting slurry contained the collagen protein along with other proteins.

The fermentations were performed at various temperature ranging from 25° to 28° C. For some fermentations, the temperature of the fermentation was maintained at a constant temperature and immediately upon completion of fermentation (OD600 of 5-10) the collagen was purified. For other fermentations, the temperature of the fermentations was maintained for a desired period of time and when cell densities of OD600 of 5-10 were reached, the temperature was reduced to induce protein production. Typically, the temperature was reduced from 28° C. to 25° C. After the fermentation at 25° C. was continued for 40-60 hours, the collagen was isolated.

The collagen was purified by acid treatment of homogenized cell broth. Additionally, acid treatment was also performed on non-homogenized whole cells recovered from the bioreactor after centrifugation and resuspension in the buffer described above. The pH of either the homogenized slurry of the resuspended whole cells was decreased to 3 using 6M Hydrochloric acid. Acidified cell slurry was incubated overnight at 4° C. with mixing, followed by centrifugation. Supernatant of the acidified slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m² each. Total area of filtration was 0.2 m² using 2 cassettes in parallel. A volume reduction of 5× and a salt reduction of 19× was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified truncated collagen obtained from homogenized cell broth or non-homogenized cells were analyzed on an SDS-PAGE gel and a thick and clear band was observed at the expected size of 27 kilodaltons. The purified collagen was also analyzed by mass spectrometry and it was confirmed that the 27 kilodalton protein was jellyfish collagen.

An alternative purification method of the full length and truncated collagens is provided below.

The fermentation broth was mixed with 0.3-0.5% w/v of Poly Ethyl Imine (PEI). After 15 minutes of incubation with PEI, the fermentation broth was centrifuged at 9000 rcf for 15 minutes to recover the supernatant, which contained the collagen protein. The pellet containing the cells was discarded and the PEI-treated collagen containing supernatant was mixed with Sodium Bentonite (0.2% final w/v) (WYOPURE™, Wyoming Bentonite) and centrifuged. The bentonite containing pellet was discarded and the supernatant was recovered.

The Bentonite treated supernatant was concentrated between 3-6 fold on a tangential flow filtration system (TFF) (EMD Millipore) using a 5 kDa cassette. The collagen was retained with almost no losses in the permeate stream. To remove salts, the retentate from the concentration step was diafiltered using the same TFF set-up. Final conductivity of the protein solution was <10 milliSiemens. The typical conductivity was between 400 microsiemens and 1.5 millisiemens. Highly concentrated collagen solutions had higher conductivities approaching 4 milliSiemens. A skilled artisan will understand that conductivities higher than 10 milliSiemens may be observed depending on the concentration of the collagen. Next, the desalted and concentrated protein was subjected to treatment with activated carbon using the W-L 9000 10×40 granulated resin (Carbon Activated Corporation). 5% w/v of the carbon resin was mixed with collagen containing protein feed and mixed at 45-50° C. with mild agitation. The carbon-treated slurry was filtered using a Buchner funnel lined with an Ertel Filter Press Pad M-953 (Ertel Alsop) in presence or absence of a filtration aid such as Diatomaceous Earth (Sigma Aldrich). Post-filtration, the collagen solution was filtered through a 0.2 micron filter followed by one to several hours of treatment with Sodium Bentonite (0.2% w/v final) (WYOPURE™, Wyoming Bentonite) and centrifuged at 9000 rcf, 15-30 minutes to obtain a highly pure, clear and particulate free collagen solution. When removal of endotoxin proteins was desired, the protein was passed through a chromatographic filter like SARTOBIND® Q (Sartorius-Stedim) to specifically remove endotoxin proteins.

The purified collagen was analyzed on an SDS-PAGE gel and a thick and clear band was observed at 30 kilodaltons. The upshift in size is due to the structure of the collagen molecule and the high glycine/proline amino acid content. The purified collagen was also analyzed by mass spectrometry and it was confirmed that the 30 kilodalton protein was the truncated collagen.

The truncated collagens were further analyzed by HPLC using an Agilent 1100 series HPLC. The column was the 50 mm Agilent PLRP-S reverse phase column with an inner diameter of 4.6 mm, μM particle size and 1000 Angstrom pore size.

The sample was prepared by diluting 1:1 in a 0.04% sodium azide solution in HPLC-grade water. After dilution, the resulting mixture was filtered through a 0.45 um filter to remove any large particles that can clog the HPLC column. For analysis, the samples are diluted appropriately with a 20 mM ammonium acetate buffer in HPLC-grade water at a pH of about 4.5. After mixing the sample, it was transferred to a 300 μL microvial that was then placed in the autosampler. Using ChemStation, the software that operates the HPLC, the analysis parameters such as sample flowrate, column temperature, mobile phase flowrate, mobile phase composition, etc. can be altered. In one exemplary, but non-limiting analysis the parameters were: sample flow rate of 1 mL/min, column temperature of 80° C., column pressure of 60-70 bar, mobile phase composition of 97.9% water/1.9% acetonitrile with 0.2% trifluoroacetic acid; UV wavelength for analysis of 214.4 nm, injection volume of 10 μL, and sample run time of 10 minutes.

Under these conditions, the truncated jellyfish collation of SEQ ID NO: 91 has an elution time of about 5.4 minutes. ChemStation quantifies the peak area of the elution peak and calculates the protein concentration using a calibration curve that directly relates peak area to protein concentration. The calibration curve is generated using a known collagen solution that is serially diluted to contain collagen concentration ranges of 0.06 mg/mL to 1.00 mg/mL.

Truncated Collagen without His Tag-Linker-Thrombin Cleavage Site

A truncated jellyfish collagen without a His tag, linker, and thrombin cleavage site is disclosed below. The codon-optimized nucleotide sequence encoding this collagen is provided in SEQ ID NO: 11. The amino acid sequence is disclosed in SEQ ID NO: 12. The DsbA secretion tag is encoded by nucleotides 1-72 and encodes amino acids 1-24. The truncated collagen sequence is encoded by nucleotides 73-639 and encodes amino acids 25-213.

(SEQ ID NO: 11)
ATGAAAAAGATTTGGCTGGCGCTGGCTGGTTTAGTTTTAGCGTTTAGCGC
ATCGGCGGCGCAGTATGAAGATGGTCCGCAGGGTGTTGTTGGTGCAGATG
GTAAAGACGGTACCCCGGGTAATGCAGGTCAGAAAGGTCCGTCAGGTGAA
CCTGGCAGCCCTGGTAAAGCAGGTAGTGCCGGTGAGCAGGGTCCGCCGGG
CAAAGATGGTAGTAATGGTGAGCCGGGTAGCCCTGGCAAAGAAGGTGAAC
GTGGTCTGGCAGGACCGCCGGGTCCTGATGGTCGCCGCGGTGAAACGGGT
TCACCGGGTATTGCCGGTGCCCTGGGTAAACCAGGTCTGGAAGGTCCGAA
AGGTTATCCTGGTCTGCGCGGTCGTGATGGTACCAATGGCAAACGTGGCG
AACAGGGCGAAACCGGTCCAGATGGTGTTCGTGGTATTCCGGGTAACGAT
GGTCAGAGCGGTAAACCGGGCATTGATGGTATTGATGGCACCAATGGTCA
GCCTGGCGAAGCAGGTTATCAGGGTGGTCGCGGTACCCGTGGTCAGCTGG
GTGAAACAGGTGATGTTGGTCAGAATGGTGATCGCGGCGCACCGGGTCCG
GATGGTAGCAAAGGTAGCGCCGGTCGTCCGGGTTTACGTtaa
(SEQ ID NO: 12)
MKKIWLALAGLVLAFSASAAQYEDGPQGVVGADGKDGTPGNAGQKGPSGE
PGSPGKAGSAGEQGPPGKDGSNGEPGSPGKEGERGLAGPPGPDGRRGETG
SPGIAGALGKPGLEGPKGYPGLRGRDGTNGKRGEQGETGPDGVRGIPGND
GQSGKPGIDGIDGTNGQPGEAGYQGGRGTRGQLGETGDVGQNGDRGAPGP
DGSKGSAGRPGLR

A polynucleotide encoding a truncated jellyfish collagen without a His tag, linker and thrombin cleavage site disclosed in SEQ ID NO: 90

(SEQ ID NO: 90)
GGTCCGCAGGGTGTTGTTGGTGCAGATGGTAAAGACGGTACCCCGGGTAA
TGCAGGTCAGAAAGGTCCGTCAGGTGAACCTGGCAGCCCTGGTAAAGCAG
GTAGTGCCGGTGAGCAGGGTCCGCCGGGCAAAGATGGTAGTAATGGTGAG
CCGGGTAGCCCTGGCAAAGAAGGTGAACGTGGTCTGGCAGGACCGCCGGG
TCCTGATGGTCGCCGCGGTGAAACGGGTTCACCGGGTATTGCCGGTGCCC
TGGGTAAACCAGGTCTGGAAGGTCCGAAAGGTTATCCTGGTCTGCGCGGT
CGTGATGGTACCAATGGCAAACGTGGCGAACAGGGCGAAACCGGTCCAGA
TGGTGTTCGTGGTATTCCGGGTAACGATGGTCAGAGCGGTAAACCGGGCA
TTGATGGTATTGATGGCACCAATGGTCAGCCTGGCGAAGCAGGTTATCAG
GGTGGTCGCGGTACCCGTGGTCAGCTGGGTGAAACAGGTGATGTTGGTCA
GAATGGTGATCGCGGCGCACCGGGTCCGGATGGTAGCAAAGGTAGCGCCG
GTCGTCCGGGTTTACGTtaa

A truncated jellyfish collagen without a His tag, linker and thrombin cleavage site disclosed in SEQ ID NO: 91

(SEQ ID NO: 91)
GPQGVVGADGKDGTPGNAGQKGPSGEPGSPGKAGSAGEQGPPGKDGSNGE
PGSPGKEGERGLAGPPGPDGRRGETGSPGIAGALGKPGLEGPKGYPGLRG
RDGTNGKRGEQGETGPDGVRGIPGNDGQSGKPGIDGIDGTNGQPGEAGYQ
GGRGTRGQLGETGDVGQNGDRGAPGPDGSKGSAGRPGLR

Truncated Collagen with GEK Repeats

A jellyfish collagen with GEK repeats is disclosed below. The codon-optimized nucleotide sequence encoding this collagen is provided in SEQ ID NO: 13. The amino acid sequence is disclosed in SEQ ID NO: 14. The DsbA secretion tag is encoded by nucleotides 1-72 and encodes amino acids 1-24. The GEK repeat is encoded by nucleotides 73-126 and encodes the GEK repeats of amino acids 25-42. The truncated collagen sequence is encoded by nucleotides 127-693 and encodes amino acids 43-231.

(SEQ ID NO: 13)
ATGAAAAAGATTTGGCTGGCGCTGGCTGGTTTAGTTTTAGCGTTTAGCGC
ATCGGCGGCGCAGTATGAAGATGGTGAAAAAGGTGAaAAGGGCGAGAAAG
GTGAGAAAGGCGAAAAGGGTGAAAAAGGTCCGCAGGGTGTTGTTGGTGCA
GATGGTAAAGACGGTACCCCGGGTAATGCAGGTCAGAAAGGTCCGTCAGG
TGAACCTGGCAGCCCTGGTAAAGCAGGTAGTGCCGGTGAGCAGGGTCCGC
CGGGCAAAGATGGTAGTAATGGTGAGCCGGGTAGCCCTGGCAAAGAAGGT
GAACGTGGTCTGGCAGGACCGCCGGGTCCTGATGGTCGCCGCGGTGAAAC
GGGTTCACCGGGTATTGCCGGTGCCCTGGGTAAACCAGGTCTGGAAGGTC
CGAAAGGTTATCCTGGTCTGCGCGGTCGTGATGGTACCAATGGCAAACGT
GGCGAACAGGGCGAAACCGGTCCAGATGGTGTTCGTGGTATTCCGGGTAA
CGATGGTCAGAGCGGTAAACCGGGCATTGATGGTATTGATGGCACCAATG
GTCAGCCTGGCGAAGCAGGTTATCAGGGTGGTCGCGGTACCCGTGGTCAG
CTGGGTGAAACAGGTGATGTTGGTCAGAATGGTGATCGCGGCGCACCGGG
TCCGGATGGTAGCAAAGGTAGCGCCGGTCGTCCGGGTTTACGTtaa
(SEQ ID NO: 14)
MKKIWLALAGLVLAFSASAAQYEDGEKGEKGEKGEKGEKGEKGPQGVVGA
DGKDGTPGNAGQKGPSGEPGSPGKAGSAGEQGPPGKDGSNGEPGSPGKEG
ERGLAGPPGPDGRRGETGSPGIAGALGKPGLEGPKGYPGLRGRDGTNGKR
GEQGETGPDGVRGIPGNDGQSGKPGIDGIDGTNGQPGEAGYQGGRGTRGQ
LGETGDVGQNGDRGAPGPDGSKGSAGRPGLR

The polynucleotides of SEQ ID NO: 13 was codon optimized and synthesized by Gen9 DNA, now Ginkgo Bioworks internal synthesis. Overlaps between the pET28 vector and SEQ ID NO: 13 was designed to be between 30 and 40 bp long and added using PCR with the enzyme PRIMESTAR® GXL polymerase (<http://www.clontech.com/US/Products/PCR/GC_Rich/PrimeSTAR_GXL_DNA_Polymerase?sitex=10020:22372:US>). The opened pET28a vector and insert DNA (SEQ ID NO: 13) was then assembled together into the final plasmid using SGI GIBSON ASSEMBLY® (<https://us.vwr.com/store/product/17613857/gibson-assembly-hifi-1-step-kit-synthetic-genomics-inc>). Sequence of plasmid was then verified through Sanger sequencing through Eurofins Genomics (<www.eurofinsgenomics.com>).

The transformed cells were cultivated in minimal media and frozen in 1.5 aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

A bioreactor was prepared with 2.7 L of minimal media+glucose and 300 ml of OD600 of 5-10 culture was added to bring the starting volume to 3 L. Cells were grown at 28° C., pH7 with Dissolved Oxygen maintained at 20% saturation using a cascade containing agitation, air and oxygen. pH was controlled using 28% w/w Ammonium hydroxide solution. Fermentation was run in a fed-batch mode using a DO-stat based feeding algorithm once the initial bolus of 40 g/L was depleted around 13 hours. After 24-26 hours of initial growth, the OD600 reached above 100. At this point, 300 mL of 500 g/L sucrose was added and temperature was reduced to 25 C. High density culture was induced for protein production using 1 mM IPTG. Fermentation was continued for another 20-24 hours and cells were harvested using a bench top centrifuge at 9000 rcf, 15C for 60 minutes. Cell pellet recovered from centrifugation was resuspended in a buffer containing 0.5M NaCl and 0.1 M KH2PO4 at pH8 in a weight by weight ratio of 2× buffer to 1× cells.

The harvested cells were disrupted in a homogenizer at 14,000 psi pressure in 2 passes. Resulting slurry contained the collagen protein along with other proteins.

The collagen was purified by acid treatment whole cells recovered from bioreactor after centrifugation and resuspension in a buffer as described above The pH of either the homogenized slurry or the resuspended suspension was decreased to 3 using 6M Hydrochloric acid. Acidified cell slurry was incubated overnight at 4° C. with mixing, followed by centrifugation. Supernatant of the acidified slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m² each. Total area of filtration was 0.2 m² using 2 cassettes in parallel. A volume reduction of 5× and a salt reduction of 19× was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified collagen was analyzed on an SDS-PAGE gel and was observed to run at an apparent molecular weight of 35 kilodaltons. The 35 kilodalton band does not correspond to the expected size of 22 kilodaltons. The upshift between the expected size and the apparent size is thought to be due to the GEK repeats interacting with the gel matrix. The 35 kDa band was confirmed by mass spectrometry to be the correct collagen with the GEK repeats.

Truncated Collagen with GDK Repeats

A jellyfish collagen with GDK repeats is disclosed below. The codon-optimized nucleotide sequence encoding this collagen is provided in SEQ ID NO: 15. The amino acid sequence is disclosed in SEQ ID NO: 16. The DsbA secretion tag is encoded by nucleotides 1-72 and encodes amino acids 1-24. The GDK repeat is encoded by nucleotides 73-126 and encodes the GDK repeats of amino acids 25-42. The truncated collagen sequence is encoded by nucleotides 127-693 and encodes amino acids 43-231.

(SEQ ID NO: 15)
ATGAAAAAGATTTGGCTGGCGCTGGCTGGTTTAGTTTTAGCGTTTAGCGC
ATCGGCGGCGCAGTATGAAGATGGTGATAAAGGTGATAAGGGCGACAAAG
GTGACAAAGGCGATAAGGGTGATAAAGGTCCGCAGGGTGTTGTTGGTGCA
GATGGTAAAGACGGTACCCCGGGTAATGCAGGTCAGAAAGGTCCGTCAGG
TGAACCTGGCAGCCCTGGTAAAGCAGGTAGTGCCGGTGAGCAGGGTCCGC
CGGGCAAAGATGGTAGTAATGGTGAGCCGGGTAGCCCTGGCAAAGAAGGT
GAACGTGGTCTGGCAGGACCGCCGGGTCCTGATGGTCGCCGCGGTGAAAC
GGGTTCACCGGGTATTGCCGGTGCCCTGGGTAAACCAGGTCTGGAAGGTC
CGAAAGGTTATCCTGGTCTGCGCGGTCGTGATGGTACCAATGGCAAACGT
GGCGAACAGGGCGAAACCGGTCCAGATGGTGTTCGTGGTATTCCGGGTAA
CGATGGTCAGAGCGGTAAACCGGGCATTGATGGTATTGATGGCACCAATG
GTCAGCCTGGCGAAGCAGGTTATCAGGGTGGTCGCGGTACCCGTGGTCAG
CTGGGTGAAACAGGTGATGTTGGTCAGAATGGTGATCGCGGCGCACCGGG
TCCGGATGGTAGCAAAGGTAGCGCCGGTCGTCCGGGTTTACGTtaa
(SEQ ID NO: 16)
MKKIWLALAGLVLAFSASAAQYEDGDKGDKGDKGDKGDKGDKGPQGVVGA
DGKDGTPGNAGQKGPSGEPGSPGKAGSAGEQGPPGKDGSNGEPGSPGKEG
ERGLAGPPGPDGRRGETGSPGIAGALGKPGLEGPKGYPGLRGRDGTNGKR
GEQGETGPDGVRGIPGNDGQSGKPGIDGIDGTNGQPGEAGYQGGRGTRGQ
LGETGDVGQNGDRGAPGPDGSKGSAGRPGLR

The polynucleotides of SEQ ID NO: 15 was codon optimized and synthesized by Gen9 DNA, now Ginkgo Bioworks internal synthesis. Overlaps between the pET28 vector and SEQ ID NO: 15 was designed to be between 30 and 40 bp long and added using PCR with the enzyme PRIMESTAR® GXL polymerase (<http://www.clontech.com/US/Products/PCR/GC_Rich/PrimeSTAR_GXL_DNA_Polymerase?sitex=10020:22372:US>). The opened pET28a vector and insert DNA (SEQ ID NO: 15) was then assembled together into the final plasmid using SGI GIBSON ASSEMBLY® (<https://us.vwr.com/store/product/17613857/gibson-assembly-hifi-1-step-kit-synthetic-genomics-inc>). Sequence of plasmid was then verified through sanger sequencing through Eurofins Genomics (<www.eurofinsgenomics.com>).

The transformed cells were cultivated in minimal media and frozen in 1.5 aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

A bioreactor was prepared with 2.7 L of minimal media+glucose and 300 ml of OD600 of 5-10 culture was added to bring the starting volume to 3 L. Cells were grown at 28° C., pH7 with Dissolved Oxygen maintained at 20% saturation using a cascade containing agitation, air and oxygen. pH was controlled using 28% w/w Ammonium hydroxide solution. Fermentation was run in a fed-batch mode using a DO-stat based feeding algorithm once the initial bolus of 40 g/L was depleted around 13 hours. After 24-26 hours of initial growth, the OD600 reached above 100. At this point, 300 mL of 500 g/L sucrose was added and temperature was reduced to 25 C. High density culture was induced for protein production using 1 mM IPTG. Fermentation was continued for another 20-24 hours and cells were harvested using a bench top centrifuge at 9000 rcf, 15C for 60 minutes. Cell pellet recovered from centrifugation was resuspended in a buffer containing 0.5M NaCl and 0.1 M KH2PO4 at pH8 in a weight by weight ratio of 2× buffer to 1× cells.

The harvested cells were disrupted in a homogenizer at 14,000 psi pressure in 2 passes. Resulting slurry contained the collagen protein along with other proteins.

The collagen was purified by acid treatment of whole cells recovered from bioreactor after centrifugation and resuspension in a buffer as described above. The pH of either the homogenized slurry was decreased to 3 using 6M Hydrochloric acid. Acidified cell slurry was incubated overnight at 4° C. with mixing, followed by centrifugation. Supernatant of the acidified slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m² each. Total area of filtration was 0.2 m² using 2 cassettes in parallel. A volume reduction of 5× and a salt reduction of 19× was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified collagen was analyzed on an SDS-PAGE gel and was observed to run at an apparent molecular weight of 35 kilodaltons. The 35 kilodalton band does not correspond to the expected size of 22 kilodaltons. The upshift between the expected size and the apparent size is thought to be due to the GDK repeats interacting with the gel matrix. The 35 kDa band was confirmed by mass spectrometry to be the correct collagen with the GDK repeats.

Truncated Collagen with DsbA Secretion Tag-His Tag-Linker-Thrombin Cleavage Site and GFP Beta-Lactamase Fusion (Version 1):

A jellyfish collagen with DsbA secretion tag-His tag-Linker-Thrombin cleavage site and GFP Beta-lactamase fusion is disclosed below. The codon-optimized nucleotide sequence encoding this collagen is provided in SEQ ID NO: 17. The amino acid sequence is disclosed in SEQ ID NO: 18. The DsbA secretion tag is encoded by nucleotides 1-72 and encodes amino acids 1-24. The His tag is encoded by nucleotides 73-99 and encodes a 9 histidine tag (SEQ ID NO: 129) of amino acids 25-33. The linker is encoded by nucleotides 100-111 and encodes amino acids 34-37. The thrombin cleavage side is encoded by nucleotides 112-135 and encodes amino acids 38-45. The green fluorescent protein (GFP) with linker is encoded by nucleotides 136-873 and encodes amino acids 46-291. The truncated collagen sequence is encoded by nucleotides 874-1440 and encodes amino acids 292-480. The Beta-lactamase with linker is encoded by nucleotides 1441-2232 and encodes amino acids 481-744. The Beta-lactamase was properly targeted to the periplasmic space even though the polypeptide did not have an independent secretion tag. The DsbA secretion tag directed the entire transcript (Truncated Collagen with DsbA secretion tag-His tag-Linker-Thrombin cleavage site and GFP Beta-lactamase fusion protein) to the periplasmic space and the Beta-lactamase functioned properly.

(SEQ ID NO: 17)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGTCTGGCTCGAGCAGT
AAAGGTGAAGAACTGTTCACCGGTGTTGTTCCGATCCTGGTTGAACTGGA
TGGTGATGTTAACGGCCACAAATTCTCTGTTCGTGGTGAAGGTGAAGGTG
ATGCAACCAACGGTAAACTGACCCTGAAATTCATCTGCACTACCGGTAAA
CTGCCGGTTCCATGGCCGACTCTGGTGACTACCCTGACCTATGGTGTTCA
GTGTTTTTCTCGTTACCCGGATCACATGAAGCAGCATGATTTCTTCAAAT
CTGCAATGCCGGAAGGTTATGTACAGGAGCGCACCATTTCTTTCAAAGAC
GATGGCACCTACAAAACCCGTGCAGAGGTTAAATTTGAAGGTGATACTCT
GGTGAACCGTATTGAACTGAAAGGCATTGATTTCAAAGAGGACGGCAACA
TCCTGGGCCACAAACTGGAATATAACTTCAACTCCCATAACGTTTACATC
ACCGCAGACAAACAGAAGAACGGTATCAAAGCTAACTTCAAAATTCGCCA
TAACGTTGAAGACGGTAGCGTACAGCTGGCGGACCACTACCAGCAGAACA
CTCCGATCGGTGATGGTCCGGTTCTGCTGCCGGATAACCACTACCTGTCC
ACCCAGTCTaaaCTGTCCAAAGACCCGAACGAAAAGCGCGACCACATGGT
GCTGCTGGAGTTCGTTACTGCAGCAGGTATCACGCACGGCATGGATGAAC
TCTACAAATCTGGCGCGCCGGGCGGTCCGCAGGGTGTTGTTGGTGCAGAT
GGTAAAGACGGTACCCCGGGTAATGCAGGTCAGAAAGGTCCGTCAGGTGA
ACCTGGCAGCCCTGGTAAAGCAGGTAGTGCCGGTGAGCAGGGTCCGCCGG
GCAAAGATGGTAGTAATGGTGAGCCGGGTAGCCCTGGCAAAGAAGGTGAA
CGTGGTCTGGCAGGACCGCCGGGTCCTGATGGTCGCCGCGGTGAAACGGG
TTCACCGGGTATTGCCGGTGCCCTGGGTAAACCAGGTCTGGAAGGTCCGA
AAGGTTATCCTGGTCTGCGCGGTCGTGATGGTACCAATGGCAAACGTGGC
GAACAGGGCGAAACCGGTCCAGATGGTGTTCGTGGTATTCCGGGTAACGA
TGGTCAGAGCGGTAAACCGGGCATTGATGGTATTGATGGCACCAATGGTC
AGCCTGGCGAAGCAGGTTATCAGGGTGGTCGCGGTACCCGTGGTCAGCTG
GGTGAAACAGGTGATGTTGGTCAGAATGGTGATCGCGGCGCACCGGGTCC
GGATGGTAGCAAAGGTAGCGCCGGTCGTCCGGGTTTACGTcacccagaaa
cgctggtgaaagtaaaagatgctgaagatcagttgggtgcacgagtgggt
tacatcgaactggatctcaacagcggtaagatccttgagagttttcgccc
cgaagaacgttttccaatgatgagcacttttaaagttctgctatgtggcg
cggtattatcccgtattgacgccgggcaagagcaactcggtcgccgcata
cactattctcagaatgacttggttgagtactcaccagtcacagaaaagca
tcttacggatggcatgacagtaagagaattatgcagtgctgccataacca
tgagtgataacactgcggccaacttacttctgacaacgatcggaggaccg
aaggagctaaccgctatagcacaacatgggggatcatgtaactcgccttg
atcgttgggaaccggagctgaatgaagccataccaaacgacgagcgtgac
accacgatgcctgtagcaatggcaacaacgttgcgcaaactattaactgg
cgaactacttactctagcttcccggcaacaattaatagactggatggagg
cggataaagttgcaggaccacttctgcgctcggcccttccggctggctgg
tttattgctgataaatctggagccggtgagcgtgggtctcgcggtatcat
tgcagcactggggccagatggtaagccctcccgtatcgtagttatctaca
cgacggggagtcaggcaactatggatgaacgaaatagacagatcgctgag
ataggtgcctcactgattaagcattggtaa
(SEQ ID NO: 18)
MKKIWLALAGLVLAFSASAAQYEDHHHHHHHHHSGSSLVPRGSHMSGSSS
KGEELFTGVVPILVELDGDVNGHKFSVRGEGEGDATNGKLTLKFICTTGK
LPVPWPTLVTTLTYGVQCFSRYPDHMKQHDFFKSAMPEGYVQERTISFKD
DGTYKTRAEVKFEGDTLVNRIELKGIDFKEDGNILGHKLEYNFNSHNVYI
TADKQKNGIKANFKIRHNVEDGSVQLADHYQQNTPIGDGPVLLPDNHYLS
TQSKLSKDPNEKRDHMVLLEFVTAAGITHGMDELYKSGAPGGPQGVVGAD
GKDGTPGNAGQKGPSGEPGSPGKAGSAGEQGPPGKDGSNGEPGSPGKEGE
RGLAGPPGPDGRRGETGSPGIAGALGKPGLEGPKGYPGLRGRDGTNGKRG
EQGETGPDGVRGIPGNDGQSGKPGIDGIDGTNGQPGEAGYQGGRGTRGQL
GETGDVGQNGDRGAPGPDGSKGSAGRPGLRHPETLVKVKDAEDQLGARVG
YIELDLNSGKILESFRPEERFPMMSTFKVLLCGAVLSRIDAGQEQLGRRI
HYSQNDLVEYSPVTEKHLTDGMTVRELCSAAITMSDNTAANLLLTTIGGP
KELTAFLHNMGDHVTRLDRWEPELNEAIPNDERDTTMPVAMATTLRKLLT
GELLTLASRQQLIDWMEADKVAGPL5LRSALPAGWFIADKSGAGERGSRG
IIAALGPDGKPSRIVVIYTTGSQATMDERNRQIAEIGASLIKHW

The polynucleotides of SEQ ID NO: 17 was constructed by assembling several DNA fragments. The collagen containing sequence was codon optimized and synthesized by Gen9 DNA, now Ginkgo Bioworks internal synthesis. The GFP was also synthesized by Gen9. The Beta-lactamase was cloned out of the plasmid pKD46 (<http://cgsc2.biology.yale.edu/Strain.php?ID=68099>) using PCR with the enzyme PRIMESTAR® GXL polymerase (<http://www.clontech.com/US/Products/PCR/GC_Rich/PrimeSTAR_GXL_DNA_Polymerase?sitex=10020:22372:US>). Overlaps between the pET28 vector, GFP, Collagen, and Beta-lactamase was designed to be between 30 and 40 bp long and added using PCR with the enzyme PRIMESTAR® GXL polymerase. The opened pET28a vector and inserts were then assembled together into the final plasmid using SGI GIBSON ASSEMBLY® (<https://us.vwr.com/store/product/17613857/gibson-assembly-hifi-1-step-kit-synthetic-genomics-inc>). Sequence of plasmid was then verified through sanger sequencing through Eurofins Genomics (<www.eurofinsgenomics.com>).

The transformed cells were cultivated in minimal media and frozen in 1.5 aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

A bioreactor was prepared with 2.7 L of minimal media+glucose and 300 ml of OD600 of 5-10 culture was added to bring the starting volume to 3 L. Cells were grown at 28° C., pH7 with Dissolved Oxygen maintained at 20% saturation using a cascade containing agitation, air and oxygen. pH was controlled using 28% w/w Ammonium hydroxide solution. Fermentation was run in a fed-batch mode using a DO-stat based feeding algorithm once the initial bolus of 40 g/L was depleted around 13 hours. After 24-26 hours of initial growth, the OD600 reached above 100. At this point, 300 mL of 500 g/L sucrose was added and temperature was reduced to 25C. High density culture was induced for protein production using 1 mM IPTG. Fermentation was continued for another 20-24 hours and cells were harvested using a bench top centrifuge at 9000 rcf, 15C for 60 minutes. Cell pellet recovered from centrifugation was resuspended in a buffer containing 0.5M NaCl and 0.1 M KH2PO4 at pH8 in a weight by weight ratio of 2× buffer to 1× cells.

The harvested cells were disrupted in a homogenizer at 14,000 psi pressure in 2 passes. Resulting slurry contained the collagen protein along with other proteins.

The collagen was purified by acid treatment of non-homogenized whole cells recovered from the bioreactor after centrifugation and resuspension in the buffer described above. The pH of the resuspended suspension—was decreased to 3 using 6M Hydrochloric acid. Acidified cell slurry was incubated overnight at 4° C. with mixing, followed by centrifugation. The pH was then raised to 9 using TON NaOH and the supernatant of the slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m2 each. Total area of filtration was 0.2 m2 using 2 cassettes in parallel. A volume reduction of 5× and a salt reduction of 19× was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified collagen-GFP-Beta-lactamase fusion protein was analyzed on an SDS-PAGE gel and was observed to run at an apparent molecular weight of 90 kilodaltons. The expected size of the fusion protein is 85 kd. The 90 kDa band was confirmed by mass spectrometry to be the correct collagen fusion protein.

Truncated Collagen with DsbA Secretion Tag-His Tag-Linker-Thrombin Cleavage Site and GFP Beta-Lactamase Fusion (Version 2):

A jellyfish collagen with DsbA secretion tag-His tag-Linker-Thrombin cleavage site and GFP Beta-lactamase fusion is disclosed below. The codon-optimized nucleotide sequence encoding this collagen is provided in SEQ ID NO: 19. The amino acid sequence is disclosed in SEQ ID NO: 20. The DsbA secretion tag is encoded by nucleotides 1-72 and encodes amino acids 1-24. The His tag is encoded by nucleotides 73-99 and encodes a 9 histidine tag (SEQ ID NO: 129) of amino acids 25-33. The linker is encoded by nucleotides 100-111 and encodes amino acids 34-37. The thrombin cleavage side is encoded by nucleotides 112-135 and encodes amino acids 38-45. The green fluorescent protein (GFP) with linker is encoded by nucleotides 136-873 and encodes amino acids 46-291 The truncated collagen sequence is encoded by nucleotides 874-1440 and encodes amino acids 292-480. The Beta-lactamase with linker is encoded by nucleotides 1441-2232 and encodes amino acids 481-744.

(SEQ ID NO: 19)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGTCTGGCTCGAGCAGT
AAAGGTGAAGAACTGTTCACCGGTGTTGTTCCGATCCTGGTTGAACTGGA
TGGTGATGTTAACGGCCACAAATTCTCTGTTCGTGGTGAAGGTGAAGGTG
ATGCAACCAACGGTAAACTGACCCTGAAATTCATCTGCACTACCGGTAAA
CTGCCGGTTCCATGGCCGACTCTGGTGACTACCCTGACCTATGGTGTTCA
GTGTTTTTCTCGTTACCCGGATCACATGAAGCAGCATGATTTCTTCAAAT
CTGCAATGCCGGAAGGTTATGTACAGGAGCGCACCATTTCTTTCAAAGAC
GATGGCACCTACAAAACCCGTGCAGAGGTTAAATTTGAAGGTGATACTCT
GGTGAACCGTATTGAACTGAAAGGCATTGATTTCAAAGAGGACGGCAACA
TCCTGGGCCACAAACTGGAATATAACTTCAACTCCCATAACGTTTACATC
ACCGCAGACAAACAGAAGAACGGTATCAAAGCTAACTTCAAAATTCGCCA
TAACGTTGAAGACGGTAGCGTACAGCTGGCGGACCACTACCAGCAGAACA
CTCCGATCGGTGATGGTCCGGTTCTGCTGCCGGATAACCACTACCTGTCC
ACCCAGTCTaaaCTGTCCAAAGACCCGAACGAAAAGCGCGACCACATGGT
GCTGCTGGAGTTCGTTACTGCAGCAGGTATCACGCACGGCATGGATGAAC
TCTACAAATCTGGCGCGCCGGGCGGTCCGCAGGGTGTTGTTGGTGCAGAT
GGTAAAGACGGTACCCCGGGTAATGCAGGTCAGAAAGGTCCGTCAGGTGA
ACCTGGCAGCCCTGGTAAAGCAGGTAGTGCCGGTGAGCAGGGTCCGCCGG
GCAAAGATGGTAGTAATGGTGAGCCGGGTAGCCCTGGCAAAGAAGGTGAA
CGTGGTCTGGCAGGACCGCCGGGTCCTGATGGTCGCCGCGGTGAAACGGG
TTCACCGGGTATTGCCGGTGCCCTGGGTAAACCAGGTCTGGAAGGTCCGA
AAGGTTATCCTGGTCTGCGCGGTCGTGATGGTACCAATGGCAAACGTGGC
GAACAGGGCGAAACCGGTCCAGATGGTGTTCGTGGTATTCCGGGTAACGA
TGGTCAGAGCGGTAAACCGGGCATTGATGGTATTGATGGCACCAATGGTC
AGCCTGGCGAAGCAGGTTATCAGGGTGGTCGCGGTACCCGTGGTCAGCTG
GGTGAAACAGGTGATGTTGGTCAGAATGGTGATCGCGGCGCACCGGGTCC
GGATGGTAGCAAAGGTAGCGCCGGTCGTCCGGGTTTACGTcacccagaaa
cgctggtgaaagtaaaagatgctgaagatcagttgggtgcacgagtgggt
tacatcgaactggatctcaacagcggtaagatccttgagagttttcgccc
cgaagaacgttttccaatgatgagcacttttaaagttctgctatgtggcg
cggtattatcccgtattgacgccgggcaagagcaactcggtcgccgcata
cactattctcagaatgacttggttgagtactcaccagtcacagaaaagca
tcttacggatggcatgacagtaagagaattatgcagtgctgccataacca
tgagtgataacactgcggccaacttacttctgacaacgatcggaggaccg
aaggagctaaccgctatagcacaacatgggggatcatgtaactcgccttg
atcgttgggaaccggagctgaatgaagccataccaaacgacgagcgtgac
accacgatgcctgtagcaatggcaacaacgttgcgcaaactattaactgg
cgaactacttactctagcttcccggcaacaattaatagactggatggagg
cggataaagttgcaggaccacttctgcgctcggcccttccggctggctgg
tttattgctgataaatctggagccggtgagcgtgggtctcgcggtatcat
tgcagcactggggccagatggtaagccctcccgtatcgtagttatctaca
cgacggggagtcaggcaactatggatgaacgaaatagacagatcgctgag
ataggtgcctcactgattaagcattggtaa
(SEQ ID NO: 20)
MKKIWLALAGLVLAFSASAAQYEDHHHHHHHHHSGSSLVPRGSHMSGSSS
KGEELFTGVVPILVELDGDVNGHKFSVRGEGEGDATNGKLTLKFICTTGK
LPVPWPTLVTTLTYGVQCFSRYPDHMKQHDFFKSAMPEGYVQERTISFKD
DGTYKTRAEVKFEGDTLVNRIELKGIDFKEDGNILGHKLEYNFNSHNVYI
TADKQKNGIKANFKIRHNVEDGSVQLADHYQQNTPIGDGPVLLPDNHYLS
TQSKLSKDPNEKRDHMVLLEFVTAAGITHGMDELYKSGAPGGPQGVVGAD
GKDGTPGNAGQKGPSGEPGSPGKAGSAGEQGPPGKDGSNGEPGSPGKEGE
RGLAGPPGPDGRRGETGSPGIAGALGKPGLEGPKGYPGLRGRDGTNGKRG
EQGETGPDGVRGIPGNDGQSGKPGIDGIDGTNGQPGEAGYQGGRGTRGQL
GETGDVGQNGDRGAPGPDGSKGSAGRPGLRHPETLVKVKDAEDQLGARVG
YIELDLNSGKILESFRPEERFPMMSTFKVLLCGAVLSRIDAGQEQLGRRI
HYSQNDLVEYSPVTEKHLTDGMTVRELCSAAITMSDNTAANLLLTTIGGP
KELTAFLHNMGDHVTRLDRWEPELNEAIPNDERDTTMPVAMATTLRKLLT
GELLTLASRQQLIDWMEADKVAGPLLRSALPAGWFIADKSGAGERGSRGI
IAALGPDGKPSRIVVIYTTGSQATMDERNRQIAEIGASLIKHW

### Example 4: Production of Full-Length Elastin

Full length human elastin were expressed as described below. The wild-type, full length amino acid sequence of human elastin is provided below.

(SEQ ID NO: 21)
MAGLTAAAPRPGVLLLLLSILHPSRPGGVPGAIPGGVPGGVFYPGAGLGA
LGGGALGPGGKPLKPVPGGLAGAGLGAGLGAFPAVTFPGALVPGGVADAA
AAYKAAKAGAGLGGVPGVGGLGVSAGAVVPQPGAGVKPGKVPGVGLPGVY
PGGVLPGARFPGVGVLPGVPTGAGVKPKAPGVGGAFAGIPGVGPFGGPQP
GVPLGYPIKAPKLPGGYGLPYTTG5KLPYGYGPGGVAGAAGKAGYPTGTG
VGPQAAAAAAAKAAAKFGAGAAGVLPGVGGAGVPGVPGAIPGIGGIAGVG
TPAAAAAAAAAAKAAKYGAAAGLVPGGPGFGPGVVGVPGAGVPGVGVPGA
GIPVVPGAGIPGAAVPGVVSPEAAAKAAAKAAKYGARPGVGVGGIPTYGV
GAGGFPGFGVGVGGIPGVAGVPGVGGVPGVGGVPGVGISPEAQAAAAAKA
AKYGAAGAGVLGGLVPGPQAAVPGVPGTGGVPGVGTPAAAAAKAAAKAAQ
oFGLVPGVGVAPGVGVAPGVGVAPGVGLAPGVGVAPGVGVAPGVGVAPGI
GPGGVAAAAKSAAKVAAKAQLRAAAGLGAGIPGLGVGVGVPGLGVGAGVP
GLGVGAGVPGFGAGADEGVRRSLSPELREGDPSSSQHLPSTPSSPRVPGA
LAAAKAAKYGAAVPGVLGGLGALGGVGIPGGVVGAGPAAAAAAAKAAAKA
AQFGLVGAAGLGGLGVGGLGVPGVGGLGGIPPAAAAKAAKYGAAGLGGVL
GGAGQFPLGGVAARPGFGLSPIFPGGACLGKACGRKRK
<http://www.uniprot.org/uniprot/P15502>

The non-codon optimized polynucleotide sequence encoding the full length elastin is disclosed below. In SEQ ID NO: 22, nucleotides 1-78 encode the DsbA secretion tag and nucleotides 79-2358 encode the full length human elastin.

(SEQ ID NO: 22)
ATGGCGGGTCTGACGGCGGCGGCCCCGCGGCCCGGAGTCCTCCTGCTCCT
GCTGTCCATCCTCCACCCCTCTCGGCCTGGAGGGGTCCCTGGGGCCATTC
CTGGTGGAGTTCCTGGAGGAGTCTTTTATCCAGGGGCTGGTCTCGGAGCC
CTTGGAGGAGGAGCGCTGGGGCCTGGAGGCAAACCTCTTAAGCCAGTTCC
CGGAGGGCTTGCGGGTGCTGGCCTTGGGGCAGGGCTCGGCGCCTTCCCCG
CAGTTACCTTTCCGGGGGCTCTGGTGCCTGGTGGAGTGGCTGACGCTGCT
GCAGCCTATAAAGCTGCTAAGGCTGGCGCTGGGCTTGGTGGTGTCCCAGG
AGTTGGTGGCTTAGGAGTGTCTGCAGGTGCGGTGGTTCCTCAGCCTGGAG
CCGGAGTGAAGCCTGGGAAAGTGCCGGGTGTGGGGCTGCCAGGTGTATAC
CCAGGTGGCGTGCTCCCAGGAGCTCGGTTCCCCGGTGTGGGGGTGCTCCC
TGGAGTTCCCACTGGAGCAGGAGTTAAGCCCAAGGCTCCAGGTGTAGGTG
GAGCTTTTGCTGGAATCCCAGGAGTTGGACCCTTTGGGGGACCGCAACCT
GGAGTCCCACTGGGGTATCCCATCAAGGCCCCCAAGCTGCCTGGTGGCTA
TGGACTGCCCTACACCACAGGGAAACTGCCCTATGGCTATGGGCCCGGAG
GAGTGGCTGGTGCAGCGGGCAAGGCTGGTTACCCAACAGGGACAGGGGTT
GGCCCCCAGGCAGCAGCAGCAGCGGCAGCTAAAGCAGCAGCAAAGTTCGG
TGCTGGAGCAGCCGGAGTCCTCCCTGGTGTTGGAGGGGCTGGTGTTCCTG
GCGTGCCTGGGGCAATTCCTGGAATTGGAGGCATCGCAGGCGTTGGGACT
CCAGCTGCAGCTGCAGCTGCAGCAGCAGCCGCTAAGGCAGCCAAGTATGG
AGCTGCTGCAGGCTTAGTGCCTGGTGGGCCAGGCTTTGGCCCGGGAGTAG
TTGGTGTCCCAGGAGCTGGCGTTCCAGGTGTTGGTGTCCCAGGAGCTGGG
ATTCCAGTTGTCCCAGGTGCTGGGATCCCAGGTGCTGCGGTTCCAGGGGT
TGTGTCACCAGAAGCAGCTGCTAAGGCAGCTGCAAAGGCAGCCAAATACG
GGGCCAGGCCCGGAGTCGGAGTTGGAGGCATTCCTACTTACGGGGTTGGA
GCTGGGGGCTTTCCCGGCTTTGGTGTCGGAGTCGGAGGTATCCCTGGAGT
CGCAGGTGTCCCTGGTGTCGGAGGTGTTCCCGGAGTCGGAGGTGTCCCGG
GAGTTGGCATTTCCCCCGAAGCTCAGGCAGCAGCTGCCGCCAAGGCTGCC
AAGTACGGTGCTGCAGGAGCAGGAGTGCTGGGTGGGCTAGTGCCAGGTCC
CCAGGCGGCAGTCCCAGGTGTGCCGGGCACGGGAGGAGTGCCAGGAGTGG
GGACCCCAGCAGCTGCAGCTGCTAAAGCAGCCGCCAAAGCCGCCCAGTTT
GGGTTAGTTCCTGGTGTCGGCGTGGCTCCTGGAGTTGGCGTGGCTCCTGG
TGTCGGTGTGGCTCCTGGAGTTGGCTTGGCTCCTGGAGTTGGCGTGGCTC
CTGGAGTTGGTGTGGCTCCTGGCGTTGGCGTGGCTCCCGGCATTGGCCCT
GGTGGAGTTGCAGCTGCAGCAAAATCCGCTGCCAAGGTGGCTGCCAAAGC
CCAGCTCCGAGCTGCAGCTGGGCTTGGTGCTGGCATCCCTGGACTTGGAG
TTGGTGTCGGCGTCCCTGGACTTGGAGTTGGTGCTGGTGTTCCTGGACTT
GGAGTTGGTGCTGGTGTTCCTGGCTTCGGGGCAGGTGCAGATGAGGGAGT
TAGGCGGAGCCTGTCCCCTGAGCTCAGGGAAGGAGATCCCTCCTCCTCTC
AGCACCTCCCCAGCACCCCCTCATCACCCAGGGTACCTGGAGCCCTGGCT
GCCGCTAAAGCAGCCAAATATGGAGCAGCAGTGCCTGGGGTCCTTGGAGG
GCTCGGGGCTCTCGGTGGAGTAGGCATCCCAGGCGGTGTGGTGGGAGCCG
GACCCGCCGCCGCCGCTGCCGCAGCCAAAGCTGCTGCCAAAGCCGCCCAG
TTTGGCCTAGTGGGAGCCGCTGGGCTCGGAGGACTCGGAGTCGGAGGGCT
TGGAGTTCCAGGTGTTGGGGGCCTTGGAGGTATACCTCCAGCTGCAGCCG
CTAAAGCAGCTAAATACGGTGCTGCTGGCCTTGGAGGTGTCCTAGGGGGT
GCCGGGCAGTTCCCACTTGGAGGAGTGGCAGCAAGACCTGGCTTCGGATT
GTCTCCCATTTTCCCAGGTGGGGCCTGCCTGGGGAAAGCTTGTGGCCGGA
AGAGAAAATGA

Codon Optimized Elastin with DsbA Secretion Tag-His Tag-Linker-Thrombin Cleavage Site

The codon optimized polynucleotide sequence encoding the full length human elastin with DsbA secretion tag-His tag-Linker-Thrombin cleavage site is disclosed below. In SEQ ID NO: 23: nucleotides 1-72 encode the DsbA secretion tag encoding amino acids 1-24 of SEQ ID NO: 24; nucleotides 73-99 encode the 9 His tag (SEQ ID NO: 129) encoding amino acids 25-33 of SEQ ID NO: 24; nucleotides 100-111 encode the linker encoding amino acids 34-37 of SEQ ID NO: 24; nucleotides 112-135 encode the thrombin cleavage tag encoding amino acids 38-45 of SEQ ID NO: 24; nucleotides 136-2415 encode the amino acids 46-805 of the full length human elastin of SEQ ID NO: 24.

(SEQ ID NO: 23)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGGGTGGCGTACCAGGC
GCAATTCCTGGGGGTGTCCCAGGCGGTGTTTTTTATCCGGGCGCCGGTCT
TGGCGCACTGGGTGGCGGTGCACTGGGCCCGGGCGGCAAACCGCTGAAAC
CGGTACCAGGTGGTTTAGCAGGCGCCGGCTTAGGCGCAGGTCTGGGAGCA
TTTCCGGCAGTTACCTTTCCAGGGGCACTGGTTCCTGGAGGTGTGGCCGA
TGCAGCCGCGGCATATAAAGCCGCTAAAGCCGGTGCGGGTTTAGGAGGCG
TCCCAGGTGTCGGTGGCCTGGGTGTTAGCGCCGGTGCAGTTGTTCCGCAG
CCGGGAGCAGGGGTTAAACCTGGTAAAGTGCCGGGAGTAGGTCTGCCAGG
CGTTTATCCTGGTGGTGTTTTGCCGGGTGCCCGTTTTCCGGGCGTTGGTG
TTCTTCCAGGCGTGCCGACCGGAGCCGGTGTTAAACCGAAAGCCCCCGGT
GTTGGAGGTGCATTTGCAGGCATCCCGGGAGTTGGCCCGTTTGGTGGTCC
GCAACCTGGGGTTCCGTTAGGTTATCCGATTAAAGCACCGAAACTGCCCG
GCGGTTATGGTCTGCCGTACACAACCGGTAAACTGCCGTATGGTTATGGC
CCGGGTGGAGTTGCGGGTGCAGCAGGTAAAGCGGGTTATCCTACCGGAAC
CGGTGTAGGTCCGCAGGCCGCTGCTGCCGCCGCCGCAAAAGCAGCGGCTA
AATTTGGCGCCGGAGCAGCGGGTGTTCTGCCTGGAGTTGGTGGTGCGGGC
GTGCCAGGGGTACCTGGTGCAATTCCGGGTATTGGTGGTATTGCCGGTGT
CGGCACCCCGGCCGCGGCAGCTGCGGCAGCGGCGGCTGCCAAAGCTGCTA
AATACGGTGCCGCGGCGGGTCTGGTGCCAGGAGGTCCGGGTTTTGGTCCG
GGAGTGGTTGGCGTGCCTGGCGCAGGCGTTCCTGGTGTGGGCGTTCCAGG
TGCAGGGATTCCTGTTGTGCCTGGTGCCGGTATTCCCGGCGCGGCCGTTC
CGGGGGTGGTTAGCCCGGAAGCCGCAGCGAAGGCTGCGGCAAAGGCAGCA
AAGTATGGCGCACGCCCAGGAGTCGGCGTGGGTGGTATCCCGACCTATGG
GGTGGGCGCAGGGGGTTTTCCTGGTTTCGGCGTAGGTGTAGGAGGTATAC
CGGGCGTGGCCGGTGTACCAGGGGTTGGTGGCGTCCCTGGTGTTGGCGGT
GTGCCAGGTGTTGGTATTTCACCGGAAGCACAGGCAGCAGCCGCAGCTAA
GGCAGCGAAATATGGTGCCGCCGGCGCAGGAGTTTTAGGTGGGCTGGTTC
CGGGCCCGCAGGCAGCTGTGCCGGGGGTTCCAGGCACCGGTGGTGTCCCT
GGAGTCGGTACGCCGGCTGCAGCGGCAGCCAAAGCGGCTGCGAAAGCAGC
ACAGTTTGGCTTAGTACCGGGTGTGGGAGTTGCCCCCGGCGTTGGCGTTG
CTCCAGGGGTGGGTGTTGCTCCTGGCGTCGGTCTGGCTCCTGGAGTGGGC
GTAGCACCCGGTGTGGGGGTGGCCCCGGGTGTTGGGGTTGCACCGGGTAT
CGGTCCGGGCGGTGTCGCAGCAGCAGCTAAAAGCGCGGCGAAAGTTGCGG
CCAAAGCCCAACTGCGCGCCGCCGCGGGCCTCGGTGCAGGTATTCCGGGG
CTGGGTGTCGGAGTTGGAGTCCCGGGTTTGGGCGTGGGCGCGGGAGTTCC
GGGACTGGGAGTGGGTGCCGGAGTTCCTGGCTTTGGTGCAGGCGCAGATG
AAGGTGTTCGTCGTAGCCTGAGTCCGGAACTGCGTGAAGGTGATCCGAGT
AGCAGCCAGCATCTGCCGAGCACCCCGAGCAGCCCGCGTGTTCCGGGTGC
ATTAGCTGCAGCAAAAGCCGCCAAGTATGGTGCAGCCGTGCCGGGCGTCT
TAGGTGGTCTGGGCGCCCTGGGTGGTGTAGGCATTCCGGGAGGTGTTGTG
GGTGCAGGACCGGCCGCCGCAGCTGCGGCCGCCAAAGCAGCTGCAAAAGC
GGCCCAGTTTGGTTTAGTGGGCGCCGCAGGTTTAGGCGGTTTAGGTGTGG
GTGGACTGGGTGTACCTGGCGTAGGCGGTCTGGGTGGAATTCCGCCCGCA
GCGGCCGCGAAAGCGGCAAAATATGGCGCGGCAGGCCTGGGCGGCGTGCT
GGGTGGGGCAGGTCAGTTTCCGCTGGGCGGGGTTGCCGCACGTCCGGGAT
TTGGTCTGAGCCCGATTTTCCCTGGCGGCGCATGTCTGGGTAAAGCATGT
GGTCGTAAACGTAAAtaa
(SEQ ID NO: 24)
MKKIWLALAGLVLAFSASAAQYEDHHHHHHHHHSGSSLVPRGSHMGGVPG
AIPGGVPGGVFYPGAGLGALGGGALGPGGKPLKPVPGGLAGAGLGAGLGA
FPAVTFPGALVPGGVADAAAAYKAAKAGAGLGGVPGVGGLGVSAGAVVPQ
PGAGVKPGKVPGVGLPGVYPGGVLPGARFPGVGVLPGVPTGAGVKPKAPG
VGGAFAGIPGVGPFGGPQPGVPLGYPIKAPKLPGGYGLPYTTGKLPYGYG
PGGVAGAAGKAGYPTGTGVGPQAAAAAAAKAAAKFGAGAAGVLPGVGGAG
VPGVPGAIPGIGGIAGVGTPAAAAAAAAAAKAAKYGAAAGLVPGGPGFGP
GVVGVPGAGVPGVGVPGAGIPVVPGAGIPGAAVPGVVSPEAAAKAAAKAA
KYGARPGVGVGGIPTYGVGAGGFPGFGVGVGGIPGVAGVPGVGGVPGVGG
VPGVGISPEAQAAAAAKAAKYGAAGAGVLGGLVPGPQAAVPGVPGTGGVP
GVGTPAAAAAKAAAKAAQFGLVPGVGVAPGVGVAPGVGVAPGVGLAPGVG
VAPGVGVAPGVGVAPGIGPGGVAAAAKSAAKVAAKAQLRAAAGLGAGIPG
LGVGVGVPGLGVGAGVPGLGVGAGVPGFGAGADEGVRRSLSPELREGDPS
SSQHLPSTPSSPRVPGALAAAKAAKYGAAVPGVLGGLGALGGVGIPGGVV
GAGPAAAAAAAKAAAKAAQFGLVGAAGLGGLGVGGLGVPGVGGLGGIPPA
AAAKAAKYGAAGLGGVLGGAGQFPLGGVAARPGFGLSPIFPGGACLGKAC
GRKRK

The polynucleotide encoding the full length human elastin without the native sequence tag is disclosed in SEQ ID NO: 87.

(SEQ ID NO: 87)
GGTGGCGTACCAGGCGCAATTCCTGGGGGTGTCCCAGGCGGTGTTTTTTA
TCCGGGCGCCGGTCTTGGCGCACTGGGTGGCGGTGCACTGGGCCCGGGCG
GCAAACCGCTGAAACCGGTACCAGGTGGTTTAGCAGGCGCCGGCTTAGGC
GCAGGTCTGGGAGCATTTCCGGCAGTTACCTTTCCAGGGGCACTGGTTCC
TGGAGGTGTGGCCGATGCAGCCGCGGCATATAAAGCCGCTAAAGCCGGTG
CGGGTTTAGGAGGCGTCCCAGGTGTCGGTGGCCTGGGTGTTAGCGCCGGT
GC5AGTTGTTCCGCAGCCGGGAGCAGGGGTTAAACCTGGTAAAGTGCCGG
GAGTAGGTCTGCCAGGCGTTTATCCTGGTGGTGTTTTGCCGGGTGCCCGT
TTTCCGGGCGTTGGTGTTCTTCCAGGCGTGCCGACCGGAGCCGGTGTTAA
ACCGAAAGCCCCCGGTGTTGGAGGTGCATTTGCAGGCATCCCGGGAGTTG
GCCCGTTTGGTGGTCCGCAACCTGGGGTTCCGTTAGGTTATCCGATTAAA
GCACCGAAACTGCCCGGCGGTTATGGTCTGCCGTACACAACCGGTAAACT
GCCGTATGGTTATGGCCCGGGTGGAGTTGCGGGTGCAGCAGGTAAAGCGG
GTTATCCTACCGGAACCGGTGTAGGTCCGCAGGCCGCTGCTGCCGCCGCC
GCAAAAGCAGCGGCTAAATTTGGCGCCGGAGCAGCGGGTGTTCTGCCTGG
AGTTGGTGGTGCGGGCGTGCCAGGGGTACCTGGTGCAATTCCGGGTATTG
GTGGTATTGCCGGTGTCGGCACCCCGGCCGCGGCAGCTGCGGCAGCGGCG
GCTGCCAAAGCTGCTAAATACGGTGCCGCGGCGGGTCTGGTGCCAGGAGG
TCCGGGTTTTGGTCCGGGAGTGGTTGGCGTGCCTGGCGCAGGCGTTCCTG
GTGTGGGCGTTCCAGGTGCAGGGATTCCTGTTGTGCCTGGTGCCGGTATT
CCCGGCGCGGCCGTTCCGGGGGTGGTTAGCCCGGAAGCCGCAGCGAAGGC
TGCGGCAAAGGCAGCAAAGTATGGCGCACGCCCAGGAGTCGGCGTGGGTG
GTATCCCGACCTATGGGGTGGGCGCAGGGGGTTTTCCTGGTTTCGGCGTA
GGTGTAGGAGGTATACCGGGCGTGGCCGGTGTACCAGGGGTTGGTGGCGT
CCCTGGTGTTGGCGGTGTGCCAGGTGTTGGTATTTCACCGGAAGCACAGG
CAGCAGCCGCAGCTAAGGCAGCGAAATATGGTGCCGCCGGCGCAGGAGTT
TTAGGTGGGCTGGTTCCGGGCCCGCAGGCAGCTGTGCCGGGGGTTCCAGG
CACCGGTGGTGTCCCTGGAGTCGGTACGCCGGCTGCAGCGGCAGCCAAAG
CGGCTGCGAAAGCAGCACAGTTTGGCTTAGTACCGGGTGTGGGAGTTGCC
CCCGGCGTTGGCGTTGCTCCAGGGGTGGGTGTTGCTCCTGGCGTCGGTCT
GGCTCCTGGAGTGGGCGTAGCACCCGGTGTGGGGGTGGCCCCGGGTGTTG
GGGTTGCACCGGGTATCGGTCCGGGCGGTGTCGCAGCAGCAGCTAAAAGC
GCGGCGAAAGTTGCGGCCAAAGCCCAACTGCGCGCCGCCGCGGGCCTCGG
TGCAGGTATTCCGGGGCTGGGTGTCGGAGTTGGAGTCCCGGGTTTGGGCG
TGGGCGCGGGAGTTCCGGGACTGGGAGTGGGTGCCGGAGTTCCTGGCTTT
GGTGCAGGCGCAGATGAAGGTGTTCGTCGTAGCCTGAGTCCGGAACTGCG
TGAAGGTGATCCGAGTAGCAGCCAGCATCTGCCGAGCACCCCGAGCAGCC
CGCGTGTTCCGGGTGCATTAGCTGCAGCAAAAGCCGCCAAGTATGGTGCA
GCCGTGCCGGGCGTCTTAGGTGGTCTGGGCGCCCTGGGTGGTGTAGGCAT
TCCGGGAGGTGTTGTGGGTGCAGGACCGGCCGCCGCAGCTGCGGCCGCCA
AAGCAGCTGCAAAAGCGGCCCAGTTTGGTTTAGTGGGCGCCGCAGGTTTA
GGCGGTTTAGGTGTGGGTGGACTGGGTGTACCTGGCGTAGGCGGTCTGGG
TGGAATTCCGCCCGCAGCGGCCGCGAAAGCGGCAAAATATGGCGCGGCAG
GCCTGGGCGGCGTGCTGGGTGGGGCAGGTCAGTTTCCGCTGGGCGGGGTT
GCCGCACGTCCGGGATTTGGTCTGAGCCCGATTTTCCCTGGCGGCGCATG
TCTGGGTAAAGCATGTGGTCGTAAACGTAAAtaa

The full length human elastin sequence without the native sequence tag is disclosed in SEQ ID NO: 88.

(SEQ ID NO: 88)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQAAAAAKAAKYGAAGAGVLGGLVPGPQAAVPGVPG
TGGVPGVGTPAAAAAKAAAKAAQFGLVPGVGVAPGVGVAPGVGVAPGVGL
APGVGVAPGVGVAPGVGVAPGIGPGGVAAAAKSAAKVAAKAQLRAAAGLG
AGIPGLGVGVGVPGLGVGAGVPGLGVGAGVPGFGAGADEGVRRSLSPELR
EGDPSSSQHLPSTPSSPRVPGALAAAKAAKYGAAVPGVLGGLGALGGVGI
PGGVVGAGPAAAAAAAKAAAKAAQFGLVGAAGLGGLGVGGLGVPGVGGLG
GIPPAAAAKAAKYGAAGLGGVLGGAGQFPLGGVAARPGFGLSPIFPGGAC
LGKACGRKRK

Codon Optimized Elastin with DsbA Secretion Tag

The codon optimized polynucleotide sequence encoding the full length human elastin with a DsbA secretion tag is disclosed in SEQ ID NO: 25. In SEQ ID NO: 25: nucleotides 1-72 encode the DsbA secretion tag encoding amino acids 1-24 of SEQ ID NO: 26; nucleotides 73-2355 encode the amino acids 25-785 of the full length human elastin of SEQ ID NO: 26.

(SEQ ID NO: 25)
ATGAAAAAGATTTGGCTGGCGCTGGCTGGTTTAGTTTTAGCGTTTAGCGC
ATCGGCGGCGCAGTATGAAGATATGGGTGGCGTACCAGGCGCAATTCCTG
GGGGTGTCCCAGGCGGTGTTTTTTATCCGGGCGCCGGTCTTGGCGCACTG
GGTGGCGGTGCACTGGGCCCGGGCGGCAAACCGCTGAAACCGGTACCAGG
TGGTTTAGCAGGCGCCGGCTTAGGCGCAGGTCTGGGAGCATTTCCGGCAG
TTACCTTTCCAGGGGCACTGGTTCCTGGAGGTGTGGCCGATGCAGCCGCG
GCATATAAAGCCGCTAAAGCCGGTGCGGGTTTAGGAGGCGTCCCAGGTGT
CGGTGGCCTGGGTGTTAGCGCCGGTGCAGTTGTTCCGCAGCCGGGAGCAG
GGGTTAAACCTGGTAAAGTGCCGGGAGTAGGTCTGCCAGGCGTTTATCCT
GGTGGTGTTTTGCCGGGTGCCCGTTTTCCGGGCGTTGGTGTTCTTCCAGG
CGTGCCGACCGGAGCCGGTGTTAAACCGAAAGCCCCCGGTGTTGGAGGTG
CATTTGCAGGCATCCCGGGAGTTGGCCCGTTTGGTGGTCCGCAACCTGGG
GTTCCGTTAGGTTATCCGATTAAAGCACCGAAACTGCCCGGCGGTTATGG
TCTGCCGTACACAACCGGTAAACTGCCGTATGGTTATGGCCCGGGTGGAG
TTGCGGGTGCAGCAGGTAAAGCGGGTTATCCTACCGGAACCGGTGTAGGT
CCGCAGGCCGCTGCTGCCGCCGCCGCAAAAGCAGCGGCTAAATTTGGCGC
CGGAGCAGCGGGTGTTCTGCCTGGAGTTGGTGGTGCGGGCGTGCCAGGGG
TACCTGGTGCAATTCCGGGTATTGGTGGTATTGCCGGTGTCGGCACCCCG
GCCGCGGCAGCTGCGGCAGCGGCGGCTGCCAAAGCTGCTAAATACGGTGC
CGCGGCGGGTCTGGTGCCAGGAGGTCCGGGTTTTGGTCCGGGAGTGGTTG
GCGTGCCTGGCGCAGGCGTTCCTGGTGTGGGCGTTCCAGGTGCAGGGATT
CCTGTTGTGCCTGGTGCCGGTATTCCCGGCGCGGCCGTTCCGGGGGTGGT
TAGCCCGGAAGCCGCAGCGAAGGCTGCGGCAAAGGCAGCAAAGTATGGCG
CACGCCCAGGAGTCGGCGTGGGTGGTATCCCGACCTATGGGGTGGGCGCA
GGGGGTTTTCCTGGTTTCGGCGTAGGTGTAGGAGGTATACCGGGCGTGGC
CGGTGTACCAGGGGTTGGTGGCGTCCCTGGTGTTGGCGGTGTGCCAGGTG
TTGGTATTTCACCGGAAGCACAGGCAGCAGCCGCAGCTAAGGCAGCGAAA
TATGGTGCCGCCGGCGCAGGAGTTTTAGGTGGGCTGGTTCCGGGCCCGCA
GGCAGCTGTGCCGGGGGTTCCAGGCACCGGTGGTGTCCCTGGAGTCGGTA
CGCCGGCTGCAGCGGCAGCCAAAGCGGCTGCGAAAGCAGCACAGTTTGGC
TTAG5TACCGGGTGTGGGAGTTGCCCCCGGCGTTGGCGTTGCTCCAGGGG
TGGGTGTTGCTCCTGGCGTCGGTCTGGCTCCTGGAGTGGGCGTAGCACCC
GGTGTGGGGGTGGCCCCGGGTGTTGGGGTTGCACCGGGTATCGGTCCGGG
CGGTGTCGCAGCAGCAGCTAAAAGCGCGGCGAAAGTTGCGGCCAAAGCCC
AACTGCGCGCCGCCGCGGGCCTCGGTGCAGGTATTCCGGGGCTGGGTGTC
GGAGTTGGAGTCCCGGGTTTGGGCGTGGGCGCGGGAGTTCCGGGACTGGG
AGTGGGTGCCGGAGTTCCTGGCTTTGGTGCAGGCGCAGATGAAGGTGTTC
GTCGTAGCCTGAGTCCGGAACTGCGTGAAGGTGATCCGAGTAGCAGCCAG
CATCTGCCGAGCACCCCGAGCAGCCCGCGTGTTCCGGGTGCATTAGCTGC
AGCAAAAGCCGCCAAGTATGGTGCAGCCGTGCCGGGCGTCTTAGGTGGTC
TGGGCGCCCTGGGTGGTGTAGGCATTCCGGGAGGTGTTGTGGGTGCAGGA
CCGGCCGCCGCAGCTGCGGCCGCCAAAGCAGCTGCAAAAGCGGCCCAGTT
TGGTTTAGTGGGCGCCGCAGGTTTAGGCGGTTTAGGTGTGGGTGGACTGG
GTGTACCTGGCGTAGGCGGTCTGGGTGGAATTCCGCCCGCAGCGGCCGCG
AAAGCGGCAAAATATGGCGCGGCAGGCCTGGGCGGCGTGCTGGGTGGGGC
AGGTCAGTTTCCGCTGGGCGGGGTTGCCGCACGTCCGGGATTTGGTCTGA
GCCCGATTTTCCCTGGCGGCGCATGTCTGGGTAAAGCATGTGGTCGTAAA
CGTAAAtaa
(SEQ ID NO: 26)
MKKIWLALAGLVLAFSASAAQYEDMGGVPGAIPGGVPGGVFYPGAGLGAL
GZSGGALGPGGKPLKPVPGGLAGAGLGAGLGAFPAVTFPGALVPGGVADA
AAAYKAAKAGAGLGGVPGVGGLGVSAGAVVPQPGAGVKPGKVPGVGLPGV
YPGGVLPGARFPGVGVLPGVPTGAGVKPKAPGVGGAFAGIPGVGPFGGPQ
PGVPLGYPIKAPKLPGGYGLPYTTGKLPYGYGPGGVAGAAGKAGYPTGTG
VGPQAAAAAAAKAAAKFGAGAAGVLPGVGGAGVPGVPGAIPGIGGIAGVG
TPAAAAAAAAAAKAAKYGAAAGLVPGGPGFGPGVVGVPGAGVPGVGVPGA
GIPVVPGAGIPGAAVPGVVSPEAAAKAAAKAAKYGARPGVGVGGIPTYGV
GAGGFPGFGVGVGGIPGVAGVPGVGGVPGVGGVPGVGISPEAQAAAAAKA
AKYGAAGAGVLGGLVPGPQAAVPGVPGTGGVPGVGTPAAAAAKAAAKAAQ
FGLVPGVGVAPGVGVAPGVGVAPGVGLAPGVGVAPGVGVAPGVGVAPGIG
PGGVAAAAKSAAKVAAKAQLRAAAGLGAGIPGLGVGVGVPGLGVGAGVPG
LGVGAGVPGFGAGADEGVRRSLSPELREGDPSSSQHLPSTPSSPRVPGAL
AAAKAAKYGAAVPGVLGGLGALGGVGIPGGVVGAGPAAAAAAAKAAAKAA
QFGLVGAAGLGGLGVGGLGVPGVGGLGGIPPAAAAKAAKYGAAGLGGVLG
GAGQFPLGGVAARPGFGLSPIFPGGACLGKACGRKRK

The polynucleotides of SEQ ID NO: 22 was codon optimized and synthesized by Gen9 DNA, now Ginkgo Bioworks internal synthesis. Overlaps between the pET28 vector and SEQ ID NO: 22 was designed to be between 30 and 40 bp long and added using PCR with the enzyme PRIMESTAR® GXL polymerase (<http://www.clontech.com/US/Products/PCR/GC_Rich/PrimeSTAR_GXL_DNA_Polymerase?sitex=10020:22372:US>). The opened pET28a vector and insert DNA (SEQ ID NO: 22) was then assembled together into the final plasmid using SGI GIBSON ASSEMBLY® (<https://us.vwr.com/store/product/17613857/gibson-assembly-hifi-1-step-kit-synthetic-genomics-inc>). Sequence of plasmid was then verified through sanger sequencing through Eurofins Genomics (<www.eurofinsgenomics.com>).

The transformed cells were cultivated in minimal media and frozen in 1.5 aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

A bioreactor was prepared with 2.7 L of minimal media+glucose and 300 ml of OD600 of 5-10 culture was added to bring the starting volume to 3 L. Cells were grown at 28° C., pH7 with Dissolved Oxygen maintained at 20% saturation using a cascade containing agitation, air and oxygen. pH was controlled using 28% w/w Ammonium hydroxide solution. Fermentation was run in a fed-batch mode using a DO-stat based feeding algorithm once the initial bolus of 40 g/L was depleted around 13 hours. After 24-26 hours of initial growth, the OD600 reached above 100. At this point, 300 mL of 500 g/L sucrose was added and temperature was reduced to 25 C. High density culture was induced for protein production using 1 mM IPTG. Fermentation was continued for another 20-24 hours and cells were harvested using a bench top centrifuge at 9000 rcf, 15C for 60 minutes. Cell pellet recovered from centrifugation was resuspended in a buffer containing 0.5M NaCl and 0.1 M KH2PO4 at pH8 in a weight by weight ratio of 2× buffer to 1× cells.

The fermentations were performed at various temperature ranging from 25° to 28° C. For some fermentations, the temperature of the fermentation was maintained at a constant temperature and immediately upon completion of fermentation (OD600 of 5-10) the elastin was purified. For other fermentations, the temperature of the fermentations is maintained for a desired period of time and when cell densities of OD600 of 5-10 are reached, the temperature is reduced to induce protein production. Typically, the temperature is reduced from 28° C. to 25° C. After the fermentation at 25° C. is continued for 40-60 hours, the elastin is isolated.

The harvested cells were disrupted in a homogenizer at 14,000 psi pressure in 2 passes. Resulting slurry contained the collagen protein along with other proteins.

The supernatant from the homogenized cells was analyzed on an SDS-PAGE gel and a clear band was observed at around 70 kilodaltons corresponding to the expected size of 68 kilodaltons. The purified elastin is analyzed by mass spectrometry.

Full Length Elastin with DsbA Secretion Tag-His Tag-Linker-Thrombin Cleavage Site and GFP Beta-Lactamase Fusion

A human elastin with DsbA secretion tag-His tag-Linker-Thrombin cleavage site and GFP Beta-lactamase fusion is disclosed below. The codon-optimized nucleotide sequence encoding this elastin is provided in SEQ ID NO: 27. The amino acid sequence is disclosed in SEQ ID NO: 28. The DsbA secretion tag is encoded by nucleotides 1-72 and encodes amino acids 1-24. The His tag is encoded by nucleotides 73-99 and encodes a 9 histidine tag (SEQ ID NO: 129) of amino acids 25-33. The linker is encoded by nucleotides 100-111 and encodes amino acids 34-37. The thrombin cleavage side is encoded by nucleotides 112-135 and encodes amino acids 38-45. The green fluorescent protein (GFP) with linker is encoded by nucleotides 136-873 and encodes amino acids 46-291. The full-length elastin sequence is encoded by nucleotides 874-3153 and encodes amino acids 292-1051. The Beta-lactamase with linker is encoded by nucleotides 3154-3945 and encodes amino acids 1052-1315.

(SEQ ID NO: 27)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGTCTGGCTCGAGCAGT
AAAGGTGAAGAACTGTTCACCGGTGTTGTTCCGATCCTGGTTGAACTGGA
TGGTGATGTTAACGGCCACAAATTCTCTGTTCGTGGTGAAGGTGAAGGTG
ATGCAACCAACGGTAAACTGACCCTGAAATTCATCTGCACTACCGGTAAA
CTGCCGGTTCCATGGCCGACTCTGGTGACTACCCTGACCTATGGTGTTCA
GTGTTTTTCTCGTTACCCGGATCACATGAAGCAGCATGATTTCTTCAAAT
CTGCAATGCCGGAAGGTTATGTACAGGAGCGCACCATTTCTTTCAAAGAC
GATGGCACCTACAAAACCCGTGCAGAGGTTAAATTTGAAGGTGATACTCT
GGTGAACCGTATTGAACTGAAAGGCATTGATTTCAAAGAGGACGGCAACA
TCCTGGGCCACAAACTGGAATATAACTTCAACTCCCATAACGTTTACATC
ACCGCAGACAAACAGAAGAACGGTATCAAAGCTAACTTCAAAATTCGCCA
TAACGTTGAAGACGGTAGCGTACAGCTGGCGGACCACTACCAGCAGAACA
CTCCGATCGGTGATGGTCCGGTTCTGCTGCCGGATAACCACTACCTGTCC
ACCCAGTCTaaaCTGTCCAAAGACCCGAACGAAAAGCGCGACCACATGGT
GCTGCTGGAGTTCGTTACTGCAGCAGGTATCACGCACGGCATGGATGAAC
TCTACAAATCTGGCGCGCCGGGCGGTGGCGTACCAGGCGCAATTCCTGGG
GGTGTCCCAGGCGGTGTTTTTTATCCGGGCGCCGGTCTTGGCGCACTGGG
TGGCGGTGCACTGGGCCCGGGCGGCAAACCGCTGAAACCGGTACCAGGTG
GTTTAGCAGGCGCCGGCTTAGGCGCAGGTCTGGGAGCATTTCCGGCAGTT
ACCTTTCCAGGGGCACTGGTTCCTGGAGGTGTGGCCGATGCAGCCGCGGC
ATATAAAGCCGCTAAAGCCGGTGCGGGTTTAGGAGGCGTCCCAGGTGTCG
GTGGCCTGGGTGTTAGCGCCGGTGCAGTTGTTCCGCAGCCGGGAGCAGGG
GTTAAACCTGGTAAAGTGCCGGGAGTAGGTCTGCCAGGCGTTTATCCTGG
TGGTGTTTTGCCGGGTGCCCGTTTTCCGGGCGTTGGTGTTCTTCCAGGCG
TGCCGACCGGAGCCGGTGTTAAACCGAAAGCCCCCGGTGTTGGAGGTGCA
TTTGCAGGCATCCCGGGAGTTGGCCCGTTTGGTGGTCCGCAACCTGGGGT
TCCGTTAGGTTATCCGATTAAAGCACCGAAACTGCCCGGCGGTTATGGTC
TGCCGTACACAACCGGTAAACTGCCGTATGGTTATGGCCCGGGTGGAGTT
GCGGGTGCAGCAGGTAAAGCGGGTTATCCTACCGGAACCGGTGTAGGTCC
GCAGGCCGCTGCTGCCGCCGCCGCAAAAGCAGCGGCTAAATTTGGCGCCG
GAGCAGCGGGTGTTCTGCCTGGAGTTGGTGGTGCGGGCGTGCCAGGGGTA
CCTGGTGCAATTCCGGGTATTGGTGGTATTGCCGGTGTCGGCACCCCGGC
CGCGGCAGCTGCGGCAGCGGCGGCTGCCAAAGCTGCTAAATACGGTGCCG
CGGCGGGTCTGGTGCCAGGAGGTCCGGGTTTTGGTCCGGGAGTGGTTGGC
GTGCCTGGCGCAGGCGTTCCTGGTGTGGGCGTTCCAGGTGCAGGGATTCC
TGTTGTGCCTGGTGCCGGTATTCCCGGCGCGGCCGTTCCGGGGGTGGTTA
GCCCGGAAGCCGCAGCGAAGGCTGCGGCAAAGGCAGCAAAGTATGGCGCA
CGCCCAGGAGTCGGCGTGGGTGGTATCCCGACCTATGGGGTGGGCGCAGG
GGGTTTTCCTGGTTTCGGCGTAGGTGTAGGAGGTATACCGGGCGTGGCCG
GTGTACCAGGGGTTGGTGGCGTCCCTGGTGTTGGCGGTGTGCCAGGTGTT
GGTATTTCACCGGAAGCACAGGCAGCAGCCGCAGCTAAGGCAGCGAAATA
TGGTGCCGCCGGCGCAGGAGTTTTAGGTGGGCTGGTTCCGGGCCCGCAGG
CAGCTGTGCCGGGGGTTCCAGGCACCGGTGGTGTCCCTGGAGTCGGTACG
CCGGCTGCAGCGGCAGCCAAAGCGGCTGCGAAAGCAGCACAGTTTGGCTT
AGTACCGGGTGTGGGAGTTGCCCCCGGCGTTGGCGTTGCTCCAGGGGTGG
GTGTTGCTCCTGGCGTCGGTCTGGCTCCTGGAGTGGGCGTAGCACCCGGT
GTGGGGGTGGCCCCGGGTGTTGGGGTTGCACCGGGTATCGGTCCGGGCGG
TGTCGCAGCAGCAGCTAAAAGCGCGGCGAAAGTTGCGGCCAAAGCCCAAC
TGCGCGCCGCCGCGGGCCTCGGTGCAGGTATTCCGGGGCTGGGTGTCGGA
GTTGGAGTCCCGGGTTTGGGCGTGGGCGCGGGAGTTCCGGGACTGGGAGT
GGGTGCCGGAGTTCCTGGCTTTGGTGCAGGCGCAGATGAAGGTGTTCGTC
GTAGCCTGAGTCCGGAACTGCGTGAAGGTGATCCGAGTAGCAGCCAGCAT
CTGCCGAGCACCCCGAGCAGCCCGCGTGTTCCGGGTGCATTAGCTGCAGC
AAAAGCCGCCAAGTATGGTGCAGCCGTGCCGGGCGTCTTAGGTGGTCTGG
GCGCCCTGGGTGGTGTAGGCATTCCGGGAGGTGTTGTGGGTGCAGGACCG
GCCGCCGCAGCTGCGGCCGCCAAAGCAGCTGCAAAAGCGGCCCAGTTTGG
TTTAGTGGGCGCCGCAGGTTTAGGCGGTTTAGGTGTGGGTGGACTGGGTG
TACCTGGCGTAGGCGGTCTGGGTGGAATTCCGCCCGCAGCGGCCGCGAAA
GCGGCAAAATATGGCGCGGCAGGCCTGGGCGGCGTGCTGGGTGGGGCAGG
TCAGTTTCCGCTGGGCGGGGTTGCCGCACGTCCGGGATTTGGTCTGAGCC
CGATTTTCCCTGGCGGCGCATGTCTGGGTAAAGCATGTGGTCGTAAACGT
AAAcacccagaaacgctggtgaaagtaaaagatgctgaagatcagagggt
gcacgagtgggttacatcgaactggatctcaacagcggtaagatccttga
gagttttcgccccgaagaacgttttccaatgatgagcacttttaaagttc
tgctatgtggcgcggtattatcccgtattgacgccgggcaagagcaactc
ggtcgccgcatacactattctcagaatgacttggttgagtactcaccagt
cacagaaaagcatcttacggatggcatgacagtaagagaattatgcagtg
ctgccataaccatgagtgataacactgcggccaacttacttctgacaacg
atcggaggaccgaaggagctaaccgcttttttgcacaacatgggggatca
tgtaactcgccttgatcgttgggaaccggagctgaatgaagccataccaa
acgacgagcgtgacaccacgatgcctgtagcaatggcaacaacgttgcgc
aaactattaactggcgaactacttactctagcttcccggcaacaattaat
agactggatggaggcggataaagttgcaggaccacttctgcgctcggccc
ttccggctggctggtttattgctgataaatctggagccggtgagcgtggg
tctcgcggtatcattgcagcactggggccagatggtaagccctcccgtat
cgtagttatctacacgacggggagtcaggcaactatggatgaacgaaata
gacagatcgctgagataggtgcctcactgattaagcattggtaa
(SEQ ID NO: 28)
MKKIWLALAGLVLAFSASAAQYEDHHHHHHHHHSGSSLVPRGSHMSGSSS
KGEELFTGVVPILVELDGDVNGHKFSVRGEGEGDATNGKLTLKFICTTGK
LPVPWPTLVTTLTYGVQCFSRYPDHMKQHDFFKSAMPEGYVQERTISFKD
DGTYKTRAEVKFEGDTLVNRIELKGIDFKEDGNILGHKLEYNFNSHNVYI
TADKQKNGIKANFKIRHNVEDGSVQLADHYQQNTPIGDGPVLLPDNHYLS
TQSKLSKDPNEKRDHMVLLEFVTAAGITHGMDELYKSGAPGGGVPGAIPG
GVPGGVFYPGAGLGALGGGALGPGGKPLKPVPGGLAGAGLGAGLGAFPAV
TFPGALVPGGVADAAAAYKAAKAGAGLGGVPGVGGLGVSAGAVVPQPGAG
VKPGKVPGVGLPGVYPGGVLPGARFPGVGVLPGVPTGAGVKPKAPGVGGA
FAGIPGVGPFGGPQPGVPLGYPIKAPKLPGGYGLPYTTGKLPYGYGPGGV
AGAAGKAGYPTGTGVGPQAAAAAAAKAAAKFGAGAAGVLPGVGGAGVPGV
PGAIPGIGGIAGVGTPAAAAAAAAAAKAAKYGAAAGLVPGGPGFGPGVVG
VPGAGVPGVGVPGAGIPVVPGAGIPGAAVPGVVSPEAAAKAAAKAAKYGA
RPGVGVGGIPTYGVGAGGFPGFGVGVGGIPGVAGVPGVGGVPGVGGVPGV
GISPEAQAAAAAKAAKYGAAGAGVLGGLVPGPQAAVPGVPGTGGVPGVGT
PAAAAAKAAAKAAQFGLVPGVGVAPGVGVAPGVGVAPGVGLAPGVGVAPG
VGVAPGVGVAPGIGPGGVAAAAKSAAKVAAKAQLRAAAGLGAGIPGLGVG
VGVPGLGVGAGVPGLGVGAGVPGFGAGADEGVRRSLSPELREGDPSSSQH
LPSTPSSPRVPGALAAAKAAKYGAAVPGVLGGLGALGGVGIPGGVVGAGP
AAAAAAAKAAAKAAQFGLVGAAGLGGLGVGGLGVPGVGGLGGIPPAAAAK
AAKYGAAGLGGVLGGAGQFPLGGVAARPGFGLSPIFPGGACLGKACGRKR
KHPETLVKVKDAEDQLGARVGYIELDLNSGKILESFRPEERFPMMSTFKV
LLCGAVLSRIDAGQEQLGRRIHYSQNDLVEYSPVTEKHLTDGMTVRELCS
AAITMSDNTAANLLLTTIGGPKELTAFLHNMGDHVTRLDRWEPELNEAIP
NDERDTTMPVAMATTLRKLLTGELLTLASRQQLIDWMEADKVAGPLLRSA
LPAGWFIADKSGAGERGSRGIIAALGPDGKPSRIVVIYTTGSQATMDERN
RQIAEIGASLIKHW

### Example 5: Production of Truncated Elastin

Truncated human elastin is produced using the expression system as described in Example 4. The full length amino acid sequence lacking the native secretion tag is disclosed in SEQ ID NO: 29.

(SEQ ID NO: 29)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQAAAAAKAAKYGAAGAGVLGGLVPGPQAAVPGVPG
TGGVPGVGTPAAAAAKAAAKAAQFGLVPGVGVAPGVGVAPGVGVAPGVGL
APGVGVAPGVGVAPGVGVAPGIGPGGVAAAAKSAAKVAAKAQLRAAAGLG
AGIPGLGVGVGVPGLGVGAGVPGLGVGAGVPGFGAGADEGVRRSLSPELR
EGDPSSSQHLPSTPSSPRVPGALAAAKAAKYGAAVPGVLGGLGALGGVGI
PGGVVGAGPAAAAAAAKAAAKAAQFGLVGAAGLGGLGVGGLGVPGVGGLG
GIPPAAAAKAAKYGAAGLGGVLGGAGQFPLGGVAARPGFGLSPIFPGGAC
LGKACGRKRK

The codon optimized polynucleotide sequence encoding the full length human elastin lacking the native secretion tag is disclosed in SEQ ID NO: 30.

(SEQ ID NO: 30)
GGTGGCGTACCAGGCGCAATTCCTGGGGGTGTCCCAGGCGGTGTTTTTTA
TCCGGGCGCCGGTCTTGGCGCACTGGGTGGCGGTGCACTGGGCCCGGGCG
GCAAACCGCTGAAACCGGTACCAGGTGGTTTAGCAGGCGCCGGCTTAGGC
GCAGGTCTGGGAGCATTTCCGGCAGTTACCTTTCCAGGGGCACTGGTTCC
TGGAGGTGTGGCCGATGCAGCCGCGGCATATAAAGCCGCTAAAGCCGGTG
CGGGTTTAGGAGGCGTCCCAGGTGTCGGTGGCCTGGGTGTTAGCGCCGGT
GCAGTTGTTCCGCAGCCGGGAGCAGGGGTTAAACCTGGTAAAGTGCCGGG
AGTAGGTCTGCCAGGCGTTTATCCTGGTGGTGTTTTGCCGGGTGCCCGTT
TTCCGGGCGTTGGTGTTCTTCCAGGCGTGCCGACCGGAGCCGGTGTTAAA
CCGAAAGCCCCCGGTGTTGGAGGTGCATTTGCAGGCATCCCGGGAGTTGG
CCCGTTTGGTGGTCCGCAACCTGGGGTTCCGTTAGGTTATCCGATTAAAG
CACCGAAACTGCCCGGCGGTTATGGTCTGCCGTACACAACCGGTAAACTG
CCGTATGGTTATGGCCCGGGTGGAGTTGCGGGTGCAGCAGGTAAAGCGGG
TTATCCTACCGGAACCGGTGTAGGTCCGCAGGCCGCTGCTGCCGCCGCCG
CAAAAGCAGCGGCTAAATTTGGCGCCGGAGCAGCGGGTGTTCTGCCTGGA
GTTGGTGGTGCGGGCGTGCCAGGGGTACCTGGTGCAATTCCGGGTATTGG
TGGTATTGCCGGTGTCGGCACCCCGGCCGCGGCAGCTGCGGCAGCGGCGG
CTGCCAAAGCTGCTAAATACGGTGCCGCGGCGGGTCTGGTGCCAGGAGGT
CCGGGTTTTGGTCCGGGAGTGGTTGGCGTGCCTGGCGCAGGCGTTCCTGG
TGTGGGCGTTCCAGGTGCAGGGATTCCTGTTGTGCCTGGTGCCGGTATTC
CCGGCGCGGCCGTTCCGGGGGTGGTTAGCCCGGAAGCCGCAGCGAAGGCT
GCGGCAAAGGCAGCAAAGTATGGCGCACGCCCAGGAGTCGGCGTGGGTGG
TATCCCGACCTATGGGGTGGGCGCAGGGGGTTTTCCTGGTTTCGGCGTAG
GTGTAGGAGGTATACCGGGCGTGGCCGGTGTACCAGGGGTTGGTGGCGTC
CCTGGTGTTGGCGGTGTGCCAGGTGTTGGTATTTCACCGGAAGCACAGGC
AGCAGCCGCAGCTAAGGCAGCGAAATATGGTGCCGCCGGCGCAGGAGTTT
TAGGTGGGCTGGTTCCGGGCCCGCAGGCAGCTGTGCCGGGGGTTCCAGGC
ACCGGTGGTGTCCCTGGAGTCGGTACGCCGGCTGCAGCGGCAGCCAAAGC
GGCTGCGAAAGCAGCACAGTTTGGCTTAGTACCGGGTGTGGGAGTTGCCC
CCGGCGTTGGCGTTGCTCCAGGGGTGGGTGTTGCTCCTGGCGTCGGTCTG
GCTCCTGGAGTGGGCGTAGCACCCGGTGTGGGGGTGGCCCCGGGTGTTGG
GGTTGCACCGGGTATCGGTCCGGGCGGTGTCGCAGCAGCAGCTAAAAGCG
CGGCGAAAGTTGCGGCCAAAGCCCAACTGCGCGCCGCCGCGGGCCTCGGT
GCAGGTATTCCGGGGCTGGGTGTCGGAGTTGGAGTCCCGGGTTTGGGCGT
GGGCGCGGGAGTTCCGGGACTGGGAGTGGGTGCCGGAGTTCCTGGCTTTG
GTGCAGGCGCAGATGAAGGTGTTCGTCGTAGCCTGAGTCCGGAACTGCGT
GAAGGTGATCCGAGTAGCAGCCAGCATCTGCCGAGCACCCCGAGCAGCCC
GCGTGTTCCGGGTGCATTAGCTGCAGCAAAAGCCGCCAAGTATGGTGCAG
CCGTGCCGGGCGTCTTAGGTGGTCTGGGCGCCCTGGGTGGTGTAGGCATT
CCGGGAGGTGTTGTGGGTGCAGGACCGGCCGCCGCAGCTGCGGCCGCCAA
AGCAGCTGCAAAAGCGGCCCAGTTTGGTTTAGTGGGCGCCGCAGGTTTAG
GCGGTTTAGGTGTGGGTGGACTGGGTGTACCTGGCGTAGGCGGTCTGGGT
GGAATTCCGCCCtaa

The amino acid sequence of a 60.7 kDa human elastin truncated at the C-terminal is disclosed in SEQ ID NO: 31. The 60.7 kDa truncated elastin has amino acids 706-761 deleted from the full length elastin.

(SEQ ID NO: 31)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQAAAAAKAAKYGAAGAGVLGGLVPGPQAAVPGVPG
TGGVPGVGTPAAAAAKAAAKAAQFGLVPGVGVAPGVGVAPGVGVAPGVGL
APGVGVAPGVGVAPGVGVAPGIGPGGVAAAAKSAAKVAAKAQLRAAAGLG
AGIPGLGVGVGVPGLGVGAGVPGLGVGAGVPGFGAGADEGVRRSLSPELR
EGDPSSSQHLPSTPSSPRVPGALAAAKAAKYGAAVPGVLGGLGALGGVGI
PGGVVGAGPAAAAAAAKAAAKAAQFGLVGAAGLGGLGVGGLGVPGVGGLG
GIPP

The codon optimized polynucleotide sequence encoding the truncated 60.7 kDa human elastin is disclosed in SEQ ID NO: 32.

(SEQ ID NO: 32)
GGTGGCGTACCAGGCGCAATTCCTGGGGGTGTCCCAGGCGGTGTTTTTTA
TCCGGGCGCCGGTCTTGGCGCACTGGGTGGCGGTGCACTGGGCCCGGGCG
GCAAACCGCTGAAACCGGTACCAGGTGGTTTAGCAGGCGCCGGCTTAGGC
GCAGGTCTGGGAGCATTTCCGGCAGTTACCTTTCCAGGGGCACTGGTTCC
TGGAGGTGTGGCCGATGCAGCCGCGGCATATAAAGCCGCTAAAGCCGGTG
CGGGTTTAGGAGGCGTCCCAGGTGTCGGTGGCCTGGGTGTTAGCGCCGGT
GCAGTTGTTCCGCAGCCGGGAGCAGGGGTTAAACCTGGTAAAGTGCCGGG
AGTAGGTCTGCCAGGCGTTTATCCTGGTGGTGTTTTGCCGGGTGCCCGTT
TTCCGGGCGTTGGTGTTCTTCCAGGCGTGCCGACCGGAGCCGGTGTTAAA
CCGAAAGCCCCCGGTGTTGGAGGTGCATTTGCAGGCATCCCGGGAGTTGG
CCCGTTTGGTGGTCCGCAACCTGGGGTTCCGTTAGGTTATCCGATTAAAG
CACCGAAACTGCCCGGCGGTTATGGTCTGCCGTACACAACCGGTAAACTG
CCGTATGGTTATGGCCCGGGTGGAGTTGCGGGTGCAGCAGGTAAAGCGGG
TTATCCTACCGGAACCGGTGTAGGTCCGCAGGCCGCTGCTGCCGCCGCCG
CAAAAGCAGCGGCTAAATTTGGCGCCGGAGCAGCGGGTGTTCTGCCTGGA
GTTGGTGGTGCGGGCGTGCCAGGGGTACCTGGTGCAATTCCGGGTATTGG
TGGTATTGCCGGTGTCGGCACCCCGGCCGCGGCAGCTGCGGCAGCGGCGG
CTGCCAAAGCTGCTAAATACGGTGCCGCGGCGGGTCTGGTGCCAGGAGGT
CCGGGTTTTGGTCCGGGAGTGGTTGGCGTGCCTGGCGCAGGCGTTCCTGG
TGTGGGCGTTCCAGGTGCAGGGATTCCTGTTGTGCCTGGTGCCGGTATTC
CCGGCGCGGCCGTTCCGGGGGTGGTTAGCCCGGAAGCCGCAGCGAAGGCT
GCGGCAAAGGCAGCAAAGTATGGCGCACGCCCAGGAGTCGGCGTGGGTGG
TATCCCGACCTATGGGGTGGGCGCAGGGGGTTTTCCTGGTTTCGGCGTAG
GTGTAGGAGGTATACCGGGCGTGGCCGGTGTACCAGGGGTTGGTGGCGTC
CCTGGTGTTGGCGGTGTGCCAGGTGTTGGTATTTCACCGGAAGCACAGGC
AGCAGCCGCAGCTAAGGCAGCGAAATATGGTGCCGCCGGCGCAGGAGTTT
TAGGTGGGCTGGTTCCGGGCCCGCAGGCAGCTGTGCCGGGGGTTCCAGGC
ACCGGTGGTGTCCCTGGAGTCGGTACGCCGGCTGCAGCGGCAGCCAAAGC
GGCTGCGAAAGCAGCACAGTTTGGCTTAGTACCGGGTGTGGGAGTTGCCC
CCGGCGTTGGCGTTGCTCCAGGGGTGGGTGTTGCTCCTGGCGTCGGTCTG
GCTCCTGGAGTGGGCGTAGCACCCGGTGTGGGGGTGGCCCCGGGTGTTGG
GGTTGCACCGGGTATCGGTCCGGGCGGTGTCGCAGCAGCAGCTAAAAGCG
CGGCGAAAGTTGCGGCCAAAGCCCAACTGCGCGCCGCCGCGGGCCTCGGT
GCAGGTATTCCGGGGCTGGGTGTCGGAGTTGGAGTCCCGGGTTTGGGCGT
GGGCGCGGGAGTTCCGGGACTGGGAGTGGGTGCCGGAGTTCCTGGCTTTG
GTGCAGGCGCAGATGAAGGTGTTCGTCGTAGCCTGAGTCCGGAACTGCGT
GAAGGTGATCCGAGTAGCAGCCAGCATCTGCCGAGCACCCCGAGCAGCCC
GCGTGTTCCGGGTGCATTAGCTGCAGCAAAAGCCGCCAAGTATGGTGCAG
CCGTGCCGGGCGTCTTAGGTGGTCTGGGCGCCCTGGGTGGTGTAGGCATT
CCGGGAGGTGTTGTGGGTGCAGGACCGGCCGCCGCAGCTGCGGCCGCCAA
AGCAGCTGCAAAAGCGGCCCAGTTTGGTTTAGTGGGCGCCGCAGGTTTAG
GCGGTTTAGGTGTGGGTGGACTGGGTGTACCTGGCGTAGGCGGTCTGGGT
GGAATTCCGCCCtaa

The amino acid sequence of a 58.8 kDa human elastin truncated at the N-50 terminal is disclosed in SEQ ID NO: 33. The 58.8 kDa truncated elastin has amino acids 2-85 deleted from the full length elastin.

(SEQ ID NO: 33)
GLGGVPGVGGLGVSAGAVVPQPGAGVKPGKVPGVGLPGVYPGGVLPGARF
PGVGVLPGVPTGAGVKPKAPGVGGAFAGIPGVGPFGGPQPGVPLGYPIKA
PKLPGGYGLPYTTGKLPYGYGPGGVAGAAGKAGYPTGTGVGPQAAAAAAA
KAAAKFGAGAAGVLPGVGGAGVPGVPGAIPGIGGIAGVGTPAAAAAAAAA
AKAAKYGAAAGLVPGGPGFGPGVVGVPGAGVPGVGVPGAGIPVVPGAGIP
GAAVPGVVSPEAAAKAAAKAAKYGARPGVGVGGIPTYGVGAGGFPGFGVG
VGGIPGVAGVPGVGGVPGVGGVPGVGISPEAQAAAAAKAAKYGAAGAGVL
GGLVPGPQAAVPGVPGTGGVPGVGTPAAAAAKAAAKAAQFGLVPGVGVAP
GVGVAPGVGVAPGVGLAPGVGVAPGVGVAPGVGVAPGIGPGGVAAAAKSA
AKVAAKAQLRAAAGLGAGIPGLGVGVGVPGLGVGAGVPGLGVGAGVPGFG
AGADEGVRRSLSPELREGDPSSSQHLPSTPSSPRVPGALAAAKAAKYGAA
VPGVLGGLGALGGVGIPGGVVGAGPAAAAAAAKAAAKAAQFGLVGAAGLG
GLGVGGLGVPGVGGLGGIPPAAAAKAAKYGAAGLGGVLGGAGQFPLGGVA
ARPGFGLSPIFPGGACLGKACGRKRK

The codon optimized polynucleotide sequence encoding the 58.8 kDa truncated human elastin is disclosed in SEQ ID NO: 34.

(SEQ ID NO: 34)
GGTTTAGGAGGCGTCCCAGGTGTCGGTGGCCTGGGTGTTAGCGCCGGTGC
AGTTGTTCCGCAGCCGGGAGCAGGGGTTAAACCTGGTAAAGTGCCGGGAG
TAGGTCTGCCAGGCGTTTATCCTGGTGGTGTTTTGCCGGGTGCCCGTTTT
CCGGGCGTTGGTGTTCTTCCAGGCGTGCCGACCGGAGCCGGTGTTAAACC
GAAAGCCCCCGGTGTTGGAGGTGCATTTGCAGGCATCCCGGGAGTTGGCC
CGTTTGGTGGTCCGCAACCTGGGGTTCCGTTAGGTTATCCGATTAAAGCA
CCGAAACTGCCCGGCGGTTATGGTCTGCCGTACACAACCGGTAAACTGCC
GTATGGTTATGGCCCGGGTGGAGTTGCGGGTGCAGCAGGTAAAGCGGGTT
ATCCTACCGGAACCGGTGTAGGTCCGCAGGCCGCTGCTGCCGCCGCCGCA
AAAGCAGCGGCTAAATTTGGCGCCGGAGCAGCGGGTGTTCTGCCTGGAGT
TGGTGGTGCGGGCGTGCCAGGGGTACCTGGTGCAATTCCGGGTATTGGTG
GTATTGCCGGTGTCGGCACCCCGGCCGCGGCAGCTGCGGCAGCGGCGGCT
GCCAAAGCTGCTAAATACGGTGCCGCGGCGGGTCTGGTGCCAGGAGGTCC
GGGTTTTGGTCCGGGAGTGGTTGGCGTGCCTGGCGCAGGCGTTCCTGGTG
TGGGCGTTCCAGGTGCAGGGATTCCTGTTGTGCCTGGTGCCGGTATTCCC
GGCGCGGCCGTTCCGGGGGTGGTTAGCCCGGAAGCCGCAGCGAAGGCTGC
GGCAAAGGCAGCAAAGTATGGCGCACGCCCAGGAGTCGGCGTGGGTGGTA
TCCCGACCTATGGGGTGGGCGCAGGGGGTTTTCCTGGTTTCGGCGTAGGT
GTAGGAGGTATACCGGGCGTGGCCGGTGTACCAGGGGTTGGTGGCGTCCC
TGGTGTTGGCGGTGTGCCAGGTGTTGGTATTTCACCGGAAGCACAGGCAG
CAGCCGCAGCTAAGGCAGCGAAATATGGTGCCGCCGGCGCAGGAGTTTTA
GGTGGGCTGGTTCCGGGCCCGCAGGCAGCTGTGCCGGGGGTTCCAGGCAC
CGGTGGTGTCCCTGGAGTCGGTACGCCGGCTGCAGCGGCAGCCAAAGCGG
CTGCGAAAGCAGCACAGTTTGGCTTAGTACCGGGTGTGGGAGTTGCCCCC
GGCGTTGGCGTTGCTCCAGGGGTGGGTGTTGCTCCTGGCGTCGGTCTGGC
TCCTGGAGTGGGCGTAGCACCCGGTGTGGGGGTGGCCCCGGGTGTTGGGG
TTGCACCGGGTATCGGTCCGGGCGGTGTCGCAGCAGCAGCTAAAAGCGCG
GCGAAAGTTGCGGCCAAAGCCCAACTGCGCGCCGCCGCGGGCCTCGGTGC
AGGTATTCCGGGGCTGGGTGTCGGAGTTGGAGTCCCGGGTTTGGGCGTGG
GCGCGGGAGTTCCGGGACTGGGAGTGGGTGCCGGAGTTCCTGGCTTTGGT
GCAGGCGCAGATGAAGGTGTTCGTCGTAGCCTGAGTCCGGAACTGCGTGA
AGGTGATCCGAGTAGCAGCCAGCATCTGCCGAGCACCCCGAGCAGCCCGC
GTGTTCCGGGTGCATTAGCTGCAGCAAAAGCCGCCAAGTATGGTGCAGCC
GTGCCGGGCGTCTTAGGTGGTCTGGGCGCCCTGGGTGGTGTAGGCATTCC
GGGAGGTGTTGTGGGTGCAGGACCGGCCGCCGCAGCTGCGGCCGCCAAAG
CAGCTGCAAAAGCGGCCCAGTTTGGTTTAGTGGGCGCCGCAGGTTTAGGC
GGTTTAGGTGTGGGTGGACTGGGTGTACCTGGCGTAGGCGGTCTGGGTGG
AATTCCGCCCGCAGCGGCCGCGAAAGCGGCAAAATATGGCGCGGCAGGCC
TGGGCGGCGTGCTGGGTGGGGCAGGTCAGTTTCCGCTGGGCGGGGTTGCC
GCACGTCCGGGATTTGGTCTGAGCCCGATTTTCCCTGGCGGCGCATGTCT
GGGTAAAGCATGTGGTCGTAAACGTAAAtaa

The amino acid sequence of a 57 kDa human elastin truncated at the C-terminal is disclosed in SEQ ID NO: 35. The 57 kDa truncated elastin has amino acids 661-761 deleted from the full length elastin.

(SEQ ID NO: 35)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQAAAAAKAAKYGAAGAGVLGGLVPGPQAAVPGVPG
TGGVPGVGTPAAAAAKAAAKAAQFGLVPGVGVAPGVGVAPGVGVAPGVGL
APGVGVAPGVGVAPGVGVAPGIGPGGVAAAAKSAAKVAAKAQLRAAAGLG
AGIPGLGVGVGVPGLGVGAGVPGLGVGAGVPGFGAGADEGVRRSLSPELR
EGDPSSSQHLPSTPSSPRVPGALAAAKAAKYGAAVPGVLGGLGALGGVGI
PGGVVGAGP

The codon optimized polynucleotide sequence encoding the 57 kDa truncated human elastin is disclosed in SEQ ID NO: 36

(SEQ ID NO: 36)
GGTGGCGTACCAGGCGCAATTCCTGGGGGTGTCCCAGGCGGTGTTTTTTA
TCCGGGCGCCGGTCTTGGCGCACTGGGTGGCGGTGCACTGGGCCCGGGCG
GCAAACCGCTGAAACCGGTACCAGGTGGTTTAGCAGGCGCCGGCTTAGGC
GCAGGTCTGGGAGCATTTCCGGCAGTTACCTTTCCAGGGGCACTGGTTCC
TGGAGGTGTGGCCGATGCAGCCGCGGCATATAAAGCCGCTAAAGCCGGTG
CGGGTTTAGGAGGCGTCCCAGGTGTCGGTGGCCTGGGTGTTAGCGCCGGT
GCAGTTGTTCCGCAGCCGGGAGCAGGGGTTAAACCTGGTAAAGTGCCGGG
AGTAGGTCTGCCAGGCGTTTATCCTGGTGGTGTTTTGCCGGGTGCCCGTT
TTCCGGGCGTTGGTGTTCTTCCAGGCGTGCCGACCGGAGCCGGTGTTAAA
CCGAAAGCCCCCGGTGTTGGAGGTGCATTTGCAGGCATCCCGGGAGTTGG
CCCGTTTGGTGGTCCGCAACCTGGGGTTCCGTTAGGTTATCCGATTAAAG
CACCGAAACTGCCCGGCGGTTATGGTCTGCCGTACACAACCGGTAAACTG
CCGTATGGTTATGGCCCGGGTGGAGTTGCGGGTGCAGCAGGTAAAGCGGG
TTATCCTACCGGAACCGGTGTAGGTCCGCAGGCCGCTGCTGCCGCCGCCG
CAAAAGCAGCGGCTAAATTTGGCGCCGGAGCAGCGGGTGTTCTGCCTGGA
GTTGGTGGTGCGGGCGTGCCAGGGGTACCTGGTGCAATTCCGGGTATTGG
TGGTATTGCCGGTGTCGGCACCCCGGCCGCGGCAGCTGCGGCAGCGGCGG
CTGCCAAAGCTGCTAAATACGGTGCCGCGGCGGGTCTGGTGCCAGGAGGT
CCGGGTTTTGGTCCGGGAGTGGTTGGCGTGCCTGGCGCAGGCGTTCCTGG
TGTGGGCGTTCCAGGTGCAGGGATTCCTGTTGTGCCTGGTGCCGGTATTC
CCGGCGCGGCCGTTCCGGGGGTGGTTAGCCCGGAAGCCGCAGCGAAGGCT
GCGGCAAAGGCAGCAAAGTATGGCGCACGCCCAGGAGTCGGCGTGGGTGG
TATCCCGACCTATGGGGTGGGCGCAGGGGGTTTTCCTGGTTTCGGCGTAG
GTGTAGGAGGTATACCGGGCGTGGCCGGTGTACCAGGGGTTGGTGGCGTC
CCTGGTGTTGGCGGTGTGCCAGGTGTTGGTATTTCACCGGAAGCACAGGC
AGCAGCCGCAGCTAAGGCAGCGAAATATGGTGCCGCCGGCGCAGGAGTTT
TAGGTGGGCTGGTTCCGGGCCCGCAGGCAGCTGTGCCGGGGGTTCCAGGC
ACCGGTGGTGTCCCTGGAGTCGGTACGCCGGCTGCAGCGGCAGCCAAAGC
GGCTGCGAAAGCAGCACAGTTTGGCTTAGTACCGGGTGTGGGAGTTGCCC
CCGGCGTTGGCGTTGCTCCAGGGGTGGGTGTTGCTCCTGGCGTCGGTCTG
GCTCCTGGAGTGGGCGTAGCACCCGGTGTGGGGGTGGCCCCGGGTGTTGG
GGTTGCACCGGGTATCGGTCCGGGCGGTGTCGCAGCAGCAGCTAAAAGCG
CGGCGAAAGTTGCGGCCAAAGCCCAACTGCGCGCCGCCGCGGGCCTCGGT
GCAGGTATTCCGGGGCTGGGTGTCGGAGTTGGAGTCCCGGGTTTGGGCGT
GGGCGCGGGAGTTCCGGGACTGGGAGTGGGTGCCGGAGTTCCTGGCTTTG
GTGCAGGCGCAGATGAAGGTGTTCGTCGTAGCCTGAGTCCGGAACTGCGT
GAAGGTGATCCGAGTAGCAGCCAGCATCTGCCGAGCACCCCGAGCAGCCC
GCGTGTTCCGGGTGCATTAGCTGCAGCAAAAGCCGCCAAGTATGGTGCAG
CCGTGCCGGGCGTCTTAGGTGGTCTGGGCGCCCTGGGTGGTGTAGGCATT
CCGGGAGGTGTTGTGGGTGCAGGACCGtaa

The amino acid sequence of a 53.9 kDa human elastin truncated at the C-terminal is disclosed in SEQ ID NO: 37. The 53.9 kDa truncated elastin has amino acids 624-761 deleted from the full length elastin.

(SEQ ID NO: 37)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQAAAAAKAAKYGAAGAGVLGGLVPGPQAAVPGVPG
TGGVPGVGTPAAAAAKAAAKAAQFGLVPGVGVAPGVGVAPGVGVAPGVGL
APGVGVAPGVGVAPGVGVAPGIGPGGVAAAAKSAAKVAAKAQLRAAAGLG
AGIPGLGVGVGVPGLGVGAGVPGLGVGAGVPGFGAGADEGVRRSLSPELR
EGDPSSSQHLPSTPSSPRVPGA

The codon optimized polynucleotide sequence encoding the 53.9 kDa truncated human elastin is disclosed in SEQ ID NO: 38

(SEQ ID NO: 38)
GGTGGCGTACCAGGCGCAATTCCTGGGGGTGTCCCAGGCGGTGTTTTTTA
TCCGGGCGCCGGTCTTGGCGCACTGGGTGGCGGTGCACTGGGCCCGGGCG
GCAAACCGCTGAAACCGGTACCAGGTGGTTTAGCAGGCGCCGGCTTAGGC
GCAGGTCTGGGAGCATTTCCGGCAGTTACCTTTCCAGGGGCACTGGTTCC
TGGAGGTGTGGCCGATGCAGCCGCGGCATATAAAGCCGCTAAAGCCGGTG
CGGGTTTAGGAGGCGTCCCAGGTGTCGGTGGCCTGGGTGTTAGCGCCGGT
GCAGTTGTTCCGCAGCCGGGAGCAGGGGTTAAACCTGGTAAAGTGCCGGG
AGTAGGTCTGCCAGGCGTTTATCCTGGTGGTGTTTTGCCGGGTGCCCGTT
TTCCGGGCGTTGGTGTTCTTCCAGGCGTGCCGACCGGAGCCGGTGTTAAA
CCGAAAGCCCCCGGTGTTGGAGGTGCATTTGCAGGCATCCCGGGAGTTGG
CCCGTTTGGTGGTCCGCAACCTGGGGTTCCGTTAGGTTATCCGATTAAAG
CACCGAAACTGCCCGGCGGTTATGGTCTGCCGTACACAACCGGTAAACTG
CCGTATGGTTATGGCCCGGGTGGAGTTGCGGGTGCAGCAGGTAAAGCGGG
TTATCCTACCGGAACCGGTGTAGGTCCGCAGGCCGCTGCTGCCGCCGCCG
CAAAAGCAGCGGCTAAATTTGGCGCCGGAGCAGCGGGTGTTCTGCCTGGA
GTTGGTGGTGCGGGCGTGCCAGGGGTACCTGGTGCAATTCCGGGTATTGG
TGGTATTGCCGGTGTCGGCACCCCGGCCGCGGCAGCTGCGGCAGCGGCGG
CTGCCAAAGCTGCTAAATACGGTGCCGCGGCGGGTCTGGTGCCAGGAGGT
CCGGGTTTTGGTCCGGGAGTGGTTGGCGTGCCTGGCGCAGGCGTTCCTGG
TGTGGGCGTTCCAGGTGCAGGGATTCCTGTTGTGCCTGGTGCCGGTATTC
CCGGCGCGGCCGTTCCGGGGGTGGTTAGCCCGGAAGCCGCAGCGAAGGCT
GCGGCAAAGGCAGCAAAGTATGGCGCACGCCCAGGAGTCGGCGTGGGTGG
TATCCCGACCTATGGGGTGGGCGCAGGGGGTTTTCCTGGTTTCGGCGTAG
GTGTAGGAGGTATACCGGGCGTGGCCGGTGTACCAGGGGTTGGTGGCGTC
CCTGGTGTTGGCGGTGTGCCAGGTGTTGGTATTTCACCGGAAGCACAGGC
AGCAGCCGCAGCTAAGGCAGCGAAATATGGTGCCGCCGGCGCAGGAGTTT
TAGGTGGGCTGGTTCCGGGCCCGCAGGCAGCTGTGCCGGGGGTTCCAGGC
ACCGGTGGTGTCCCTGGAGTCGGTACGCCGGCTGCAGCGGCAGCCAAAGC
GGCTGCGAAAGCAGCACAGTTTGGCTTAGTACCGGGTGTGGGAGTTGCCC
CCGGCGTTGGCGTTGCTCCAGGGGTGGGTGTTGCTCCTGGCGTCGGTCTG
GCTCCTGGAGTGGGCGTAGCACCCGGTGTGGGGGTGGCCCCGGGTGTTGG
GGTTGCACCGGGTATCGGTCCGGGCGGTGTCGCAGCAGCAGCTAAAAGCG
CGGCGAAAGTTGCGGCCAAAGCCCAACTGCGCGCCGCCGCGGGCCTCGGT
GCAGGTATTCCGGGGCTGGGTGTCGGAGTTGGAGTCCCGGGTTTGGGCGT
GGGCGCGGGAGTTCCGGGACTGGGAGTGGGTGCCGGAGTTCCTGGCTTTG
GTGCAGGCGCAGATGAAGGTGTTCGTCGTAGCCTGAGTCCGGAACTGCGT
GAAGGTGATCCGAGTAGCAGCCAGCATCTGCCGAGCACCCCGAGCAGCCC
GCGTGTTCCGGGTGCAtaa

The amino acid sequence of a 45.3 kDa human elastin truncated at the C-terminal is disclosed in SEQ ID NO: 39. The 45.3 kDa truncated elastin has amino acids 529-761 deleted from the full length elastin.

(SEQ ID NO: 39)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQAAAAAKAAKYGAAGAGVLGGLVPGPQAAVPGVPG
TGGVPGVGTPAAAAAKAAAKAAQFGLVPGVGVAPGVGVAPGVGVAPGVGL
APGVGVAPGVGVAPGVGVAPGIGPGGV

The codon optimized polynucleotide sequence encoding the 45.3 kDa truncated human elastin is disclosed in SEQ ID NO: 40

(SEQ ID NO: 40)
GGTGGCGTACCAGGCGCAATTCCTGGGGGTGTCCCAGGCGGTGTTTTTTA
TCCGGGCGCCGGTCTTGGCGCACTGGGTGGCGGTGCACTGGGCCCGGGCG
GCAAACCGCTGAAACCGGTACCAGGTGGTTTAGCAGGCGCCGGCTTAGGC
GCAGGTCTGGGAGCATTTCCGGCAGTTACCTTTCCAGGGGCACTGGTTCC
TGGAGGTGTGGCCGATGCAGCCGCGGCATATAAAGCCGCTAAAGCCGGTG
CGGGTTTAGGAGGCGTCCCAGGTGTCGGTGGCCTGGGTGTTAGCGCCGGT
GCAGTTGTTCCGCAGCCGGGAGCAGGGGTTAAACCTGGTAAAGTGCCGGG
AGTAGGTCTGCCAGGCGTTTATCCTGGTGGTGTTTTGCCGGGTGCCCGTT
TTCCGGGCGTTGGTGTTCTTCCAGGCGTGCCGACCGGAGCCGGTGTTAAA
CCGAAAGCCCCCGGTGTTGGAGGTGCATTTGCAGGCATCCCGGGAGTTGG
CCCGTTTGGTGGTCCGCAACCTGGGGTTCCGTTAGGTTATCCGATTAAAG
CACCGAAACTGCCCGGCGGTTATGGTCTGCCGTACACAACCGGTAAACTG
CCGTATGGTTATGGCCCGGGTGGAGTTGCGGGTGCAGCAGGTAAAGCGGG
TTATCCTACCGGAACCGGTGTAGGTCCGCAGGCCGCTGCTGCCGCCGCCG
CAAAAGCAGCGGCTAAATTTGGCGCCGGAGCAGCGGGTGTTCTGCCTGGA
GTTGGTGGTGCGGGCGTGCCAGGGGTACCTGGTGCAATTCCGGGTATTGG
TGGTATTGCCGGTGTCGGCACCCCGGCCGCGGCAGCTGCGGCAGCGGCGG
CTGCCAAAGCTGCTAAATACGGTGCCGCGGCGGGTCTGGTGCCAGGAGGT
CCGGGTTTTGGTCCGGGAGTGGTTGGCGTGCCTGGCGCAGGCGTTCCTGG
TGTGGGCGTTCCAGGTGCAGGGATTCCTGTTGTGCCTGGTGCCGGTATTC
CCGGCGCGGCCGTTCCGGGGGTGGTTAGCCCGGAAGCCGCAGCGAAGGCT
GCGGCAAAGGCAGCAAAGTATGGCGCACGCCCAGGAGTCGGCGTGGGTGG
TATCCCGACCTATGGGGTGGGCGCAGGGGGTTTTCCTGGTTTCGGCGTAG
GTGTAGGAGGTATACCGGGCGTGGCCGGTGTACCAGGGGTTGGTGGCGTC
CCTGGTGTTGGCGGTGTGCCAGGTGTTGGTATTTCACCGGAAGCACAGGC
AGCAGCCGCAGCTAAGGCAGCGAAATATGGTGCCGCCGGCGCAGGAGTTT
TAGGTGGGCTGGTTCCGGGCCCGCAGGCAGCTGTGCCGGGGGTTCCAGGC
ACCGGTGGTGTCCCTGGAGTCGGTACGCCGGCTGCAGCGGCAGCCAAAGC
GGCTGCGAAAGCAGCACAGTTTGGCTTAGTACCGGGTGTGGGAGTTGCCC
CCGGCGTTGGCGTTGCTCCAGGGGTGGGTGTTGCTCCTGGCGTCGGTCTG
GCTCCTGGAGTGGGCGTAGCACCCGGTGTGGGGGTGGCCCCGGGTGTTGG
GGTTGCACCGGGTATCGGTCCGGGCGGTGTCtaa

The amino acid sequence of a 44.4 kDa human elastin truncated at the N-terminal is disclosed in SEQ ID NO: 41. The 44.4 kDa truncated elastin has amino acids 2-246 deleted from the full length elastin.

(SEQ ID NO: 41)
GVLPGVGGAGVPGVPGAIPGIGGIAGVGTPAAAAAAAAAAKAAKYGAAAG
LVPGGPGFGPGVVGVPGAGVPGVGVPGAGIPVVPGAGIPGAAVPGVVSPE
AAAKAAAKAAKYGARPGVGVGGIPTYGVGAGGFPGFGVGVGGIPGVAGVP
GVGGVPGVGGVPGVGISPEAQAAAAAKAAKYGAAGAGVLGGLVPGPQAAV
PGVPGTGGVPGVGTPAAAAAKAAAKAAQFGLVPGVGVAPGVGVAPGVGVA
PGVGLAPGVGVAPGVGVAPGVGVAPGIGPGGVAAAAKSAAKVAAKAQLRA
AAGLGAGIPGLGVGVGVPGLGVGAGVPGLGVGAGVPGFGAGADEGVRRSL
SPELREGDPSSSQHLPSTPSSPRVPGALAAAKAAKYGAAVPGVLGGLGAL
GGVGIPGGVVGAGPAAAAAAAKAAAKAAQFGLVGAAGLGGLGVGGLGVPG
VGGLGGIPPAAAAKAAKYGAAGLGGVLGGAGQFPLGGVAARPGFGLSPIF
PGGACLGKACGRKRK

The codon optimized polynucleotide sequence encoding the 44.4 kDa truncated human elastin is disclosed in SEQ ID NO: 42

(SEQ ID NO: 42)
GGTGTTCTGCCTGGAGTTGGTGGTGCGGGCGTGCCAGGGGTACCTGGTGC
AATTCCGGGTATTGGTGGTATTGCCGGTGTCGGCACCCCGGCCGCGGCAG
CTGCGGCAGCGGCGGCTGCCAAAGCTGCTAAATACGGTGCCGCGGCGGGT
CTGGTGCCAGGAGGTCCGGGTTTTGGTCCGGGAGTGGTTGGCGTGCCTGG
CGCAGGCGTTCCTGGTGTGGGCGTTCCAGGTGCAGGGATTCCTGTTGTGC
CTGGTGCCGGTATTCCCGGCGCGGCCGTTCCGGGGGTGGTTAGCCCGGAA
GCCGCAGCGAAGGCTGCGGCAAAGGCAGCAAAGTATGGCGCACGCCCAGG
AGTCGGCGTGGGTGGTATCCCGACCTATGGGGTGGGCGCAGGGGGTTTTC
CTGGTTTCGGCGTAGGTGTAGGAGGTATACCGGGCGTGGCCGGTGTACCA
GGGGTTGGTGGCGTCCCTGGTGTTGGCGGTGTGCCAGGTGTTGGTATTTC
ACCGGAAGCACAGGCAGCAGCCGCAGCTAAGGCAGCGAAATATGGTGCCG
CCGGCGCAGGAGTTTTAGGTGGGCTGGTTCCGGGCCCGCAGGCAGCTGTG
CCGGGGGTTCCAGGCACCGGTGGTGTCCCTGGAGTCGGTACGCCGGCTGC
AGCGGCAGCCAAAGCGGCTGCGAAAGCAGCACAGTTTGGCTTAGTACCGG
GTGTGGGAGTTGCCCCCGGCGTTGGCGTTGCTCCAGGGGTGGGTGTTGCT
CCTGGCGTCGGTCTGGCTCCTGGAGTGGGCGTAGCACCCGGTGTGGGGGT
GGCCCCGGGTGTTGGGGTTGCACCGGGTATCGGTCCGGGCGGTGTCGCAG
CAGCAGCTAAAAGCGCGGCGAAAGTTGCGGCCAAAGCCCAACTGCGCGCC
GCCGCGGGCCTCGGTGCAGGTATTCCGGGGCTGGGTGTCGGAGTTGGAGT
CCCGGGTTTGGGCGTGGGCGCGGGAGTTCCGGGACTGGGAGTGGGTGCCG
GAGTTCCTGGCTTTGGTGCAGGCGCAGATGAAGGTGTTCGTCGTAGCCTG
AGTCCGGAACTGCGTGAAGGTGATCCGAGTAGCAGCCAGCATCTGCCGAG
CACCCCGAGCAGCCCGCGTGTTCCGGGTGCATTAGCTGCAGCAAAAGCCG
CCAAGTATGGTGCAGCCGTGCCGGGCGTCTTAGGTGGTCTGGGCGCCCTG
GGTGGTGTAGGCATTCCGGGAGGTGTTGTGGGTGCAGGACCGGCCGCCGC
AGCTGCGGCCGCCAAAGCAGCTGCAAAAGCGGCCCAGTTTGGTTTAGTGG
GCGCCGCAGGTTTAGGCGGTTTAGGTGTGGGTGGACTGGGTGTACCTGGC
GTAGGCGGTCTGGGTGGAATTCCGCCCGCAGCGGCCGCGAAAGCGGCAAA
ATATGGCGCGGCAGGCCTGGGCGGCGTGCTGGGTGGGGCAGGTCAGTTTC
CGCTGGGCGGGGTTGCCGCACGTCCGGGATTTGGTCTGAGCCCGATTTTC
CCTGGCGGCGCATGTCTGGGTAAAGCATGTGGTCGTAAACGTAAAtaa

The amino acid sequence of a 40.4 kDa human elastin truncated at the N-terminal is disclosed in SEQ ID NO: 43. The 40.4 kDa truncated elastin has amino acids 2-295 deleted from the full length elastin.

(SEQ ID NO: 43)
GLVPGGPGFGPGVVGVPGAGVPGVGVPGAGIPVVPGAGIPGAAVPGVVSP
EAAAKAAAKAAKYGARPGVGVGGIPTYGVGAGGFPGFGVGVGGIPGVAGV
PGVGGVPGVGGVPGVGISPEAQAAAAAKAAKYGAAGAGVLGGLVPGPQAA
VPGVPGTGGVPGVGTPAAAAAKAAAKAAQFGLVPGVGVAPGVGVAPGVGV
APGVGLAPGVGVAPGVGVAPGVGVAPGIGPGGVAAAAKSAAKVAAKAQLR
AAAGLGAGIPGLGVGVGVPGLGVGAGVPGLGVGAGVPGFGAGADEGVRRS
LSPELREGDPSSSQHLPSTPSSPRVPGALAAAKAAKYGAAVPGVLGGLGA
LGGVGIPGGVVGAGPAAAAAAAKAAAKAAQFGLVGAAGLGGLGVGGLGVP
GVGGLGGIPPAAAAKAAKYGAAGLGGVLGGAGQFPLGGVAARPGFGLSPI
FPGGACLGKACGRKRK

The codon optimized polynucleotide sequence encoding the 40.4 kDa truncated human elastin is disclosed in SEQ ID NO: 44

(SEQ ID NO: 44)
GGTCTGGTGCCAGGAGGTCCGGGTTTTGGTCCGGGAGTGGTTGGCGTGCC
TGGCGCAGGCGTTCCTGGTGTGGGCGTTCCAGGTGCAGGGATTCCTGTTG
TGCCTGGTGCCGGTATTCCCGGCGCGGCCGTTCCGGGGGTGGTTAGCCCG
GAAGCCGCAGCGAAGGCTGCGGCAAAGGCAGCAAAGTATGGCGCACGCCC
AGGAGTCGGCGTGGGTGGTATCCCGACCTATGGGGTGGGCGCAGGGGGTT
TTCCTGGTTTCGGCGTAGGTGTAGGAGGTATACCGGGCGTGGCCGGTGTA
CCAGGGGTTGGTGGCGTCCCTGGTGTTGGCGGTGTGCCAGGTGTTGGTAT
TTCACCGGAAGCACAGGCAGCAGCCGCAGCTAAGGCAGCGAAATATGGTG
CCGCCGGCGCAGGAGTTTTAGGTGGGCTGGTTCCGGGCCCGCAGGCAGCT
GTGCCGGGGGTTCCAGGCACCGGTGGTGTCCCTGGAGTCGGTACGCCGGC
TGCAGCGGCAGCCAAAGCGGCTGCGAAAGCAGCACAGTTTGGCTTAGTAC
CGGGTGTGGGAGTTGCCCCCGGCGTTGGCGTTGCTCCAGGGGTGGGTGTT
GCTCCTGGCGTCGGTCTGGCTCCTGGAGTGGGCGTAGCACCCGGTGTGGG
GGTGGCCCCGGGTGTTGGGGTTGCACCGGGTATCGGTCCGGGCGGTGTCG
CAGCAGCAGCTAAAAGCGCGGCGAAAGTTGCGGCCAAAGCCCAACTGCGC
GCCGCCGCGGGCCTCGGTGCAGGTATTCCGGGGCTGGGTGTCGGAGTTGG
AGTCCCGGGTTTGGGCGTGGGCGCGGGAGTTCCGGGACTGGGAGTGGGTG
CCGGAGTTCCTGGCTTTGGTGCAGGCGCAGATGAAGGTGTTCGTCGTAGC
CTGAGTCCGGAACTGCGTGAAGGTGATCCGAGTAGCAGCCAGCATCTGCC
GAGCACCCCGAGCAGCCCGCGTGTTCCGGGTGCATTAGCTGCAGCAAAAG
CCGCCAAGTATGGTGCAGCCGTGCCGGGCGTCTTAGGTGGTCTGGGCGCC
CTGGGTGGTGTAGGCATTCCGGGAGGTGTTGTGGGTGCAGGACCGGCCGC
CGCAGCTGCGGCCGCCAAAGCAGCTGCAAAAGCGGCCCAGTTTGGTTTAG
TGGGCGCCGCAGGTTTAGGCGGTTTAGGTGTGGGTGGACTGGGTGTACCT
GGCGTAGGCGGTCTGGGTGGAATTCCGCCCGCAGCGGCCGCGAAAGCGGC
AAAATATGGCGCGGCAGGCCTGGGCGGCGTGCTGGGTGGGGCAGGTCAGT
TTCCGCTGGGCGGGGTTGCCGCACGTCCGGGATTTGGTCTGAGCCCGATT
TTCCCTGGCGGCGCATGTCTGGGTAAAGCATGTGGTCGTAAACGTAAAta
a

The amino acid sequence of a 39.8 kDa human elastin truncated at the C-terminal is disclosed in SEQ ID NO: 45. The 39.8 kDa truncated elastin has amino acids 462-761 deleted from the full length elastin.

(SEQ ID NO: 45)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQAAAAAKAAKYGAAGAGVLGGLVPGPQAAVPGVPG
TGGVPGVGTP

The codon optimized polynucleotide sequence encoding the 39.8 kDa truncated human elastin is disclosed in SEQ ID NO: 46

(SEQ ID NO: 46)
GGTGGCGTACCAGGCGCAATTCCTGGGGGTGTCCCAGGCGGTGTTTTTTAT
CCGGGCGCCGGTCTTGGCGCACTGGGTGGCGGTGCACTGGGCCCGGGCGGC
AAACCGCTGAAACCGGTACCAGGTGGTTTAGCAGGCGCCGGCTTAGGCGCA
GGTCTGGGAGCATTTCCGGCAGTTACCTTTCCAGGGGCACTGGTTCCTGGA
GGTGTGGCCGATGCAGCCGCGGCATATAAAGCCGCTAAAGCCGGTGCGGGT
TTAGGAGGCGTCCCAGGTGTCGGTGGCCTGGGTGTTAGCGCCGGTGCAGTT
GTTCCGCAGCCGGGAGCAGGGGTTAAACCTGGTAAAGTGCCGGGAGTAGGT
CTGCCAGGCGTTTATCCTGGTGGTGTTTTGCCGGGTGCCCGTTTTCCGGGC
GTTGGTGTTCTTCCAGGCGTGCCGACCGGAGCCGGTGTTAAACCGAAAGCC
CCCGGTGTTGGAGGTGCATTTGCAGGCATCCCGGGAGTTGGCCCGTTTGGT
GGTCCGCAACCTGGGGTTCCGTTAGGTTATCCGATTAAAGCACCGAAACTG
CCCGGCGGTTATGGTCTGCCGTACACAACCGGTAAACTGCCGTATGGTTAT
GGCCCGGGTGGAGTTGCGGGTGCAGCAGGTAAAGCGGGTTATCCTACCGGA
ACCGGTGTAGGTCCGCAGGCCGCTGCTGCCGCCGCCGCAAAAGCAGCGGCT
AAATTTGGCGCCGGAGCAGCGGGTGTTCTGCCTGGAGTTGGTGGTGCGGGC
GTGCCAGGGGTACCTGGTGCAATTCCGGGTATTGGTGGTATTGCCGGTGTC
GGCACCCCGGCCGCGGCAGCTGCGGCAGCGGCGGCTGCCAAAGCTGCTAAA
TACGGTGCCGCGGCGGGTCTGGTGCCAGGAGGTCCGGGTTTTGGTCCGGGA
GTGGTTGGCGTGCCTGGCGCAGGCGTTCCTGGTGTGGGCGTTCCAGGTGCA
GGGATTCCTGTTGTGCCTGGTGCCGGTATTCCCGGCGCGGCCGTTCCGGGG
GTGGTTAGCCCGGAAGCCGCAGCGAAGGCTGCGGCAAAGGCAGCAAAGTAT
GGCGCACGCCCAGGAGTCGGCGTGGGTGGTATCCCGACCTATGGGGTGGGC
GCAGGGGGTTTTCCTGGTTTCGGCGTAGGTGTAGGAGGTATACCGGGCGTG
GCCGGTGTACCAGGGGTTGGTGGCGTCCCTGGTGTTGGCGGTGTGCCAGGT
GTTGGTATTTCACCGGAAGCACAGGCAGCAGCCGCAGCTAAGGCAGCGAAA
TATGGTGCCGCCGGCGCAGGAGTTTTAGGTGGGCTGGTTCCGGGCCCGCAG
GCAGCTGTGCCGGGGGTTCCAGGCACCGGTGGTGTCCCTGGAGTCGGTACG
CCGtaa

The amino acid sequence of a 36.1 kDa human elastin truncated at the C-terminal is disclosed in SEQ ID NO: 47. The 36.1 kDa truncated elastin has amino acids 418-761 deleted from the full length elastin.

(SEQ ID NO: 47)
GGVPGAIPGGVPGGVFYPGAGLGALGGGALGPGGKPLKPVPGGLAGAGLG
AGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVK
PKAPGVGGAFAGIPGVGPFGGPQPGVPLGYPIKAPKLPGGYGLPYTTGKL
PYGYGPGGVAGAAGKAGYPTGTGVGPQAAAAAAAKAAAKFGAGAAGVLPG
VGGAGVPGVPGAIPGIGGIAGVGTPAAAAAAAAAAKAAKYGAAAGLVPGG
PGFGPGVVGVPGAGVPGVGVPGAGIPVVPGAGIPGAAVPGVVSPEAAAKA
AAKAAKYGARPGVGVGGIPTYGVGAGGFPGFGVGVGGIPGVAGVPGVGGV
PGVGGVPGVGISPEAQ

The codon optimized polynucleotide sequence encoding the 36.1 kDa truncated human elastin is disclosed in SEQ ID NO: 48

(SEQ ID NO: 48)
GGTGGCGTACCAGGCGCAATTCCTGGGGGTGTCCCAGGCGGTGTTTTTTA
TCCGGGCGCCGGTCTTGGCGCACTGGGTGGCGGTGCACTGGGCCCGGGCG
GCAAACCGCTGAAACCGGTACCAGGTGGTTTAGCAGGCGCCGGCTTAGGC
GCAGGTCTGGGAGCATTTCCGGCAGTTACCTTTCCAGGGGCACTGGTTCC
TGGAGGTGTGGCCGATGCAGCCGCGGCATATAAAGCCGCTAAAGCCGGTG
CGGGTTTAGGAGGCGTCCCAGGTGTCGGTGGCCTGGGTGTTAGCGCCGGT
GCAGTTGTTCCGCAGCCGGGAGCAGGGGTTAAACCTGGTAAAGTGCCGGG
AGTAGGTCTGCCAGGCGTTTATCCTGGTGGTGTTTTGCCGGGTGCCCGTT
TTCCGGGCGTTGGTGTTCTTCCAGGCGTGCCGACCGGAGCCGGTGTTAAA
CCGAAAGCCCCCGGTGTTGGAGGTGCATTTGCAGGCATCCCGGGAGTTGG
CCCGTTTGGTGGTCCGCAACCTGGGGTTCCGTTAGGTTATCCGATTAAAG
CACCGAAACTGCCCGGCGGTTATGGTCTGCCGTACACAACCGGTAAACTG
CCGTATGGTTATGGCCCGGGTGGAGTTGCGGGTGCAGCAGGTAAAGCGGG
TTATCCTACCGGAACCGGTGTAGGTCCGCAGGCCGCTGCTGCCGCCGCCG
CAAAAGCAGCGGCTAAATTTGGCGCCGGAGCAGCGGGTGTTCTGCCTGGA
GTTGGTGGTGCGGGCGTGCCAGGGGTACCTGGTGCAATTCCGGGTATTGG
TGGTATTGCCGGTGTCGGCACCCCGGCCGCGGCAGCTGCGGCAGCGGCGG
CTGCCAAAGCTGCTAAATACGGTGCCGCGGCGGGTCTGGTGCCAGGAGGT
CCGGGTTTTGGTCCGGGAGTGGTTGGCGTGCCTGGCGCAGGCGTTCCTGG
TGTGGGCGTTCCAGGTGCAGGGATTCCTGTTGTGCCTGGTGCCGGTATTC
CCGGCGCGGCCGTTCCGGGGGTGGTTAGCCCGGAAGCCGCAGCGAAGGCT
GCGGCAAAGGCAGCAAAGTATGGCGCACGCCCAGGAGTCGGCGTGGGTGG
TATCCCGACCTATGGGGTGGGCGCAGGGGGTTTTCCTGGTTTCGGCGTAG
GTGTAGGAGGTATACCGGGCGTGGCCGGTGTACCAGGGGTTGGTGGCGTC
CCTGGTGTTGGCGGTGTGCCAGGTGTTGGTATTTCACCGGAAGCACAGta
a

The amino acid sequence of a 34.9 kDa human elastin truncated at the N-terminal is disclosed in SEQ ID NO: 49. The 34.9 kDa truncated elastin has amino acids 2-360 deleted from the full length elastin.

(SEQ ID NO: 49)
RPGVGVGGIPTYGVGAGGFPGFGVGVGGIPGVAGVPGVGGVPGVGGV
PGVGISPEAQAAAAAKAAKYGAAGAGVLGGLVPGPQAAVPGVPGTGG
VPGVGTPAAAAAKAAAKAAQFGLVPGVGVAPGVGVAPGVGVAPGVGL
APGVGVAPGVGVAPGVGVAPGIGPGGVAAAAKSAAKVAAKAQLRAAA
GLGAGIPGLGVGVGVPGLGVGAGVPGLGVGAGVPGFGAGADEGVRRS
LSPELREGDPSSSQHLPSTPSSPRVPGALAAAKAAKYGAAVPGVLGG
LGALGGVGIPGGVVGAGPAAAAAAAKAAAKAAQFGLVGAAGLGGLGV
GGLGVPGVGGLGGIPPAAAAKAAKYGAAGLGGVLGGAGQFPLGGVAA
RPGFGLSPIFPGGACLGKACGRKRK

The codon optimized polynucleotide sequence encoding the 34.9 kDa truncated human elastin is disclosed in SEQ ID NO: 50

(SEQ ID NO: 50)
CGCCCAGGAGTCGGCGTGGGTGGTATCCCGACCTATGGGGTGGGCGC
AGGGGGTTTTCCTGGTTTCGGCGTAGGTGTAGGAGGTATACCGGGCG
TGGCCGGTGTACCAGGGGTTGGTGGCGTCCCTGGTGTTGGCGGTGTG
CCAGGTGTTGGTATTTCACCGGAAGCACAGGCAGCAGCCGCAGCTAA
GGCAGCGAAATATGGTGCCGCCGGCGCAGGAGTTTTAGGTGGGCTGG
TTCCGGGCCCGCAGGCAGCTGTGCCGGGGGTTCCAGGCACCGGTGGT
GTCCCTGGAGTCGGTACGCCGGCTGCAGCGGCAGCCAAAGCGGCTGC
GAAAGCAGCACAGTTTGGCTTAGTACCGGGTGTGGGAGTTGCCCCCG
GCGTTGGCGTTGCTCCAGGGGTGGGTGTTGCTCCTGGCGTCGGTCTG
GCTCCTGGAGTGGGCGTAGCACCCGGTGTGGGGGTGGCCCCGGGTGT
TGGGGTTGCACCGGGTATCGGTCCGGGCGGTGTCGCAGCAGCAGCTA
AAAGCGCGGCGAAAGTTGCGGCCAAAGCCCAACTGCGCGCCGCCGCG
GGCCTCGGTGCAGGTATTCCGGGGCTGGGTGTCGGAGTTGGAGTCCC
GGGTTTGGGCGTGGGCGCGGGAGTTCCGGGACTGGGAGTGGGTGCCG
GAGTTCCTGGCTTTGGTGCAGGCGCAGATGAAGGTGTTCGTCGTAGC
CTGAGTCCGGAACTGCGTGAAGGTGATCCGAGTAGCAGCCAGCATCT
GCCGAGCACCCCGAGCAGCCCGCGTGTTCCGGGTGCATTAGCTGCAG
CAAAAGCCGCCAAGTATGGTGCAGCCGTGCCGGGCGTCTTAGGTGGT
CTGGGCGCCCTGGGTGGTGTAGGCATTCCGGGAGGTGTTGTGGGTGC
AGGACCGGCCGCCGCAGCTGCGGCCGCCAAAGCAGCTGCAAAAGCGG
CCCAGTTTGGTTTAGTGGGCGCCGCAGGTTTAGGCGGTTTAGGTGTG
GGTGGACTGGGTGTACCTGGCGTAGGCGGTCTGGGTGGAATTCCGCC
CGCAGCGGCCGCGAAAGCGGCAAAATATGGCGCGGCAGGCCTGGGCG
GCGTGCTGGGTGGGGCAGGTCAGTTTCCGCTGGGCGGGGTTGCCGCA
CGTCCGGGATTTGGTCTGAGCCCGATTTTCCCTGGCGGCGCATGTCT
GGGTAAAGCATGTGGTCGTAAACGTAAAtaa

The amino acid sequence of a 32 kDa human elastin truncated at the C-terminal is disclosed in SEQ ID NO: 51. The 32 kDa truncated elastin has amino acids 373-761 deleted from the full length elastin.

(SEQ ID NO: 51)
GGVPGAIPGGVPGGVFYPGAGLGALGGGALGPGGKPLKPVPGGLAGA
GLGAGLGAFPAVTFPGALVPGGVADAAAAYKAAKAGAGLGGVPGVGG
LGVSAGAVVPQPGAGVKPGKVPGVGLPGVYPGGVLPGARFPGVGVLP
GVPTGAGVKPKAPGVGGAFAGIPGVGPFGGPQPGVPLGYPIKAPKLP
GGYGLPYTTGKLPYGYGPGGVAGAAGKAGYPTGTGVGPQAAAAAAAK
AAAKFGAGAAGVLPGVGGAGVPGVPGAIPGIGGIAGVGTPAAAAAAA
AAAKAAKYGAAAGLVPGGPGFGPGVVGVPGAGVPGVGVPGAGIPVVP
GAGIPGAAVPGVVSPEAAAKAAAKAAKYGARPGVGVGGIPTY

The codon optimized polynucleotide sequence encoding the 32 kDa truncated human elastin is disclosed in SEQ ID NO: 52

(SEQ ID NO: 52)
GGTGGCGTACCAGGCGCAATTCCTGGGGGTGTCCCAGGCGGTGTTTT
TTATCCGGGCGCCGGTCTTGGCGCACTGGGTGGCGGTGCACTGGGCC
CGGGCGGCAAACCGCTGAAACCGGTACCAGGTGGTTTAGCAGGCGCC
GGCTTAGGCGCAGGTCTGGGAGCATTTCCGGCAGTTACCTTTCCAGG
GGCACTGGTTCCTGGAGGTGTGGCCGATGCAGCCGCGGCATATAAAG
CCGCTAAAGCCGGTGCGGGTTTAGGAGGCGTCCCAGGTGTCGGTGGC
CTGGGTGTTAGCGCCGGTGCAGTTGTTCCGCAGCCGGGAGCAGGGGT
TAAACCTGGTAAAGTGCCGGGAGTAGGTCTGCCAGGCGTTTATCCTG
GTGGTGTTTTGCCGGGTGCCCGTTTTCCGGGCGTTGGTGTTCTTCCA
GGCGTGCCGACCGGAGCCGGTGTTAAACCGAAAGCCCCCGGTGTTGG
AGGTGCATTTGCAGGCATCCCGGGAGTTGGCCCGTTTGGTGGTCCGC
AACCTGGGGTTCCGTTAGGTTATCCGATTAAAGCACCGAAACTGCCC
GGCGGTTATGGTCTGCCGTACACAACCGGTAAACTGCCGTATGGTTA
TGGCCCGGGTGGAGTTGCGGGTGCAGCAGGTAAAGCGGGTTATCCTA
CCGGAACCGGTGTAGGTCCGCAGGCCGCTGCTGCCGCCGCCGCAAAA
GCAGCGGCTAAATTTGGCGCCGGAGCAGCGGGTGTTCTGCCTGGAGT
TGGTGGTGCGGGCGTGCCAGGGGTACCTGGTGCAATTCCGGGTATTG
GTGGTATTGCCGGTGTCGGCACCCCGGCCGCGGCAGCTGCGGCAGCG
GCGGCTGCCAAAGCTGCTAAATACGGTGCCGCGGCGGGTCTGGTGCC
AGGAGGTCCGGGTTTTGGTCCGGGAGTGGTTGGCGTGCCTGGCGCAG
GCGTTCCTGGTGTGGGCGTTCCAGGTGCAGGGATTCCTGTTGTGCCT
GGTGCCGGTATTCCCGGCGCGGCCGTTCCGGGGGTGGTTAGCCCGGA
AGCCGCAGCGAAGGCTGCGGCAAAGGCAGCAAAGTATGGCGCACGCC
CAGGAGTCGGCGTGGGTGGTATCCCGACCTATtaa

The amino acid sequence of a 29.9 kDa human elastin truncated at the C-terminal is disclosed in SEQ ID NO: 53. The 60.7 kDa truncated elastin has amino acids 347-761 deleted from the full length elastin.

(SEQ ID NO: 53)
GGVPGAIPGGVPGGVFYPGAGLGALGGGALGPGGKPLKPVPGGLAGA
GLGAGLGAFPAVTFPGALVPGGVADAAAAYKAAKAGAGLGGVPGVGG
LGVSAGAVVPQPGAGVKPGKVPGVGLPGVYPGGVLPGARFPGVGVLP
GVPTGAGVKPKAPGVGGAFAGIPGVGPFGGPQPGVPLGYPIKAPKLP
GGYGLPYTTGKLPYGYGPGGVAGAAGKAGYPTGTGVGPQAAAAAAAK
AAAKFGAGAAGVLPGVGGAGVPGVPGAIPGIGGIAGVGTPAAAAAAA
AAAKAAKYGAAAGLVPGGPGFGPGVVGVPGAGVPGVGVPGAGIPVVP
GAGIPGAAVPGVVSPE

The codon optimized polynucleotide sequence encoding the 29.9 kDa truncated human elastin is disclosed in SEQ ID NO: 54

(SEQ ID NO: 54)
GGTGGCGTACCAGGCGCAATTCCTGGGGGTGTCCCAGGCGGTGTTTT
TTATCCGGGCGCCGGTCTTGGCGCACTGGGTGGCGGTGCACTGGGCC
CGGGCGGCAAACCGCTGAAACCGGTACCAGGTGGTTTAGCAGGCGCC
GGCTTAGGCGCAGGTCTGGGAGCATTTCCGGCAGTTACCTTTCCAGG
GGCACTGGTTCCTGGAGGTGTGGCCGATGCAGCCGCGGCATATAAAG
CCGCTAAAGCCGGTGCGGGTTTAGGAGGCGTCCCAGGTGTCGGTGGC
CTGGGTGTTAGCGCCGGTGCAGTTGTTCCGCAGCCGGGAGCAGGGGT
TAAACCTGGTAAAGTGCCGGGAGTAGGTCTGCCAGGCGTTTATCCTG
GTGGTGTTTTGCCGGGTGCCCGTTTTCCGGGCGTTGGTGTTCTTCCA
GGCGTGCCGACCGGAGCCGGTGTTAAACCGAAAGCCCCCGGTGTTGG
AGGTGCATTTGCAGGCATCCCGGGAGTTGGCCCGTTTGGTGGTCCGC
AACCTGGGGTTCCGTTAGGTTATCCGATTAAAGCACCGAAACTGCCC
GGCGGTTATGGTCTGCCGTACACAACCGGTAAACTGCCGTATGGTTA
TGGCCCGGGTGGAGTTGCGGGTGCAGCAGGTAAAGCGGGTTATCCTA
CCGGAACCGGTGTAGGTCCGCAGGCCGCTGCTGCCGCCGCCGCAAAA
GCAGCGGCTAAATTTGGCGCCGGAGCAGCGGGTGTTCTGCCTGGAGT
TGGTGGTGCGGGCGTGCCAGGGGTACCTGGTGCAATTCCGGGTATTG
GTGGTATTGCCGGTGTCGGCACCCCGGCCGCGGCAGCTGCGGCAGCG
GCGGCTGCCAAAGCTGCTAAATACGGTGCCGCGGCGGGTCTGGTGCC
AGGAGGTCCGGGTTTTGGTCCGGGAGTGGTTGGCGTGCCTGGCGCAG
GCGTTCCTGGTGTGGGCGTTCCAGGTGCAGGGATTCCTGTTGTGCCT
GGTGCCGGTATTCCCGGCGCGGCCGTTCCGGGGGTGGTTAGCCCGGA
Ataa

The amino acid sequence of a 29.4 kDa human elastin truncated at the N-terminal is disclosed in SEQ ID NO: 55. The 29.4 kDa truncated elastin has amino acids 2-425 deleted from the full length elastin.

(SEQ ID NO: 55)
KYGAAGAGVLGGLVPGPQAAVPGVPGTGGVPGVGTPAAAAAKAAAKA
AQFGLVPGVGVAPGVGVAPGVGVAPGVGLAPGVGVAPGVGVAPGVGV
APGIGPGGVAAAAKSAAKVAAKAQLRAAAGLGAGIPGLGVGVGVPGL
GVGAGVPGLGVGAGVPGFGAGADEGVRRSLSPELREGDPSSSQHLPS
TPSSPRVPGALAAAKAAKYGAAVPGVLGGLGALGGVGIPGGVVGAGP
AAAAAAAKAAAKAAQFGLVGAAGLGGLGVGGLGVPGVGGLGGIPPAA
AAKAAKYGAAGLGGVLGGAGQFPLGGVAARPGFGLSPIFPGGACLGK
ACGRKRK

The codon optimized polynucleotide sequence encoding the 29.4 kDa truncated human elastin is disclosed in SEQ ID NO: 56

(SEQ ID NO: 56)
AAATATGGTGCCGCCGGCGCAGGAGTTTTAGGTGGGCTGGTTCCGGG
CCCGCAGGCAGCTGTGCCGGGGGTTCCAGGCACCGGTGGTGTCCCTG
GAGTCGGTACGCCGGCTGCAGCGGCAGCCAAAGCGGCTGCGAAAGCA
GCACAGTTTGGCTTAGTACCGGGTGTGGGAGTTGCCCCCGGCGTTGG
CGTTGCTCCAGGGGTGGGTGTTGCTCCTGGCGTCGGTCTGGCTCCTG
GAGTGGGCGTAGCACCCGGTGTGGGGGTGGCCCCGGGTGTTGGGGTT
GCACCGGGTATCGGTCCGGGCGGTGTCGCAGCAGCAGCTAAAAGCGC
GGCGAAAGTTGCGGCCAAAGCCCAACTGCGCGCCGCCGCGGGCCTCG
GTGCAGGTATTCCGGGGCTGGGTGTCGGAGTTGGAGTCCCGGGTTTG
GGCGTGGGCGCGGGAGTTCCGGGACTGGGAGTGGGTGCCGGAGTTCC
TGGCTTTGGTGCAGGCGCAGATGAAGGTGTTCGTCGTAGCCTGAGTC
CGGAACTGCGTGAAGGTGATCCGAGTAGCAGCCAGCATCTGCCGAGC
ACCCCGAGCAGCCCGCGTGTTCCGGGTGCATTAGCTGCAGCAAAAGC
CGCCAAGTATGGTGCAGCCGTGCCGGGCGTCTTAGGTGGTCTGGGCG
CCCTGGGTGGTGTAGGCATTCCGGGAGGTGTTGTGGGTGCAGGACCG
GCCGCCGCAGCTGCGGCCGCCAAAGCAGCTGCAAAAGCGGCCCAGTT
TGGTTTAGTGGGCGCCGCAGGTTTAGGCGGTTTAGGTGTGGGTGGAC
TGGGTGTACCTGGCGTAGGCGGTCTGGGTGGAATTCCGCCCGCAGCG
GCCGCGAAAGCGGCAAAATATGGCGCGGCAGGCCTGGGCGGCGTGCT
GGGTGGGGCAGGTCAGTTTCCGCTGGGCGGGGTTGCCGCACGTCCGG
GATTTGGTCTGAGCCCGATTTTCCCTGGCGGCGCATGTCTGGGTAAA
GCATGTGGTCGTAAACGTAAAtaa

The amino acid sequence of a 25.3 kDa human elastin truncated at the N-terminal is disclosed in SEQ ID NO: 57. The 25.3 kDa truncated elastin has amino acids 2-473 deleted from the full length elastin.

(SEQ ID NO: 57)
QFGLVPGVGVAPGVGVAPGVGVAPGVGLAPGVGVAPGVGVAPGVGVA
PGIGPGGVAAAAKSAAKVAAKAQLRAAAGLGAGIPGLGVGVGVPGLG
VGAGVPGLGVGAGVPGFGAGADEGVRRSLSPELREGDPSSSQHLPST
PSSPRVPGALAAAKAAKYGAAVPGVLGGLGALGGVGIPGGVVGAGPA
AAAAAAKAAAKAAQFGLVGAAGLGGLGVGGLGVPGVGGLGGIPPAAA
AKAAKYGAAGLGGVLGGAGQFPLGGVAARPGFGLSPIFPGGACLGKA
CGRKRK

The codon optimized polynucleotide sequence encoding the 25.3 kDa truncated human elastin is disclosed in SEQ ID NO: 58

(SEQ ID NO: 58)
CAGTTTGGCTTAGTACCGGGTGTGGGAGTTGCCCCCGGCGTTGGCGT
TGCTCCAGGGGTGGGTGTTGCTCCTGGCGTCGGTCTGGCTCCTGGAG
TGGGCGTAGCACCCGGTGTGGGGGTGGCCCCGGGTGTTGGGGTTGCA
CCGGGTATCGGTCCGGGCGGTGTCGCAGCAGCAGCTAAAAGCGCGGC
GAAAGTTGCGGCCAAAGCCCAACTGCGCGCCGCCGCGGGCCTCGGTG
CAGGTATTCCGGGGCTGGGTGTCGGAGTTGGAGTCCCGGGTTTGGGC
GTGGGCGCGGGAGTTCCGGGACTGGGAGTGGGTGCCGGAGTTCCTGG
CTTTGGTGCAGGCGCAGATGAAGGTGTTCGTCGTAGCCTGAGTCCGG
AACTGCGTGAAGGTGATCCGAGTAGCAGCCAGCATCTGCCGAGCACC
CCGAGCAGCCCGCGTGTTCCGGGTGCATTAGCTGCAGCAAAAGCCGC
CAAGTATGGTGCAGCCGTGCCGGGCGTCTTAGGTGGTCTGGGCGCCC
TGGGTGGTGTAGGCATTCCGGGAGGTGTTGTGGGTGCAGGACCGGCC
GCCGCAGCTGCGGCCGCCAAAGCAGCTGCAAAAGCGGCCCAGTTTGG
TTTAGTGGGCGCCGCAGGTTTAGGCGGTTTAGGTGTGGGTGGACTGG
GTGTACCTGGCGTAGGCGGTCTGGGTGGAATTCCGCCCGCAGCGGCC
GCGAAAGCGGCAAAATATGGCGCGGCAGGCCTGGGCGGCGTGCTGGG
TGGGGCAGGTCAGTTTCCGCTGGGCGGGGTTGCCGCACGTCCGGGAT
TTGGTCTGAGCCCGATTTTCCCTGGCGGCGCATGTCTGGGTAAAGCA
TGTGGTCGTAAACGTAAAtaa

The amino acid sequence of a 24.1 kDa human elastin truncated at the C-terminal is disclosed in SEQ ID NO: 59. The 24.1 kDa truncated elastin has amino acids 277-761 deleted from the full length elastin.

(SEQ ID NO: 59)
GGVPGAIPGGVPGGVFYPGAGLGALGGGALGPGGKPLKPVPGGLAGA
GLGAGLGAFPAVTFPGALVPGGVADAAAAYKAAKAGAGLGGVPGVGG
LGVSAGAVVPQPGAGVKPGKVPGVGLPGVYPGGVLPGARFPGVGVLP
GVPTGAGVKPKAPGVGGAFAGIPGVGPFGGPQPGVPLGYPIKAPKLP
GGYGLPYTTGKLPYGYGPGGVAGAAGKAGYPTGTGVGPQAAAAAAAK
AAAKFGAGAAGVLPGVGGAGVPGVPGAIPGIGGIAGVGTP

The codon optimized polynucleotide sequence encoding the 24.1 kDa truncated human elastin is disclosed in SEQ ID NO: 60

(SEQ ID NO: 60)
GGTGGCGTACCAGGCGCAATTCCTGGGGGTGTCCCAGGCGGTGTTTT
TTATCCGGGCGCCGGTCTTGGCGCACTGGGTGGCGGTGCACTGGGCC
CGGGCGGCAAACCGCTGAAACCGGTACCAGGTGGTTTAGCAGGCGCC
GGCTTAGGCGCAGGTCTGGGAGCATTTCCGGCAGTTACCTTTCCAGG
GGCACTGGTTCCTGGAGGTGTGGCCGATGCAGCCGCGGCATATAAAG
CCGCTAAAGCCGGTGCGGGTTTAGGAGGCGTCCCAGGTGTCGGTGGC
CTGGGTGTTAGCGCCGGTGCAGTTGTTCCGCAGCCGGGAGCAGGGGT
TAAACCTGGTAAAGTGCCGGGAGTAGGTCTGCCAGGCGTTTATCCTG
GTGGTGTTTTGCCGGGTGCCCGTTTTCCGGGCGTTGGTGTTCTTCCA
GGCGTGCCGACCGGAGCCGGTGTTAAACCGAAAGCCCCCGGTGTTGG
AGGTGCATTTGCAGGCATCCCGGGAGTTGGCCCGTTTGGTGGTCCGC
AACCTGGGGTTCCGTTAGGTTATCCGATTAAAGCACCGAAACTGCCC
GGCGGTTATGGTCTGCCGTACACAACCGGTAAACTGCCGTATGGTTA
TGGCCCGGGTGGAGTTGCGGGTGCAGCAGGTAAAGCGGGTTATCCTA
CCGGAACCGGTGTAGGTCCGCAGGCCGCTGCTGCCGCCGCCGCAAAA
GCAGCGGCTAAATTTGGCGCCGGAGCAGCGGGTGTTCTGCCTGGAGT
TGGTGGTGCGGGCGTGCCAGGGGTACCTGGTGCAATTCCGGGTATTG
GTGGTATTGCCGGTGTCGGCACCCCGtaa

The amino acid sequence of a 20.3 kDa human elastin truncated at the C-terminal is disclosed in SEQ ID NO: 61. The 20.3 kDa truncated elastin has amino acids 229-761 deleted from the full length elastin.

(SEQ ID NO: 61)
GGVPGAIPGGVPGGVFYPGAGLGALGGGALGPGGKPLKPVPGGLAGA
GLGAGLGAFPAVTFPGALVPGGVADAAAAYKAAKAGAGLGGVPGVGG
LGVSAGAVVPQPGAGVKPGKVPGVGLPGVYPGGVLPGARFPGVGVLP
GVPTGAGVKPKAPGVGGAFAGIPGVGPFGGPQPGVPLGYPIKAPKLP
GGYGLPYTTGKLPYGYGPGGVAGAAGKAGYPTGTGVGPQ

The codon optimized polynucleotide sequence encoding the 20.3 kDa truncated human elastin is disclosed in SEQ ID NO: 62

(SEQ ID NO: 62)
GGTGGCGTACCAGGCGCAATTCCTGGGGGTGTCCCAGGCGGTGTTTT
TTATCCGGGCGCCGGTCTTGGCGCACTGGGTGGCGGTGCACTGGGCC
CGGGCGGCAAACCGCTGAAACCGGTACCAGGTGGTTTAGCAGGCGCC
GGCTTAGGCGCAGGTCTGGGAGCATTTCCGGCAGTTACCTTTCCAGG
GGCACTGGTTCCTGGAGGTGTGGCCGATGCAGCCGCGGCATATAAAG
CCGCTAAAGCCGGTGCGGGTTTAGGAGGCGTCCCAGGTGTCGGTGGC
CTGGGTGTTAGCGCCGGTGCAGTTGTTCCGCAGCCGGGAGCAGGGGT
TAAACCTGGTAAAGTGCCGGGAGTAGGTCTGCCAGGCGTTTATCCTG
GTGGTGTTTTGCCGGGTGCCCGTTTTCCGGGCGTTGGTGTTCTTCCA
GGCGTGCCGACCGGAGCCGGTGTTAAACCGAAAGCCCCCGGTGTTGG
AGGTGCATTTGCAGGCATCCCGGGAGTTGGCCCGTTTGGTGGTCCGC
AACCTGGGGTTCCGTTAGGTTATCCGATTAAAGCACCGAAACTGCCC
GGCGGTTATGGTCTGCCGTACACAACCGGTAAACTGCCGTATGGTTA
TGGCCCGGGTGGAGTTGCGGGTGCAGCAGGTAAAGCGGGTTATCCTA
CCGGAACCGGTGTAGGTCCGCAGtaa

The amino acid sequence of a 19.6 kDa human elastin truncated at the N-terminal is disclosed in SEQ ID NO: 63. The 19.6 kDa truncated elastin has amino acids 2-542 deleted from the full length elastin.

(SEQ ID NO: 63)
QLRAAAGLGAGIPGLGVGVGVPGLGVGAGVPGLGVGAGVPGFGAGAD
EGVRRSLSPELREGDPSSSQHLPSTPSSPRVPGALAAAKAAKYGAAV
PGVLGGLGALGGVGIPGGVVGAGPAAAAAAAKAAAKAAQFGLVGAAG
LGGLGVGGLGVPGVGGLGGIPPAAAAKAAKYGAAGLGGVLGGAGQFP
LGGVAARPGFGLSPIFPGGACLGKACGRKRK

The codon optimized polynucleotide sequence encoding the 19.6 kDa truncated human elastin is disclosed in SEQ ID NO: 64

(SEQ ID NO: 64)
CAACTGCGCGCCGCCGCGGGCCTCGGTGCAGGTATTCCGGGGCTGGG
TGTCGGAGTTGGAGTCCCGGGTTTGGGCGTGGGCGCGGGAGTTCCGG
GACTGGGAGTGGGTGCCGGAGTTCCTGGCTTTGGTGCAGGCGCAGAT
GAAGGTGTTCGTCGTAGCCTGAGTCCGGAACTGCGTGAAGGTGATCC
GAGTAGCAGCCAGCATCTGCCGAGCACCCCGAGCAGCCCGCGTGTTC
CGGGTGCATTAGCTGCAGCAAAAGCCGCCAAGTATGGTGCAGCCGTG
CCGGGCGTCTTAGGTGGTCTGGGCGCCCTGGGTGGTGTAGGCATTCC
GGGAGGTGTTGTGGGTGCAGGACCGGCCGCCGCAGCTGCGGCCGCCA
AAGCAGCTGCAAAAGCGGCCCAGTTTGGTTTAGTGGGCGCCGCAGGT
TTAGGCGGTTTAGGTGTGGGTGGACTGGGTGTACCTGGCGTAGGCGG
TCTGGGTGGAATTCCGCCCGCAGCGGCCGCGAAAGCGGCAAAATATG
GCGCGGCAGGCCTGGGCGGCGTGCTGGGTGGGGCAGGTCAGTTTCCG
CTGGGCGGGGTTGCCGCACGTCCGGGATTTGGTCTGAGCCCGATTTT
CCCTGGCGGCGCATGTCTGGGTAAAGCATGTGGTCGTAAACGTAAAt
aa

The amino acid sequence of a 11 kDa human elastin truncated at the N-terminal is disclosed in SEQ ID NO: 65. The 11 kDa truncated elastin has amino acids 2-635 deleted from the full length elastin.

(SEQ ID NO: 65)
VPGVLGGLGALGGVGIPGGVVGAGPAAAAAAAKAAAKAAQFGLVGAA
GLGGLGVGGLGVPGVGGLGGIPPAAAAKAAKYGAAGLGGVLGGAGQF
PLGGVAARPGFGLSPIFPGGACLGKACGRKRK

The codon optimized polynucleotide sequence encoding the 11 kDa truncated human elastin is disclosed in SEQ ID NO: 66

(SEQ ID NO: 66)
GTGCCGGGCGTCTTAGGTGGTCTGGGCGCCCTGGGTGGTGTAGGCAT
TCCGGGAGGTGTTGTGGGTGCAGGACCGGCCGCCGCAGCTGCGGCCG
CCAAAGCAGCTGCAAAAGCGGCCCAGTTTGGTTTAGTGGGCGCCGCA
GGTTTAGGCGGTTTAGGTGTGGGTGGACTGGGTGTACCTGGCGTAGG
CGGTCTGGGTGGAATTCCGCCCGCAGCGGCCGCGAAAGCGGCAAAAT
ATGGCGCGGCAGGCCTGGGCGGCGTGCTGGGTGGGGCAGGTCAGTTT
CCGCTGGGCGGGGTTGCCGCACGTCCGGGATTTGGTCTGAGCCCGAT
TTTCCCTGGCGGCGCATGTCTGGGTAAAGCATGTGGTCGTAAACGTA
AAtaa

The amino acid sequence of a 7.9 kDa human elastin truncated at the N-terminal is disclosed in SEQ ID NO: 67. The 7.9 kDa truncated elastin has amino acids 2-674 deleted from the full length elastin.

(SEQ ID NO: 67)
QFGLVGAAGLGGLGVGGLGVPGVGGLGGIPPAAAAKAAKYGAAGLGG
VLGGAGQFPLGGVAARPGFGLSPIFPGGACLGKACGRKRK

The codon optimized polynucleotide sequence encoding the 7.9 kDa truncated human elastin is disclosed in SEQ ID NO: 68

(SEQ ID NO: 68)
CAGTTTGGTTTAGTGGGCGCCGCAGGTTTAGGCGGTTTAGGTGTGGG
TGGACTGGGTGTACCTGGCGTAGGCGGTCTGGGTGGAATTCCGCCCG
CAGCGGCCGCGAAAGCGGCAAAATATGGCGCGGCAGGCCTGGGCGGC
GTGCTGGGTGGGGCAGGTCAGTTTCCGCTGGGCGGGGTTGCCGCACG
TCCGGGATTTGGTCTGAGCCCGATTTTCCCTGGCGGCGCATGTCTGG
GTAAAGCATGTGGTCGTAAACGTAAAtaa

The amino acid sequence of a 6.3 kDa human elastin truncated at the C-terminal is disclosed in SEQ ID NO: 69. The 6.3 kDa truncated elastin has amino acids 74-761 deleted from the full length elastin.

(SEQ ID NO: 69)
GGVPGAIPGGVPGGVFYPGAGLGALGGGALGPGGKPLKPVPGGLAGA
GLGAGLGAFPAVTFPGALVPGGVAD

The codon optimized polynucleotide sequence encoding the 6.3 kDa truncated human elastin is disclosed in SEQ ID NO: 70:

(SEQ ID NO: 70)
GGTGGCGTACCAGGCGCAATTCCTGGGGGTGTCCCAGGCGGTGTTTT
TTATCCGGGCGCCGGTCTTGGCGCACTGGGTGGCGGTGCACTGGGCC
CGGGCGGCAAACCGCTGAAACCGGTACCAGGTGGTTTAGCAGGCGCC
GGCTTAGGCGCAGGTCTGGGAGCATTTCCGGCAGTTACCTTTCCAGG
GGCACTGGTTCCTGGAGGTGTGGCCGATtaa

The amino acid sequence of a 4.3 kDa human elastin truncated at the N-terminal is disclosed in SEQ ID NO: 71. The 4.3 kDa truncated elastin has amino acids 2-717 deleted from the full length elastin.

(SEQ ID NO: 71)
GLGGVLGGAGQFPLGGVAARPGFGLSPIFPGGACLGKACGRKRK

The codon optimized polynucleotide sequence encoding the 4.3 kDa truncated human elastin is disclosed in SEQ ID NO: 72

(SEQ ID NO: 72)
GGCCTGGGCGGCGTGCTGGGTGGGGCAGGTCAGTTTCCGCTGGG
CGGGGTTGCCGCACGTCCGGGATTTGGTCTGAGCCCGATTTTCC
CTGGCGGCGCATGTCTGGGTAAAGCATGTGGTCGTAAACGTAAA
taa

Truncated Human Elastin 1 with DsbA Secretion and FLAG Tag

The amino acid sequence of truncated human elastin 1 with DsbA secretion and FLAG tag is disclosed in SEQ ID NO: 98. The DsbA secretion tag is encoded by nucleotides 1-57 of SEQ ID NO: 99 and the amino acid sequences are amino acids 1-19 of SEQ ID NO: 98. The elastin nucleotide sequences are nucleotides 58-657 of SEQ ID NO: 99 and the amino acid sequences are amino acids 20-219 of SEQ ID NO: 98. The FLAG nucleotide sequences are nucleotides 658-684 of SEQ ID NO: 99 and the amino acid sequences are amino acids 220-228 of SEQ ID NO: 98.

(SEQ ID NO: 98)
MKKIWLALAGLVLAFSASAGGVPGAIPGGVPGGVFYPGAGLGAL
GGGALGPGGKPLKPVPGGLAGAGLGAGLGAFPAVTFPGALVPGG
VADAAAAYKAAKAGAGLGGVPGVGGLGVSAGAVVPQPGAGVKPG
KVPGVGLPGVYPGGVLPGARFPGVGVLPGVPTGAGVKPKAPGVG
GAFAGIPGVGPFGGPQPGVPLGYPIKAPKLPGGYGLPYTTGKLG
DYKDDDDK

The nucleic acid sequence of truncated human elastin 1 with DsbA secretion and FLAG tag is disclosed in SEQ ID NO: 99.

(SEQ ID NO: 99)
ATGAAAAAGATTTGGCTGGCGCTGGCTGGTTTAGTTTTAGCGTTTAG
CGCATCGGCGGGTGGCGTACCAGGCGCAATTCCTGGGGGTGTCCCAG
GCGGTGTTTTTTATCCGGGCGCCGGTCTTGGCGCACTGGGTGGCGGT
GCACTGGGCCCGGGCGGCAAACCGCTGAAACCGGTACCAGGTGGTTT
AGCAGGCGCCGGCTTAGGCGCAGGTCTGGGAGCATTTCCGGCAGTTA
CCTTTCCAGGGGCACTGGTTCCTGGAGGTGTGGCCGATGCAGCCGCG
GCATATAAAGCCGCTAAAGCCGGTGCGGGTTTAGGAGGCGTCCCAGG
TGTCGGTGGCCTGGGTGTTAGCGCCGGTGCAGTTGTTCCGCAGCCGG
GAGCAGGGGTTAAACCTGGTAAAGTGCCGGGAGTAGGTCTGCCAGGC
GTTTATCCTGGTGGTGTTTTGCCGGGTGCCCGTTTTCCGGGCGTTGG
TGTTCTTCCAGGCGTGCCGACCGGAGCCGGTGTTAAACCGAAAGCCC
CCGGTGTTGGAGGTGCATTTGCAGGCATCCCGGGAGTTGGCCCGTTT
GGTGGTCCGCAACCTGGGGTTCCGTTAGGTTATCCGATTAAAGCACC
GAAACTGCCCGGCGGTTATGGTCTGCCGTACACAACCGGTAAACTGG
GTGACTACAAAGACGACGACGACAAAtaa

The polynucleotide of SEQ ID NO: 99 was subcloned into vector pET28a, expressed host E. coli cells and the truncated elastin was purified as described herein. The purified elastin produced a clear band on SDS-PAGE and an anti-FLAG western was observed at around 25 kilodaltons. There were no existing bands that appear at that location on the gel in the absence of expression of this protein.

Truncated Human Elastin 2 with DsbA Secretion and FLAG Tag

The amino acid sequence of truncated human elastin type 2 with DsbA secretion and FLAG tag is disclosed in SEQ ID NO: 100. The DsbA secretion tag is encoded by nucleotides 1-57 of SEQ ID NO: 101 and the amino acid sequences are amino acids 1-19 of SEQ ID NO: 100. The elastin nucleotide sequences are nucleotides 58-657 of SEQ ID NO: 101 and the amino acid sequences are amino acids 20-219 of SEQ ID NO: 100. The FLAG nucleotide sequences are nucleotides 658-684 of SEQ ID NO: 101 and the amino acid sequences are amino acids 220-228 of SEQ ID NO: 100.

(SEQ ID NO: 100)
MKKIWLALAGLVLAFSASAPYGYGPGGVAGAAGKAGYPTGTGVGPQA
AAAAAAKAAAKFGAGAAGVLPGVGGAGVPGVPGAIPGIGGIAGVGTP
AAAAAAAAAAKAAKYGAAAGLVPGGPGFGPGVVGVPGAGVPGVGVPG
AGIPVVPGAGIPGAAVPGVVSPEAAAKAAAKAAKYGARPGVGVGGIP
TYGVGAGGFPGFGVGVGGIPGVAGVPGVGGVGDYKDDDDK

The nucleic acid sequence of truncated human elastin 2 with DsbA secretion and FLAG tag is disclosed in SEQ ID NO: 101.

(SEQ ID NO: 101)
ATGAAAAAGATTTGGCTGGCGCTGGCTGGTTTAGTTTTAGCGTTTAG
CGCATCGGCGCCGTATGGTTATGGCCCGGGTGGAGTTGCGGGTGCAG
CAGGTAAAGCGGGTTATCCTACCGGAACCGGTGTAGGTCCGCAGGCC
GCTGCTGCCGCCGCCGCAAAAGCAGCGGCTAAATTTGGCGCCGGAGC
AGCGGGTGTTCTGCCTGGAGTTGGTGGTGCGGGCGTGCCAGGGGTAC
CTGGTGCAATTCCGGGTATTGGTGGTATTGCCGGTGTCGGCACCCCG
GCCGCGGCAGCTGCGGCAGCGGCGGCTGCCAAAGCTGCTAAATACGG
TGCCGCGGCGGGTCTGGTGCCAGGAGGTCCGGGTTTTGGTCCGGGAG
TGGTTGGCGTGCCTGGCGCAGGCGTTCCTGGTGTGGGCGTTCCAGGT
GCAGGGATTCCTGTTGTGCCTGGTGCCGGTATTCCCGGCGCGGCCGT
TCCGGGGGTGGTTAGCCCGGAAGCCGCAGCGAAGGCTGCGGCAAAGG
CAGCAAAGTATGGCGCACGCCCAGGAGTCGGCGTGGGTGGTATCCCG
ACCTATGGGGTGGGCGCAGGGGGTTTTCCTGGTTTCGGCGTAGGTGT
AGGAGGTATACCGGGCGTGGCCGGTGTACCAGGGGTTGGTGGCGTCG
GTGACTACAAAGACGACGACGACAAAtaa

The polynucleotide of SEQ ID NO: 101 was subcloned into vector pET28a, expressed host E. coli cells and the truncated elastin was purified as described herein. The purified elastin produced a clear band on SDS-PAGE and an anti-FLAG western was observed at around 25 kilodaltons. There were no existing bands that appear at that location on the gel in the absence of expression of this protein.

### Example 6: Effect of Truncated Collagen on Fibroblast Cell Viability, Procollagen Synthesis, and Elastin Synthesis

A human fibroblast cell culture was used to assess the ability of the truncated jellyfish collagen molecule of Example 2 to determine its effect on procollagen, and elastin synthesis. The human fibroblast cell culture was also used to determine the increased viability of the human fibroblast cells after exposure to the truncated jellyfish collagen.

A stock solution of 2% w/w truncated collagen was prepared from the histidine tagged truncated collagen of example 3. Aliquots from the 2% stock truncated collagen solution were then used in the experiments described below.

### Preparation of Fibroblasts

Fibroblasts were seeded into the individual wells of a 24-well plate in 0.5 ml of Fibroblast Growth Media (FGM) and incubated overnight at 37±2° C. and 5±1% CO₂. On the following day the media was removed via aspiration to eliminate any non-adherent cells and replaced with 0.5 ml of fresh FGM. The cells were grown until confluent, with a media change every 48 to 72 hours. Upon reaching confluency the cells were treated for 24 hours with DMEM supplemented with 1.5% FBS to wash out any effects from the growth factors included in the normal culture media. After the 24-hour wash out period the cells were treated with the truncated jellyfish collagen at specified concentrations dissolved in FGM with 1.5% FBS. Transforming Growth Factor Beta (TGF-β) (20 ng/ml) was used as a positive control for collagen and elastin synthesis. Untreated cells (negative controls) just received DMEM with 1.5% FBS. The cells were incubated for 48 hours and at the end of the incubation period cell culture medium was collected and either stored frozen (−75° C.) or assayed immediately. Materials were tested in triplicate.

### MTT Assay

The MTT (3-(4,5-Dimethylthiazol-2-yl)-2,5-diphenyltetrazolium bromide, a tetrazole) assay is a colorimetric assay used to determine the metabolic activity of cells. Changes in cell number were assessed via an MTT assay. When cells are exposed to MTT, reduction of MTT by mitochondria in viable cells results in the formation of insoluble purple formazin crystals that are extracted from the cells with isopropanol and quantified spectrophotometrically. Non-living cells cannot reduce MTT and therefore cannot produce the purple formazin crystals. The intensity of the purple color is directly proportional to the number of living cells (metabolically active cells). The intensity of the purple color is directly proportional to the metabolic activity of the cells and is inversely proportional to the toxicity of the test material.

After the 2-day incubation discussed above, the cell culture medium was removed (see above) and the fibroblasts were washed twice with PBS to remove any remaining jellyfish glycogen molecules. After the final wash, 500 μl of DMEM supplemented with 0.5 mg/ml MTT was added to each well and the cells were incubated for 1 hour at 37±2° C. and 5±1% CO₂. After the incubation, the DMEM/MTT solution was removed and the cells were washed again once with PBS and then 0.5 ml of isopropyl alcohol was added to the well to extract the purple formazin crystals. Two hundred microliters of the isopropyl extracts was transferred to a 96-well plate and the plate was read at 540 nm using isopropyl alcohol as a blank.

The mean MTT absorbance value for the negative control cells was calculated and used to represent 100% cell viability. The individual MTT absorbance values from the cells undergoing the various treatments were then divided by the mean value for the negative control cells and expressed as a percent to determine the change in cell viability caused by each treatment.

In Tables 1, 2 and 3 of this example, the experiments were performed by using the designated aliquots of the 2% stock truncated collagen solution in the assays. For example, in the samples that tested the “10% Collagen Solution,” an aliquot of the 2% truncated collagens in an amount sufficient to provide 10% of the assay volume was used. For a total assay volume of 1.0 ml, 100 μl of the 2% stock truncated collagen solution was used. In Tables 1, 2 and 3, “10% Collagen Solution” is 0.2% collagen, “5% Collagen Solution” is 0.1% collagen, “1% Collagen Solution” is 0.02% collagen, “0.5% Collagen Solution” is 0.01% collagen, “0.1% Collagen Solution” is 0.002% collagen, “0.05% Collagen Solution” is 0.001% collagen, “0.01% Collagen Solution” is 0.0002% collagen, “0.005% Collagen Solution” is 0.0001% collagen.

The results for the MTT assay are presented in Table 3. The values are presented as the mean percent viability the deviation from the mean.

TABLE 3
MTT Assay

| | |
|---|---|
| Untreated | 100 ± 6.1 |
| 20 ng/ml TGF-B | 110 ± 2.9 |
| 10% Collagen Solution | 131 ± 7.8* |
| 5% Collagen Solution | 140 ± 8.3* |
| 1% Collagen Solution | 116 ± 0.9 |
| 0.5% Collagen Solution | 105 ± 4.8 |
| 0.1% Collagen Solution | 102 ± 1.1 |
| 0.05% Collagen Solution | 106 ± 3.4 |
| 0.01% Collagen Solution | 112 ± 1.9 |
| 0.005% Collagen Solution | 103 ± 3.9 |

*Denotes values that are significantly different from the Untreated group (p < 0.05).

The histidine tagged truncated jellyfish collagen showed protective effect by increasing the cell viability of human fibroblast cells.

### Procollagen Synthesis

Fibroblasts are the main source of the extracellular matrix peptides, including the structural proteins collagen and elastin. Procollagen is a large peptide synthesized by fibroblasts in the dermal layer of the skin and is the precursor for collagen. As the peptide is processed to form a mature collagen protein, the propeptide portion is cleaved off (type I C-peptide). Both the mature collagen protein and the type I C-peptide fragment are then released into the extracellular environment. As collagen is synthesized the type I C-peptide fragment accumulates into the tissue culture medium. Since there is a 1:1 stoichiometric ratio between the two parts of the procollagen peptide, assaying for type I C-peptide will reflect the amount of collagen synthesized. Type 1 C-peptide can be assayed via an ELISA based method.

A series of type I C-peptide standards was prepared ranging from 0 ng/ml to 640 ng/ml. Next, an ELISA microplate was prepared by removing any unneeded strips from the plate frame followed by the addition of 100 μl of peroxidase-labeled anti procollagen type I-C peptide antibody to each well used in the assay. Twenty (20) μl of either sample (collected tissue culture media) or standard was then added to appropriate wells and the microplate was covered and allowed to incubate for 3±0.25 hours at 37° C. After the incubation the wells were aspirated and washed three times with 400 μl of wash buffer. After the last wash was removed 100 μl of peroxidase substrate solution (hydrogen peroxide+tetramethylbenzidine as a chromagen) was added to each well and the plate was incubated for 15±5 minutes at room temperature. After the incubation 100 μl of stop solution (1 N sulfuric acid) was added to each well and the plate was read using a microplate reader at 450 nm.

To quantify the amount of each substance present, a standard curve was generated using known concentrations of each substance. A regression analysis was performed to establish the line that best fits these data points. Absorbance values for the test materials and untreated samples were used to estimate the amount of each substance present in each sample.

The results for the ELISA assays are presented in Table 4.

TABLE 4
Type I Collagen Assay.
The values presented are mean concentration (ng/ml) ±
the deviation from the mean.

| Treatment | Type I C-Peptide (ng/ml) |
|---|---|
| Untreated | 1718 ± 94 |
| 20 ng/ml TGF-B | 3028 ± 332* |
| 10% Collagen Solution | 1940 ± 100 |
| 5% Collagen Solution | 2394 ± 125* |
| 1% Collagen Solution | 1773 ± 183 |
| 0.5% Collagen Solution | 1127 ± 19* |
| 0.1% Collagen Solution | 1158 ± 10* |
| 0.05% Collagen Solution | 1416 ± 64 |
| 0.01% Collagen Solution | 1835 ± 404 |
| 0.005% Collagen Solution | 1551 ± 149 |

*Denotes values that are significantly different from the Untreated group (p < 0.05).

The truncated histidine tagged jellyfish collagen was observed to have a biphasic effect on collagen synthesis. At the 1%, 5% and the 10% levels, collagen synthesis increased. At the 5% concentration truncated jellyfish collagen significantly increased collagen synthesis with a p-value of less than 0.05.

### Elastin Synthesis

Elastin is the main component of a network of elastic fibers that give tissues their ability to recoil after a transient stretch. This protein is released by fibroblasts (soluble elastin) into the extracellular space where it is then cross-linked to other elastin proteins to form an extensive network of fibers and sheets (insoluble elastin). Soluble elastin can be readily measured from cell culture medium via an ELISA based method.

Soluble α-elastin was dissolved in 0.1 M sodium carbonate (pH 9.0) at a concentration of 1.25 μg/ml. 150 μl of this solution was then applied to the wells of a 96-well maxisorp Nunc plate and the plate was incubated overnight at 4° C. On the following day the wells were saturated with PBS containing 0.25% BSA and 0.05% TWEEN® 20. The plate was then incubated with this blocking solution for 1 hour at 37° C. and then washed two times with PBS containing 0.05% TWEEN® 20.

A set of α-elastin standards was generated ranging from 0 to 100 ng/ml. 180 μl of either standard or truncated jellyfish collagen was then transferred to a 650 μl microcentrifuge tube. An anti-elastin antibody solution was prepared (the antibody was diluted 1:100 in PBS containing 0.25% BSA and 0.05% TWEEN® 20) and 20 μl of the solution was added to the tube. The tubes were then incubated overnight at 4±2° C. On the following day, 150 μl was transferred from each tube to the 96-well elastin ELISA plate, and the plate was incubated for 1 hour at room temperature. The plate was then washed 3 times with PBS containing 0.05% TWEEN® 20. After washing, 200 μl of a solution containing a peroxidase linked secondary antibody diluted in PBS containing 0.25% BSA and 0.05% TWEEN® 20 was added, and the plate was incubated for 1 hour at room temperature. After washing the plate three times, 200 μl of a substrate solution was added and the plate was incubated for 10 to 30 minutes in the dark at room temperature. After this final incubation the plate was read at 460 nm using a plate reader.

TABLE 5
The values are also presented as mean concentration
(ng/ml) ± deviation from the mean.

| Treatment | Elastin (ng/ml) |
|---|---|
| Untreated | 79 ± 19 |
| 20 ng/ml TGFB1 | 243 ± 35* |
| 10% Collagen Solution | 68 ± 18 |
| 5% Collagen Solution | 99 ± 13 |
| 1% Collagen Solution | 126 ± 21 |
| 0.5% Collagen Solution | 145 ± 21* |
| 0.1% Collagen Solution | 76 ± 14 |
| 0.05% Collagen Solution | 58 ± 6 |
| 0.01% Collagen Solution | 53 ± 5 |
| 0.005% Collagen Solution | 56 ± 24 |

*Denotes values that are significantly different from the Untreated group (p < 0.05).

Truncated his-tagged jellyfish collagen significantly increased elastin production when it was used at the 0.5% concentration as shown in Table 5.

### Example 7: Effect of Truncated Collagen on Keratinocyte Proliferation and UVB Protection

A human keratinocyte cell culture model was used to assess the ability of the test materials to exert an effect on cell proliferation. In addition, the impact of the test materials on the cell viability after an exposure to UVB was also assessed.

A stock solution of 2% w/w truncated collagen was prepared from the truncated collagen of example 1. Aliquots from the 2% stock truncated collagen solution was then used in the experiments described below.

This study was conducted in two parts. In the first part cultured keratinocytes were incubated with the test materials for 48 hours, after which changes in the number of viable cells were assessed using an MTT assay. In the second part of the study cultured keratinocytes were irradiated with UVB and then treated with the test materials for 48 hours. At the end of the 48 hour period the number of viable cells was again assessed via an MTT assay.

Changes in cell number of viable cells can be determined using an MTT (3-(4,5-Dimethylthiazol-2-yl)-2,5-diphenyltetrazolium bromide, a tetrazole) assay. The MTT assay is a colorimetric analysis of the metabolic activity of the cell, which is a reflection of the number of viable cells. Reduction of MTT by mitochondria in viable cells results in the formation of insoluble purple formazin crystals that are extracted from the cells with isopropanol and quantified spectrophotometrically. The intensity of the purple color is directly proportional to the number of metabolically active cells.

### Proliferation Assay

For the proliferation assay the keratinocytes were seeded into 96-well plates using without growth factors and incubated for 24 hours at 37±2° C. and 5±1% CO2. After this initial incubation the media was replaced with media supplemented with the test materials. Normal media (with growth factors) was used as a positive control. After the addition of the test materials the cells were cultured for 48 hours as described above. At the end of the incubation period changes in the number of viable cells was determined using an MTT assay.

### UVB Protection Assay

For the UVB protection assay the keratinocytes were seeded into 96-well plates using normal media and incubated for 24 hours at 37±2° C. and 5±1% CO2. After this initial incubation the media was replaced with 100 μl of phosphate buffered saline (PBS) and the cells were exposed to UVB (40 mJ/cm2). After the UVB exposure, the PBS was replaced with fresh media supplemented with the test materials (ascorbic acid at 100 μg/ml served as the positive control) and the cells were cultured for 48 hours at 37±2° C. and 5±1% CO2. At the end of the 48 hour incubation cell viability was determined using an MTT assay.

### MTT Assay

After the 48-hour incubation the cell culture medium was removed and replaced with 200 μl of culture media supplemented with 0.5 mg/ml MTT. The well plates were incubated for 1 hour at 37±2° C. and 5±1% CO2. After the incubation, the MTT solution was removed and the cells were washed once with phosphate buffered saline and then 200 μl of isopropyl alcohol was added to the well to extract the purple formazin crystals. The 96-well plate was read at 540 nm using isopropyl alcohol as a blank.

The mean absorbance value for the cells not treated with test materials (proliferation assay: Untreated Group) or not exposed to UVB (UVB protection assay: Non-UVB Exposed Group) was calculated and used to represent 100% cell viability. The individual absorbance values from the cells undergoing the various treatments were then divided by the mean absorbance value representing 100% cell viability and expressed as a percent to determine the change in cell viability caused by each treatment.

The results for the Proliferation assay using the his-tagged truncated jellyfish collagen are presented in Table 6. The results for the UVB Protection assay using the his-tagged truncated jellyfish collagen are presented in Table 7. The values for both assays are presented as mean viability±standard deviation.

TABLE 6
Proliferation Assay

| | |
|---|---|
| Untreated | 100 ± 3.4 |
| Positive Control (Growth Factors) | 139 ± 3.8* |
| 10% Collagen Solution | 103 ± 9.4 |
| 5% Collagen Solution | 97 ± 7.3 |
| 1% Collagen Solution | 94 ± 5.0 |
| 0.5% Collagen Solution | 93 ± 7.0 |
| 0.1% Collagen Solution | 95 ± 2.5 |
| 0.05% Collagen Solution | 99 ± 6.0 |
| 0.01% Collagen Solution | 96 ± 6.4 |
| 0.005% Collagen Solution | 96 ± 2.8 |

*Denotes values which are significantly different from the Untreated Group (p < 0.05)

TABLE 7
UVB Protection Assay

| | |
|---|---|
| Non-UVB Exposed | 100 ± 1.7* |
| Untreated | 77 ± 1.8 |
| 100 ug/ml Trolox | 92 ± 2.0* |
| 10% Collagen Solution | 76 ± 8.6 |
| 5% Collagen Solution | 92 ± 3.9* |
| 1% Collagen Solution | 91 ± 2.9* |
| 0.5% Collagen Solution | 100 ± 4.5* |
| 0.1% Collagen Solution | 86 ± 4.8 |
| 0.05% Collagen Solution | 91 ± 1.6* |
| 0.01% Collagen Solution | 83 ± 7.5 |
| 0.005% Collagen Solution | 82 ± 4.7 |

*Denotes values which are significantly different from the Untreated Group (p < 0.05)

For the proliferation assay the Untreated group was used to represent 100% cell viability. Values above 100% reflect an increase in the number of viable cells and hence are indicative of cell proliferation. In this study, the test material was not observed to promote cell proliferation.

In addition, the keratinocyte proliferation assay was performed using the truncated collagen of SEQ ID NO: 91. A 1% and 0.5% collagen solution of a 5% stock solution was prepared according to Example 8 and tested. The truncated collagen of SEQ ID NO: 91 had keratinocyte cell viability assay values of 102±2.9 and 102±2.0, respectively. The observed values were statistically significant (p<0.05).

In addition to its effects on cell proliferation, the test material was also screened to determine if it had an impact on cell recovery after UVB exposure. In this study, exposure to UVB was observed to result in a significant reduction in the number of viable cells 48 hours post exposure. However, treatment with the test material prevented this decrease in cell viability. The effect was evident within a concentration range between 0.05% and 5% of the test material, with an optimal effect at a concentration of 0.05%. Within this range of concentrations cell viability was significantly greater than the untreated group (with the lone exception of the 0.01% concentration), demonstrating that the material has UVB protective effect. Since this material was added after the UVB exposure it could be acting to reduce the damaging effects of the UVB irradiation, or it could be helping the damaged cells to recover at a faster rate. With respect to the latter, then truncated collagen is beneficial when applied topically to the skin and has a regenerative effect on skin cells damaged by UVB.

In addition, the UVB protection assay was performed using the truncated collagen of SEQ ID NO: 91. A 1% and 0.5% collagen solution of SEQ ID NO: 91 had keratinocyte cell viability assay values of 80±4.6 and 78±2.5, respectively. The observed values were statistically significant (p<0.05).

### Example 8: Effect of Truncated Collagen on Thymine Dimer Formation

Upon exposure to ultraviolet radiation the thymine dimer (TT dimer) content in DNA present in cells increases. Increases in TT dimer formation are correlated with skin damage and certain types of cell proliferative diseases including skin cancer.

The polynucleotide of SEQ ID NO: 11 was expressed in the expression system of Example 1 and purified as described in this example. The encoded polypeptide includes the DsbA secretion tag. As the polypeptide is processed through the secretion pathway, the DsbA tag, amino acids 1-24 of SEQ ID NO: 12 is cleaved by the host cell. The truncated collagen without the DsbA secretion tag is provided in SEQ ID NO: 91.

The truncated collagen of SEQ ID NO: 91 was tested to determine if it could reduce TT dimer formation is human epidermal keratinocytes. For this study, the cells were exposed to UVB (25 mJ/cm2). Following the exposures cells were treated with the test materials or Trolox (100 μg/ml) and incubated overnight. On the following day cellular DNA was extracted and assayed for thymine dimer content using an ELISA based method.

Human keratinocytes were seeded into 12-well plates using normal media and incubated for 24 hours at 37±2° C. and 5±1% CO2. After this initial incubation the media was replaced with 100 μl of phosphate buffered saline (PBS) and the cells were exposed to UVB (25 mJ/cm2). After the UVB exposure, the PBS was replaced with fresh media supplemented with the test materials or Trolox (100 μg/ml, this served as the positive control) and the cells were cultured overnight at 37±2° C. and 5±1% CO2. At the end of the incubation cellular DNA was extracted.

After the overnight incubation the cell culture media was removed from the wells and replaced with 200 μl of PBS and 20 μl of Proteinase K. After swirling the plate to mix the PBS and Proteinase K, 200 μl of buffer AL was added to each well. After again swirling the plate to mix the reagents, the plates were incubated for 10 minutes at 55±2° C. After cooling the plate to room temperature, the DNA was precipitated by the addition of 200 μl of 100% ethanol. The precipitated DNA mixtures were then transferred to DNEASY® Spin Columns in 2 ml collection tubes and centrifuged at 8,000 RPM for 1 minute. The flow through and collection tubes were discarded, and 500 μl of Wash Buffer One was added to the spin column and the column was placed into a new collection tube and centrifuged at 8,000 RPM for 1 minute. The flow through and collection tube were again discarded, and 500 μl of Wash Buffer Two was added to the spin column and the column was placed into a new collection tube and centrifuged at 14,000 RPM for 3 minutes. The spin column was then placed into a new 1.5 ml centrifuge tube and 110 μl of ultrapure water was added to the column. The column was incubated for 1 minute at room temperature and then centrifuged at 8,000 RPM for 1 minute.

Extracted DNA was quantified via a fluorometric assay. A 2 μl aliquot of the DNA sample was mixed with 100 μl TE buffer in a 96-well plate. A series of DNA standards was also transferred to wells in a 96-well plate (in duplicate). Finally, 100 μl of dilute CYQUANT® Green dye was added to each well and the fluorescence intensity of each well was determined using an excitation wavelength of 480 nm and an emission wavelength of 520 nm.

Thymine Dimer Detection was determined using an OXISELECT™ UV-Induced DNA Damage ELISA Kit)

Aliquots of genomic DNA samples or standards were converted to single stranded DNA by incubating the samples at 95° C. for 10 minutes and then chilled on ice. 100 μl or each sample or standard was transferred to a DNA binding ELISA plate and incubated overnight at 4° C. On the following day the wells were rinsed once with 100 μl of PBS and then blocked with 150 μl of Assay Diluent for one hour at room temperature. After removing the Assay Diluent, 100 μl of anti-CPD antibody was added to each well and the plate was incubated for one hour at room temperature. After this incubation, the plate was washed three times with 250 μl of wash buffer per well, and then 150 μl of Blocking Reagent was added to the plate. The plate will be blocked again for one hour at room temperature, and then washed three times as described before. 100 μl of Secondary Antibody was then added to each well and the plate was incubated for 1 hour at room temperature. After washing the plate again, 100 μl of substrate was added to each well and the plate was incubated for 5-20 minutes to allow for color generation in the plate. The color generation reaction was stopped by the addition of 100 μl of stop solution and the plate was read at 460 nm using a plate reader.

To quantify the amount of DNA present, a standard curve was generated using known concentrations of DNA and their respective fluorescence intensity (measured in RFUs or relative fluorescence units). A regression analysis was performed to establish the line that best fits these data points. The Relative Fluorescence Units (RFU) for each unknown sample was then used to estimate the amount of DNA.

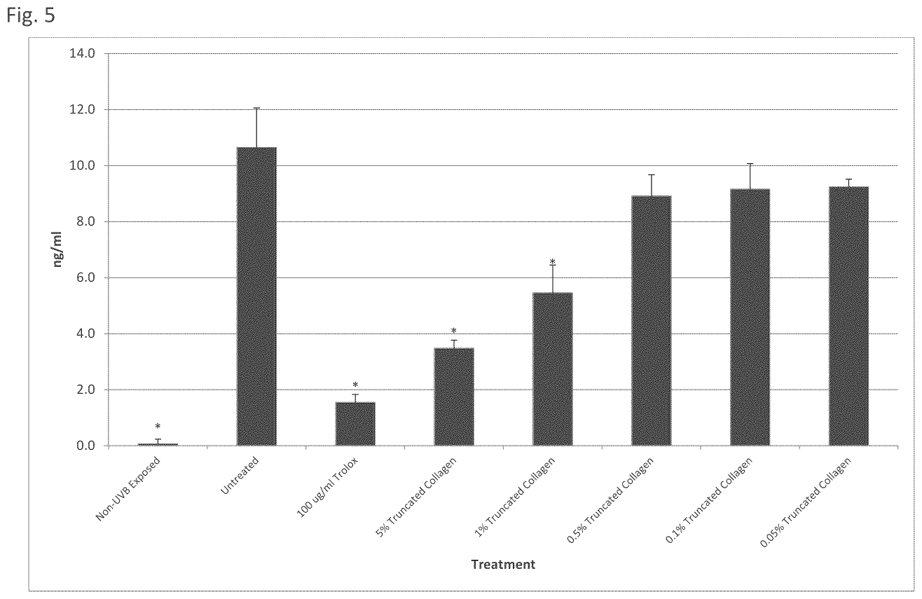

A series of DNA standards with known amounts of thymine dimer content were used to generate a standard curve. This standard curve was used to determine the amount of DNA damage in the sample DNA. Means for each treatment group were calculated and compared using an ANOVA. In table 8 and FIG. 5, a standard 5% collagen solution (5 g truncated collagen in 95 ml deionized water was further diluted with phosphate buffered saline (PBS) to the indicated percent solution.

TABLE 8
Thymine Dimer Assay

| Treatment | Thymine Dimer, ng/ml |
|---|---|
| Non-UVB Exposed | 0.1 ± 0.2* |
| Untreated | 10.7 ± 1.4* |
| 100 μg/ml Trolox | 1.6 ± 0.3* |
| 5% Truncated Collagen | 3.5 ± 0.3* |
| 1% Truncated Collagen | 5.5 ± 1.0* |
| 0.5% Truncated Collagen | 8.9 ± 0.8 |
| 0.1% Truncated Collagen | 9.2 ± 0.9 |
| 0.05% Truncated Collagen | 9.2 ± 0.3 |

*Denotes values which are statistically significantly different from untreated with P < 0.05

Table 8 shows that the 5% and the 1% truncated collagen solution reduced TT dimer formation with statistical significance (p<0.05). The data is presented graphically in FIG. 5.

The experiment was repeated with a different lot of truncated collagen (SEQ ID NO: 91). The amount of TT dimer in ng/ml in non-UVB exposed cells was 1.3±1.2, untreated cells was 18.1±0.4, 100 μg/ml Trolox treated cells was 7.9±0.3, 5% collagen was 13.1±0.2, and 1% collagen was 17.4±0.7. The reduction in TT dimer formation for non-UVB exposed cells, Trolox treated cells, 5% collagen treated cells and 1% collagen treated cells was statistically significant compared to untreated cells (p<0.05).

### Example 9: Human Collagens

### Truncated Human Collagen Type 21 Alpha 1

A truncated human collagen type 21 alpha 1 without a His tag, linker, and thrombin cleavage site is disclosed below. The codon-optimized nucleotide sequence encoding this collagen and the amino acid sequence are disclosed below. In SEQ ID NOs: 73 and 74, the DsbA secretion tag is encoded by nucleotides 1-72 and encodes amino acids 1-24. In SEQ ID NOs: 73 and 74, the truncated collagen sequence is encoded by nucleotides 73-633 and encodes amino acids 25-211.

The codon-optimized nucleotide sequence encoding this collagen is provided in SEQ ID NO: 73.

(SEQ ID NO: 73)
ATGAAAAAGATTTGGCTGGCGCTGGCTGGTTTAGTTTTAGCGTTTAG
CGCATCGGCGGCGCAGTATGAAGATGCAGGTTTTCCGGGTCTGCCTG
GTCCGGCAGGCGAACCGGGTCGTCATGGTAAAGATGGTCTGATGGGT
AGTCCGGGTTTTAAAGGTGAAGCAGGTTCACCGGGTGCACCTGGTCA
GGATGGCACCCGTGGTGAACCGGGTATTCCGGGATTTCCGGGTAATC
GTGGCCTGATGGGTCAGAAAGGTGAAATTGGTCCGCCTGGTCAGCAG
GGTAAAAAAGGCGCACCGGGTATGCCAGGACTGATGGGTTCAAATGG
CAGTCCGGGTCAGCCAGGCACACCGGGTTCAAAAGGTAGCAAAGGCG
AACCTGGTATTCAGGGTATGCCTGGTGCAAGCGGTCTGAAAGGCGAG
CCAGGTGCCACCGGTTCTCCGGGTGAACCAGGTTATATGGGTCTGCC
AGGTATCCAAGGCAAAAAAGGTGATAAAGGTAATCAGGGCGAAAAAG
GCATTCAGGGCCAGAAAGGCGAAAATGGCCGTCAGGGTATTCCAGGC
CAGCAGGGCATCCAGGGTCATCATGGTGCAAAAGGTGAACGTGGTGA
AAAGGGCGAACCAGGTGTTCGTtaa

The amino acid sequence is disclosed in SEQ ID NO: 74.

(SEQ ID NO: 74)
MKKIWLALAGLVLAFSASAAQYEDAGFPGLPGPAGEPGRHGKDGLMG
SPGFKGEAGSPGAPGQDGTRGEPGIPGFPGNRGLMGQKGEIGPPGQQ
GKKGAPGMPGLMGSNGSPGQPGTPGSKGSKGEPGIQGMPGASGLKGE
PGATGSPGEPGYMGLPGIQGKKGDKGNQGEKGIQGQKGENGRQGIPG
QQGIQGHHGAKGERGEKGEPGVR

The codon-optimized nucleotide sequence encoding the truncated human collagen type 21 alpha 1 without the DsbA secretion tag collagen is provided in SEQ ID NO: 75.

(SEQ ID NO: 75)
TGCAGGTTTTCCGGGTCTGCCTGGTCCGGCAGGCGAACCGGGTCGTC
ATGGTAAAGATGGTCTGATGGGTAGTCCGGGTTTTAAAGGTGAAGCA
GGTTCACCGGGTGCACCTGGTCAGGATGGCACCCGTGGTGAACCGGG
TATTCCGGGATTTCCGGGTAATCGTGGCCTGATGGGTCAGAAAGGTG
AAATTGGTCCGCCTGGTCAGCAGGGTAAAAAAGGCGCACCGGGTATG
CCAGGACTGATGGGTTCAAATGGCAGTCCGGGTCAGCCAGGCACACC
GGGTTCAAAAGGTAGCAAAGGCGAACCTGGTATTCAGGGTATGCCTG
GTGCAAGCGGTCTGAAAGGCGAGCCAGGTGCCACCGGTTCTCCGGGT
GAACCAGGTTATATGGGTCTGCCAGGTATCCAAGGCAAAAAAGGTGA
TAAAGGTAATCAGGGCGAAAAAGGCATTCAGGGCCAGAAAGGCGAAA
ATGGCCGTCAGGGTATTCCAGGCCAGCAGGGCATCCAGGGTCATCAT
GGTGCAAAAGGTGAACGTGGTGAAAAGGGCGAACCAGGTGTTCGTta
a

The amino acid sequence of truncated human collagen type 21 alpha 1 without the DsbA secretion tag is disclosed in SEQ ID NO: 76.

(SEQ ID NO: 76)
AGFPGLPGPAGEPGRHGKDGLMGSPGFKGEAGSPGAPGQDGTRGEPG
IPGFPGNRGLMGQKGEIGPPGQQGKKGAPGMPGLMGSNGSPGQPGTP
GSKGSKGEPGIQGMPGASGLKGEPGATGSPGEPGYMGLPGIQGKKGD
KGNQGEKGIQGQKGENGRQGIPGQQGIQGHHGAKGERGEKGEPGVR

### Truncated Human Collagen Type 1 Alpha 2 (1)

A truncated human collagen type 1 alpha 2 without a His tag, linker, and thrombin cleavage site is disclosed below. The codon-optimized nucleotide sequence and the amino acid sequences are disclosed below. In SEQ ID NOs: 78 and 79, The DsbA secretion tag is encoded by nucleotides 1-72 and encodes amino acids 1-24. The truncated collagen sequence is encoded by nucleotides 73-636 and encodes amino acids 25-212.

The codon-optimized nucleotide sequence encoding this collagen is provided in SEQ ID NO: 77.

(SEQ ID NO: 77)
ATGAAAAAGATTTGGCTGGCGCTGGCTGGTTTAGTTTTAGCGTTTAG
CGCATCGGCGGCGCAGTATGAAGATATGGGTCCGCCTGGTAGCCGTG
GTGCAAGTGGTCCGGCAGGCGTTCGTGGTCCGAATGGTGATGCAGGT
CGTCCGGGTGAACCGGGTCTGATGGGTCCTCGTGGTCTGCCTGGTTC
ACCGGGTAATATTGGTCCTGCAGGTAAAGAAGGTCCGGTTGGTCTGC
CAGGTATTGATGGCCGTCCGGGTCCGATTGGTCCAGCCGGTGCACGT
GGTGAACCTGGCAATATTGGTTTTCCGGGTCCTAAAGGTCCGACCGG
TGATCCGGGTAAAAATGGTGATAAAGGTCATGCAGGTCTGGCAGGCG
CACGCGGTGCACCTGGTCCGGATGGTAATAATGGTGCACAGGGTCCA
CCGGGTCCGCAGGGTGTTCAAGGTGGTAAAGGCGAACAGGGTCCTGC
CGGTCCTCCGGGTTTTCAGGGACTGCCTGGTCCGAGCGGTCCTGCGG
GTGAAGTTGGTAAACCTGGTGAACGCGGTCTGCATGGTGAATTTGGC
CTGCCTGGGCCTGCAGGTCCGCGTGGCGAACGTGGTCCGCCAGGTGA
AAGCGGTGCAGCAGGTCCGACAGGTtaa

The amino acid sequence is disclosed in SEQ ID NO: 78.

(SEQ ID NO: 78)
MKKIWLALAGLVLAFSASAAQYEDMGPPGSRGASGPAGVRGPNGDAG
RPGEPGLMGPRGLPGSPGNIGPAGKEGPVGLPGIDGRPGPIGPAGAR
GEPGNIGFPGPKGPTGDPGKNGDKGHAGLAGARGAPGPDGNNGAQGP
PGPQGVQGGKGEQGPAGPPGFQGLPGPSGPAGEVGKPGERGLHGEFG
LPGPAGPRGERGPPGESGAAGPTG

The nucleic acid sequence of truncated human collagen type 1 alpha 2(1) without the DsbA secretion tag is disclosed in SEQ ID NO: 79.

(SEQ ID NO: 79)
ATGGGTCCGCCTGGTAGCCGTGGTGCAAGTGGTCCGGCAGGCGTTCG
TGGTCCGAATGGTGATGCAGGTCGTCCGGGTGAACCGGGTCTGATGG
GTCCTCGTGGTCTGCCTGGTTCACCGGGTAATATTGGTCCTGCAGGT
AAAGAAGGTCCGGTTGGTCTGCCAGGTATTGATGGCCGTCCGGGTCC
GATTGGTCCAGCCGGTGCACGTGGTGAACCTGGCAATATTGGTTTTC
CGGGTCCTAAAGGTCCGACCGGTGATCCGGGTAAAAATGGTGATAAA
GGTCATGCAGGTCTGGCAGGCGCACGCGGTGCACCTGGTCCGGATGG
TAATAATGGTGCACAGGGTCCACCGGGTCCGCAGGGTGTTCAAGGTG
GTAAAGGCGAACAGGGTCCTGCCGGTCCTCCGGGTTTTCAGGGACTG
CCTGGTCCGAGCGGTCCTGCGGGTGAAGTTGGTAAACCTGGTGAACG
CGGTCTGCATGGTGAATTTGGCCTGCCTGGGCCTGCAGGTCCGCGTG
GCGAACGTGGTCCGCCAGGTGAAAGCGGTGCAGCAGGTCCGACAGGT
taa

The amino acid sequence of truncated human collagen type 1 alpha 2(1) without the DsbA secretion tag is disclosed in SEQ ID NO: 80.

(SEQ ID NO: 80)
MGPPGSRGASGPAGVRGPNGDAGRPGEPGLMGPRGLPGSPGNIGPAG
KEGPVGLPGIDGRPGPIGPAGARGEPGNIGFPGPKGPTGDPGKNGDK
GHAGLAGARGAPGPDGNNGAQGPPGPQGVQGGKGEQGPAGPPGFQGL
PGPSGPAGEVGKPGERGLHGEFGLPGPAGPRGERGPPGESGAAGPTG

### Truncated Human Collagen Type 1 Alpha 2 (2)

A truncated human collagen type 1 alpha 2 without a His tag, linker, and thrombin cleavage site is disclosed below. The codon-optimized nucleotide sequence and the amino acid sequences are disclosed below. in SEQ ID NO: 82 and 83, the DsbA secretion tag is encoded by nucleotides 1-72 and encodes amino acids 1-24. The truncated collagen sequence is encoded by nucleotides 73-609 and encodes amino acids 25-203.

The codon-optimized nucleotide sequence encoding this collagen is provided in SEQ ID NO: 81.

(SEQ ID NO: 81)
ATGAAAAAGATTTGGCTGGCGCTGGCTGGTTTAGTTTTAGCGTTTAG
CGCATCGGCGGCGCAGTATGAAGATGGTTTTCAGGGTCCTGCCGGTG
AACCGGGTGAACCTGGTCAGACAGGTCCGGCAGGCGCACGTGGTCCT
GCAGGTCCTCCTGGTAAAGCCGGTGAAGATGGTCATCCGGGTAAACC
GGGTCGTCCTGGTGAACGTGGTGTTGTTGGTCCGCAGGGTGCCCGTG
GTTTTCCGGGTACTCCGGGTCTGCCAGGTTTTAAAGGTATTCGTGGT
CATAATGGTCTGGATGGTCTGAAAGGTCAGCCTGGTGCACCGGGTGT
TAAAGGTGAACCAGGTGCTCCGGGTGAAAATGGCACACCGGGTCAGA
CCGGTGCGCGTGGTCTGCCTGGCGAACGCGGTCGTGTTGGTGCACCT
GGTCCAGCCGGTGCACGCGGTAGTGATGGTAGCGTTGGTCCGGTTGG
TCCAGCGGGTCCGATTGGTAGCGCAGGTCCACCGGGTTTTCCAGGCG
CACCGGGTCCGAAAGGTGAAATTGGTGCAGTTGGTAATGCAGGCCCT
GCCGGTCCAGCAGGACCGCGTGGTGAAGTTGGCCTGCCTGGTCTGta
a

The amino acid sequence is disclosed in SEQ ID NO: 82.

(SEQ ID NO: 82)
MKKIWLALAGLVLAFSASAAQYEDGFQGPAGEPGEPGQTGPAGARGP
AGPPGKAGEDGHPGKPGRPGERGVVGPQGARGFPGTPGLPGFKGIRG
HNGLDGLKGQPGAPGVKGEPGAPGENGTPGQTGARGLPGERGRVGAP
GPAGARGSDGSVGPVGPAGPIGSAGPPGFPGAPGPKGEIGAVGNAGP
AGPAGPRGEVGLPGL

The nucleic acid sequence of truncated human collagen type 1 alpha 2(2) without the DsbA secretion tag is disclosed in SEQ ID NO: 83.

(SEQ ID NO: 83)
GGTTTTCAGGGTCCTGCCGGTGAACCGGGTGAACCTGGTCAGACAGG
TCCGGCAGGCGCACGTGGTCCTGCAGGTCCTCCTGGTAAAGCCGGTG
AAGATGGTCATCCGGGTAAACCGGGTCGTCCTGGTGAACGTGGTGTT
GTTGGTCCGCAGGGTGCCCGTGGTTTTCCGGGTACTCCGGGTCTGCC
AGGTTTTAAAGGTATTCGTGGTCATAATGGTCTGGATGGTCTGAAAG
GTCAGCCTGGTGCACCGGGTGTTAAAGGTGAACCAGGTGCTCCGGGT
GAAAATGGCACACCGGGTCAGACCGGTGCGCGTGGTCTGCCTGGCGA
ACGCGGTCGTGTTGGTGCACCTGGTCCAGCCGGTGCACGCGGTAGTG
ATGGTAGCGTTGGTCCGGTTGGTCCAGCGGGTCCGATTGGTAGCGCA
GGTCCACCGGGTTTTCCAGGCGCACCGGGTCCGAAAGGTGAAATTGG
TGCAGTTGGTAATGCAGGCCCTGCCGGTCCAGCAGGACCGCGTGGTG
AAGTTGGCCTGCCTGGTCTGtaa

The amino acid sequence of truncated human collagen type 1 alpha 2(2) without the DsbA secretion tag is disclosed in SEQ ID NO: 84.

(SEQ ID NO: 84)
GFQGPAGEPGEPGQTGPAGARGPAGPPGKAGEDGHPGKPGRPGERGV
VGPQGARGFPGTPGLPGFKGIRGHNGLDGLKGQPGAPGVKGEPGAPG
ENGTPGQTGARGLPGERGRVGAPGPAGARGSDGSVGPVGPAGPIGSA
GPPGFPGAPGPKGEIGAVGNAGPAGPAGPRGEVGLPGL

The polynucleotides of SEQ ID NO: 73, 77 or 81 were subcloned in vector pET28a as described herein to prepare a transformation vector. Host cells were transformed with the vector the polynucleotides were expressed as described in Example 2.

After the fermentation was completed, the truncated human collagen was purified from the fermentation broth using the procedures disclosed in Example 3. The purified truncated human collagens were analyzed using SDS-PAGE and HPLC as disclosed in Example 3.

All three truncated human collagens ran at the expected molecular weights in the SDS-PAGE analysis. In analyzing the truncated human collagens using HPLC, a standard curve using the jellyfish collagen of Example 3 was utilized. The retention times of the human collagens were slightly different than the jellyfish collagen. The retention time of SEQ ID NO: 76 was 5.645 minutes, the retention time of SEQ ID NO: 80 was 5.631 minutes, and SEQ ID NO: 84 ran at two peaks and the retention times were 5.531 and 5.7 minutes.

Truncated Human Collagen Type 1 Alpha 2 Truncation 5 with DsbA Secretion and FLAG Tag

The amino acid sequence of truncated human collagen type 1 alpha 2 truncation 5 with DsbA secretion and FLAG tag is disclosed in SEQ ID NO: 92. The DsbA secretion tag is encoded by nucleotides 1-57 of SEQ ID NO: 93 and the amino acid sequences are amino acids 1-19 of SEQ ID NO: 92. The collagen nucleotide sequences are nucleotides 58-657 of SEQ ID NO: 93 and the amino acid sequences are amino acids 20-219 of SEQ ID NO: 92. The FLAG nucleotide sequences are nucleotides 658-684 of SEQ ID NO: 93 and the amino acid sequences are amino acids 220-228.

(SEQ ID NO: 92)
MKKIWLALAGLVLAFSASAGDQGPVGRTGEVGAVGPPGFAGEKGPSG
EAGTAGPPGTPGPQGLLGAPGILGLPGSRGERGLPGVAGAVGEPGPL
GIAGPPGARGPPGAVGSPGVNGAPGEAGRDGNPGNDGPPGRDGQPGH
KGERGYPGNIGPVGAAGAPGPHGPVGPAGKHGNRGETGPSGPVGPAG
AVGPRGPSGPQGIRGDKGEPGEKGPRGLPGLGDYKDDDDK

The nucleic acid sequence of truncated human collagen type 1 alpha 2 truncation 5 with DsbA secretion and FLAG tag is disclosed in SEQ ID NO: 93.

(SEQ ID NO: 93)
ATGAAAAAGATTTGGCTGGCGCTGGCTGGTTTAGTTTTAGCGTTTAG
CGCATCGGCGGGTGATCAGGGTCCGGTTGGTCGTACCGGTGAAGTTG
GTGCAGTCGGGCCGCCGGGTTTTGCGGGTGAAAAAGGCCCGTCAGGT
GAAGCAGGCACCGCTGGCCCTCCTGGCACGCCTGGCCCACAGGGTTT
ACTGGGCGCACCTGGAATTCTGGGACTGCCGGGCAGCCGTGGAGAAC
GCGGTTTACCAGGTGTTGCCGGTGCCGTTGGTGAACCTGGTCCACTG
GGCATTGCAGGGCCGCCTGGCGCACGGGGACCGCCTGGTGCTGTTGG
TAGTCCGGGTGTGAATGGTGCTCCGGGTGAAGCCGGTCGTGACGGTA
ATCCGGGAAATGACGGCCCGCCAGGCCGCGATGGTCAGCCGGGTCAT
AAAGGTGAGCGTGGTTACCCAGGTAATATTGGTCCAGTCGGTGCCGC
CGGTGCGCCGGGTCCTCATGGCCCTGTCGGTCCAGCCGGTAAACATG
GTAATCGCGGTGAGACAGGTCCGTCAGGACCAGTGGGCCCTGCTGGC
GCAGTCGGTCCGCGCGGGCCGAGTGGCCCTCAGGGTATTCGTGGCGA
TAAAGGGGAACCGGGCGAAAAAGGGCCGCGGGGTCTGCCAGGCCTGG
GTGACTACAAAGACGACGACGACAAAtaa

The polynucleotide of SEQ ID NO: 93 was subcloned into vector pET28a, expressed host E. coli cells and the truncated collagen was purified as described herein. The purified collagen produced a clear band on SDS-PAGE and an anti-FLAG western was observed at around 100 kilodaltons. There were no existing bands that appear at that location on the gel in the absence of expression of this protein.

Truncated Human Collagen Type 1 Alpha 2 Truncation 6 with DsbA Secretion and FLAG Tag

The amino acid sequence of truncated human collagen type 1 alpha 2 truncation 6 with DsbA secretion and FLAG tag is disclosed in SEQ ID NO: 94. The DsbA secretion tag is encoded by nucleotides 1-57 of SEQ ID NO: 95 and the amino acid sequences are amino acids 1-19 of SEQ ID NO: 94. The collagen nucleotide sequences are nucleotides 58-657 of SEQ ID NO: 95 and the amino acid sequences are amino acids 20-219 of SEQ ID NO: 94. The FLAG nucleotide sequences are nucleotides 658-684 of SEQ ID NO: 95 and the amino acid sequences are amino acids 220-228 of SEQ ID NO: 94.

(SEQ ID NO: 94)
MKKIWLALAGLVLAFSASAKGHNGLQGLPGIAGHHGDQGAPGSVGPA
GPRGPAGPSGPAGKDGRTGHPGTVGPAGIRGPQGHQGPAGPPGPPGP
PGPPGVSGGGYDFGYDGDFYRADQPRSAPSLRPKDYEVDATLKSLNN
QIETLLTPEGSRKNPARTCRDLRLSHPEWSSGYYWIDPNQGCTMDAI
KVYCDFSTGETCIRAQPENIPAKNWYRSSKDGDYKDDDDK

The nucleic acid sequence of truncated human collagen type 1 alpha 2 truncation 6 with DsbA secretion and FLAG tag is disclosed in SEQ ID NO: 95.

(SEQ ID NO: 95)
ATGAAAAAGATTTGGCTGGCGCTGGCTGGTTTAGTTTTAGCGTTTAG
CGCATCGGCGAAAGGTCACAATGGACTGCAAGGCCTGCCAGGTATTG
CAGGTCATCATGGTGATCAAGGTGCCCCGGGAAGCGTTGGTCCGGCG
GGGCCGAGAGGCCCTGCGGGACCTTCAGGTCCGGCAGGCAAAGATGG
TCGGACAGGCCATCCGGGCACCGTTGGCCCTGCAGGAATTCGTGGAC
CGCAGGGTCATCAGGGACCTGCTGGTCCGCCAGGTCCCCCGGGCCCT
CCGGGACCACCGGGTGTTAGTGGTGGTGGTTATGATTTTGGCTATGA
TGGTGATTTTTATCGTGCAGATCAGCCGCGTAGCGCACCGAGCCTGC
GTCCTAAAGATTATGAAGTTGATGCAACCCTGAAAAGCCTGAATAAT
CAGATTGAAACACTGCTGACACCGGAAGGTAGCCGTAAAAATCCGGC
CCGTACCTGTCGTGATCTGCGTCTGAGCCACCCGGAATGGAGCAGCG
GTTATTATTGGATTGATCCGAATCAAGGTTGTACCATGGATGCAATT
AAAGTTTATTGTGATTTTAGCACAGGTGAAACATGTATCCGTGCACA
GCCGGAAAATATTCCGGCCAAAAATTGGTATCGTAGTAGCAAAGATG
GTGACTACAAAGACGACGACGACAAAtaa

The polynucleotide of SEQ ID NO: 94 was subcloned into vector pET28a, expressed host E. coli cells and the truncated collagen was purified as described herein. The purified collagen produced a clear band on SDS-PAGE and an anti-FLAG western was observed at around 25 kilodaltons. There were no existing bands that appear at that location on the gel in the absence of expression of this protein.

Truncated Human Collagen Type 1 Alpha 2 Truncation 7 with DsbA Secretion and FLAG Tag

The amino acid sequence of truncated human collagen type 1 alpha 2 truncation 7 with DsbA secretion and FLAG tag is disclosed in SEQ ID NO: 96. The DsbA secretion tag is encoded by nucleotides 1-57 of SEQ ID NO: 97 and the amino acid sequences are amino acids 1-19 of SEQ ID NO: 96. The collagen nucleotide sequences are nucleotides 58-759 of SEQ ID NO: 96 and the amino acid sequences are amino acids 20-253 of SEQ ID NO: 96. The FLAG nucleotide sequences are nucleotides 760-786 of SEQ ID NO: 97 and the amino acid sequences are amino acids 254-262 of SEQ ID NO: 96.

(SEQ ID NO: 96)
MKKIWLALAGLVLAFSASAYEVDATLKSLNNQIETLLTPEGSRKNPA
RTCRDLRLSHPEWSSGYYWIDPNQGCTMDAIKVYCDFSTGETCIRAQ
PENIPAKNWYRSSKDKKHVWLGETINAGSQFEYNVEGVTSKEMATQL
AFMRLLANYASQNITYHCKNSIAYMDEETGNLKKAVILQGSNDVELV
AEGNSRFTYTVLVDGCSKKTNEWGKTIIEYKTNKPSRLPFLDIAPLD
IGGADQEFFVDIGPVCFKGDYKDDDDK

The nucleic acid sequence of truncated human collagen type 1 alpha 2 truncation 7 with DsbA secretion and FLAG tag is disclosed in SEQ ID NO: 97.

(SEQ ID NO: 97)
TGAAAAAGATTTGGCTGGCGCTGGCTGGTTTAGTTTTAGCGTTTAGCGCAT
CGGCGTATGAAGTTGATGCAACCCTGAAAAGCCTGAATAATCAGATTGAA
ACACTGCTGACACCGGAAGGTAGCCGTAAAAATCCGGCCCGTACCTGTCG
TGATCTGCGTCTGAGCCACCCGGAATGGAGCAGCGGTTATTATTGGATTG
ATCCGAATCAAGGTTGTACCATGGATGCAATTAAAGTTTATTGTGATTTTA
GCACAGGTGAAACATGTATCCGTGCACAGCCGGAAAATATTCCGGCCAAA
AATTGGTATCGTAGTAGCAAAGATAAAAAACATGTGTGGCTGGGTGAAAC
CATTAATGCAGGTAGCCAGTTTGAATACAATGTTGAAGGTGTTACCAGCA
AAGAAATGGCAACACAGCTGGCATTTATGCGTCTGCTGGCAAATTATGCA
AGCCAGAATATTACATATCATTGTAAAAATAGCATTGCATATATGGATGA
AGAAACCGGTAATCTGAAAAAAGCAGTTATTCTGCAGGGTAGCAATGATG
TTGAACTGGTTGCCGAAGGTAATAGCCGTTTTACATATACCGTTCTGGTTG
ATGGTTGTAGCAAAAAAACCAATGAATGGGGTAAAACCATCATTGAATAT
AAAACCAACAAACCGAGCCGTCTGCCGTTTCTGGATATCGCTCCGCTGGA
TATTGGTGGTGCCGATCAGGAATTTTTTGTCGATATCGGTCCTGTGTGTTT
TAAAGGTGACTACAAAGACGACGACGACAAAtaa

The polynucleotide of SEQ ID NO: 97 was subcloned into vector pET28a, expressed host E. coli cells and the truncated collagen was purified as described herein. The purified collagen produced a clear band on SDS-PAGE and an anti-FLAG western was observed at around 30 kilodaltons. There were no existing bands that appear at that location on the gel in the absence of expression of this protein.

### Example 10: Protective Effect of Truncated Human Collagen on Fibroblasts

The effect of truncated human collagen on fibroblast cell viability, procollagen synthesis, and elastin Synthesis is determined according to the methods of Example 6.

The effect of truncated human collagen on Keratinocyte proliferation and UVB protection is determined according to the methods of Example 7.

The effect of truncated collagen on thymine dimer formation after exposure to UV radiation is determined according to the methods of Example 8.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 11: Effect of Truncated Collagen on Inflammatory Cytokines

Keratinocytes and dermal fibroblasts play an important role in the immune response of the skin. In response to irritating chemicals or UV radiation (pro-inflammatory/pro-irriation stimuli), keratinocytes can release a vast array of cytokines. These cytokines are thought to help engage immune cells to the site of inflammation. Cytokines released by the keratinocytes include TNFα, IL-1α, IL-1β, IL-3, IL-6, IL-7, IL-8, IL-10, IL-18, and IL-1RA.

The testing model used for this study was the MatTek EPIDERM®. This skin model consists of normal human-derived epidermal keratinocytes that have been cultured to form a multilayered, highly differentiated model of the human epidermis. Ultrastructural analysis has revealed the presence of keratohyalin granules, tonofilament bundles, desmosomes, and a multi-layered stratum comeum containing intercellular lamellar lipid layers arranged in patterns characteristic of in vivo epidermis. Markers of mature epidermis specific differentiation such as pro-filaggrin, the K1/K10 cytokeratin pair, involucrin, and type I epidermal transglutaminase have been localized in this model. The MatTek EPIDERM® is also mitotically and metabolically active.

The MatTek EPIDERM® tissues were used to assess the ability of various test materials to inhibit the release the inflammatory mediator IL-1α. Test materials were compared to an over the counter topical hydrocortisone preparation (positive control) as well as to untreated tissues (negative control 1) and untreated, non-inflamed tissues (negative control 2). This test was also used to assess the viability of the tissues after exposure to the test materials.

IL-1α, IL-6 and IL-8 are synthesized and stored in keratinocytes and have been identified as a mediators of skin irritation and inflammation. Release of these cytokines can be directly measured in tissue culture media via a colorimetric based enzyme linked immunosorbent assay (ELISA). Briefly, antibodies covalently linked to a solid support will bind IL-1a, IL-6 or IL-8 present in spent culture media samples. A second antibody that is covalently attached to an acetylcholinesterase enzyme will in turn detect the specific bound cytokines. Upon addition of an appropriate color substrate the acetylcholinesterase enzyme will generate a colored end product that can be measured spectrophotometrically.

MatTek EPIDERM® Tissues were purchased from MatTek corporation and were stored at 4° C. until used. Prior to use, the tissues to be used were removed from the agarose-shipping tray and placed into a 6-well plate containing 0.9 ml of hydrocortisone free assay medium (37±2° C.). The tissues were allowed to incubate overnight at 37±2° C. and 5±1% CO2. After this initial incubation, the assay medium was replaced with 0.9 ml of fresh hydrocortisone free medium (37±2° C.). Three tissues were prepared for each test material.

An inflammatory response in the tissues was initiated via UV irradiation (UVB). A UV lamp was used to give a 300 mJ/cm² dose of UVB radiation to the tissues. Immediately after the application of the inflammatory stimuli 50 μl or mg of test material was applied directly onto the surface of the tissue. An over the counter hydrocortisone cream was used as a positive control. For a negative control tissues were exposed to the inflammatory stimuli but were not treated with any type of anti-inflammatory material. One additional set of tissues was left without exposure to the inflammatory stimuli to provide a baseline measurement for the cytokines. The tissues were incubated at 37±2° C. and 5±1% CO₂ for 24 hours after exposure to the inflammatory stimuli. After the 24-hour incubation the cell culture medium was collect and stored at −75° C. until analyzed for cytokines.

The ELISA plates were prepared by diluting the appropriate capture antibody in PBS. Next, 100 μl of the diluted capture antibody was added to the wells of a 96-well ELISA plate and the plate was incubated overnight at room temperature. On the following day the plate was washed three times with 300 μl wash buffer (0.05% TWEEN®20 in PBS) and then blocked by adding 300 μl of blocking buffer (1% BSA in PBS) to each well. The plate was incubated with the blocking buffer for at least one hour. After the incubation the blocking buffer was removed and the plate was washed three times as described above.

A series of standards was prepared and 100 μl of each of these standards was dispensed into two wells (duplicates) in the appropriate 96-well plate. Subsequently, 100 μl of each sample was added to additional wells and the plate was incubated for two hours at room temperature. After the incubation the plate was washed three times as described above. Once the last wash was removed, 100 μl of a biotin conjugated detection antibody was added. After incubating the plate for two hours at room temperature the plate was washed again as described above. 100 μl of HRP-streptavidin was then added to each well and the plate was incubated for 20 minutes at room temperature. Once the last wash was removed, 100 μl of substrate solution (hydrogen peroxide+tetramethylbenzidine as a chromagen) was added to each well. Once a sufficient level of color development had occurred, 50 μl of stop solution (2N sulfuric acid) was added to each well and the plate was read at 460 nm.

After the 24 hour incubation, the tissues were rinsed twice with at least 100 μl of phosphate buffered saline to remove the test material and then transferred to a 6-well plate containing 1.0 ml of assay medium supplemented with MTT (1 mg/ml) and allowed to incubate for 3±0.25 hours at 37±2° C. and 5±1% CO2. After the incubation, the tissues were rinsed at least twice with 100 μl of phosphate buffered saline, blotted dry, and then placed into a 24-well plate containing 2 ml of isopropanol per well. The 24-well plate was covered and allowed to incubate at room temperature for at least 2 hours on a rocking platform to extract the reduced MTT from the tissues. After the extraction, a 200 μl sample of the isopropanol/MTT mixture was transferred to a 96-well plate and the absorbance of the sample was read at 540 nm with a plate reader using 200 μl of isopropanol as the blank. The MTT assay is described in Example 6 herein. The cell viability results of the MTT assay were similar to the results obtained in Example 6

The results of the IL-1a assay are shown in Table 9 below. A 2% stock solution of the jellyfish collagen of SEQ ID NO: 91 is Sample 4 and Sample 3 is a 2% stock solution of the truncated jellyfish collagen of SEQ ID NO: 10. In Table 9 below, the indicated percentage is the percent dilution of the stock solution used for the test. For example, the 1% Sample 4 treatment is a 1% solution of the 2% stock truncated collagen solution. The untreated cells produced 18.2 pg/ml of Il-1a. Upon treatment with truncated collagen, all samples showed decreases in Il-1a production. The 1% Sample 4 treatment reduced IL-1A production to 13.4 pg/ml, which is significant with a p value of less than 0.05. The decrease in IL-1a production indicates that the truncated collagen has anti-inflammatory effects.

TABLE 9
IL-1a Assay

| Treatment | II-1a pg/ml |
|---|---|
| Non-UVB Exposed | 4.9 ± 2.1 |
| Untreated | 18.2 ± 1.4 |
| 1% Hydrocortisone | 6.7 ± 0.6 |
| 5% Sample 4 | 18.1 ± 1.1 |
| 1% Sample 4 | 13.4 ± 0.9* |
| 0.5% Sample 4 | 15.7 ± 0.7 |
| 0.1% Sample 4 | 13.9 ± 1.7* |
| 5% Sample 3 | 15.8 ± 2.0 |

Denotes values that are significantly different from Untreated (p < 0.05)

### Example 11: Urban Dust Protection by Truncated Collagen

A keratinocyte cell culture model was used to assess the ability of truncated collagens to exert a protective effect by promoting cell survival after exposure to urban dust.

Human epidermal keratinocytes were pretreated with the test materials and then exposed to urban dust. At the end of the treatment period changes in cell viability were then determined via an MTT assay.

Keratinocytes were seeded into the individual wells of a 96 well plate in 100 μl of medium and incubated overnight at 37±2° C. and 5±1% CO₂. On the following day the media was removed via aspiration to eliminate any non-adherent cells and replaced with 100 μl of fresh medium. The cells were grown until confluent, with a media change every 48 to 72 hours.

Pretreatment with Test Material Followed by Urban Dust Treatment

Test materials were prepared at 2× their final desired concentrations in cell culture media. Urban dust (NIST 1649B from Sigma Chemicals) was also prepared at 2× solutions. For the pretreatment, 50 μl of 2× test material was combined with 50 μl of culture media and the cells were incubated for 24 hours. At the end of the pretreatment period the test material containing culture media was removed and replaced with 50 μl of 2× urban dust and 50 μl of media. Another set of cells was treated with media alone (non-dust exposed) and used as a reference control to represent 100% cell viability. The cells were then incubated for 24 hours and then subjected to an MTT assay to determine changes in cell viability.

At the end of the treatment period, the cell culture medium was removed and the cells were washed with PBS. After the wash, 100 μl of cell culture media supplemented with 0.5 mg/ml MTT was added to each well and the cells were incubated for 30 minutes at 37±2° C. and 5±1% CO2. After the incubation, the media/MTT solution was removed and the cells were washed again once with PBS and then 100 μl of isopropyl alcohol was added to the wells to extract the purple formazin crystals. The 96-well plate was then read at 540 nm using isopropyl alcohol as a blank.

The mean MTT absorbance value for the non-dust exposed cells was calculated and used to represent 100% value for cell number. The individual MTT values from the cells undergoing the various treatments was then divided by the mean value for the non-dust exposed cells and expressed as a percent to determine the change in cell number caused by each treatment.

The MTT results for the pretreatment with the test material then dust treatments are presented in Table 10. Table 10 shows that as the cells were treated with increasing amounts of collagen, cell viability increased upon pretreatment with truncated collagen and subsequent exposure to urban dust. These results show that truncated collagen protects against the decline in cell viability associated with urban dust exposure.

TABLE 10
MTT Assay, Truncated Collagen Pretreatment

| | Viability (% Non-Dust | Viability (% Non-Dust |
|---|---|---|
| | Exposed) | Exposed) |
| Treatment | 4 mg/ml Urban Dust | 2 mg/ml Urban Dust |
| Non-Dust | 100 ± 4.4* | 100 ± 1.8 |
| Exposed | | |
| Untreated | 59 ± 3.8 | 70 ± 0.9 |
| 0.1% Collagen | 61 ± 4.7 | 72 ± 3.5 |
| 0.5% Collagen | 59 ± 2.2 | 73 ± 2.2 |
| 1% Collagen | 58 ± 0.5 | 74 ± 2.6 |
| 5% Collagen | 66 ± 1.0 | 83 ± 6.5* |

*Denotes values that are significantly different from untreated group (p < 0.05)

### Example 12: Truncated Chondrosia reniformis (Kidney Sponge) Collagen

Truncated Chondrosia reniformis (Kidney Sponge) Fibrillar Collagen 1 with DsbA Secretion and FLAG Tag

The amino acid sequence of truncated Chondrosia reniformis fibrillar collagen 1 with DsbA secretion and FLAG tag is disclosed in SEQ ID NO: 102. The DsbA secretion tag is encoded by nucleotides 1-57 of SEQ ID NO: 103 and the amino acid sequences are amino acids 1-19 of SEQ ID NO: 102. The fibrillary collagen nucleotide sequences are nucleotides 58-792 of SEQ ID NO: 103 and the amino acid sequences are amino acids 20-264 of SEQ ID NO: 102. The FLAG nucleotide sequences are nucleotides 793-819 of SEQ ID NO: 103 and the amino acid sequences are amino acids 265-273 of SEQ ID NO: 102.

(SEQ ID NO: 102)
MKKIWLALAGLVLAFSASAPVGRRGPKGSRGDPGDGGAAGPKGPEGVDGLI
GEPGQPGPIGAEGSSGLEGFLGDKGSKGARGGPGNRGRPGQDGVPGQDGRA
GEKGEGGETGDRGQQGLRGKVGDPGLVGDLGAQGPQGSQGLVGPPGIPGEP
GSGGEPGDQGPRGPEGPQGSPGVRGGRGERGTPGAVGPKGPPGKNGADGPR
GLPGASGPPGSPGNQGPEGSRGADGNNGFPGDDGENGLVGIPGEPGPKGAR
GTRGELGKTGDYKDDDDK

The nucleic acid sequence of truncated Chondrosia reniformis fibrillar collagen 1 with DsbA secretion and FLAG tag is disclosed in SEQ ID NO: 103.

(SEQ ID NO: 103)
ATGAAAAAGATTTGGCTGGCGCTGGCTGGTTTAGTTTTAGCGTTTAGCGCA
TCGGCGCCGGTTGGTCGTCGTGGTCCGAAAGGTAGCCGTGGTGATCCTGG
TGATGGTGGTGCAGCAGGTCCTAAAGGTCCGGAAGGTGTTGATGGTCTGA
TTGGTGAACCGGGTCAGCCTGGTCCGATTGGCGCAGAAGGTAGCAGCGGT
CTGGAAGGTTTTCTGGGTGATAAAGGTAGCAAAGGTGCACGTGGTGGTCC
GGGTAATCGCGGTCGTCCTGGTCAGGATGGTGTTCCGGGTCAAGATGGTC
GTGCCGGTGAAAAAGGTGAAGGTGGTGAAACCGGTGATCGCGGTCAGCA
GGGTCTGCGTGGTAAAGTTGGTGATCCAGGTCTGGTGGGTGATCTGGGTG
CACAGGGTCCGCAGGGTAGCCAAGGTCTGGTTGGTCCGCCTGGTATTCCG
GGTGAACCTGGTAGCGGTGGCGAACCGGGTGATCAGGGTCCTCGCGGTCC
AGAAGGTCCTCAGGGTTCACCGGGTGTTCGCGGTGGTCGTGGTGAACGTG
GTACACCGGGTGCAGTTGGACCGAAAGGTCCGCCAGGTAAAAATGGTGCA
GATGGTCCGCGTGGTCTGCCTGGTGCAAGCGGTCCTCCGGGTAGTCCTGGT
AACCAGGGTCCTGAAGGTTCTCGTGGTGCCGATGGTAATAATGGTTTTCCA
GGTGATGATGGTGAAAATGGCCTGGTTGGTATCCCTGGCGAACCAGGTCC
AAAAGGCGCACGCGGTACACGCGGTGAACTGGGTAAAACCGGTGACTAC
AAAGACGACGACGACAAAtaa

The polynucleotide of SEQ ID NO: 103 was subcloned into vector pET28a, expressed host E. coli cells and the truncated Chondrosia reniformis fibrillar collagen 1 was purified as described herein. The purified fibrillary collagen produced a clear band on SDS-PAGE and an anti-FLAG western was observed at around 40 kilodaltons. There were no existing bands that appear at that location on the gel in the absence of expression of this protein.

Truncated Chondrosia reniformis (Kidney Sponge) Fibrillar Collagen 2 with DsbA Secretion and FLAG Tag

The amino acid sequence of truncated Chondrosia reniformis fibrillar collagen 2 with DsbA secretion and FLAG tag is disclosed in SEQ ID NO: 104. The DsbA secretion tag is encoded by nucleotides 1-57 of SEQ ID NO: 105 and the amino acid sequences are amino acids 1-19 of SEQ ID NO: 104. The fibrillary collagen nucleotide sequences are nucleotides 58-1323 of SEQ ID NO: 105 and the amino acid sequences are amino acids 20-441 of SEQ ID NO: 104. The FLAG nucleotide sequences are nucleotides 1324-1350 of SEQ ID NO: 105 and the amino acid sequences are amino acids 442-450 of SEQ ID NO: 105.

(SEQ ID NO: 104)
MKKIWLALAGLVLAFSASAGRGGPAGLQGAAGNPGDPGDRGQAGEIGLPGT
EGQRGQGGSRGDDGIGGQSGTDGDPGNDGVAGIRGARGEPGATGPEGAAGQ
KGDRGRFGEQGRPGNDGPPGRRGRVGNLGETGAEGDEGTRGYTGDRGPEGA
IGISGVTGNPGPQGIKGPPGDTGHPGRQGPSGPQGPPGIPGTDGLTIHNLI
KPPSQFFDATSSSDPLTDAVVESILKSFQYAELEIDLTKKPDGTMKYPAIS
CDDLHKDYPQLPSGNYTLDPNGGCKNDAFETYCEFNNSVKMCLTPKIPTLL
PMGTYKYYVNSEGYYSPNDFGLNLRFFEYYGSVTQLKFLQTKATRVTQTIR
VLCKNYDPLHKQPVFIGMNDETVMDEPRMEENQCQYFNGLSAHVELELSSN
DPSYLPIYEMRLYLGRKTNEELGIELGDLCFEYGDYKDDDDK

The nucleic acid sequence of truncated Chondrosia reniformis fibrillar collagen 2 with DsbA secretion and FLAG tag is disclosed in SEQ ID NO: 105.

(SEQ ID NO: 105)
ATGAAAAAGATTTGGCTGGCGCTGGCTGGTTTAGTTTTAGCGTTTAGCGCA
TCGGCGGGTCGTGGCGGTCCGGCAGGTCTGCAGGGTGCTGCAGGTAATCC
TGGCGACCCTGGCGATCGTGGTCAGGCAGGCGAAATTGGTCTGCCAGGCA
CCGAAGGTCAGCGTGGTCAAGGTGGTTCACGTGGTGATGACGGTATTGGT
GGTCAGAGCGGCACCGATGGCGATCCGGGTAACGATGGTGTTGCAGGTAT
TCGTGGTGCACGCGGAGAACCTGGTGCCACCGGACCTGAAGGTGCAGCCG
GTCAGAAAGGTGATCGTGGCCGTTTTGGCGAACAGGGTCGTCCGGGAAAT
GATGGTCCACCGGGTCGCCGTGGCCGTGTGGGCAATCTGGGTGAAACAGG
TGCCGAAGGTGATGAAGGCACCCGTGGTTATACAGGTGACCGTGGACCGG
AAGGCGCAATTGGTATTAGCGGTGTGACCGGTAATCCGGGTCCACAGGGC
ATTAAAGGCCCTCCGGGTGATACGGGTCATCCGGGTCGTCAGGGACCGAG
CGGTCCGCAAGGACCACCGGGTATTCCAGGTACAGATGGCCTGACCATTC
ATAATCTGATTAAACCGCCTAGCCAGTTTTTTGATGCAACCAGCAGCAGC
GATCCGCTGACCGATGCAGTTGTTGAAAGCATTCTGAAATCTTTTCAGTAT
GCCGAGCTGGAAATTGACCTGACCAAAAAACCGGATGGCACCATGAAAT
ATCCGGCAATTAGCTGTGATGATCTGCACAAAGATTATCCGCAGCTGCCG
AGCGGTAATTATACCCTGGATCCGAATGGTGGTTGTAAAAATGATGCCTTT
GAAACCTATTGCGAGTTCAACAATAGCGTGAAAATGTGTCTGACCCCGAA
AATTCCGACACTGCTGCCGATGGGCACCTATAAATACTATGTTAATAGCG
AGGGTTACTACAGCCCGAATGATTTTGGTCTGAATCTGCGCTTTTTTGAGT
ATTATGGTAGCGTTACCCAGCTGAAATTTCTGCAGACCAAAGCAACCCGT
GTTACCCAGACCATTCGTGTTCTGTGTAAAAACTATGATCCGCTGCATAAA
CAGCCGGTTTTTATTGGTATGAATGACGAAACCGTTATGGATGAACCGCG
TATGGAAGAAAATCAGTGCCAGTATTTTAACGGTCTGAGCGCACATGTTG
AACTGGAACTGAGCAGCAATGATCCGAGCTATCTGCCGATTTATGAAATG
CGTCTGTATCTGGGTCGTAAAACCAATGAAGAACTGGGCATTGAACTGGG
CGATCTGTGTTTTGAATATGGTGACTACAAAGACGACGACGACAAAtaa

The polynucleotide of SEQ ID NO: 105 was subcloned into vector pET28a, expressed host E. coli cells and the truncated Chondrosia reniformis fibrillar collagen 2 was purified as described herein. The purified fibrillary collagen produced a clear band on SDS-PAGE and an anti-FLAG western was observed at around 55 kilodaltons. There were no existing bands that appear at that location on the gel in the absence of expression of this protein.

Truncated Chondrosia reniformis (Kidney Sponge) Non-Fibrillar Collagen 1 with DsbA Secretion and FLAG Tag

The amino acid sequence of truncated Chondrosia reniformis non-fibrillar collagen 1 with DsbA secretion and FLAG tag is disclosed in SEQ ID NO: 106. The DsbA secretion tag is encoded by nucleotides 1-57 of SEQ ID NO: 107 and the amino acid sequences are amino acids 1-19 of SEQ ID NO: 106. The non-fibrillar collagen nucleotide sequences are nucleotides 58-831 of SEQ ID NO: 107 and the amino acid sequences are amino acids 20-277 of SEQ ID NO: 106. The FLAG nucleotide sequences are nucleotides 832-858 of SEQ ID NO: 107 and the amino acid sequences are amino acids 278-286 of SEQ ID NO: 106.

(SEQ ID NO: 106)
MKKIWLALAGLVLAFSASAEKTSSKVALMTVLVVITGALIIEGTSITRGST
HVNRGLRKRQTSEDNCEAVKVGLPGRDGREGPPGPPGPAGRDGRDAVCSNQ
TTGLGAKGDRGPPGTPGFPGEVGRPGPPGADGIPGPQGERGAVGPGGKPGP
RGEVGTPGADGADGATGATGVQGPDGAKGEKGASGTAGLKGEKGDTCIPDS
NSTLGMPGTPGAGGSKGQKGESGIVGPKGERGEIGTPGHPGFRGADGEPGH
KGVPGRAGAQGDRGDPGDDGLTGDYKDDDDK

The nucleic acid sequence of truncated Chondrosia reniformis non-fibrillar collagen 1 with DsbA secretion and FLAG tag is disclosed in SEQ ID NO: 107.

(SEQ ID NO: 107)
ATGAAAAAGATTTGGCTGGCGCTGGCTGGTTTAGTTTTAGCGTTTAGCGCA
TCGGCGGAAAAAACCAGCAGCAAAGTTGCACTGATGACCGTTCTGGTTGT
TATTACCGGTGCACTGATTATTGAAGGCACCAGCATTACCCGTGGTAGCA
CCCATGTTAATCGTGGTCTGCGTAAACGTCAGACCAGCGAAGATAATTGT
GAAGCAGTTAAAGTTGGTCTGCCAGGTCGTGATGGTCGTGAAGGTCCTCC
GGGTCCGCCTGGTCCGGCTGGCAGAGATGGCCGTGATGCAGTTTGTAGCA
ATCAGACCACCGGTCTGGGTGCAAAAGGTGATCGTGGTCCGCCAGGTACA
CCGGGTTTTCCGGGTGAAGTTGGCCGTCCGGGTCCACCGGGTGCAGATGG
TATTCCGGGTCCTCAGGGTGAACGTGGTGCAGTTGGTCCTGGTGGTAAAC
CTGGTCCGCGTGGTGAAGTGGGCACCCCTGGTGCCGATGGCGCAGATGGT
GCAACCGGTGCGACCGGTGTTCAGGGTCCTGATGGTGCCAAAGGCGAAAA
AGGTGCAAGCGGCACCGCAGGTCTGAAAGGTGAGAAAGGCGATACCTGT
ATTCCGGATAGCAATAGCACCCTGGGTATGCCTGGTACACCAGGTGCCGG
TGGTAGCAAAGGCCAGAAAGGTGAAAGTGGTATTGTTGGTCCGAAAGGC
GAACGCGGTGAAATTGGCACACCGGGTCATCCTGGTTTTCGTGGTGCGGA
TGGTGAACCAGGTCATAAAGGTGTTCCGGGTCGTGCCGGTGCGCAGGGTG
ATCGCGGTGATCCGGGTGATGATGGTCTGACCGGTGACTACAAAGACGAC
GACGACAAAtaa

The polynucleotide of SEQ ID NO: 107 was subcloned into vector pET28a, expressed host E. coli cells and the truncated Chondrosia reniformis non-fibrillar collagen 1 was purified as described herein. The purified non-fibrillar collagen produced a clear band on SDS-PAGE and an anti-FLAG western was observed at around 30 kilodaltons. There were no existing bands that appear at that location on the gel in the absence of expression of this protein.

Truncated Chondrosia reniformis (Kidney Sponge) Non Fibrillar Collagen 2 with DsbA Secretion and FLAG Tag

The amino acid sequence of truncated Chondrosia reniformis non-fibrillar collagen 2 with DsbA secretion and FLAG tag is disclosed in SEQ ID NO: 108. The DsbA secretion tag is encoded by nucleotides 1-57 of SEQ ID NO: 109 and the amino acid sequences are amino acids 1-19 of SEQ ID NO: 108. The non-fibrillar collagen nucleotide sequences are nucleotides 58-1509 of SEQ ID NO: 109 and the amino acid sequences are amino acids 20-503 of SEQ ID NO: 108. The FLAG nucleotide sequences are nucleotides 1510-1536 of SEQ ID NO: 109 and the amino acid sequences are amino acids 504-512 of SEQ ID NO: 108.

(SEQ ID NO: 108)
MKKIWLALAGLVLAFSASAGFPGAPGADGAPGQKGELGAVGPQGTPGLSGP
SGPTGPPGPKGVRGAPGSSGAKGDAGNPGDDGPVGPQGVPGVDGSPGQKGE
TGRVGPRGHDGINGTPGEDGATGFPGPDGAKGEKGTSGTAGLKGEKGDTCI
PDSNSTLGMPGTPGAGWSKGQKGESGIVGPKGEKGEIGTPGPPGFRGADGE
PGQRGEPGRAGAQGERGAPGNNGRDGFPGDPGADGAPGQKGELGAIGHPGF
SGPSGPSGPTGPPGPKGVRGAQGRPGDRGSPGDVGPIGAPGPPGADGVPGL
TGVQGRDGPKGESASSGAVYVRWGRTTCPSGADVVYSGRAAGAKYDHSGGT
SDHHCLPNNPQYLSEDDTNALGAQLYGVEYEIRDRSSPYNSLDQSDMPCVV
CNANGRSQLLMVPARYTCPTGWSREYYGYMMSEGKAKNREGRKTTICMDFS
AEAVPGSGANTNPSPGIMMRANCNGLACPPYQSNTPLTCAVCTKGDYKDDD
DK

The nucleic acid sequence of truncated Chondrosia reniformis non-fibrillar collagen 2 with DsbA secretion and FLAG tag is disclosed in SEQ ID NO: 109.

(SEQ ID NO: 109)
ATGAAAAAGATTTGGCTGGCGCTGGCTGGTTTAGTTTTAGCGTTTAGCGCA
TCGGCGGGTTTTCCTGGCGCTCCGGGTGCCGACGGTGCTCCGGGTCAAAA
AGGTGAACTGGGTGCCGTGGGTCCGCAGGGCACTCCGGGTCTGAGTGGTC
CTAGTGGTCCGACCGGTCCACCAGGTCCAAAAGGCGTGCGTGGTGCACCG
GGTAGCAGCGGAGCCAAAGGTGATGCAGGTAACCCTGGTGATGACGGTCC
GGTTGGTCCACAGGGCGTTCCAGGTGTTGATGGTAGCCCTGGCCAAAAGG
GTGAAACCGGTCGTGTGGGTCCTCGTGGTCATGATGGTATTAATGGCACC
CCAGGTGAAGATGGTGCGACAGGCTTTCCAGGTCCGGATGGCGCAAAGGG
TGAGAAGGGCACCAGCGGTACAGCTGGCCTGAAGGGCGAAAAGGGCGAT
ACATGCATCCCGGATTCAAATTCAACACTGGGCATGCCAGGTACGCCTGG
CGCAGGTTGGAGTAAAGGACAAAAAGGCGAATCAGGCATTGTGGGACCT
AAAGGCGAGAAGGGTGAGATTGGTACTCCGGGACCGCCAGGCTTTCGCGG
TGCAGACGGCGAACCGGGTCAGCGTGGCGAACCTGGTCGTGCAGGCGCA
CAAGGTGAACGCGGAGCCCCTGGTAATAATGGACGTGATGGCTTTCCTGG
TGATCCAGGTGCAGATGGCGCACCTGGCCAGAAAGGCGAACTGGGAGCA
ATTGGTCATCCGGGATTTAGCGGTCCGTCAGGTCCGAGCGGACCGACAGG
TCCTCCTGGACCGAAAGGTGTACGTGGCGCACAGGGTCGTCCTGGCGATC
GTGGCAGTCCAGGTGATGTGGGTCCGATTGGTGCACCTGGTCCTCCAGGT
GCGGACGGCGTGCCTGGTTTAACAGGTGTGCAGGGTCGCGACGGTCCTAA
AGGTGAATCAGCAAGCAGCGGTGCAGTTTATGTTCGTTGGGGTCGTACCA
CCTGTCCTAGCGGAGCAGATGTTGTTTATAGCGGTCGCGCAGCCGGTGCA
AAATATGATCATTCAGGTGGCACCTCAGATCATCATTGTCTGCCGAATAAT
CCGCAGTATCTGAGCGAAGATGATACCAATGCACTGGGTGCACAGCTGTA
TGGTGTGGAATATGAAATTCGTGATCGTAGCAGCCCGTATAATAGCCTGG
ATCAGAGCGATATGCCGTGTGTTGTTTGTAATGCAAATGGTCGTAGCCAG
CTGCTGATGGTTCCGGCACGTTATACATGCCCGACCGGTTGGAGCCGTGA
ATATTATGGTTATATGATGAGCGAAGGCAAAGCCAAAAATCGCGAAGGTC
GTAAAACCACCATTTGTATGGATTTTAGCGCAGAAGCAGTTCCTGGTAGC
GGTGCAAATACCAATCCGAGTCCGGGTATTATGATGCGTGCAAATTGTAA
TGGTCTGGCATGTCCGCCTTATCAGAGCAATACACCGCTGACCTGTGCCGT
TTGTACCAAAGGTGACTACAAAGACGACGACGACAAAtaa

The polynucleotide of SEQ ID NO: 109 was subcloned into vector pET28a, expressed host E. coli cells and the truncated Chondrosia reniformis non-fibrillar collagen 2 was purified as described herein. The purified fibrillary collagen produced a clear band on SDS-PAGE and an anti-FLAG western was observed at around 60 kilodaltons. There were no existing bands that appear at that location on the gel in the absence of expression of this protein.

### Example 13: Truncated Rhincodon typus (Whale Shark) Collagen

Truncated Rhincodon typus (Whale Shark) Collagen Type 1 Alpha 1 Truncation 1 with DsbA Secretion and FLAG Tag

The amino acid sequence of truncated Rhincodon typus collagen type 1 truncation 1 with DsbA secretion and FLAG tag is disclosed in SEQ ID NO: 110. The DsbA secretion tag is encoded by nucleotides 1-57 of SEQ ID NO: 111 and the amino acid sequences are amino acids 1-19 of SEQ ID NO: 110. The collagen nucleotide sequences are nucleotides 58-630 of SEQ ID NO: 111 and the amino acid sequences are amino acids 20-210 of SEQ ID NO: 110. The FLAG nucleotide sequences are nucleotides 631-657 of SEQ ID NO: 111 and the amino acid sequences are amino acids 211-219 of SEQ ID NO: 110.

(SEQ ID NO: 110)
MKKIWLALAGLVLAFSASAGPAGAKGPSGDIGRPGESGSPGARGHSGQPGR
TGIAGNQGLPGTAGEEGRTGPPGPAGLRGQAGMMGFPGPKGAAGLPGKPGD
RGNVGLAGPRGAPGKDGEVGAQGPPGVAGPTGPRGETGLAGSVGFQGMPGP
SGAAGEPGKPGNQGLRGDAGSPGMIGPRGERGLPGERGASGAQGLLGPRGT
SGAPGLGDYKDDDDK

The nucleic acid sequence of truncated Rhincodon typus collagen type 1 truncation 1 with DsbA secretion and FLAG tag is disclosed in SEQ ID NO: 111.

(SEQ ID NO: 111)
ATGAAAAAGATTTGGCTGGCGCTGGCTGGTTTAGTTTTAGCGTTTAGCGCA
TCGGCGGGTCCGGCAGGCGCAAAAGGTCCGAGCGGTGATATTGGTCGTCC
GGGTGAAAGCGGTAGTCCGGGTGCACGTGGTCATAGCGGTCAGCCTGGTC
GTACCGGTATTGCAGGTAATCAGGGTCTGCCTGGTACAGCCGGTGAAGAA
GGTCGCACCGGTCCGCCAGGTCCTGCAGGTCTGCGTGGTCAGGCAGGTAT
GATGGGTTTTCCGGGTCCGAAAGGTGCAGCGGGTCTGCCAGGCAAACCGG
GTGATCGTGGTAATGTTGGTCTGGCTGGTCCGCGTGGTGCACCGGGTAAA
GATGGTGAAGTTGGTGCACAGGGTCCTCCGGGTGTTGCAGGTCCGACCGG
TCCTCGTGGTGAAACCGGTCTGGCAGGTAGCGTTGGTTTTCAGGGTATGCC
AGGTCCGTCAGGTGCAGCAGGCGAACCTGGTAAACCGGGTAACCAGGGC
CTGCGTGGTGATGCCGGTTCACCGGGTATGATTGGTCCACGCGGTGAACG
TGGCCTGCCTGGCGAACGTGGTGCAAGCGGTGCACAAGGTCTGCTGGGTC
CACGTGGCACCTCAGGCGCACCAGGTCTGGGTGACTACAAAGACGACGAC
GACAAAtaa

The polynucleotide of SEQ ID NO: 111 was subcloned into vector pET28a, expressed host E. coli cells and the truncated Rhincodon typus collagen type 1 truncation 1 was purified as described herein. The purified collagen produced a clear band on SDS-PAGE and an anti-FLAG western was observed at around 25 kilodaltons. There were no existing bands that appear at that location on the gel in the absence of expression of this protein.

Truncated Rhincodon typus (Whale Shark) Collagen Type 6 Alpha 1 Truncation 2 with DsbA Secretion and FLAG Tag

The amino acid sequence of truncated Rhincodon typus collagen type 6 truncation 2 with DsbA secretion and FLAG tag is disclosed in SEQ ID NO: 112. The DsbA secretion tag is encoded by nucleotides 1-57 of SEQ ID NO: 113 and the amino acid sequences are amino acids 1-19 of SEQ ID NO: 112. The collagen nucleotide sequences are nucleotides 58-684 of SEQ ID NO: 113 and the amino acid sequences are amino acids 20-228 of SEQ ID NO: 112. The FLAG nucleotide sequences are nucleotides 685-711 of SEQ ID NO: 113 and the amino acid sequences are amino acids 229-237 of SEQ ID NO: 112.

(SEQ ID NO: 112)
MKKIWLALAGLVLAFSASAQGIPGSAGKEGGKGDPGPLGSPGKPGPDGLRG
FAGARGLPGAAGPPGLKGAEGPMGAPGLTGSTGERGPNGPAGAIGLPGRPG
GPGPPGPVGEKGDPGDKGLPGPAGDDGVQGAMGLPGPIGSQGPPGDYGDKG
ELGKPGQKGSKGDKGESGPPGPIGIQGPIGHPGPIGSDGSPGLRGYLGMRG
QKGDDGIRGLPGSAGPVGLQGLPGGDYKDDDDK

The nucleic acid sequence of truncated Rhincodon typus collagen type 6 truncation 2 with DsbA secretion and FLAG tag is disclosed in SEQ ID NO: 113.

(SEQ ID NO: 113)
ATGAAAAAGATTTGGCTGGCGCTGGCTGGTTTAGTTTTAGCGTTTAGCGC
ATCGGCGCAGGGTATTCCGGGTAGCGCAGGTAAAGAAGGTGGTAAAGGCG
ATCCGGGTCCGCTGGGTTCACCGGGTAAACCGGGTCCTGATGGTCTGCGT
GGTTTTGCCGGTGCACGTGGTCTGCCTGGTGCAGCAGGTCCGCCTGGTCT
GAAAGGTGCCGAAGGTCCGATGGGTGCTCCGGGTCTGACCGGTAGCACCG
GTGAACGCGGTCCGAATGGTCCGGCAGGCGCAATTGGTCTGCCAGGTCGT
CCTGGTGGTCCGGGTCCTCCTGGTCCGGTTGGTGAAAAAGGTGATCCTGG
TGATAAAGGCCTGCCTGGTCCTGCCGGTGATGATGGTGTTCAGGGTGCCA
TGGGCTTACCGGGTCCGATTGGTAGCCAGGGTCCTCCGGGTGATTATGGC
GATAAAGGTGAACTGGGTAAACCTGGCCAGAAAGGTAGCAAAGGTGACAA
AGGCGAAAGCGGTCCGCCAGGTCCGATCGGCATTCAGGGTCCTATTGGTC
ATCCAGGTCCAATTGGTTCAGATGGCTCACCGGGACTGCGTGGCTATCTG
GGTATGCGTGGACAGAAAGGTGATGACGGTATTCGTGGCCTGCCAGGTAG
TGCAGGTCCGGTGGGTCTGCAGGGACTGCCTGGTGGTGACTACAAAGACG
ACGACGACAAAtaa

The polynucleotide of SEQ ID NO: 113 was subcloned into vector pET28a, expressed host E. coli cells and the truncated Rhincodon typus collagen type 6 truncation 2 was purified as described herein. The purified collagen produced a clear band on SDS-PAGE and an anti-FLAG western was observed at around 35 kilodaltons. There were no existing bands that appear at that location on the gel in the absence of expression of this protein.

Truncated Rhincodon typus (Whale Shark) Collagen Type 6 Alpha 1 Truncation 3 with DsbA Secretion and FLAG Tag

The amino acid sequence of truncated Rhincodon typus collagen type 6 alpha 1 truncation 3 with DsbA secretion and FLAG tag is disclosed in SEQ ID NO: 114. The DsbA secretion tag is encoded by nucleotides 1-57 of SEQ ID NO: 115 and the amino acid sequences are amino acids 1-19 of SEQ ID NO: 114. The collagen nucleotide sequences are nucleotides 58-735 of SEQ ID NO: 115 and the amino acid sequences are amino acids 20-245 of SEQ ID NO: 114. The FLAG nucleotide sequences are nucleotides 736-762 of SEQ ID NO: 115 and the amino acid sequences are amino acids 246-254 of SEQ ID NO: 114.

(SEQ ID NO: 114)
MKKIWLALAGLVLAFSASAKGETGEAGDPGTPGEPGIAGPKGDVGDKGDA
GPPGAAGPAGVKGPPGEDGAKGDVGPAGFPGDPGPTGEPGVPGMDGGVGE
KGSLGDPGLTGPRGASGEPGPPGSPGKRGPPGPAGPEGREGLKGSKGSPG
QEGPVGRTGPIGPQGSPGNVGPKGLRGIPGPTGEQGLLGPPGQAGPPGPM
GPPGMPGLRGAQGLKGDKGHVGLIGLIGPPGEMGEKGDQGLPGIQGDYKD
DDDK

The nucleic acid sequence of truncated Rhincodon typus collagen type 6 alpha 1 truncation 3 with DsbA secretion and FLAG tag is disclosed in SEQ ID NO: 115.

(SEQ ID NO: 115)
ATGAAAAAGATTTGGCTGGCGCTGGCTGGTTTAGTTTTAGCGTTTAGCGC
ATCGGCGAAAGGTGAAACCGGTGAAGCGGGTGATCCGGGTACACCGGGTG
AACCTGGTATTGCAGGTCCGAAAGGTGATGTTGGTGATAAAGGTGACGCA
GGTCCGCCTGGTGCAGCAGGTCCGGCAGGCGTTAAAGGTCCTCCGGGTGA
AGATGGTGCAAAAGGCGACGTTGGTCCTGCAGGTTTTCCTGGCGATCCGG
GTCCGACTGGTGAACCGGGTGTGCCAGGTATGGATGGTGGTGTGGGTGAA
AAAGGTAGCCTGGGTGATCCTGGTCTGACCGGTCCGCGTGGCGCAAGTGG
TGAACCAGGTCCACCGGGTAGTCCGGGTAAACGTGGTCCTCCTGGACCGG
CTGGTCCGGAAGGTCGTGAAGGTCTGAAAGGTAGCAAAGGTTCACCGGGT
CAAGAAGGTCCGGTTGGTCGTACCGGTCCGATTGGTCCGCAGGGCTCACC
GGGTAATGTTGGTCCTAAAGGTCTGCGTGGTATTCCGGGTCCTACAGGCG
AACAGGGTCTGCTGGGTCCGCCAGGCCAAGCAGGTCCTCCAGGTCCTATG
GGTCCACCTGGTATGCCTGGCCTGCGTGGTGCCCAGGGCCTGAAAGGCGA
TAAAGGCCATGTTGGTCTGATTGGCCTGATTGGTCCACCAGGTGAAATGG
GAGAAAAAGGCGATCAGGGCCTGCCTGGTATTCAGGGTGACTACAAAGA
CGACGACGACAAAtaa

The polynucleotide of SEQ ID NO: 115 was subcloned into vector pET28a, expressed host E. coli cells and the truncated Rhincodon typus collagen type 1 truncation 1 was purified as described herein. The purified collagen produced a clear band on SDS-PAGE and an anti-FLAG western was observed at around 25 kilodaltons. There were no existing bands that appear at that location on the gel in the absence of expression of this protein.

1-25. (canceled)

26. A recombinant human type 21 alpha 1 collagen polypeptide comprising:
an N-terminal and/or a C-terminal truncation; and
no more than one glycine-proline-proline triplet sequence.

27. The recombinant polypeptide of claim 26, wherein the recombinant polypeptide comprises an N-terminal truncation that is between 1 and 85 amino acids relative to full-length human type 21 alpha 1 collagen.

28. The recombinant polypeptide of claim 26, wherein the recombinant polypeptide comprises an N-terminal truncation that is between 1 and 212 amino acids relative to full-length human type 21 alpha 1 collagen.

29. The recombinant polypeptide of claim 26, wherein the recombinant polypeptide is produced in a microbial host.

30. The recombinant polypeptide of claim 29, wherein the microbial host cell is a bacterial cell, a yeast cell, or a fungal cell.

31. The recombinant polypeptide of claim 30, wherein the microbial host cell is Escherichia coli.

32. The recombinant polypeptide of claim 26, wherein the recombinant polypeptide lacks proline hydroxylation.

33. The recombinant polypeptide of claim 26, wherein the recombinant polypeptide is monomeric and does not form a stable triple helix structure formed by natural collagen at 37° C.

34. The recombinant polypeptide of claim 26, wherein the recombinant polypeptide comprises at least 95% amino acid sequence identity to full length SEQ ID NO: 76.

35. The recombinant polypeptide of claim 26, wherein the recombinant polypeptide comprises at least 98% amino acid sequence identity to full length SEQ ID NO: 76.

36. A fusion protein comprising the recombinant polypeptide of claim 26 and a secretion tag.

37. The fusion protein of claim 36, wherein the secretion tag comprises at least one of DsbA, PelB, OmpA, TolB, MalE, lpp, TorA, HylA, DegP, or Skp, or a hybrid secretion tag comprising a portion of a first secretion tag fused to a portion of a second secretion tag.

38. The fusion protein of claim 36, wherein the secretion tag comprises DsbA.

39. The fusion protein of claim 36, further comprising a purification tag and/or a fluorescent protein tag.

40. A composition comprising from about 0.005% to about 30% w/w of the recombinant polypeptide of claim 26.

41. The composition of claim 40, wherein the composition is formulated for topical application.

42. The composition of claim 40, wherein the composition comprises a topical carrier and/or a preservative.

43. A method of treating the skin of a subject comprising topically administering the composition of claim 40 to the skin of the subject.

44. The method of claim 43, wherein treating the skin of the subject comprises increasing viability of keratinocytes and/or fibroblasts in the skin of the subject after exposure to ultraviolet (UV) radiation and/or urban dust.

45. The method of claim 43, wherein treating the skin of the subject comprises decreasing production of inflammatory cytokines.
